(19) United States
(12) Patent Application Publication
(10) Pub. No.: US 2024/0270697 A1
(43) Pub. Date: Aug. 15, 2024

(54) HETEROCYCLIC COMPOUND AND ORGANIC LIGHT-EMITTING DEVICE COMPRISING SAME

(71) Applicant: LT MATERIALS CO., LTD., Yongin-si, Gyeonggi-do (KR)

(72) Inventors: Jun-Tae MO, Yongin-si, Gyeonggi-do (KR); Yong-Hui LEE, Yongin-si, Gyeonggi-do (KR); Dong-Jun KIM, Yongin-si, Gyeonggi-do (KR)

(73) Assignee: LT MATERIALS CO., LTD., Yongin-si, Gyeonggi-do (KR)

(21) Appl. No.: 18/563,596

(22) PCT Filed: Jan. 25, 2022
(86) PCT No.: PCT/KR2022/001296

§ 371 Date: Nov. 22, 2023

(30) Foreign Application Priority Data
Jun. 28, 2021 (KR) 10-2021-0083664

(51) Int. Cl. C07D 239/26; C07D 239/74; C07D 251/24; C07D 403/06; C07D 403/14; C07D 405/04; C07D 405/12; C07D 405/14; C07D 409/04; C07D 409/12; C07D 487/04; C07D 491/048; C07D 495/04; H10K 50/11; H10K 85/60

(52) U.S. Cl. CPC C07D 239/26; C07D 239/74; C07D 251/24; C07D 403/06; C07D 403/14; C07D 405/04; C07D 405/12; C07D 405/14; C07D 409/04; C07D 409/12; C07D 487/04; C07D 491/048; C07D 495/04; H10K 50/11; H10K 85/636; H10K 85/654; H10K 85/6572; H10K 85/6574

(57) ABSTRACT

The present application provides a heterocyclic compound and an organic light emitting device in which the heterocyclic compound is contained in an organic material layer.

### TECHNICAL FIELD

This application claims priority to and the benefit of Korean Patent Application No. 10-2021-0083664 filed in the Korean Intellectual Property Office on Jun. 28, 2021, the entire contents of which are incorporated herein by reference.

The present specification relates to a heterocyclic compound and an organic light emitting device including the same.

### BACKGROUND ART

An electroluminescence device is a kind of self-emitting type display device, and has an advantage in that the viewing angle is wide, the contrast is excellent, and the response speed is fast.

An organic light emitting device has a structure in which an organic thin film is disposed between two electrodes. When a voltage is applied to an organic light emitting device having the structure, electrons and holes injected from the two electrodes combine with each other in an organic thin film to make a pair, and then, emit light while being extinguished. The organic thin film may be composed of a single layer or multi layers, if necessary.

A material for the organic thin film may have a light emitting function, if necessary. For example, as the material for the organic thin film, it is also possible to use a compound, which may itself constitute a light emitting layer alone, or it is also possible to use a compound, which may serve as a host or a dopant of a host-dopant-based light emitting layer. In addition, as a material for the organic thin film, it is also possible to use a compound, which may perform a function such as hole injection, hole transport, electron blocking, hole blocking, electron transport or electron injection.

In order to improve the performance, service life, or efficiency of the organic light emitting device, there is a continuous need for developing a material for an organic thin film.

### RELATED ART DOCUMENT

### Patent Document

U.S. Pat. No. 4,356,429

### DISCLOSURE

### Technical Problem

The present invention has been made in an effort to provide a heterocyclic compound and an organic light emitting device including the same.

### Technical Solution

An exemplary embodiment of the present application provides a heterocyclic compound represented by the following Chemical Formula 1.

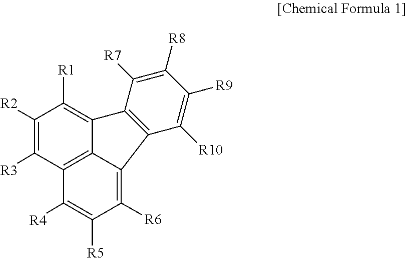

In Chemical Formula 1,
R1 to R10 are the same as or different from each other, and are each independently hydrogen; deuterium; -(L1)a-Ar1; or -(L2)b-Ar2, at least one of R1 to R10 is -(L1)a-Ar1, and at least one of the others is -(L2)b-Ar2,
L1 and L2 are the same as or different from each other, and are each independently a direct bond; a substituted or unsubstituted arylene group having 6 to 60 carbon atoms; or a substituted or unsubstituted heteroarylene group having 2 to 60 carbon atoms, a and b are an integer from 0 to 3, and when a and b are each 2 or higher, substituents in the parenthesis are each independent,
Ar1 is a monocyclic or polycyclic heterocyclic group which is substituted or unsubstituted, and includes one or more N's, and
Ar2 is —NAr3Ar4; a substituted or unsubstituted aryl group having 6 to 60 carbon atoms; or a substituted or unsubstituted heteroaryl group having 2 to 60 carbon atoms, and Ar3 and Ar4 are the same as or different from each other, and are each independently a substituted or unsubstituted aryl group having 6 to 60 carbon atoms; or a substituted or unsubstituted heteroaryl group having 2 to 60 carbon atoms.

Further, another exemplary embodiment of the present application provides an organic light emitting device including a first electrode, a second electrode and an organic material layer having one or more layers provided between the first electrode and the second electrode, in which one or more layers of the organic material layer include the heterocyclic compound represented by Chemical Formula 1.

### Advantageous Effects

A heterocyclic compound according to an exemplary embodiment of the present application can be used as a material for an organic material layer of an organic light emitting device. The heterocyclic compound can be used as a material for a hole injection layer, a hole transport layer, a light emitting layer, an electron transport layer, an electron injection layer, a charge generation layer, and the like in an organic light emitting device. In particular, the heterocyclic compound represented by Chemical Formula 1 can be used as a material for the light emitting layer of the organic light emitting device. In addition, when the heterocyclic compound represented by Chemical Formula 1 is used for an organic light emitting device, the driving voltage of the device can be lowered, the light efficiency of the device can be improved, and the service life characteristics of the device can be improved due to the thermal stability of the compound.

### DESCRIPTION OF DRAWINGS

FIGS. 1 to 3 each are views schematically illustrating a stacking structure of an organic light emitting device according to an exemplary embodiment of the present application.

### MODE FOR INVENTION

Hereinafter, the present application will be described in detail.

An exemplary embodiment of the present application provides a heterocyclic compound represented by the following Chemical Formula 1.

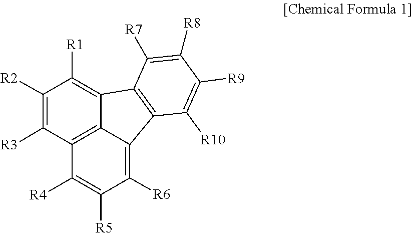

In Chemical Formula 1,
R1 to R10 are the same as or different from each other, and are each independently hydrogen; deuterium; -(L1)a-Ar1; or -(L2)b-Ar2, at least one of R1 to R10 is -(L1)a-Ar1, and at least one of the others is -(L2)b-Ar2,
L1 and L2 are the same as or different from each other, and are each independently a direct bond; a substituted or unsubstituted arylene group having 6 to 60 carbon atoms; or a substituted or unsubstituted heteroarylene group having 2 to 60 carbon atoms, a and b are an integer from 0 to 3, and when a and b are each 2 or higher, substituents in the parenthesis are each independent,
Ar1 is a monocyclic or polycyclic heterocyclic group which is substituted or unsubstituted, and includes one or more N's, and
Ar2 is —NAr3Ar4; a substituted or unsubstituted aryl group having 6 to 60 carbon atoms; or a substituted or unsubstituted heteroaryl group having 2 to 60 carbon atoms, and Ar3 and Ar4 are the same as or different from each other, and are each independently a substituted or unsubstituted aryl group having 6 to 60 carbon atoms; or a substituted or unsubstituted heteroaryl group having 2 to 60 carbon atoms.

In an exemplary embodiment of the present invention, when a and b are each 2 or higher, substituents in the parenthesis are the same as or different from each other.

Since the molecular structure of a host used for the light emitting layer (EML) in the organic light emitting device needs to simultaneously have the injection/transport properties of electrons and the injection/transport properties of holes, it is an essential factor to have bipolarity. Since it is quite difficult to make a balance between electrons and holes of these bipolar molecules, recently, the balance of electrons and holes in the light emitting layer has been adjusted by adjusting the ratio using p-type molecules having hole characteristics and n-type molecules having electronic characteristics. Although such a method can easily adjust the balance between holes and electrons, there is difficulty in that an organic material needs to be uniformly deposited on the device.

The compound represented by Chemical Formula 1 has fluoranthene, which is a structure in which a pentagonal ring is formed between naphthalene and a benzene ring structure, as a basic skeleton, and when used as a linker in a bulky form, the compound may serve as a node which prevents the conjugation of an acceptor and a donor. Furthermore, the band-gap of the compound can be reduced due to a basic skeletal structure of fluoranthene of the compound represented by Chemical Formula 1 and a structure having a specific substituent.

Due to such structural features, when the compound represented by Chemical Formula 1 is used as a material for the organic material layer of the organic light emitting device, it is possible to facilitate intramolecular charge separation when the device is driven.

Further, the compound represented by Chemical Formula 1 has an advantage in that it is easy to make a bipolar host such as n-Host or p-Host, and it is easy to adjust the mobility of charge by adjusting the planarity of the stereostructure of a molecular structure according to the substituted position.

Due to such features, when the compound represented by Chemical Formula 1 is used as a material for an organic material layer of an organic light emitting device, the efficiency and service life of the device can be improved.

In addition, the compound represented by Chemical Formula 1, which simultaneously has hole/electron characteristics in one molecule to adjust the balance between holes and electrons, has an advantage in that the organic material is also easily uniformly deposited because the service life and efficiency of the device are excellent even though the compound is used as a single host.

Furthermore, since the compound represented by Chemical Formula 1 simultaneously has hole/electronic characteristics, and there is an advantage in that the service life of the device can be further improved when a p-type organic material is commonly used (co-dep).

In the present specification, the term “substitution” means that a hydrogen atom bonded to a carbon atom of a compound is changed into another substituent, and a position to be substituted is not limited as long as the position is a position at which the hydrogen atom is substituted, that is, a position at which the substituent may be substituted, and when two or more substituents are substituted, the two or more substituents may be the same as or different from each other.

In the present specification, “substituted or unsubstituted” means being unsubstituted or substituted with one or more substituents selected from the group consisting of a straight-chained or branched alkyl having 1 to 60 carbon atoms; a straight-chained or branched alkenyl having 2 to 60 carbon atoms; a straight-chained or branched alkynyl having 2 to 60 carbon atoms; a monocyclic or polycyclic cycloalkyl having 3 to 60 carbon atoms; a monocyclic or polycyclic heterocycloalkyl having 2 to 60 carbon atoms; a monocyclic or polycyclic aryl having 6 to 60 carbon atoms; a monocyclic or polycyclic heteroaryl having 2 to 60 carbon atoms; —SiRR′R″; —P(═O)RR′; an alkylamine having 1 to 20 carbon atoms; a monocyclic or polycyclic arylamine having 6 to 60 carbon atoms; and a monocyclic or polycyclic heteroarylamine having 2 to 60 carbon atoms, or being unsubstituted or substituted with a substituent to which two or more substituents selected from the above exemplified substituents are linked, and means that R, R′ and R″ are the same as or different from each other, and are each independently hydrogen; deuterium; a halogen; a substituted or unsubstituted alkyl having 1 to 60 carbon atoms; a substituted or unsubstituted aryl having 6 to 60 carbon atoms; or a substituted or unsubstituted heteroaryl having 2 to 60 carbon atoms.

In the present specification, “when a substituent is not indicated in the structure of a chemical formula or compound” means that a hydrogen atom is bonded to a carbon atom. However, since deuterium (²H) is an isotope of hydrogen, some hydrogen atoms may be deuterium.

In an exemplary embodiment of the present application, “when a substituent is not indicated in the structure of a chemical formula or compound” may mean that all the positions that may be reached by the substituent are hydrogen; or deuterium. That is, deuterium is an isotope of hydrogen, and some hydrogen atoms may be deuterium which is an isotope, and in this case, the content of deuterium may be 0% to 100%.

In an exemplary embodiment of the present application, in “the case where a substituent is not indicated in the structure of a chemical formula or compound”, when the content of deuterium is 0%, the content of hydrogen is 100%, and all the substituents do not explicitly exclude deuterium such as hydrogen, hydrogen and deuterium may be mixed and used in the compound.

In an exemplary embodiment of the present application, deuterium is one of the isotopes of hydrogen, is an element that has a deuteron composed of one proton and one neutron as a nucleus, and may be represented by hydrogen-2, and the element symbol may also be expressed as D or 2H.

In an exemplary embodiment of the present application, the isotope means an atom with the same atomic number (Z), but different mass numbers (A), and the isotope may be interpreted as an element which has the same number of protons, but different number of neutrons.

In an exemplary embodiment of the present application, when the total number of substituents of a basic compound is defined as T1 and the number of specific substituents among the substituents is defined as T2, the content T % of the specific substituent may be defined as T2/T1×100=T %.

That is, in an example, the deuterium content of 20% in a phenyl group represented by

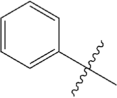 [SMILES: *c1ccccc1]

may be represented by 20% when the total number of substituents that the phenyl group can have is 5 (T1 in the formula) and the number of deuteriums among the substituents is 1 (T2 in the formula). That is, a deuterium content of 20% in the phenyl group may be represented by the following structural formula.

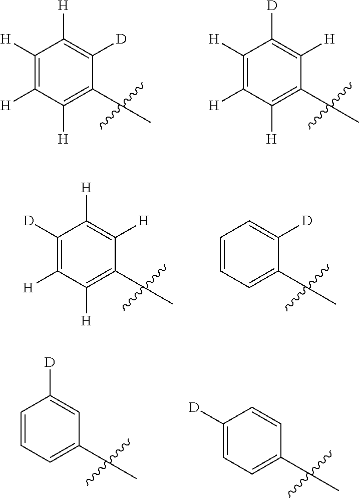 [SMILES: *c1ccccc1[2H] ; *c1cccc([2H])c1 ; *c1ccc([2H])cc1 ; *c1ccccc1[2H] ; *c1cccc([2H])c1 ; *c1ccc([2H])cc1]

Further, in an exemplary embodiment of the present application, “a phenyl group having a deuterium content of 0%” may mean a phenyl group that does not include a deuterium atom, that is, has five hydrogen atoms.

In the present specification, the halogen may be fluorine, chlorine, bromine or iodine.

In the present specification, the alkyl group includes a straight-chain or branched-chain having 1 to 60 carbon atoms, and may be additionally substituted with another substituent. The number of carbon atoms of the alkyl group may be 1 to 60, specifically 1 to 40, and more specifically 1 to 20. Specific examples thereof include a methyl group, an ethyl group, a propyl group, an n-propyl group, an isopropyl group, a butyl group, an n-butyl group, an isobutyl group, a tert-butyl group, a sec-butyl group, a 1-methyl-butyl group, a 1-ethyl-butyl group, a pentyl group, an n-pentyl group, an isopentyl group, a neopentyl group, a tert-pentyl group, a hexyl group, an n-hexyl group, a 1-methylpentyl group, a 2-methylpentyl group, a 4-methyl-2-pentyl group, a 3,3-dimethylbutyl group, a 2-ethylbutyl group, a heptyl group, an n-heptyl group, a 1-methylhexyl group, a cyclopentylmethyl group, a cyclohexylmethyl group, an octyl group, an n-octyl group, a tert-octyl group, a 1-methylheptyl group, a 2-ethylhexyl group, a 2-propylpentyl group, an n-nonyl group, a 2,2-dimethylheptyl group, a 1-ethyl-propyl group, a 1,1-dimethyl-propyl group, an isohexyl group, a 2-methylpentyl group, a 4-methylhexyl group, a 5-methylhexyl group, and the like, but are not limited thereto.

In the present specification, the alkenyl group includes a straight-chain or branched-chain having 2 to 60 carbon atoms, and may be additionally substituted with another substituent. The number of carbon atoms of the alkenyl group may be 2 to 60, specifically 2 to 40, and more specifically 2 to 20. Specific examples thereof include a vinyl group, a 1-propenyl group, an isopropenyl group, a 1-butenyl group, a 2-butenyl group, a 3-butenyl group, a 1-pentenyl group, a 2-pentenyl group, a 3-pentenyl group, a 3-methyl-1-butenyl group, a 1,3-butadienyl group, an allyl group, a 1-phenylvinyl-1-yl group, a 2-phenylvinyl-1-yl group, a 2,2-diphenylvinyl-1-yl group, a 2-phenyl-2-(naphthyl-1-yl)vinyl-1-yl group, a 2,2-bis(diphenyl-1-yl)vinyl-1-yl group, a stilbenyl group, a styrenyl group, and the like, but are not limited thereto. In the present specification, the alkynyl group includes a straight-chain or branched-chain having 2 to 60 carbon atoms, and may be additionally substituted with another substituent. The number of carbon atoms of the alkynyl group may be 2 to 60, specifically 2 to 40, and more specifically 2 to 20.

In the present specification, an alkoxy group may be straight-chained, branched, or cyclic. The number of carbon atoms of the alkoxy group is not particularly limited, but is preferably 1 to 20. Specific examples thereof include methoxy, ethoxy, n-propoxy, isopropoxy, n-butoxy, isobutoxy, tert-butoxy, sec-butoxy, n-pentyloxy, neopentyloxy, isopentyloxy, n-hexyloxy, 3,3-dimethylbutyloxy, 2-ethylbutyloxy, n-octyloxy, n-nonyloxy, n-decyloxy, benzyloxy, p-methylbenzyloxy, and the like, but are not limited thereto.

In the present specification, the cycloalkyl group includes a monocycle or polycycle having 3 to 60 carbon atoms, and may be additionally substituted with another substituent. Here, the polycycle means a group in which a cycloalkyl group is directly linked to or fused with another cyclic group. Here, another cyclic group may also be a cycloalkyl group, but may also be another kind of cyclic group, for example, a heterocycloalkyl group, an aryl group, a heteroaryl group, and the like. The number of carbon atoms of the cycloalkyl group may be 3 to 60, specifically 3 to 40, and more specifically 5 to 20. Specific examples thereof include a cyclopropyl group, a cyclobutyl group, a cyclopentyl group, a 3-methylcyclopentyl group, a 2,3-dimethylcyclopentyl group, a cyclohexyl group, a 3-methylcyclohexyl group, a 4-methylcyclohexyl group, a 2,3-dimethylcyclohexyl group, a 3,4,5-trimethylcyclohexyl group, a 4-tert-butylcyclohexyl group, a cycloheptyl group, a cyclooctyl group, and the like, but are not limited thereto.

In the present specification, the heterocycloalkyl group includes O, S, Se, N, or Si as a heteroatom, includes a monocycle or polycycle having 2 to 60 carbon atoms, and may be additionally substituted with another substituent. Here, the polycycle means a group in which a heterocycloalkyl group is directly linked to or fused with another cyclic group. Here, another cyclic group may also be a heterocycloalkyl group, but may also be another kind of cyclic group, for example, a cycloalkyl group, an aryl group, a heteroaryl group, and the like. The number of carbon atoms of the heterocycloalkyl group may be 2 to 60, specifically 2 to 40, and more specifically 3 to 20.

In the present specification, the aryl group includes a monocycle or polycycle having 6 to 60 carbon atoms, and may be additionally substituted with another substituent. Here, the polycycle means a group in which an aryl group is directly linked to or fused with another cyclic group. Here, another cyclic group may also be an aryl group, but may also be another kind of cyclic group, for example, a cycloalkyl group, a heterocycloalkyl group, a heteroaryl group, and the like. The aryl group includes a spiro group. The number of carbon atoms of the aryl group may be 6 to 60, specifically 6 to 40, and more specifically 6 to 25. Specific examples of the aryl group include a phenyl group, a biphenyl group, a terphenyl group, a naphthyl group, an anthryl group, a chrysenyl group, a phenanthrenyl group, a perylenyl group, a fluoranthenyl group, a triphenylenyl group, a phenalenyl group, a pyrenyl group, a tetracenyl group, a pentacenyl group, a fluorenyl group, an indenyl group, an acenaphthylenyl group, a benzofluorenyl group, a spirobifluorenyl group, a 2,3-dihydro-1H-indenyl group, a fused cyclic group thereof, and the like, but are not limited thereto.

In the present specification, a silyl group includes Si and is a substituent to which the Si atom is directly linked as a radical, and is represented by —SiR101R102R103, and R101 to R103 are the same as or different from each other, and may be each independently a substituent composed of at least one of hydrogen; deuterium; a halogen group; an alkyl group; an alkenyl group; an alkoxy group; a cycloalkyl group; an aryl group; and a heterocyclic group. Specific examples of the silyl group include a trimethylsilyl group, a triethylsilyl group, a t-butyldimethylsilyl group, a vinyldimethylsilyl group, a propyldimethylsilyl group, a triphenylsilyl group, a diphenylsilyl group, a phenylsilyl group, and the like, but are not limited thereto.

In the present specification, a phosphine oxide group is represented by —P(═O)R104R105, and R104 and R105 are the same as or different from each other, and may be each independently a substituent composed of at least one of hydrogen; deuterium; a halogen group; an alkyl group; an alkenyl group; an alkoxy group; a cycloalkyl group; an aryl group; and a heterocyclic group. Specific examples of the phosphine oxide group include a diphenylphosphine oxide group, dinaphthylphosphine oxide, and the like, but are not limited thereto.

In the present specification, the fluorenyl group may be substituted, and adjacent substituents may be bonded to each other to form a ring.

In the present specification, the spiro group is a group including a spiro structure, and may have 15 to 60 carbon atoms. For example, the spiro group may include a structure in which a 2,3-dihydro-1H-indene group or a cyclohexane group is spiro-bonded to a fluorenyl group. Specifically, the spiro group may include any one of the groups of the following structural formulae.

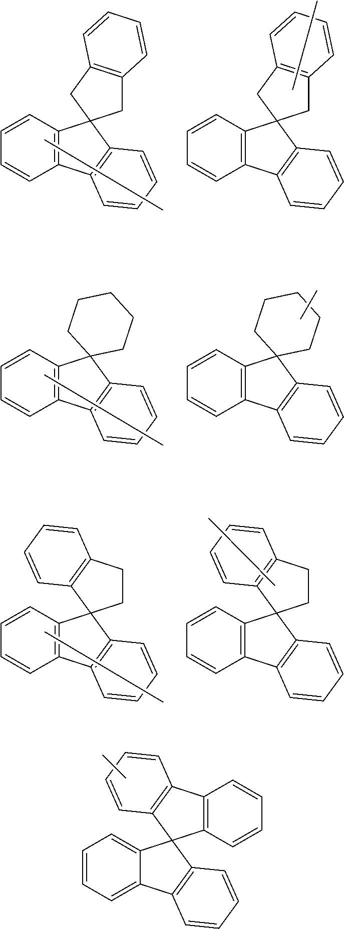

In the present specification, the heteroaryl group includes S, O, Se, N, or Si as a heteroatom, includes a monocycle or polycycle having 2 to 60 carbon atoms, and may be additionally substituted with another substituent. Here, the polycycle means a group in which a heteroaryl group is directly linked to or fused with another cyclic group. Here, another cyclic group may also be a heteroaryl group, but may also be another kind of cyclic group, for example, a cycloalkyl group, a heterocycloalkyl group, an aryl group, and the like. The number of carbon atoms of the heteroaryl group may be 2 to 60, specifically 2 to 40, and more specifically 3 to 25. Specific examples of the heteroaryl group include a pyridyl group, a pyrrolyl group, a pyrimidyl group, a pyridazinyl group, a furanyl group, a thiophene group, an imidazolyl group, a pyrazolyl group, an oxazolyl group, an isoxazolyl group, a thiazolyl group, an isothiazolyl group, a triazolyl group, a furazanyl group, an oxadiazolyl group, a thiadiazolyl group, a dithiazolyl group, a tetrazolyl group, a pyranyl group, a thiopyranyl group, a diazinyl group, an oxazinyl group, a thiazinyl group, a dioxynyl group, a triazinyl group, a tetrazinyl group, a quinolyl group, an isoquinolyl group, a quinazolinyl group, an isoquinazolinyl group, a quinozolilyl group, a naphthyridyl group, an acridinyl group, a phenanthridinyl group, an imidazopyridinyl group, a diaza naphthalenyl group, a triazaindene group, an indolyl group, an indolizinyl group, a benzothiazolyl group, a benzoxazolyl group, a benzimidazolyl group, a benzothiophene group, a benzofuran group, a dibenzothiophene group, a dibenzofuran group, a carbazolyl group, a benzocarbazolyl group, a dibenzocarbazolyl group, a phenazinyl group, a dibenzosilole group, spirobi (dibenzosilole), a dihydrophenazinyl group, a phenoxazinyl group, a phenanthridyl group, an imidazopyridinyl group, a thienyl group, an indolo[2,3-a]carbazolyl group, an indolo[2,3-b]carbazolyl group, an indolinyl group, a 10,11-dihydro-dibenzo[b,f]azepin group, a 9,10-dihydroacridinyl group, a phenanthrazinyl group, a phenothiazinyl group, a phthalazinyl group, a naphthylidinyl group, a phenanthrolinyl group, a benzo[c][1,2,5]thiadiazolyl group, a 5,10-dihydrodibenzo[b,e][1,4]azasilinyl, a pyrazolo[1,5-c]quinazolinyl group, a pyrido[1,2-b]indazolyl group, a pyrido[1,2-a]imidazo[1,2-e]indolinyl group, a 5,11-dihydroindeno[1,2-b]carbazolyl group, and the like, but are not limited thereto.

In the present specification, the amine group may be selected from the group consisting of a monoalkylamine group; a monoarylamine group; a monoheteroarylamine group; —NH₂; a dialkylamine group; a diarylamine group; a diheteroarylamine group; an alkylarylamine group; an alkylheteroarylamine group; and an arylheteroarylamine group, and the number of carbon atoms thereof is not particularly limited, but is preferably 1 to 30. Specific examples of the amine group include a methylamine group, a dimethylamine group, an ethylamine group, a diethylamine group, a phenylamine group, a naphthylamine group, a biphenylamine group, a dibiphenylamine group, an anthracenylamine group, a 9-methyl-anthracenylamine group, a diphenylamine group, a phenylnaphthylamine group, a ditolylamine group, a phenyltolylamine group, a triphenylamine group, a biphenylnaphthylamine group, a phenylbiphenylamine group, a biphenylfluorenylamine group, a phenyltriphenylenylamine group, a biphenyltriphenylenylamine group, and the like, but are not limited thereto.

In the present specification, an arylene group means that there are two bonding positions in an aryl group, that is, a divalent group. The above-described description on the aryl group may be applied to the arylene group, except that the arylene groups are each a divalent group. Further, a heteroarylene group means that there are two bonding positions in a heteroaryl group, that is, a divalent group. The above-described description on the heteroaryl group may be applied to the heteroarylene group, except for a divalent heteroarylene group.

In the present specification, the “adjacent” group may mean a substituent substituted with an atom directly linked to an atom in which the corresponding substituent is substituted, a substituent disposed to be sterically closest to the corresponding substituent, or another substituent substituted with an atom in which the corresponding substituent is substituted. For example, two substituents substituted at the ortho position in a benzene ring and two substituents substituted with the same carbon in an aliphatic ring may be interpreted as groups which are “adjacent” to each other.

A heterocyclic compound according to an exemplary embodiment of the present application is represented by Chemical Formula 1. More specifically, the heterocyclic compound represented by Chemical Formula 1 may be used as a material for an organic material layer of an organic light emitting device by the structural characteristics of the core structure and the substituent as described above.

More specifically, the heterocyclic compound represented by Chemical Formula 1 may be used as a material for an organic material layer of an organic light emitting device by the structural characteristics of the core structure and the substituent as described above.

In an exemplary embodiment of the present application, the deuterium content of the heterocyclic compound represented by Chemical Formula 1 may be 0% to 100%.

In an exemplary embodiment of the present application, the deuterium content of the heterocyclic compound represented by Chemical Formula 1 may be more than 10% and 100% or less.

In an exemplary embodiment of the present application, R1 to R10 of Chemical Formula 1 are the same as or different from each other, and are each independently hydrogen; deuterium; -(L1)a-Ar1; or -(L2)b-Ar2, at least one of R1 to R10 is -(L1)a-Ar1, and at least one of the others may be -(L2)b-Ar2.

In an exemplary embodiment of the present application, R1 to R10 are the same as or different from each other, and are each independently hydrogen; deuterium; -(L1)a-Ar1; or -(L2)b-Ar2, at least one of R7 to R10 is -(L2)b-Ar, and at least one of the others may be -(L1)a-Ar1.

In an exemplary embodiment of the present application, R1 to R10 are the same as or different from each other, and are each independently hydrogen; deuterium; -(L1)a-Ar1; or -(L2)b-Ar2, at least one of R1 to R10 is -(L2)b-Ar, and at least one of the others may be -(L1)a-Ar1.

In an exemplary embodiment of the present application, one or more of R1 to R10 are -(L1)a-Ar1, one or more of the others are -(L2)b-Ar2, and when there are two or more -(L1)a-Ar1's, -(L1)a-Ar1's may be the same as or different from each other, and there are two or more -(L2)b-Ar2's, -(L2)b-Ar2's may be the same as or different from each other.

In an exemplary embodiment of the present application, there may be one or more and nine or less -(L1)a-Ar1's in R1 to R10.

In an exemplary embodiment of the present application, there may be one or more and nine or less -(L2)b-Ar2's in R1 to R10.

In an exemplary embodiment of the present application, R1 to R10 are the same as or different from each other, and are each independently hydrogen; deuterium; -(L1)a-Ar1; or -(L2)b-Ar2, one of R1 to R10 is -(L2)b-Ar2, one of the others is (L1)a-Ar1, and all of the others may be hydrogen or deuterium.

In an exemplary embodiment of the present application, R1 to R10 are the same as or different from each other, and are each independently hydrogen; deuterium; -(L1)a-Ar1; or -(L2)b-Ar2, one of R1 to R10 is -(L2)b-Ar2, two of the others are (L1)a-Ar1, and all of the others may be hydrogen or deuterium.

In an exemplary embodiment of the present application, L1 and L2 are the same as or different from each other, and may be each independently a direct bond; a substituted or unsubstituted arylene group having 6 to 60 carbon atoms or a substituted or unsubstituted heteroarylene group having 2 to 60 carbon atoms.

In an exemplary embodiment of the present application, L1 and L2 are the same as or different from each other, and may be each independently a direct bond; a substituted or unsubstituted arylene group having 6 to 40 carbon atoms or a substituted or unsubstituted heteroarylene group having 2 to 40 carbon atoms.

In an exemplary embodiment of the present application, L1 and L2 are the same as or different from each other, and may be each independently a direct bond; a substituted or unsubstituted arylene group having 6 to 20 carbon atoms or a substituted or unsubstituted heteroarylene group having 2 to 20 carbon atoms.

In another exemplary embodiment, L1 is a direct bond; a phenylene group; a biphenylene group; a naphthylene group; a dibenzofuranrene group.

In still another exemplary embodiment, L1 is a direct bond.

In yet another exemplary embodiment, L1 is a phenylene group.

In yet another exemplary embodiment, L1 is a biphenylene group.

In yet another exemplary embodiment, L1 is a naphthylene group.

In yet another exemplary embodiment, L1 is a dibenzofuranrene group.

In an exemplary embodiment of the present application, a of Chemical Formula 1 may be an integer from 0 to 3.

In another exemplary embodiment, a is 0.

In still another exemplary embodiment, a is 1.

In yet another exemplary embodiment, a is 2.

In yet another exemplary embodiment, a is 3.

In an exemplary embodiment of the present application, when a is 2 or higher, L1 in the parenthesis is each independent.

In an exemplary embodiment of the present application, when a is 2 or higher, L1's in the parenthesis may be the same as or different from each other.

In an exemplary embodiment of the present application, L2 may be a direct bond; or a substituted or unsubstituted arylene group having 6 to 60 carbon atoms.

In an exemplary embodiment of the present application, L2 may be a direct bond; or a substituted or unsubstituted arylene group having 6 to 40 carbon atoms.

In an exemplary embodiment of the present application, L2 may be a direct bond; or a substituted or unsubstituted arylene group having 6 to 20 carbon atoms.

In another exemplary embodiment, L2 is a direct bond; a phenylene group; or a naphthylene group.

In still another exemplary embodiment, L2 is a direct bond.

In yet another exemplary embodiment, L2 is a phenylene group.

In an exemplary embodiment of the present application, b of Chemical Formula 1 may be an integer from 0 to 3.

In another exemplary embodiment, b is 0.

In still another exemplary embodiment, b is 1.

In yet another exemplary embodiment, b is 2.

In yet another exemplary embodiment, b is 3.

In an exemplary embodiment of the present application, when b is 2 or higher, L2 in the parenthesis is each independent.

In an exemplary embodiment of the present application, when b is 2 or higher, L2's in the parenthesis may be the same as or different from each other.

In an exemplary embodiment of the present application, Ar1 may be a monocyclic or polycyclic heterocyclic group which is substituted or unsubstituted and includes one or more N's.

In another exemplary embodiment, N-Het is a monocyclic or polycyclic hetero ring which is unsubstituted or substituted with one or more substituents selected from the group consisting of an aryl group and a heteroaryl group, and includes one or more N's.

In still another exemplary embodiment, N-Het is a monocyclic or polycyclic hetero ring which is unsubstituted or substituted with one or more substituents selected from the group consisting of a phenyl group, a biphenyl group, a naphthyl group, a dimethylfluorene group, a dibenzofuran group and a dibenzothiophene group, and includes one or more N's.

In yet another exemplary embodiment, N-Het is a monocyclic or polycyclic hetero ring which is unsubstituted or substituted with one or more substituents selected from the group consisting of a phenyl group, a biphenyl group, a naphthyl group, a dimethylfluorene group, a dibenzofuran group and a dibenzothiophene group, and includes one or more and three or less N's.

In an exemplary embodiment of the present application, N-Het is a monocyclic hetero ring which is substituted or unsubstituted, and includes one or more N's.

In an exemplary embodiment of the present application, N-Het is a bicyclic or more hetero ring which is substituted or unsubstituted, and includes one or more N's.

In an exemplary embodiment of the present application, N-Het is a monocyclic or polycyclic hetero ring which is substituted or unsubstituted, and includes two or more N's.

In an exemplary embodiment of the present application, N-Het is a bicyclic or more polycyclic hetero ring which includes two or more N's.

In an exemplary embodiment of the present application, Ar1 is a substituted or unsubstituted heteroaryl group having 2 to 60 carbon atoms, which includes N as a hetero atom.

In an exemplary embodiment of the present application, Ar1 may be a group represented by the following Chemical Formula 2.

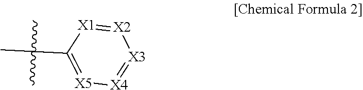

In Chemical Formula 2,
X1 is CR21 or N, X2 is CR22 or N, X3 is CR23 or N, X4 is CR24 or N, and X5 is CR25 or N, and
R21 to R25 are the same as or different from each other, and are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or hetero ring.

Here,

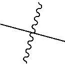

is a site which is linked to L1.

In an exemplary embodiment of the present application, Chemical Formula 2 may be represented by one of the following Chemical Formulae 3 to 6. Here,

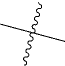

is a site which is linked to L1.

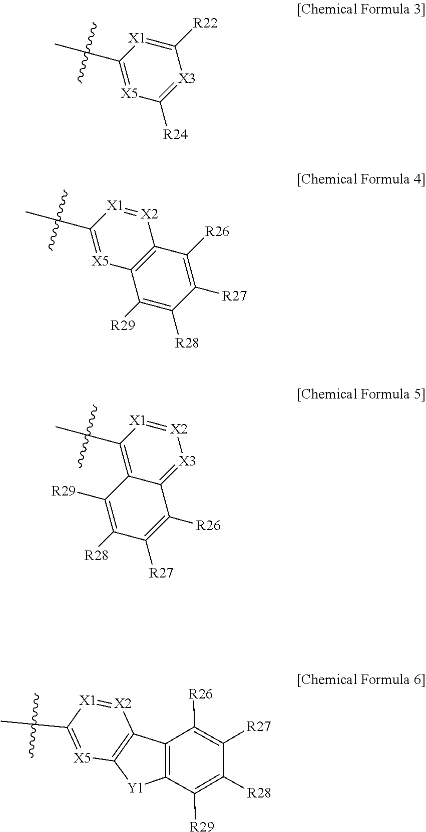

In Chemical Formula 3, one or more of X1, X3 and X5 are N, and the others are the same as those defined in Chemical Formula 2,
in Chemical Formula 4, one or more of X1, X2 and X5 are N, and the others are the same as those defined in Chemical Formula 2,
in Chemical Formula 5, one or more of X1 to X3 are N, and the others are the same as those defined in Chemical Formula 2,
in Chemical Formula 6, one or more of X1, X2 and X5 are N, the others are the same as those defined in Chemical Formula 2, and Y1 is O; or S, and
R22, R24 and R26 to R29 are the same as or different from each other, and are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or hetero ring.

In an exemplary embodiment of the present application, Chemical Formula 3 may be selected by one of the structural formulae of the following Group A.

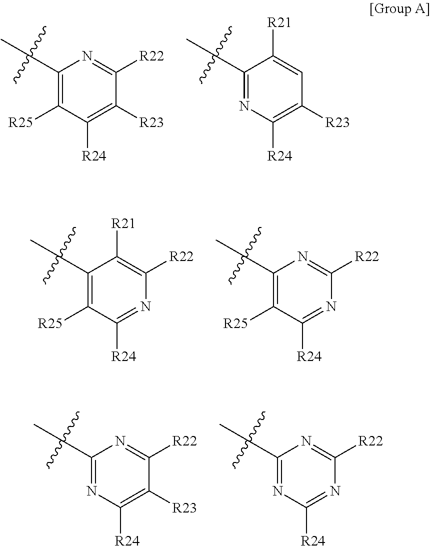

The definitions of the substituents of Group A are the same as those in Chemical Formula 3.

In an exemplary embodiment of the present application, Chemical Formula 4 may be represented by the following Chemical Formula 7.

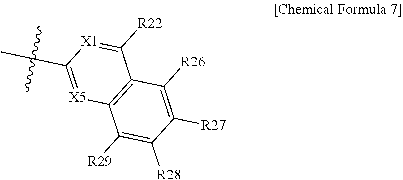

The substituents of Chemical Formula 7 are the same as those defined in Chemical Formula 4.

In an exemplary embodiment of the present application, Chemical Formula 5 may be represented by the following Chemical Formula 8.

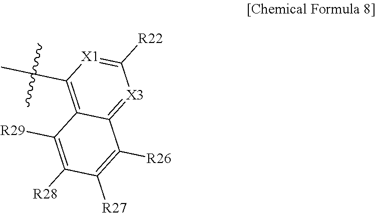

The substituents of Chemical Formula 8 are the same as those defined in Chemical Formula 5.

In an exemplary embodiment of the present application, Chemical Formula 6 may be represented by the following Chemical Formula 9.

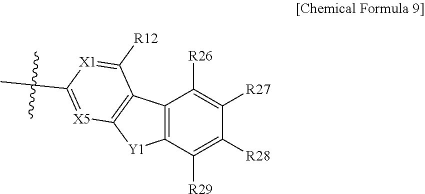

The substituents of Chemical Formula 9 are the same as those defined in Chemical Formula 6.

In an exemplary embodiment of the present application, Ar2 is —NAr3Ar4; a substituted or unsubstituted aryl group having 6 to 60 carbon atoms; or a substituted or unsubstituted heteroaryl group having 2 to 60 carbon atoms, and Ar3 and Ar4 are the same as or different from each other, and may be each independently a substituted or unsubstituted aryl group having 6 to 60 carbon atoms; or a substituted or unsubstituted heteroaryl group having 2 to 60 carbon atoms.

In an exemplary embodiment of the present application, Ar2 may be —NAr3Ar4; a substituted or unsubstituted aryl group having 6 to 40 carbon atoms; or a substituted or unsubstituted heteroaryl group having 2 to 40 carbon atoms.

In an exemplary embodiment of the present application, Ar2 may be —NAr3Ar4; a substituted or unsubstituted aryl group having 6 to 20 carbon atoms; or a substituted or unsubstituted heteroaryl group having 2 to 20 carbon atoms.

In an exemplary embodiment of the present application, Ar2 may be selected from the group consisting of —NAr3Ar4; a substituted or unsubstituted phenyl group; a substituted or unsubstituted biphenyl group; a substituted or unsubstituted dibenzofuran group; and a substituted or unsubstituted carbazole group, or a fused cyclic group thereof.

In an exemplary embodiment of the present application, examples of a fused cyclic group which may be Ar2 include the following Group B, but are not limited thereto.

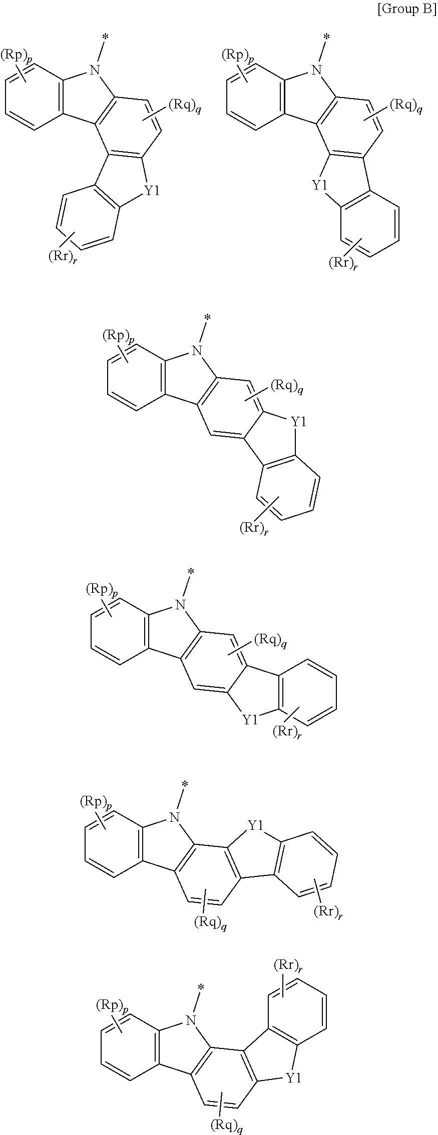

In Group B, Y1 is CRaRb; NRc; O; or S, Ra and Rb are the same as or different from each other, and are each independently a substituted or unsubstituted alkyl group having 1 to 20 carbon atoms; or a substituted or unsubstituted aryl group having 6 to 20 carbon atoms, Rc is a substituted or unsubstituted aryl group having 6 to 20 carbon atoms, Rp, Rq and Rr are each independently hydrogen; or deuterium, p and r are an integer from 0 to 4, q is an integer from 0 to 2, when p, q and r are each 2 or higher, substituents in the parenthesis are the same as or different from each other, and * is a position which is bonded to L2 of Chemical Formula 1.

In an exemplary embodiment of the present application, Ar3 and Ar4 are the same as or different from each other, and may be each independently a substituted or unsubstituted aryl group having 6 to 60 carbon atoms; or a substituted or unsubstituted heteroaryl group having 2 to 60 carbon atoms.

In an exemplary embodiment of the present application, Ar3 and Ar4 are the same as or different from each other, and may be each independently a substituted or unsubstituted aryl group having 6 to 40 carbon atoms; or a substituted or unsubstituted heteroaryl group having 2 to 40 carbon atoms.

In an exemplary embodiment of the present application, Ar3 and Ar4 are the same as or different from each other, and may be each independently a substituted or unsubstituted aryl group having 6 to 20 carbon atoms; or a substituted or unsubstituted heteroaryl group having 2 to 20 carbon atoms.

In an exemplary embodiment of the present application, Ar3 and Ar4 are the same as or different from each other, and may be each independently selected from the group consisting of a substituted or unsubstituted phenyl group; a substituted or unsubstituted biphenyl group; a substituted or unsubstituted terphenyl group; a substituted or unsubstituted naphthyl group; a substituted or unsubstituted fluorenyl group; a substituted or unsubstituted dibenzofuran group; a substituted or unsubstituted dibenzothiophene group; and a substituted or unsubstituted carbazole group.

In an exemplary embodiment of the present application, all other than the cases corresponding to -(L1)a-Ar1 or -(L2)b-Ar2 in R1 to R10 are hydrogen; or deuterium.

In an exemplary embodiment of the present application, the meaning of “all other than the cases corresponding to -(L1)a-Ar1 or -(L2)b-Ar2 in R1 to R10 are hydrogen; or deuterium” includes, for example, a case where R1 and R10 in R1 to R10 of Chemical Formula 1 are -(L1)a-Ar1 and -(L2)b-Ar2, respectively, and all of the others are hydrogen or deuterium, and this case may be represented as in the following Chemical Formula A.

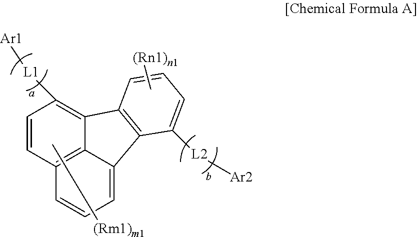

In Chemical Formula A,
Rm1 and Rn1 are the same as or different from each other, and are each independently hydrogen; or deuterium, m1 is an integer from 0 to 5, n1 is an integer from 0 to 3, and when m1 and n1 are each 2 or higher, substituents in the parenthesis are the same as or different from each other, and the definitions of L1, L2, Ar1 Ar2, a and b are the same as those in Chemical Formula 1.

The heterocyclic compound represented by Chemical Formula A corresponds to an example of the heterocyclic compound represented by Chemical Formula 1, and may be represented by a compound having a wider variety of structures according to the definition of Chemical Formula 1.

In an exemplary embodiment of the present application, Chemical Formula 1 may be represented by any one of the following Chemical Formulae 1-1 to 1-4.

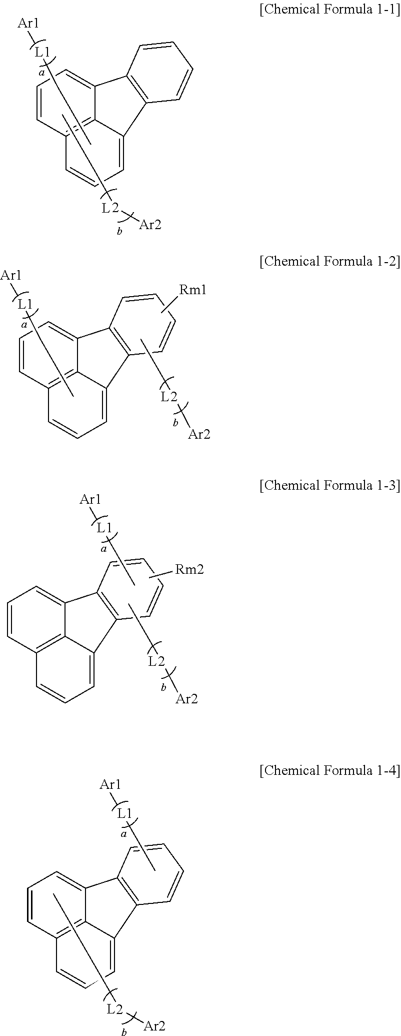

In Chemical Formulae 1-1 to 1-4,
hydrogen is unsubstituted or substituted with deuterium, and
Rm1 and Rm2 are the same as or different from each other, and are each independently hydrogen; deuterium; an aryl group having 6 to 60 carbon atoms; or a substituted or unsubstituted heteroaryl group having 2 to 60 carbon atoms, and the definitions of L1, L2, Ar1 Ar2, a and b are the same as those in Chemical Formula 1.

The deuterium content of the heterocyclic compound represented by Chemical Formulae 1-1 to 1-4 may be 0% to 100%.

The deuterium content of the heterocyclic compound represented by Chemical Formulae 1-1 to 1-4 may be more than 10% and 100% or less.

According to an exemplary embodiment of the present application, Chemical Formula 1 may be represented by any one of the following compounds, but is not limited thereto.

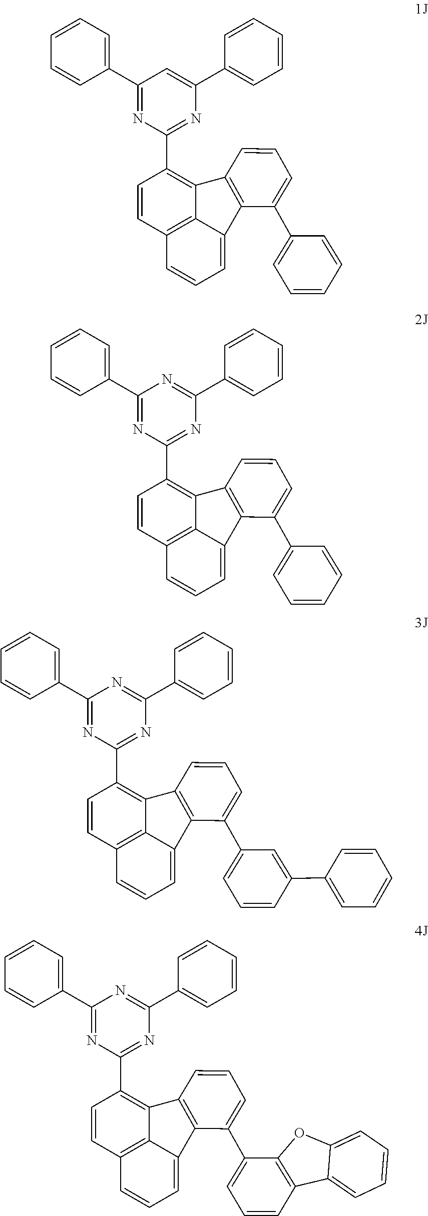

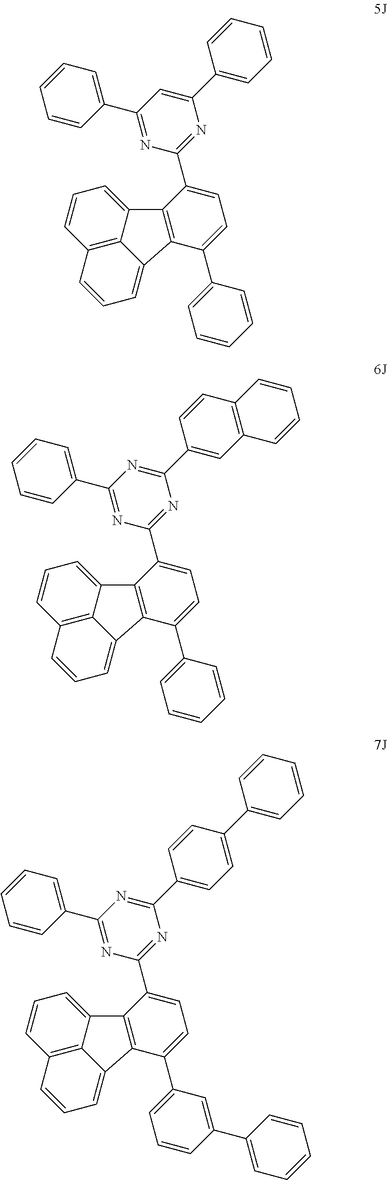

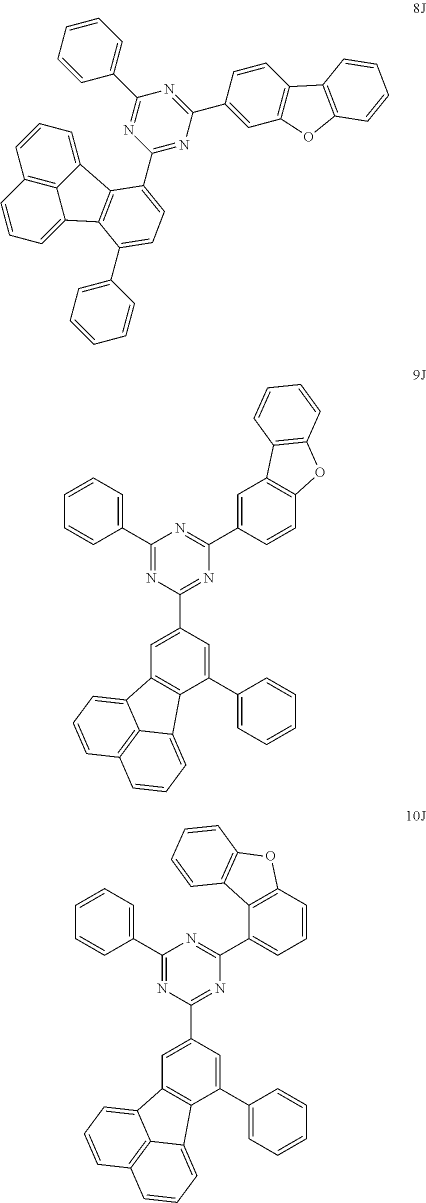

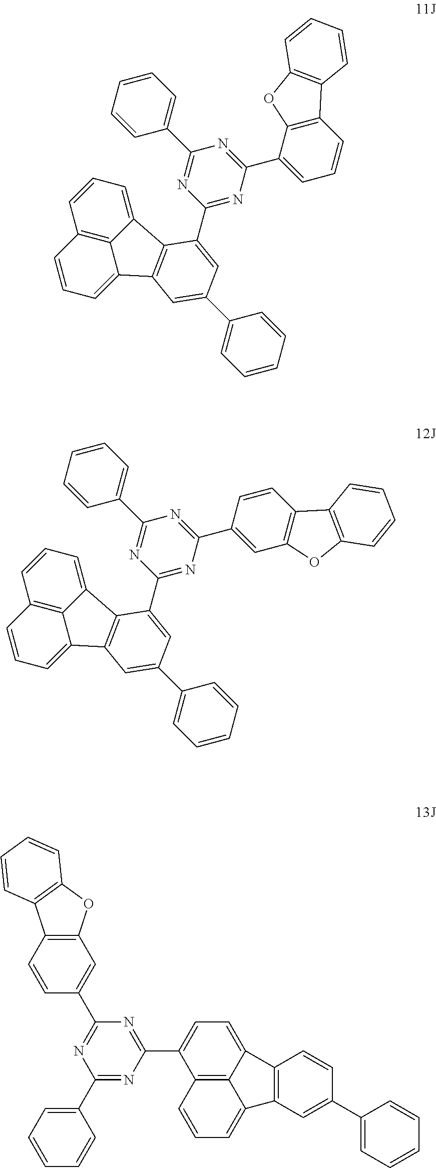 [SMILES: c1ccc(-c2cc(-c3nc(-c4ccccc4)nc(-c4cccc5c4oc4ccccc45)n3)c3c(c2)-c2cccc4cccc-3c24)cc1 ; c1ccc(-c2cc(-c3nc(-c4ccccc4)nc(-c4ccc5c(c4)oc4ccccc45)n3)c3c(c2)-c2cccc4cccc-3c24)cc1 ; c1ccc(-c2ccc3c(c2)-c2cccc4c(-c5nc(-c6ccccc6)nc(-c6ccc7c(c6)oc6ccccc67)n5)ccc-3c24)cc1]

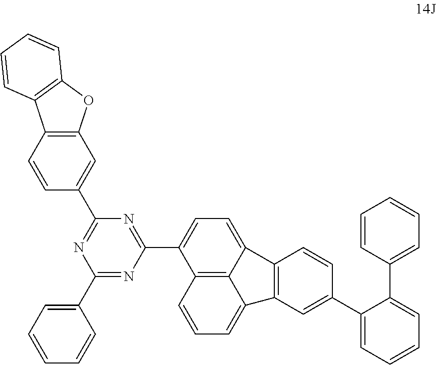 [SMILES: c1ccc(-c2nc(-c3ccc4c(c3)oc3ccccc34)nc(-c3ccc4c5c(cccc35)-c3cc(-c5ccccc5-c5ccccc5)ccc3-4)n2)cc1]

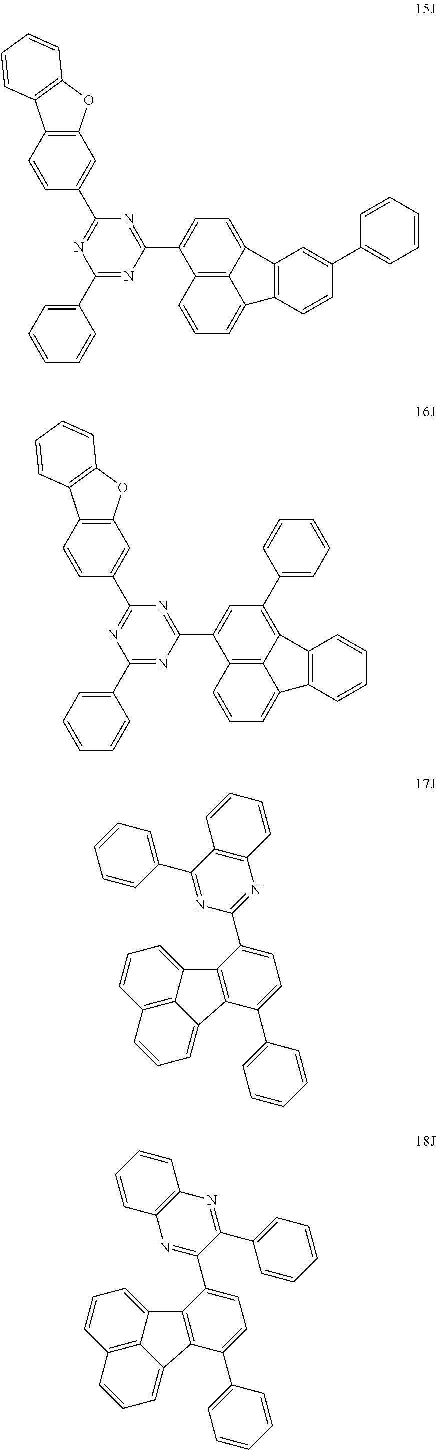 [SMILES: c1ccc(-c2ccc3c(c2)-c2ccc(-c4nc(-c5ccccc5)nc(-c5ccc6c(c5)oc5ccccc56)n4)c4cccc-3c24)cc1 ; c1ccc(-c2nc(-c3ccc4c(c3)oc3ccccc34)nc(-c3cc(-c4ccccc4)c4c5c(cccc35)-c3ccccc3-4)n2)cc1 ; c1ccc(-c2ccc(-c3nc(-c4ccccc4)c4ccccc4n3)c3c2-c2cccc4cccc-3c24)cc1 ; c1ccc(-c2ccc(-c3nc4ccccc4nc3-c3ccccc3)c3c2-c2cccc4cccc-3c24)cc1]

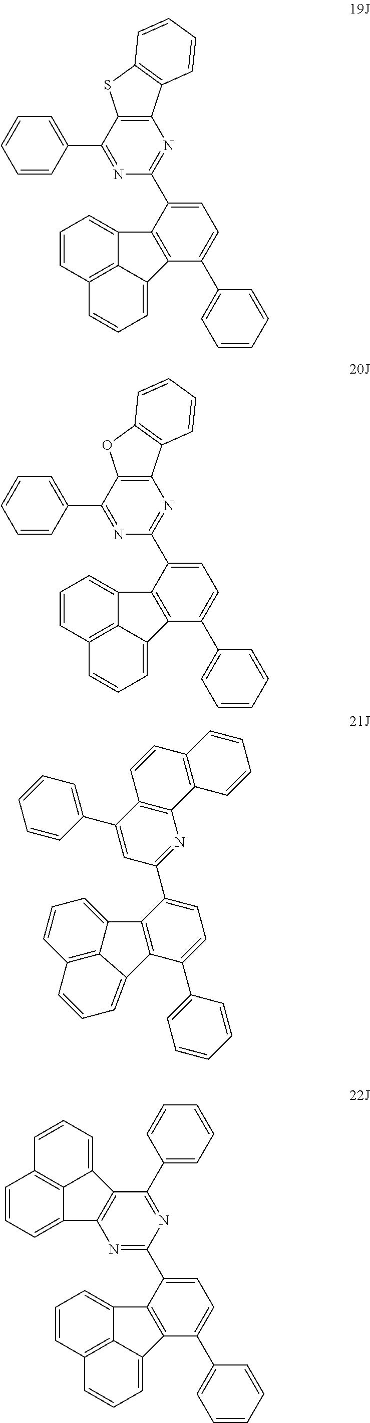

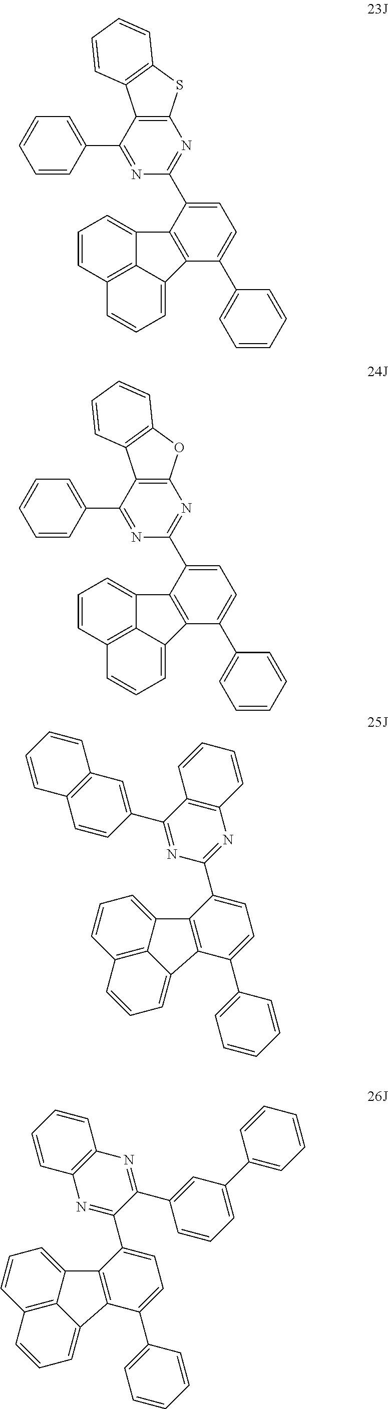

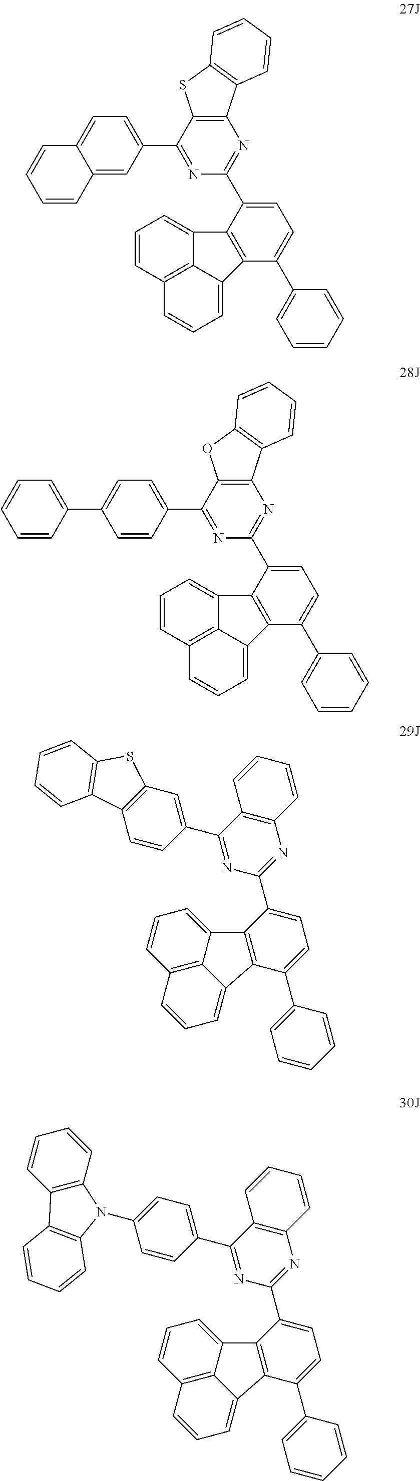

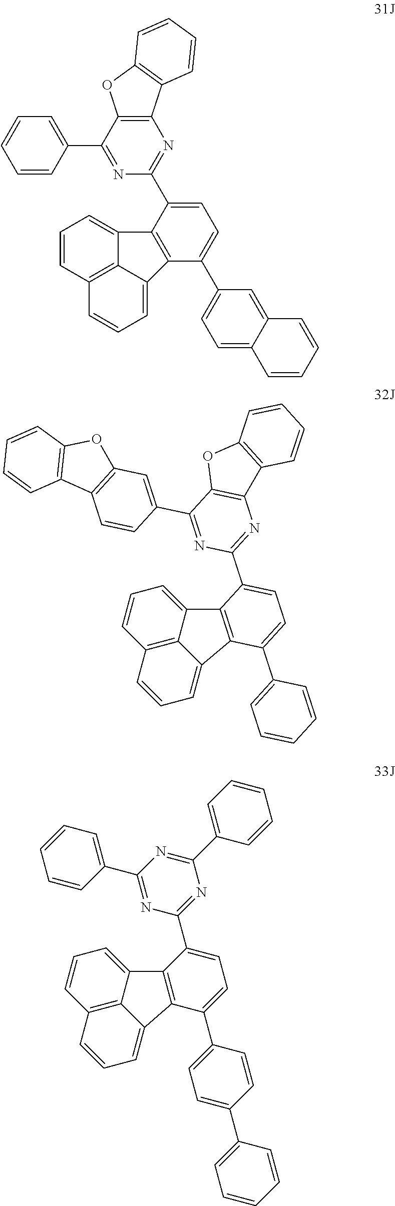

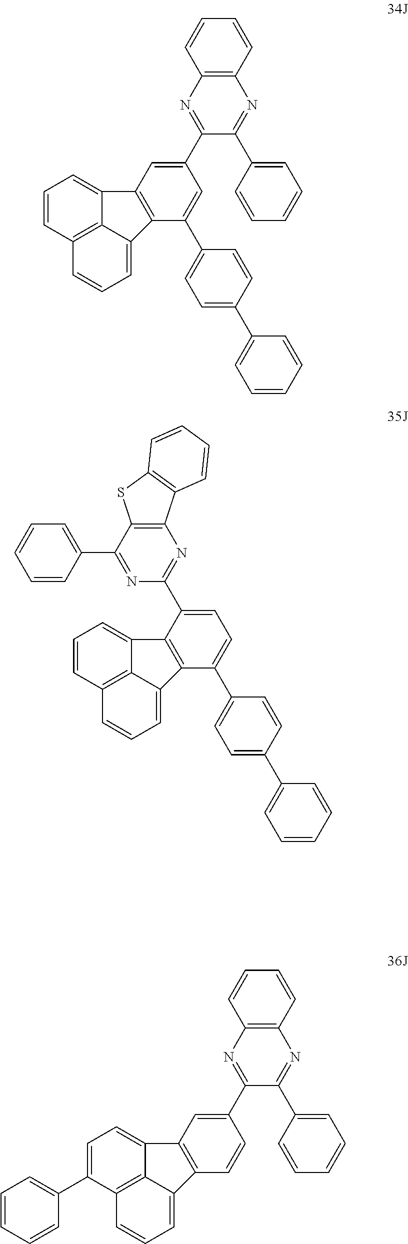 [SMILES: c1ccc(-c2ccc(-c3cc(-c4nc5ccccc5nc4-c4ccccc4)cc4c3-c3cccc5cccc-4c35)cc2)cc1 ; c1ccc(-c2ccc(-c3ccc(-c4nc(-c5ccccc5)c5sc6ccccc6c5n4)c4c3-c3cccc5cccc-4c35)cc2)cc1 ; c1ccc(-c2nc3ccccc3nc2-c2ccc3c(c2)-c2ccc(-c4ccccc4)c4cccc-3c24)cc1]

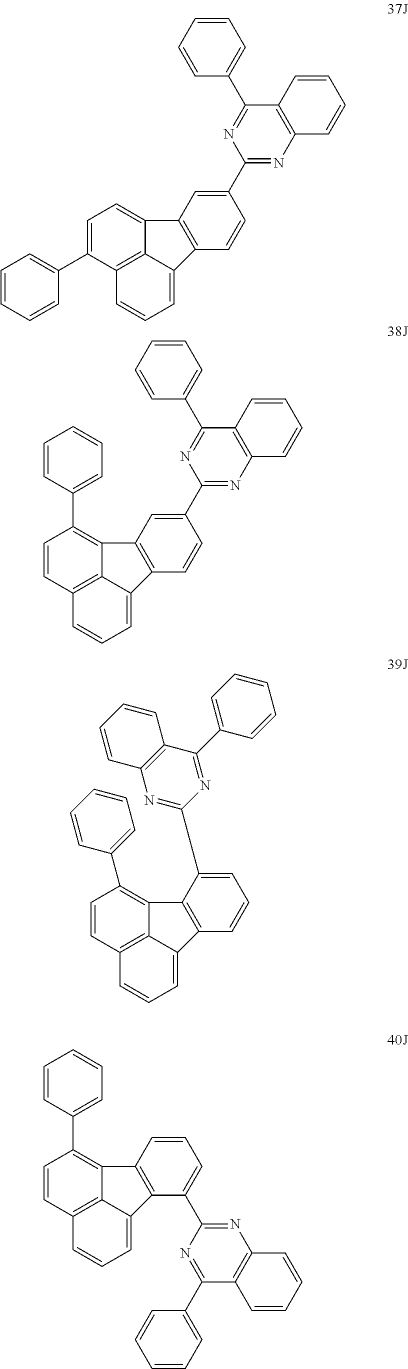

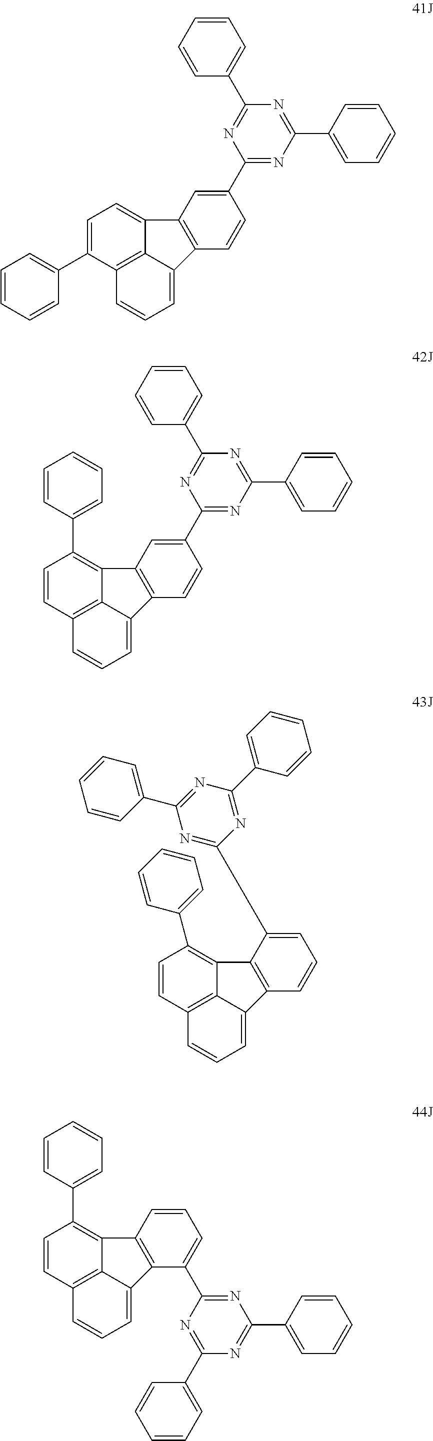 [SMILES: c1ccc(-c2nc(-c3ccccc3)nc(-c3ccc4c(c3)-c3ccc(-c5ccccc5)c5cccc-4c35)n2)cc1 ; c1ccc(-c2nc(-c3ccccc3)nc(-c3ccc4c(c3)-c3c(-c5ccccc5)ccc5cccc-4c35)n2)cc1 ; c1ccc(-c2nc(CCc3cccc4c3-c3c(-c5ccccc5)ccc5cccc-4c35)nc(-c3ccccc3)n2)cc1 ; c1ccc(-c2nc(-c3ccccc3)nc(-c3cccc4c3-c3cccc5ccc(-c6ccccc6)c-4c35)n2)cc1]

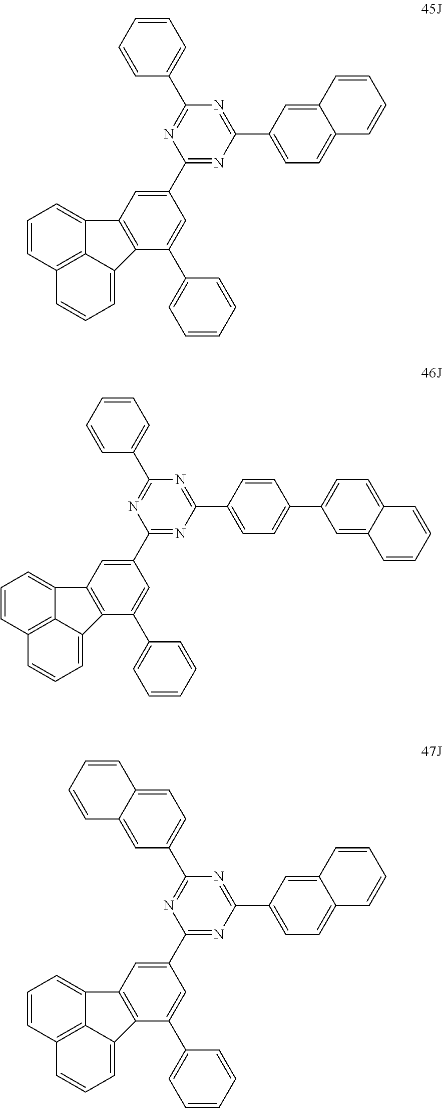 [SMILES: c1ccc(-c2nc(-c3cc(-c4ccccc4)c4c(c3)-c3cccc5cccc-4c35)nc(-c3ccc4ccccc4c3)n2)cc1 ; c1ccc(-c2nc(-c3ccc(-c4ccc5ccccc5c4)cc3)nc(-c3cc(-c4ccccc4)c4c(c3)-c3cccc5cccc-4c35)n2)cc1 ; c1ccc(-c2cc(-c3nc(-c4ccc5ccccc5c4)nc(-c4ccc5ccccc5c4)n3)cc3c2-c2cccc4cccc-3c24)cc1]

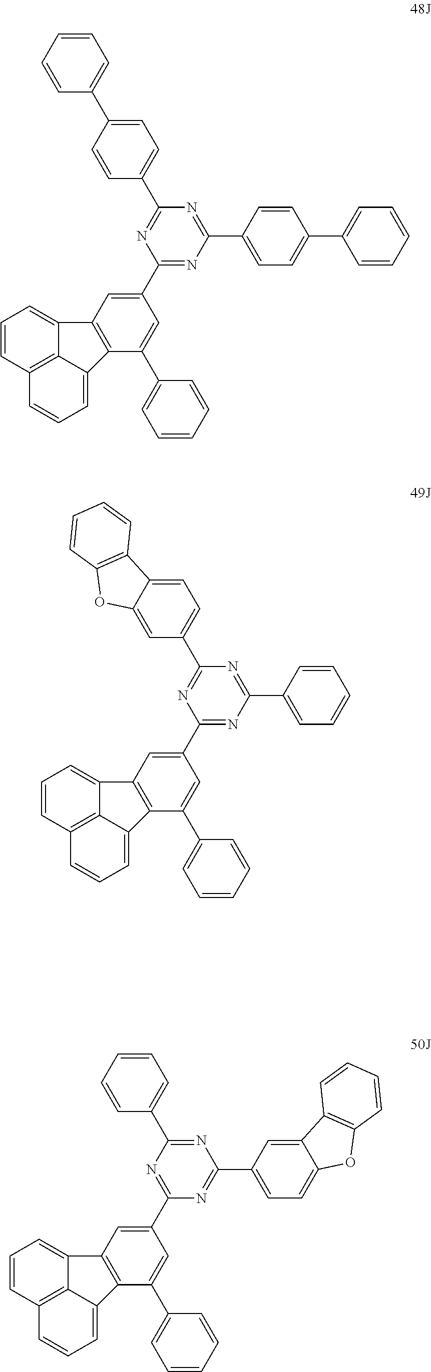 [SMILES: c1ccc(-c2ccc(-c3nc(-c4ccc(-c5ccccc5)cc4)nc(-c4cc(-c5ccccc5)c5c(c4)-c4cccc6cccc-5c46)n3)cc2)cc1 ; c1ccc(-c2nc(-c3cc(-c4ccccc4)c4c(c3)-c3cccc5cccc-4c35)nc(-c3ccc4c(c3)oc3ccccc34)n2)cc1 ; c1ccc(-c2nc(-c3cc(-c4ccccc4)c4c(c3)-c3cccc5cccc-4c35)nc(-c3ccc4oc5ccccc5c4c3)n2)cc1]

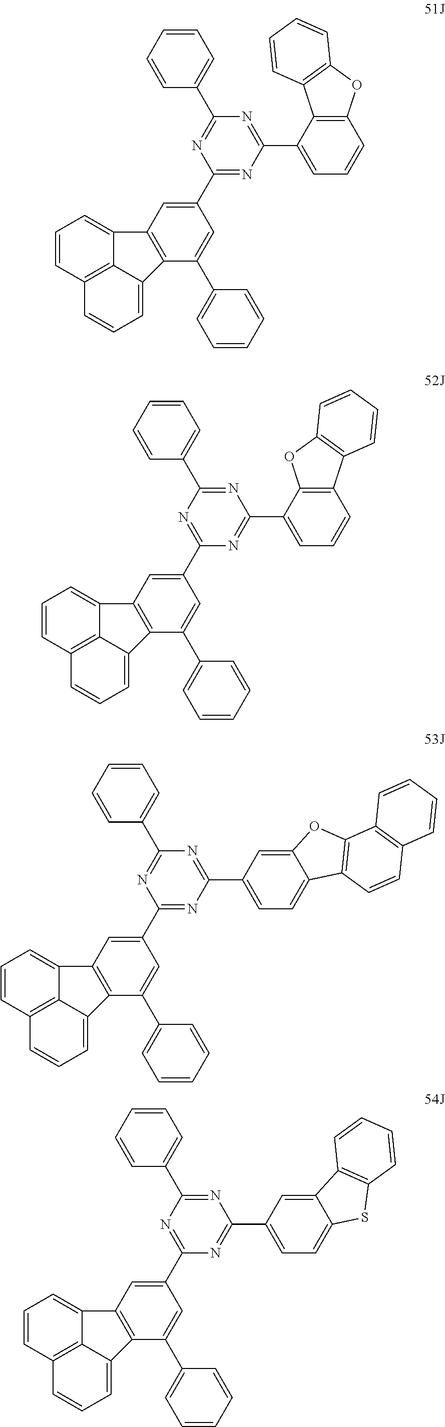

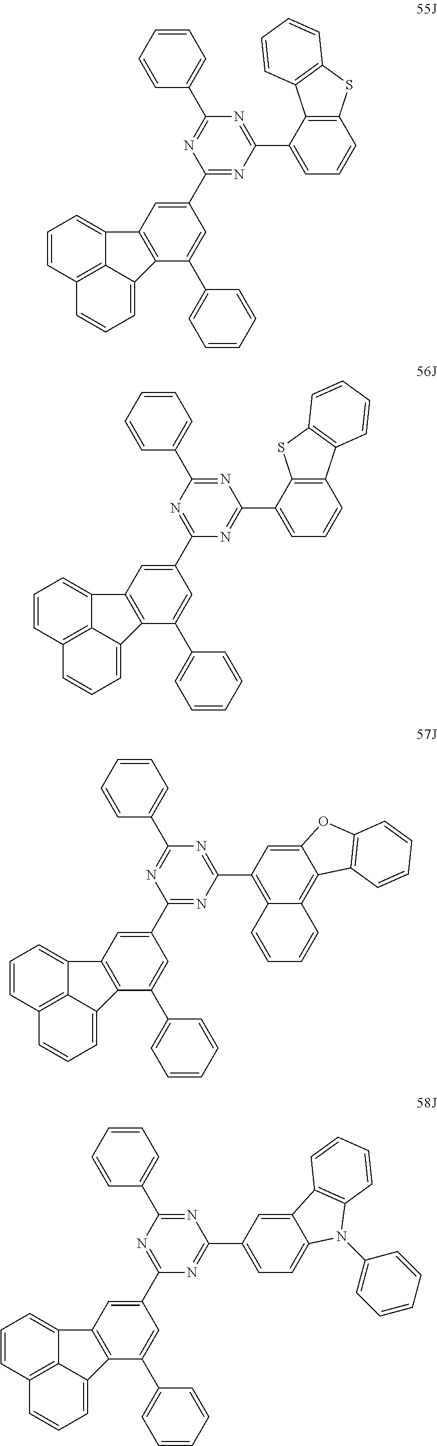 [SMILES: c1ccc(-c2nc(-c3cc(-c4ccccc4)c4c(c3)-c3cccc5cccc-4c35)nc(-c3cccc4sc5ccccc5c34)n2)cc1 ; c1ccc(-c2nc(-c3cc(-c4ccccc4)c4c(c3)-c3cccc5cccc-4c35)nc(-c3cccc4c3sc3ccccc34)n2)cc1 ; c1ccc(-c2nc(-c3cc(-c4ccccc4)c4c(c3)-c3cccc5cccc-4c35)nc(-c3cc4oc5ccccc5c4c4ccccc34)n2)cc1 ; c1ccc(-c2nc(-c3cc(-c4ccccc4)c4c(c3)-c3cccc5cccc-4c35)nc(-c3ccc4c(c3)c3ccccc3n4-c3ccccc3)n2)cc1]

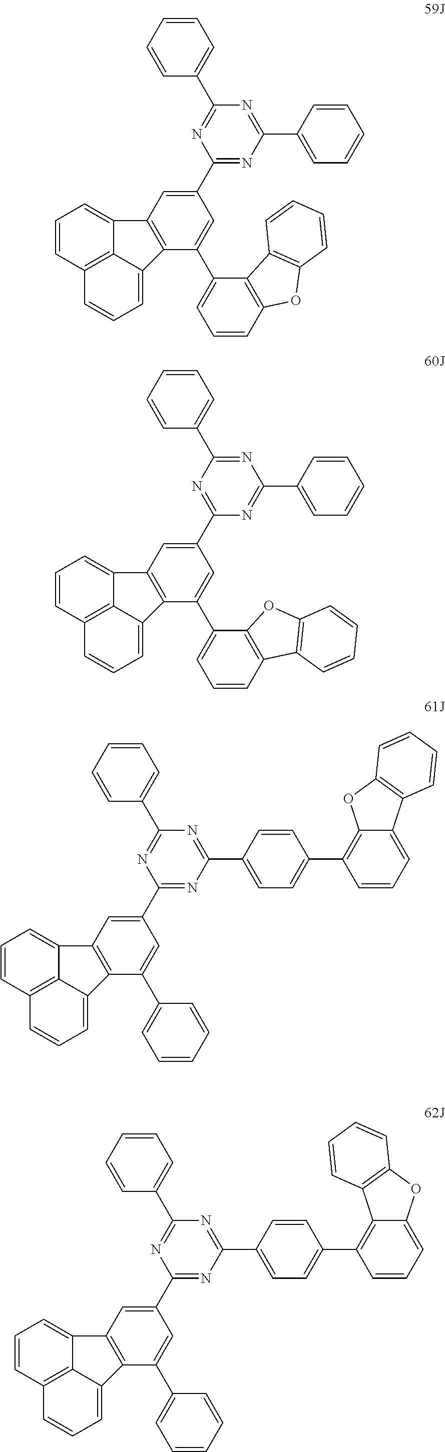

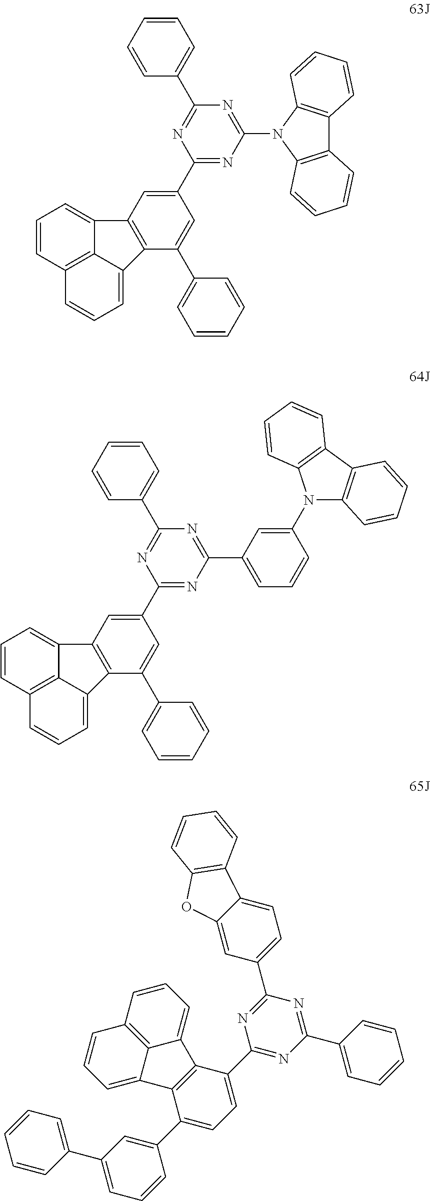 [SMILES: c1ccc(-c2nc(-c3cc(-c4ccccc4)c4c(c3)-c3cccc5cccc-4c35)nc(-n3c4ccccc4c4ccccc43)n2)cc1 ; c1ccc(-c2nc(-c3cccc(-n4c5ccccc5c5ccccc54)c3)nc(-c3cc(-c4ccccc4)c4c(c3)-c3cccc5cccc-4c35)n2)cc1 ; c1ccc(-c2cccc(-c3ccc(-c4nc(-c5ccccc5)nc(-c5ccc6c(c5)oc5ccccc56)n4)c4c3-c3cccc5cccc-4c35)c2)cc1]

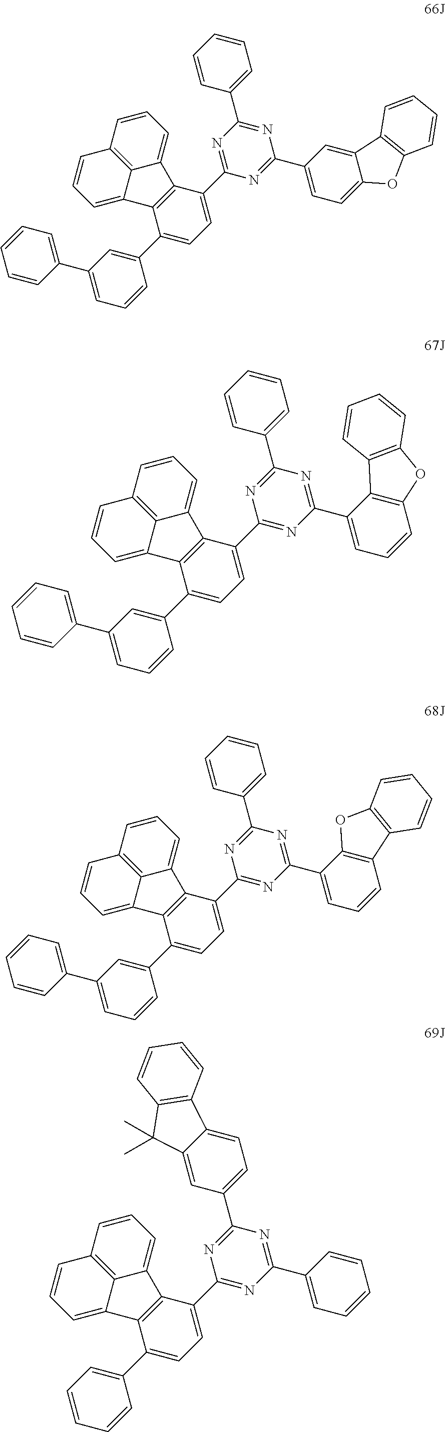

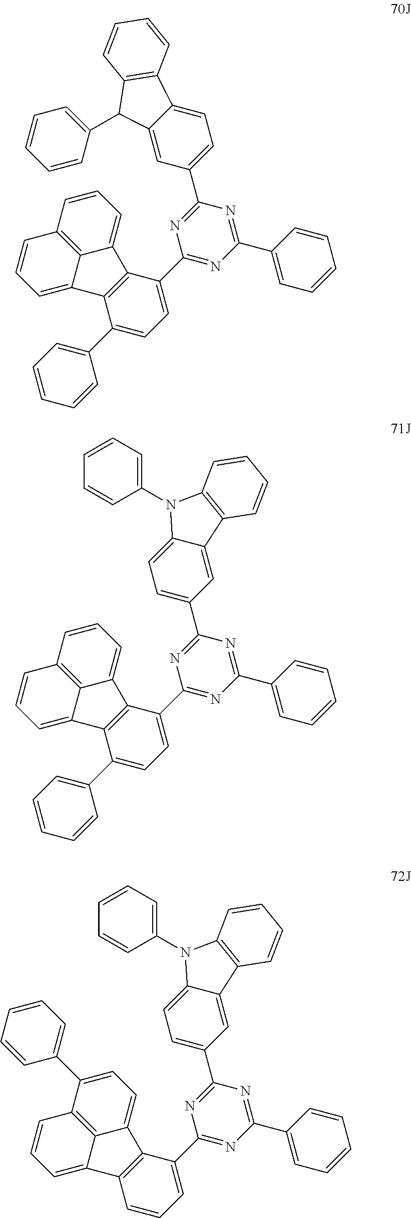 [SMILES: c1ccc(-c2nc(-c3ccc4c(c3)C(c3ccccc3)c3ccccc3-4)nc(-c3ccc(-c4ccccc4)c4c3-c3cccc5cccc-4c35)n2)cc1 ; c1ccc(-c2nc(-c3ccc4c(c3)c3ccccc3n4-c3ccccc3)nc(-c3ccc(-c4ccccc4)c4c3-c3cccc5cccc-4c35)n2)cc1 ; c1ccc(-c2nc(-c3ccc4c(c3)c3ccccc3n4-c3ccccc3)nc(-c3cccc4c3-c3ccc(-c5ccccc5)c5cccc-4c35)n2)cc1]

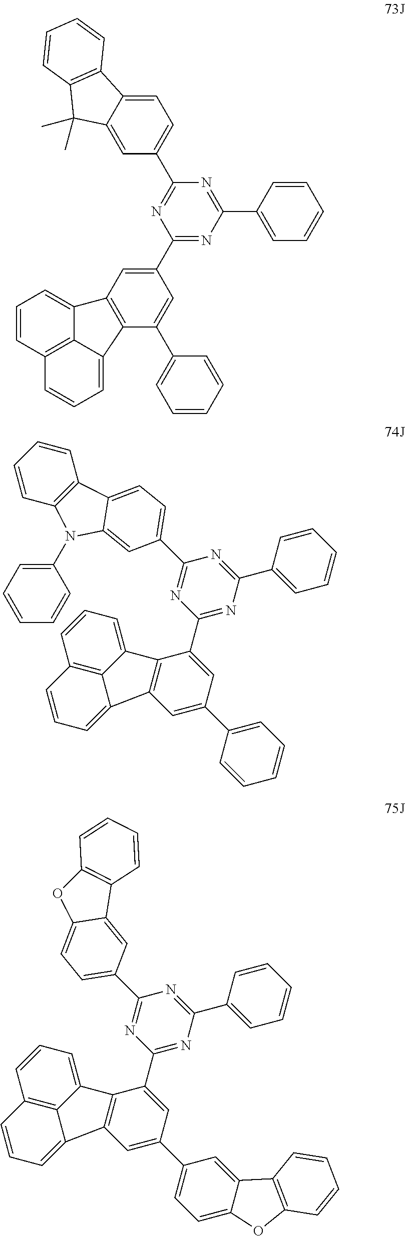

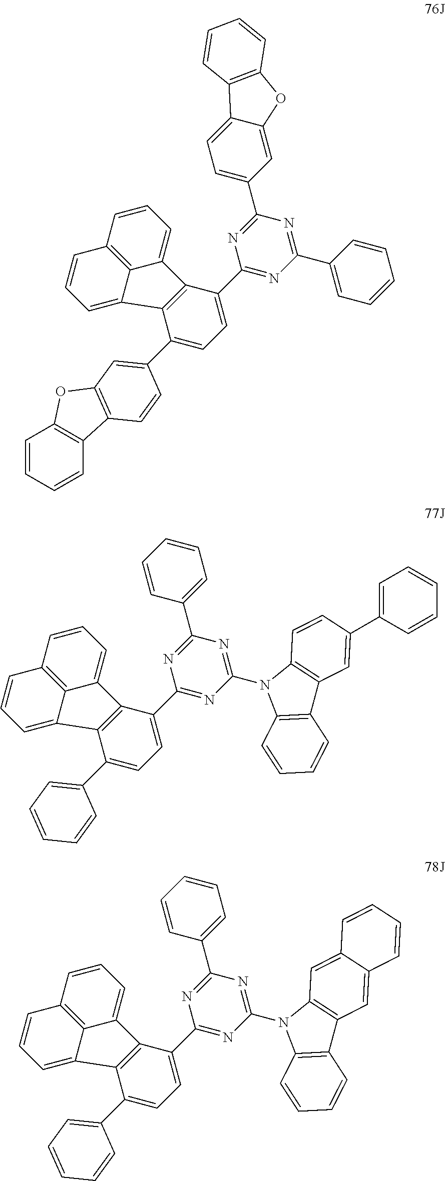 [SMILES: c1ccc(-c2nc(-c3ccc4c(c3)oc3ccccc34)nc(-c3ccc(-c4ccc5c(c4)oc4ccccc45)c4c3-c3cccc5cccc-4c35)n2)cc1 ; c1ccc(-c2ccc3c(c2)c2ccccc2n3-c2nc(-c3ccccc3)nc(-c3ccc(-c4ccccc4)c4c3-c3cccc5cccc-4c35)n2)cc1 ; c1ccc(-c2nc(-c3ccc(-c4ccccc4)c4c3-c3cccc5cccc-4c35)nc(-n3c4ccccc4c4cc5ccccc5cc43)n2)cc1]

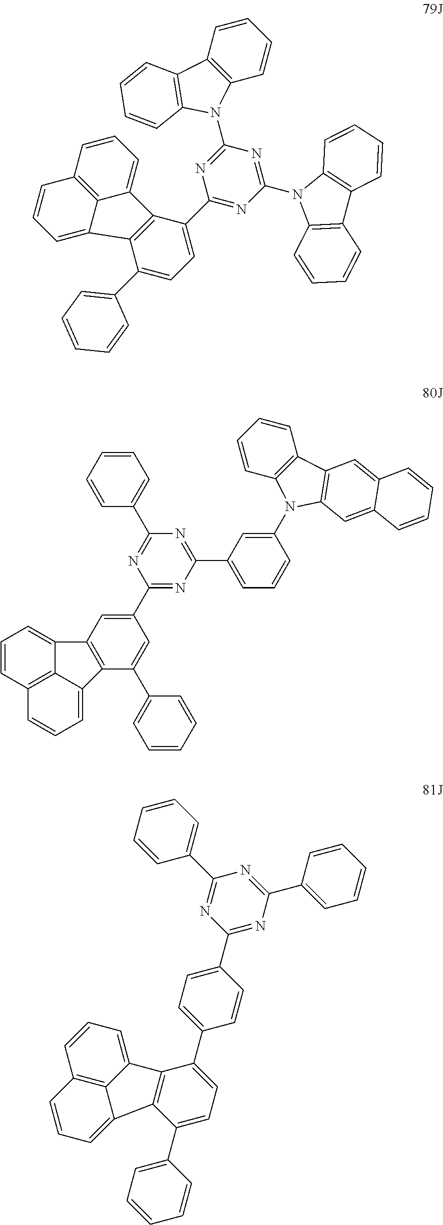 [SMILES: c1ccc(-c2ccc(-c3nc(-n4c5ccccc5c5ccccc54)nc(-n4c5ccccc5c5ccccc54)n3)c3c2-c2cccc4cccc-3c24)cc1 ; c1ccc(-c2nc(-c3cccc(-n4c5ccccc5c5cc6ccccc6cc54)c3)nc(-c3cc(-c4ccccc4)c4c(c3)-c3cccc5cccc-4c35)n2)cc1 ; c1ccc(-c2nc(-c3ccccc3)nc(-c3ccc(-c4ccc(-c5ccccc5)c5c4-c4cccc6cccc-5c46)cc3)n2)cc1]

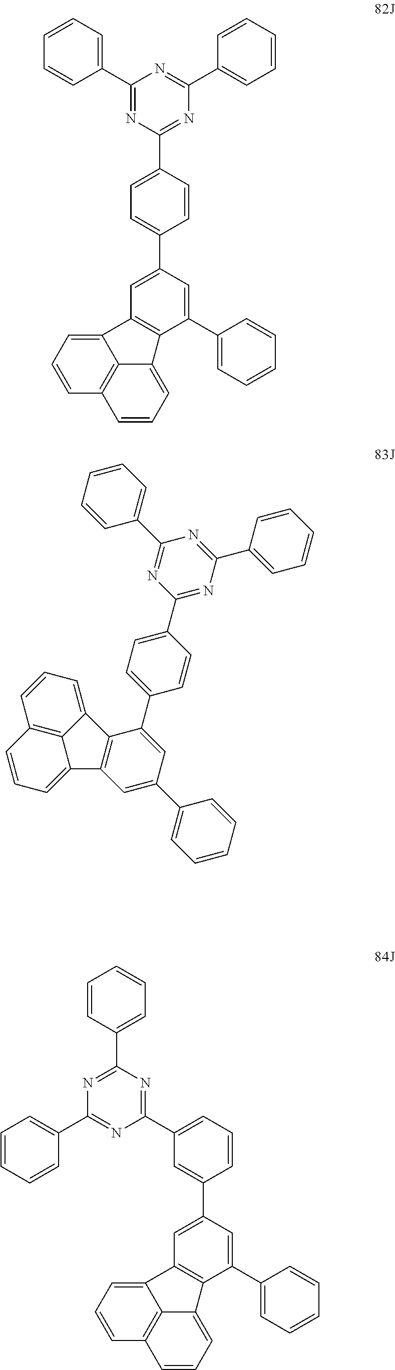

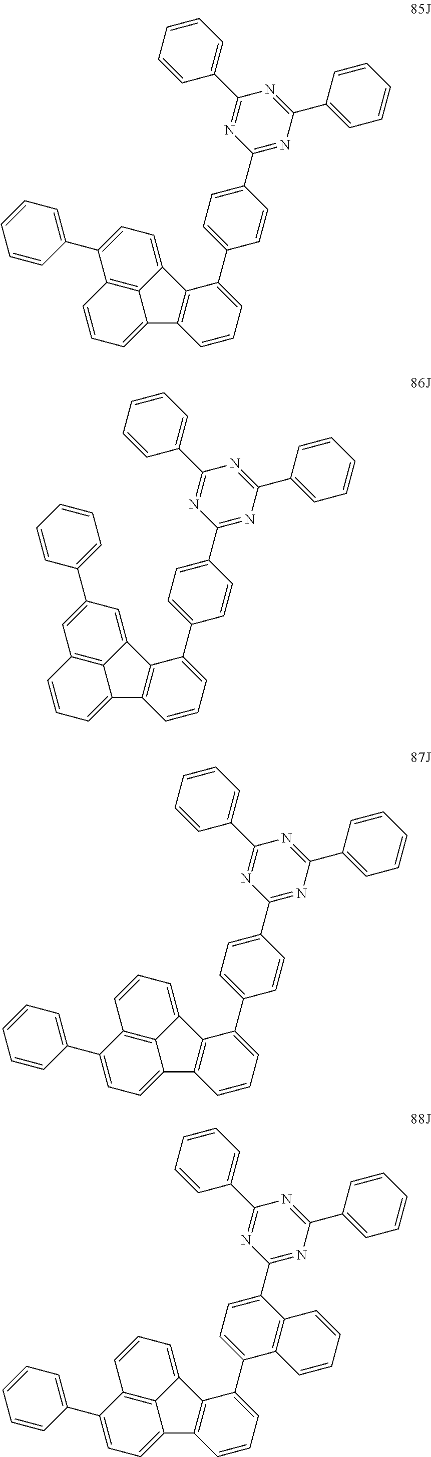

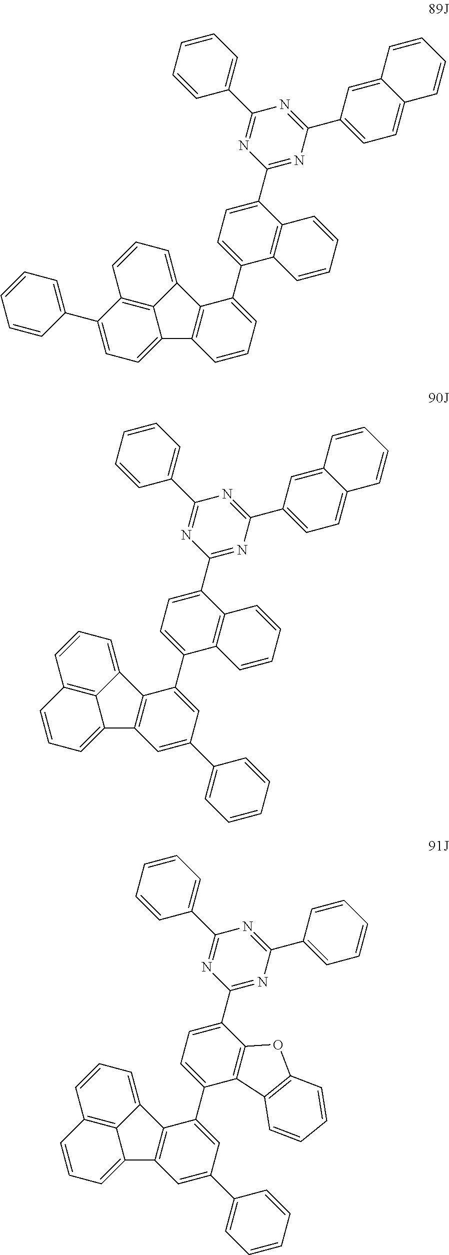 [SMILES: c1ccc(-c2nc(-c3ccc4ccccc4c3)nc(-c3ccc(-c4cccc5c4-c4cccc6c(-c7ccccc7)ccc-5c46)c4ccccc34)n2)cc1 ; c1ccc(-c2cc(-c3ccc(-c4nc(-c5ccccc5)nc(-c5ccc6ccccc6c5)n4)c4ccccc34)c3c(c2)-c2cccc4cccc-3c24)cc1 ; c1ccc(-c2cc3c(c(-c4ccc(-c5nc(-c6ccccc6)nc(-c6ccccc6)n5)c5oc6ccccc6c45)c2)-c2cccc4cccc-3c24)cc1]

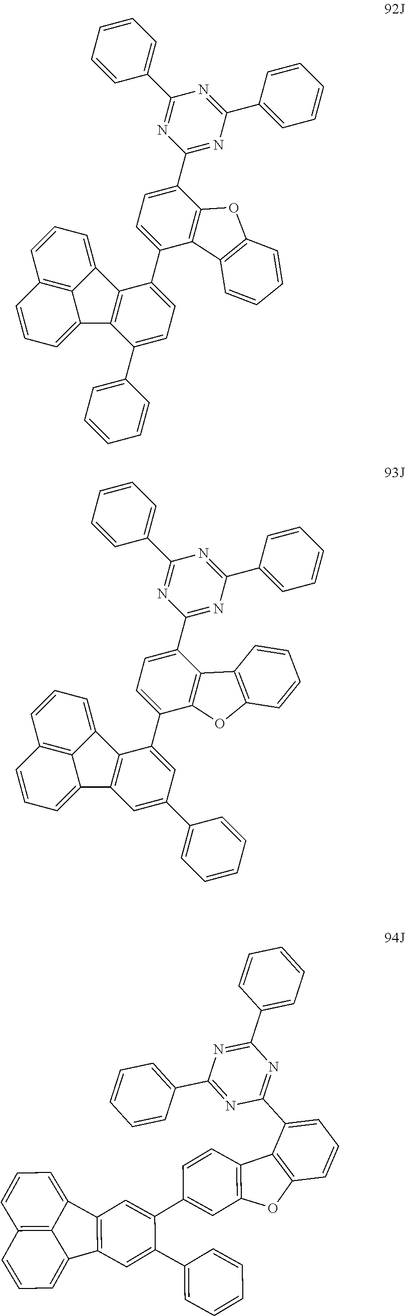

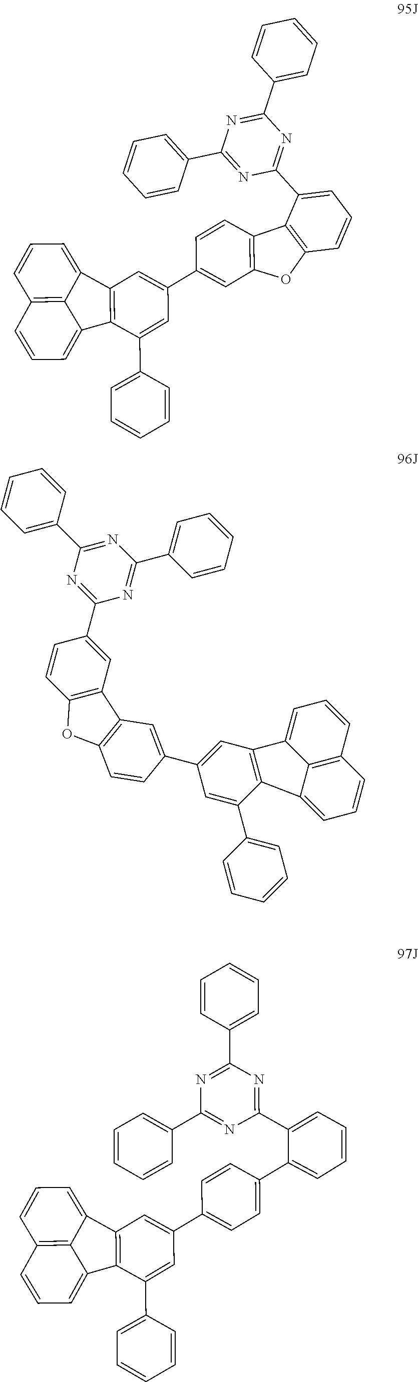 [SMILES: c1ccc(-c2nc(-c3ccccc3)nc(-c3cccc4oc5cc(-c6cc(-c7ccccc7)c7c(c6)-c6cccc8cccc-7c68)ccc5c34)n2)cc1 ; c1ccc(-c2nc(-c3ccccc3)nc(-c3ccc4oc5ccc(-c6cc(-c7ccccc7)c7c(c6)-c6cccc8cccc-7c68)cc5c4c3)n2)cc1 ; c1ccc(-c2nc(-c3ccccc3)nc(-c3ccccc3-c3ccc(-c4cc(-c5ccccc5)c5c(c4)-c4cccc6cccc-5c46)cc3)n2)cc1]

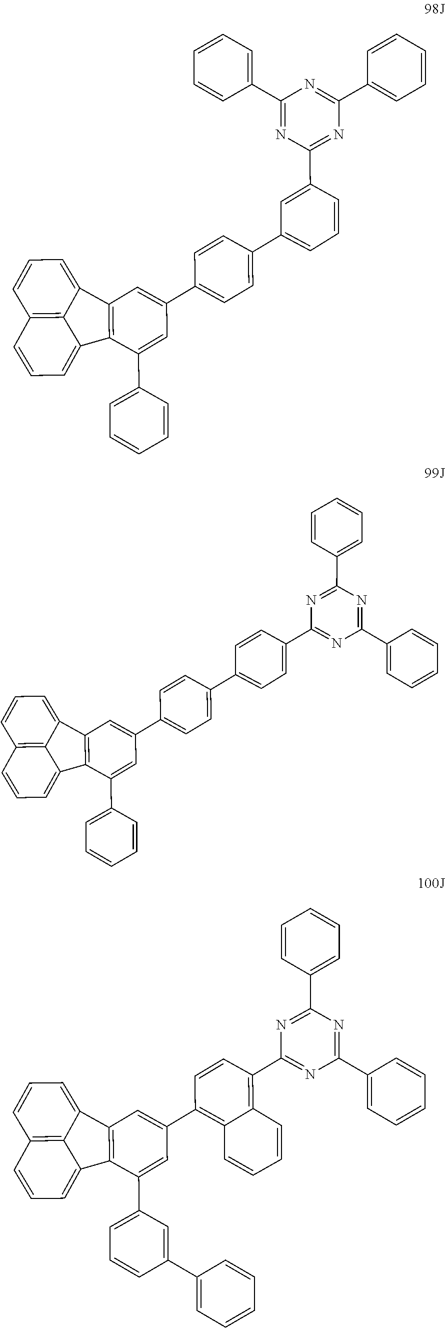 [SMILES: c1ccc(-c2nc(-c3ccccc3)nc(-c3cccc(-c4ccc(-c5cc(-c6ccccc6)c6c(c5)-c5cccc7cccc-6c57)cc4)c3)n2)cc1 ; c1ccc(-c2nc(-c3ccccc3)nc(-c3ccc(-c4ccc(-c5cc(-c6ccccc6)c6c(c5)-c5cccc7cccc-6c57)cc4)cc3)n2)cc1 ; c1ccc(-c2cccc(-c3cc(-c4ccc(-c5nc(-c6ccccc6)nc(-c6ccccc6)n5)c5ccccc45)cc4c3-c3cccc5cccc-4c35)c2)cc1]

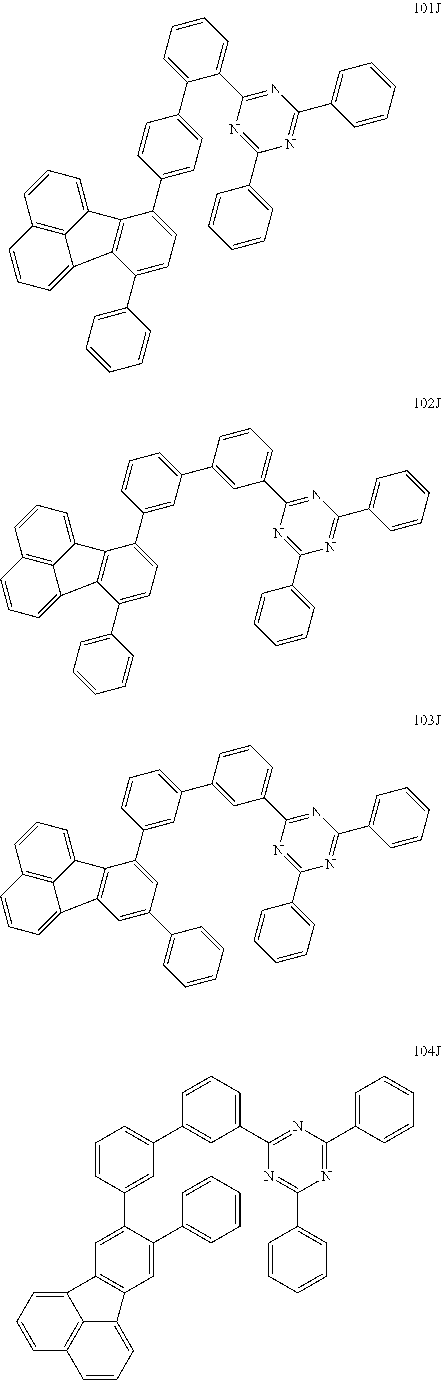 [SMILES: c1ccc(-c2nc(-c3ccccc3)nc(-c3ccccc3-c3ccc(-c4ccc(-c5ccccc5)c5c4-c4cccc6cccc-5c46)cc3)n2)cc1 ; c1ccc(-c2nc(-c3ccccc3)nc(-c3cccc(-c4cccc(-c5ccc(-c6ccccc6)c6c5-c5cccc7cccc-6c57)c4)c3)n2)cc1 ; c1ccc(-c2cc(-c3cccc(-c4cccc(-c5nc(-c6ccccc6)nc(-c6ccccc6)n5)c4)c3)c3c(c2)-c2cccc4cccc-3c24)cc1 ; c1ccc(-c2nc(-c3ccccc3)nc(-c3cccc(-c4cccc(-c5cc6c(cc5-c5ccccc5)-c5cccc7cccc-6c57)c4)c3)n2)cc1]

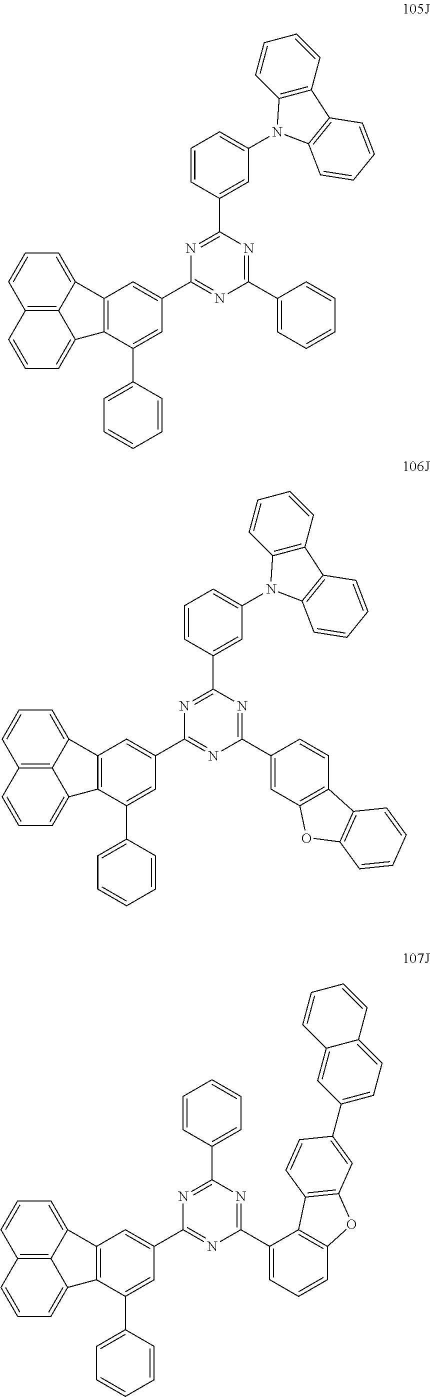 [SMILES: c1ccc(-c2nc(-c3cccc(-n4c5ccccc5c5ccccc54)c3)nc(-c3cc(-c4ccccc4)c4c(c3)-c3cccc5cccc-4c35)n2)cc1 ; c1ccc(-c2cc(-c3nc(-c4cccc(-n5c6ccccc6c6ccccc65)c4)nc(-c4ccc5c(c4)oc4ccccc45)n3)cc3c2-c2cccc4cccc-3c24)cc1 ; c1ccc(-c2nc(-c3cc(-c4ccccc4)c4c(c3)-c3cccc5cccc-4c35)nc(-c3cccc4oc5cc(-c6ccc7ccccc7c6)ccc5c34)n2)cc1]

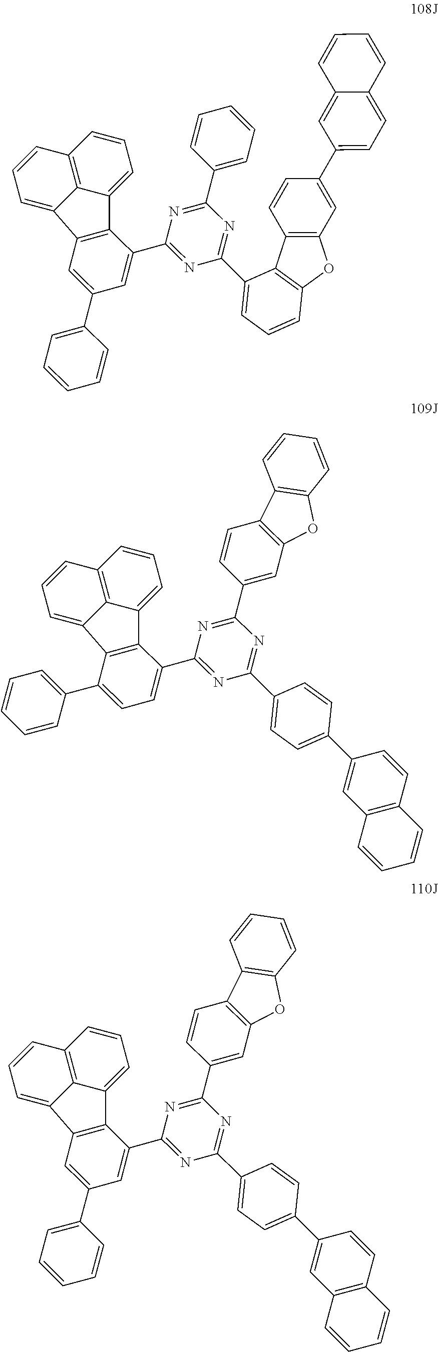 [SMILES: c1ccc(-c2cc(-c3nc(-c4ccccc4)nc(-c4cccc5oc6cc(-c7ccc8ccccc8c7)ccc6c45)n3)c3c(c2)-c2cccc4cccc-3c24)cc1 ; c1ccc(-c2ccc(-c3nc(-c4ccc(-c5ccc6ccccc6c5)cc4)nc(-c4ccc5c(c4)oc4ccccc45)n3)c3c2-c2cccc4cccc-3c24)cc1 ; c1ccc(-c2cc(-c3nc(-c4ccc(-c5ccc6ccccc6c5)cc4)nc(-c4ccc5c(c4)oc4ccccc45)n3)c3c(c2)-c2cccc4cccc-3c24)cc1]

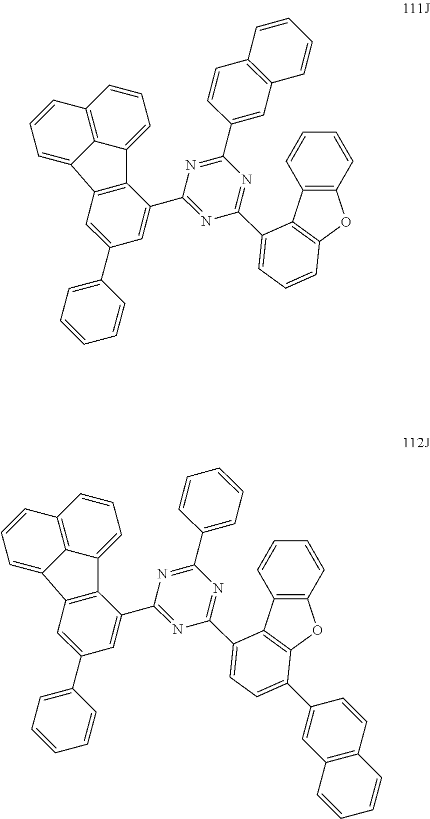 [SMILES: c1ccc(-c2cc(-c3nc(-c4ccc5ccccc5c4)nc(-c4cccc5oc6ccccc6c45)n3)c3c(c2)-c2cccc4cccc-3c24)cc1 ; c1ccc(-c2cc(-c3nc(-c4ccccc4)nc(-c4ccc(-c5ccc6ccccc6c5)c5oc6ccccc6c45)n3)c3c(c2)-c2cccc4cccc-3c24)cc1]

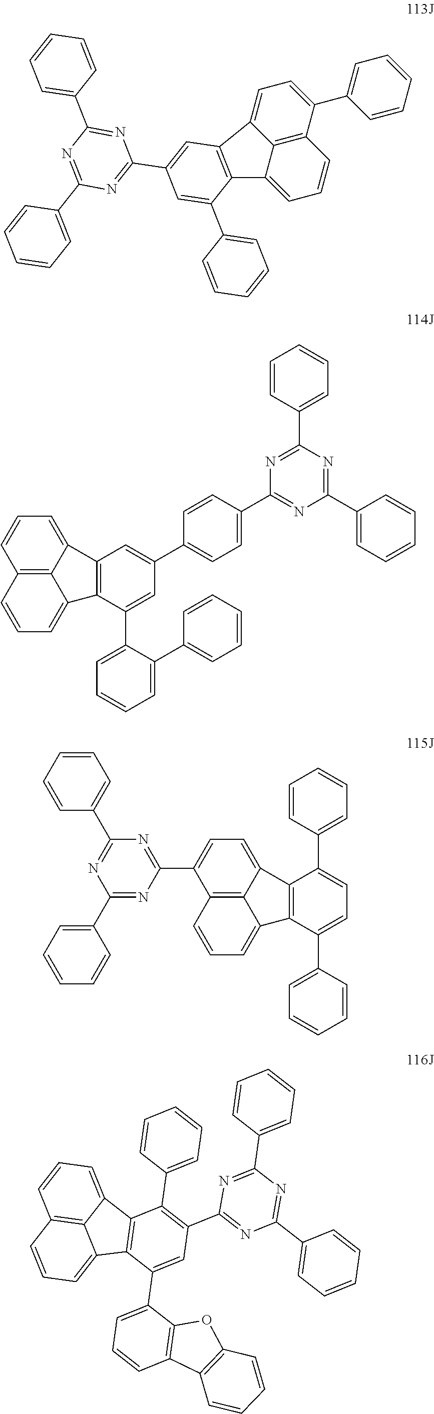 [SMILES: c1ccc(-c2nc(-c3ccccc3)nc(-c3cc(-c4ccccc4)c4c(c3)-c3ccc(-c5ccccc5)c5cccc-4c35)n2)cc1 ; c1ccc(-c2nc(-c3ccccc3)nc(-c3ccc(-c4cc(-c5ccccc5-c5ccccc5)c5c(c4)-c4cccc6cccc-5c46)cc3)n2)cc1 ; c1ccc(-c2nc(-c3ccccc3)nc(-c3ccc4c5c(cccc35)-c3c(-c5ccccc5)ccc(-c5ccccc5)c3-4)n2)cc1 ; c1ccc(-c2nc(-c3ccccc3)nc(-c3cc(-c4cccc5c4oc4ccccc45)c4c(c3-c3ccccc3)-c3cccc5cccc-4c35)n2)cc1]

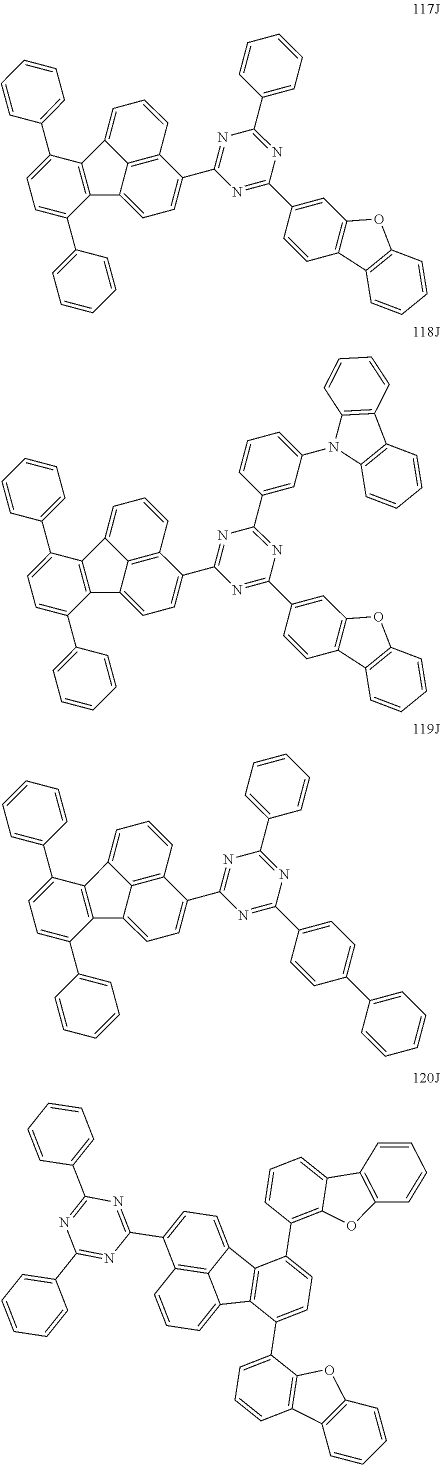 [SMILES: c1ccc(-c2nc(-c3ccc4c(c3)oc3ccccc34)nc(-c3ccc4c5c(cccc35)-c3c(-c5ccccc5)ccc(-c5ccccc5)c3-4)n2)cc1 ; c1ccc(-c2ccc(-c3ccccc3)c3c2-c2cccc4c(-c5nc(-c6cccc(-n7c8ccccc8c8ccccc87)c6)nc(-c6ccc7c(c6)oc6ccccc67)n5)ccc-3c24)cc1 ; c1ccc(-c2ccc(-c3nc(-c4ccccc4)nc(-c4ccc5c6c(cccc46)-c4c(-c6ccccc6)ccc(-c6ccccc6)c4-5)n3)cc2)cc1 ; c1ccc(-c2nc(-c3ccccc3)nc(-c3ccc4c5c(cccc35)-c3c(-c5cccc6c5oc5ccccc56)ccc(-c5cccc6c5oc5ccccc56)c3-4)n2)cc1]

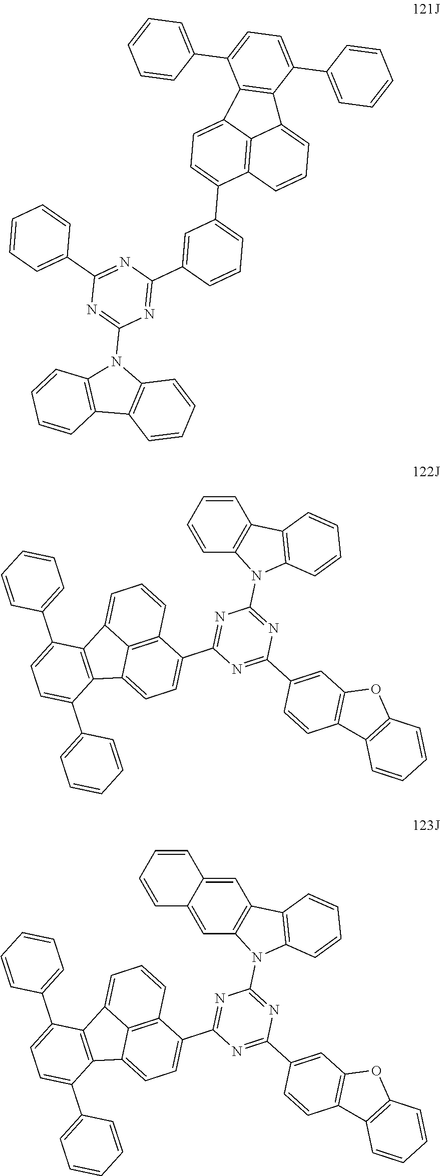 [SMILES: c1ccc(-c2nc(-c3cccc(-c4ccc5c6c(cccc46)-c4c(-c6ccccc6)ccc(-c6ccccc6)c4-5)c3)nc(-n3c4ccccc4c4ccccc43)n2)cc1 ; c1ccc(-c2ccc(-c3ccccc3)c3c2-c2cccc4c(-c5nc(-c6ccc7c(c6)oc6ccccc67)nc(-n6c7ccccc7c7ccccc76)n5)ccc-3c24)cc1 ; c1ccc(-c2ccc(-c3ccccc3)c3c2-c2cccc4c(-c5nc(-c6ccc7c(c6)oc6ccccc67)nc(-n6c7ccccc7c7cc8ccccc8cc76)n5)ccc-3c24)cc1]

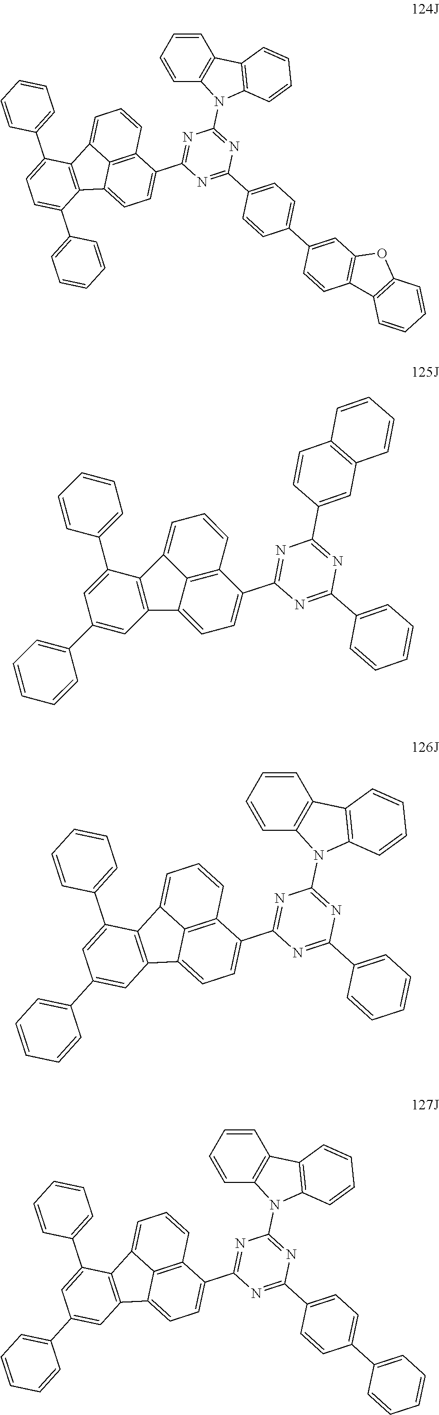 [SMILES: c1ccc(-c2ccc(-c3ccccc3)c3c2-c2cccc4c(-c5nc(-c6ccc(-c7ccc8c(c7)oc7ccccc78)cc6)nc(-n6c7ccccc7c7ccccc76)n5)ccc-3c24)cc1 ; c1ccc(-c2cc(-c3ccccc3)c3c(c2)-c2ccc(-c4nc(-c5ccccc5)nc(-c5ccc6ccccc6c5)n4)c4cccc-3c24)cc1 ; c1ccc(-c2cc(-c3ccccc3)c3c(c2)-c2ccc(-c4nc(-c5ccccc5)nc(-n5c6ccccc6c6ccccc65)n4)c4cccc-3c24)cc1 ; c1ccc(-c2ccc(-c3nc(-c4ccc5c6c(cccc46)-c4c(-c6ccccc6)cc(-c6ccccc6)cc4-5)nc(-n4c5ccccc5c5ccccc54)n3)cc2)cc1]

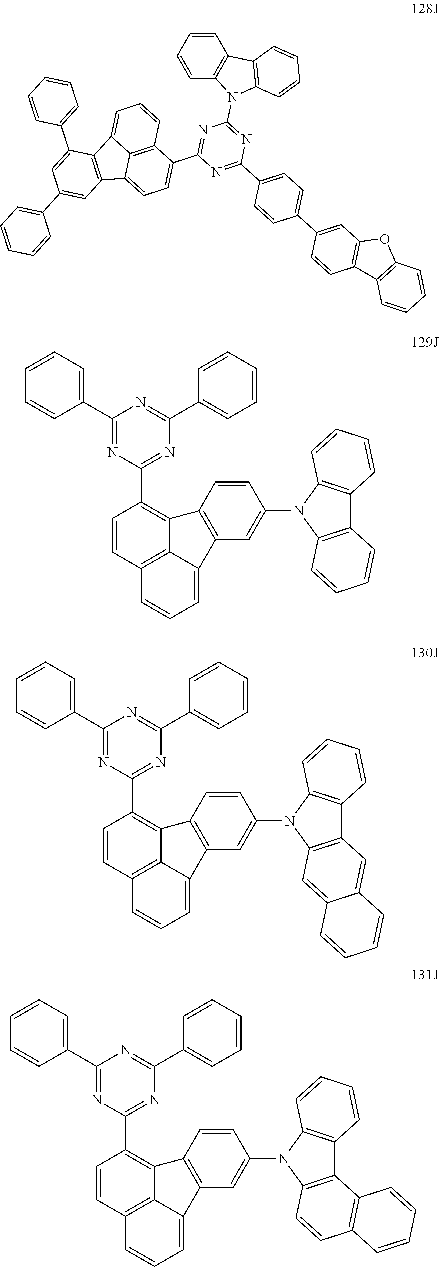 [SMILES: c1ccc(-c2cc(-c3ccccc3)c3c(c2)-c2ccc(-c4nc(-c5ccc(-c6ccc7c(c6)oc6ccccc67)cc5)nc(-n5c6ccccc6c6ccccc65)n4)c4cccc-3c24)cc1 ; c1ccc(-c2nc(-c3ccccc3)nc(-c3ccc4cccc5c4c3-c3ccc(-n4c6ccccc6c6ccccc64)cc3-5)n2)cc1 ; c1ccc(-c2nc(-c3ccccc3)nc(-c3ccc4cccc5c4c3-c3ccc(-n4c6ccccc6c6cc7ccccc7cc64)cc3-5)n2)cc1 ; c1ccc(-c2nc(-c3ccccc3)nc(-c3ccc4cccc5c4c3-c3ccc(-n4c6ccccc6c6c7ccccc7ccc64)cc3-5)n2)cc1]

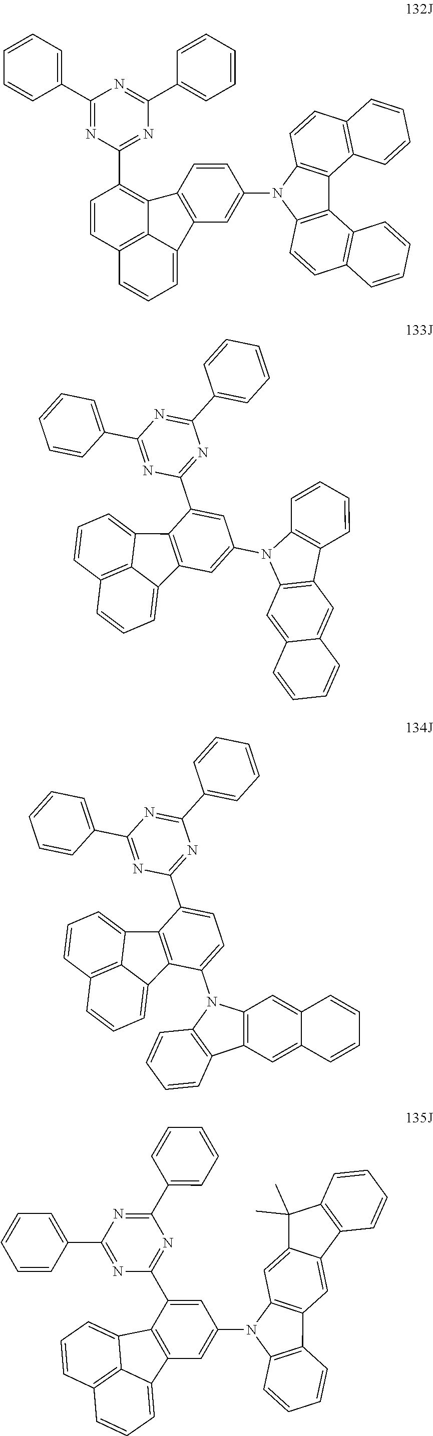

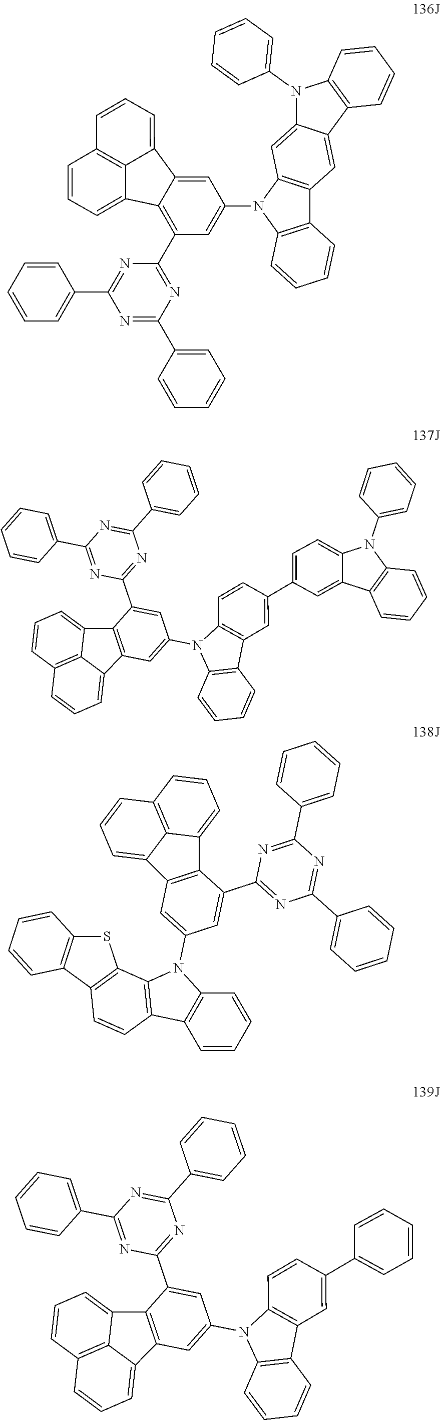 [SMILES: c1ccc(-c2nc(-c3ccccc3)nc(-c3cc(-n4c5ccccc5c5cc6c7ccccc7n(-c7ccccc7)c6cc54)cc4c3-c3cccc5cccc-4c35)n2)cc1 ; c1ccc(-c2nc(-c3ccccc3)nc(-c3cc(-n4c5ccccc5c5cc(-c6ccc7c(c6)c6ccccc6n7-c6ccccc6)ccc54)cc4c3-c3cccc5cccc-4c35)n2)cc1 ; c1ccc(-c2nc(-c3ccccc3)nc(-c3cc(-n4c5ccccc5c5ccc6c7ccccc7sc6c54)cc4c3-c3cccc5cccc-4c35)n2)cc1 ; c1ccc(-c2ccc3c(c2)c2ccccc2n3-c2cc(-c3nc(-c4ccccc4)nc(-c4ccccc4)n3)c3c(c2)-c2cccc4cccc-3c24)cc1]

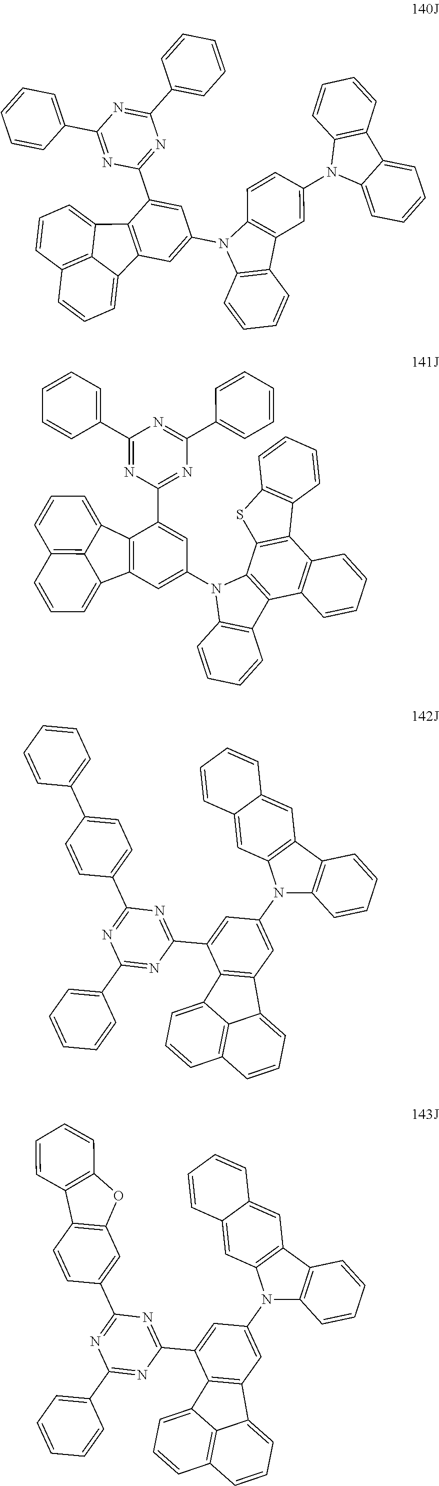

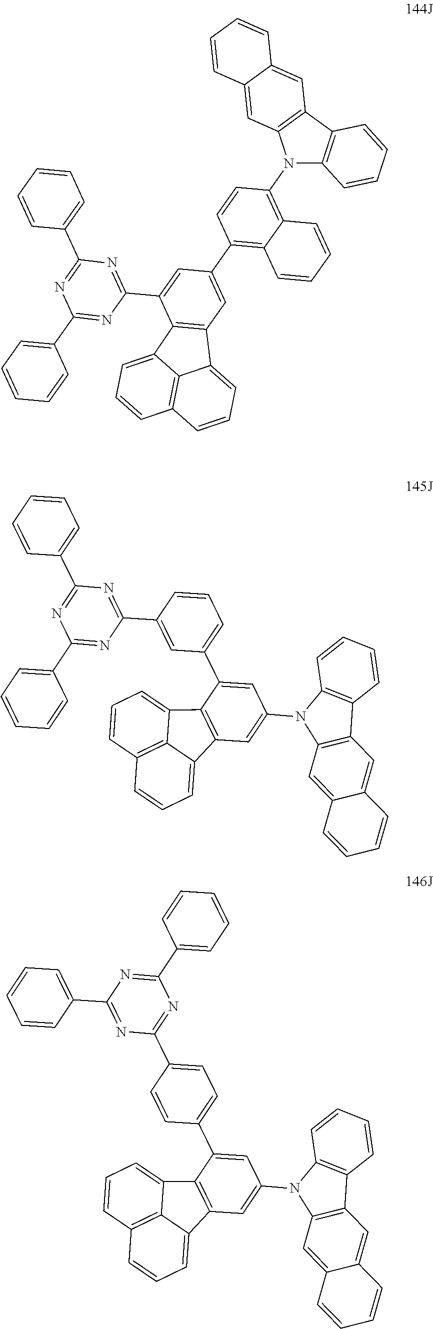 [SMILES: c1ccc(-c2nc(-c3ccccc3)nc(-c3cc(-c4ccc(-n5c6ccccc6c6cc7ccccc7cc65)c5ccccc45)cc4c3-c3cccc5cccc-4c35)n2)cc1 ; c1ccc(-c2nc(-c3ccccc3)nc(-c3cccc(-c4cc(-n5c6ccccc6c6cc7ccccc7cc65)cc5c4-c4cccc6cccc-5c46)c3)n2)cc1 ; c1ccc(-c2nc(-c3ccccc3)nc(-c3ccc(-c4cc(-n5c6ccccc6c6cc7ccccc7cc65)cc5c4-c4cccc6cccc-5c46)cc3)n2)cc1]

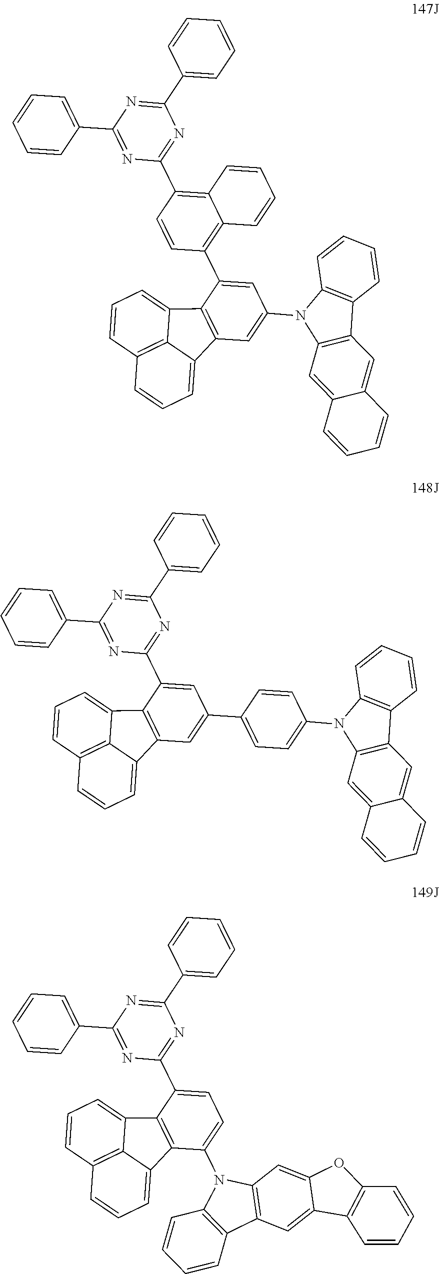 [SMILES: c1ccc(-c2nc(-c3ccccc3)nc(-c3ccc(-c4cc(-n5c6ccccc6c6cc7ccccc7cc65)cc5c4-c4cccc6cccc-5c46)c4ccccc34)n2)cc1 ; c1ccc(-c2nc(-c3ccccc3)nc(-c3cc(-c4ccc(-n5c6ccccc6c6cc7ccccc7cc65)cc4)cc4c3-c3cccc5cccc-4c35)n2)cc1 ; c1ccc(-c2nc(-c3ccccc3)nc(-c3ccc(-n4c5ccccc5c5cc6c(cc54)oc4ccccc46)c4c3-c3cccc5cccc-4c35)n2)cc1]

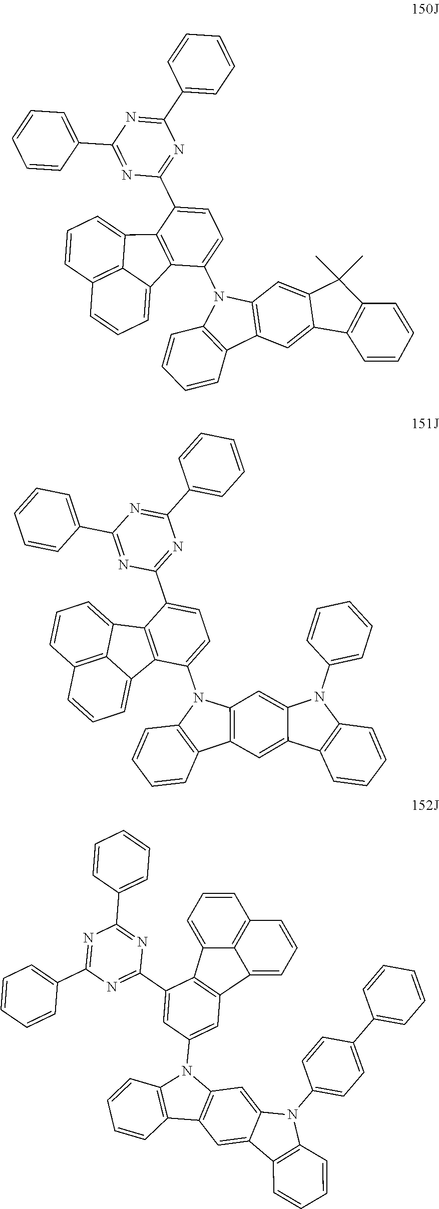

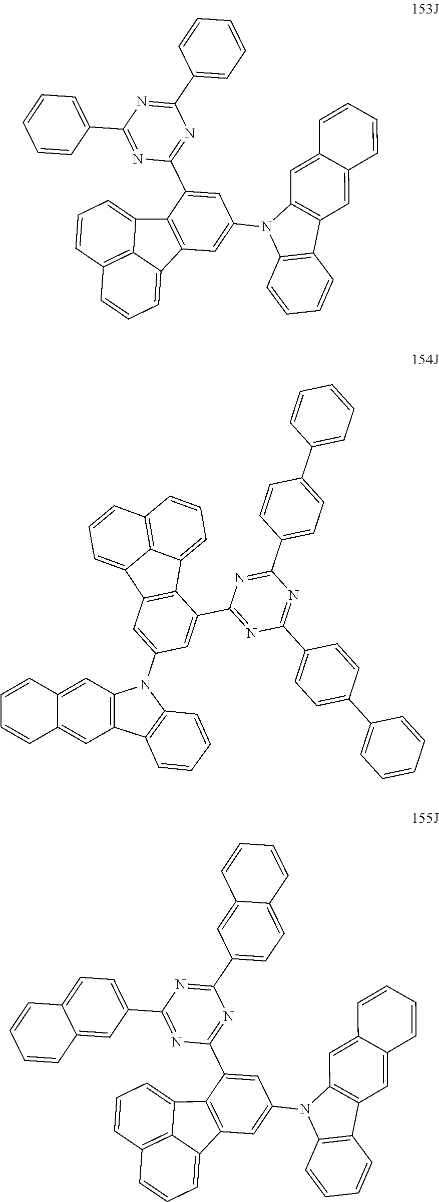 [SMILES: c1ccc(-c2nc(-c3ccccc3)nc(-c3cc(-n4c5ccccc5c5cc6ccccc6cc54)cc4c3-c3cccc5cccc-4c35)n2)cc1 ; c1ccc(-c2ccc(-c3nc(-c4ccc(-c5ccccc5)cc4)nc(-c4cc(-n5c6ccccc6c6cc7ccccc7cc65)cc5c4-c4cccc6cccc-5c46)n3)cc2)cc1 ; c1ccc2cc(-c3nc(-c4ccc5ccccc5c4)nc(-c4cc(-n5c6ccccc6c6cc7ccccc7cc65)cc5c4-c4cccc6cccc-5c46)n3)ccc2c1]

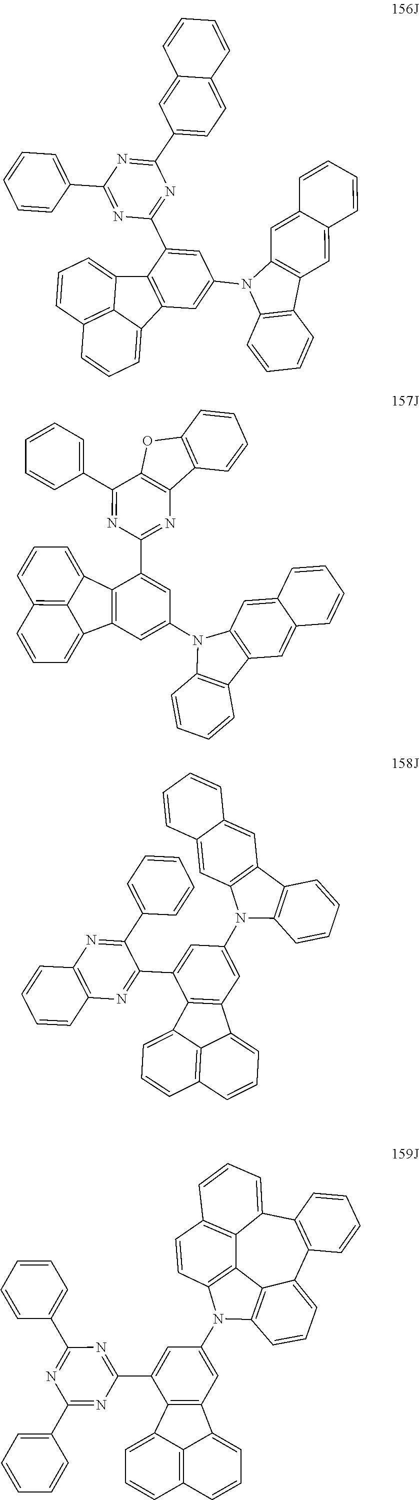

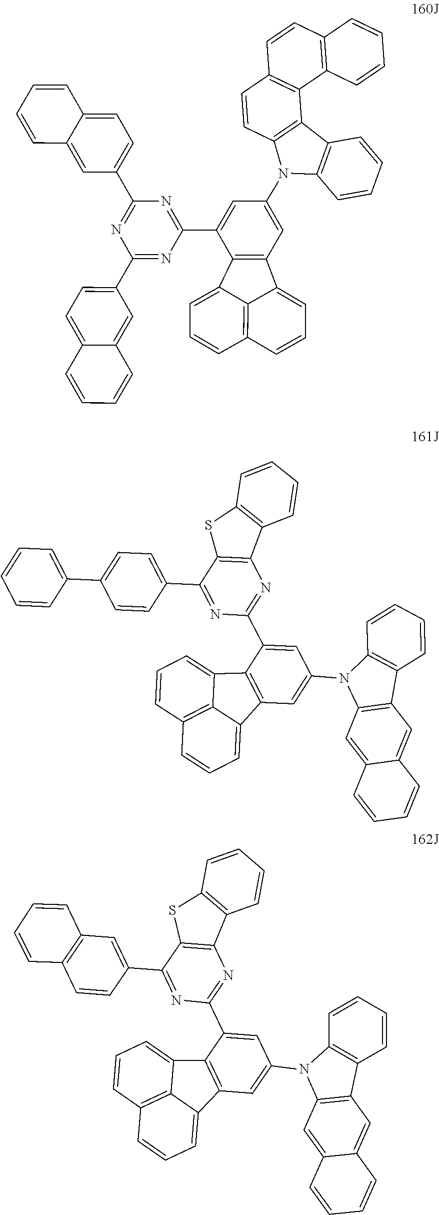 [SMILES: c1ccc2cc(-c3nc(-c4ccc5ccccc5c4)nc(-c4cc(-n5c6ccccc6c6c7c(ccc8ccccc87)ccc65)cc5c4-c4cccc6cccc-5c46)n3)ccc2c1 ; c1ccc(-c2ccc(-c3nc(-c4cc(-n5c6ccccc6c6cc7ccccc7cc65)cc5c4-c4cccc6cccc-5c46)nc4c3sc3ccccc34)cc2)cc1 ; c1ccc2cc(-c3nc(-c4cc(-n5c6ccccc6c6cc7ccccc7cc65)cc5c4-c4cccc6cccc-5c46)nc4c3sc3ccccc34)ccc2c1]

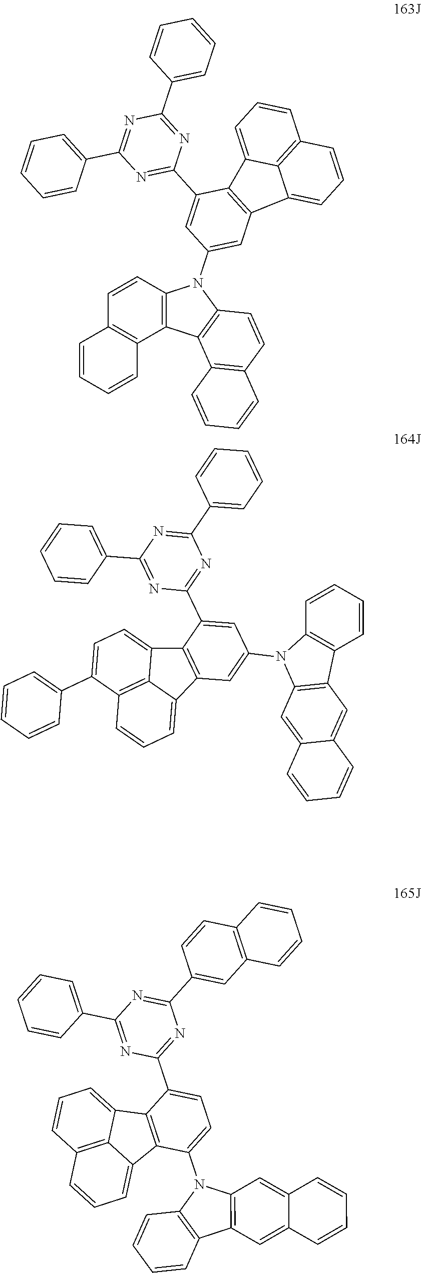

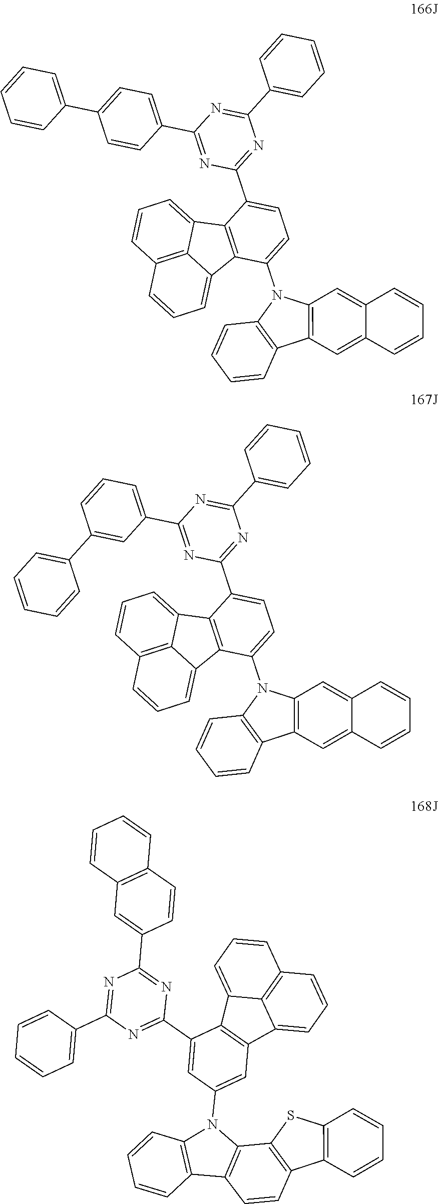 [SMILES: c1ccc(-c2ccc(-c3nc(-c4ccccc4)nc(-c4ccc(-n5c6ccccc6c6cc7ccccc7cc65)c5c4-c4cccc6cccc-5c46)n3)cc2)cc1 ; c1ccc(-c2cccc(-c3nc(-c4ccccc4)nc(-c4ccc(-n5c6ccccc6c6cc7ccccc7cc65)c5c4-c4cccc6cccc-5c46)n3)c2)cc1 ; c1ccc(-c2nc(-c3ccc4ccccc4c3)nc(-c3cc(-n4c5ccccc5c5ccc6c7ccccc7sc6c54)cc4c3-c3cccc5cccc-4c35)n2)cc1]

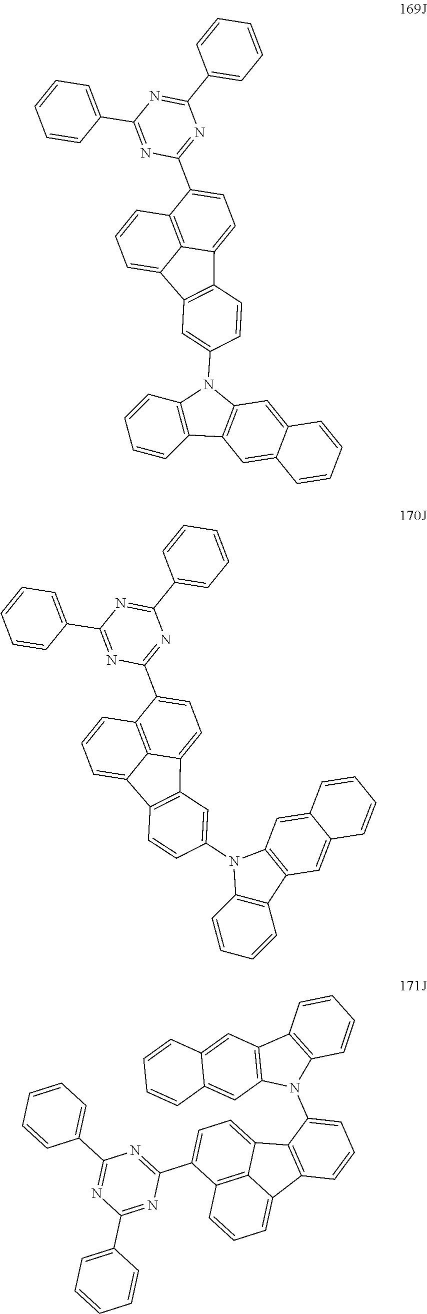 [SMILES: c1ccc(-c2nc(-c3ccccc3)nc(-c3ccc4c5c(cccc35)-c3cc(-n5c6ccccc6c6cc7ccccc7cc65)ccc3-4)n2)cc1 ; c1ccc(-c2nc(-c3ccccc3)nc(-c3ccc4c5c(cccc35)-c3ccc(-n5c6ccccc6c6cc7ccccc7cc65)cc3-4)n2)cc1 ; c1ccc(-c2nc(-c3ccccc3)nc(-c3ccc4c5c(cccc35)-c3cccc(-n5c6ccccc6c6cc7ccccc7cc65)c3-4)n2)cc1]

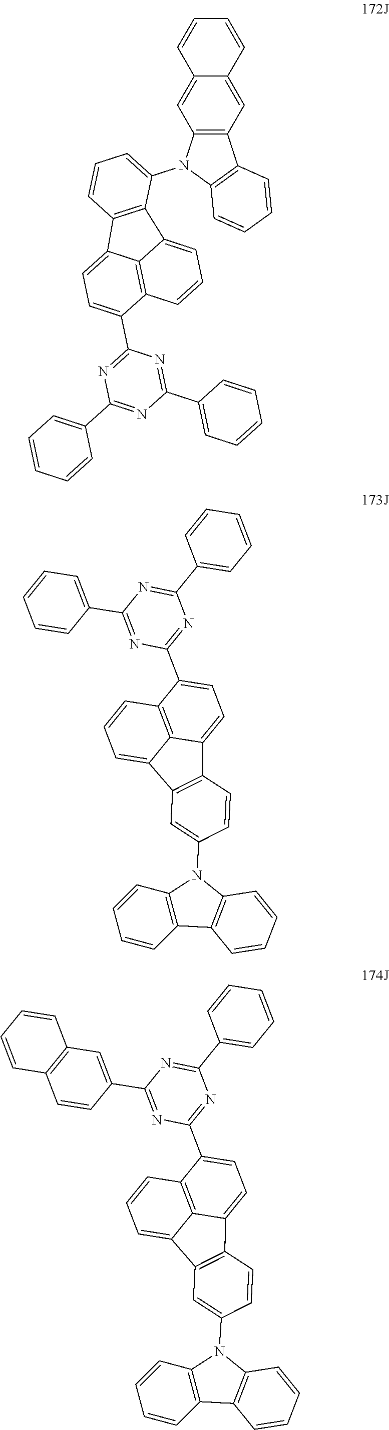

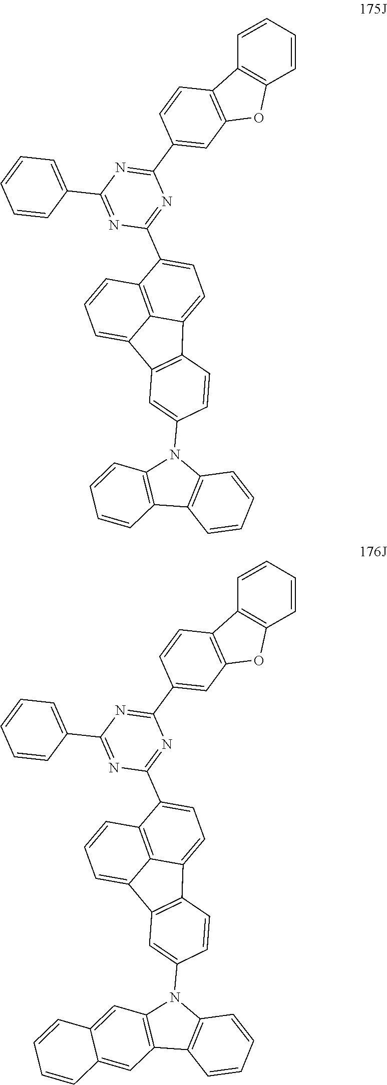

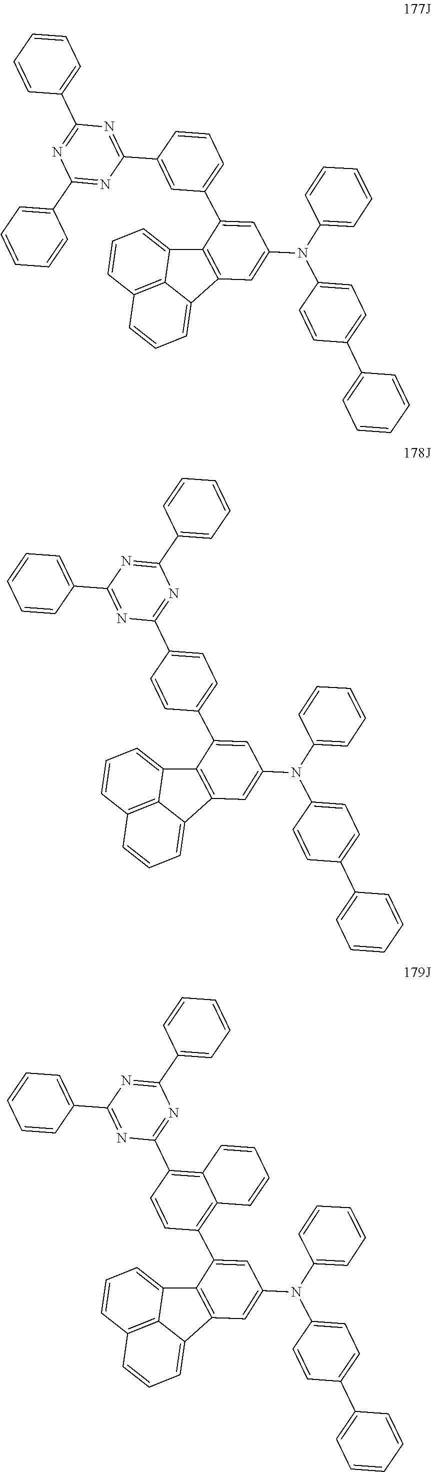 [SMILES: c1ccc(-c2ccc(N(c3ccccc3)c3cc(-c4cccc(-c5nc(-c6ccccc6)nc(-c6ccccc6)n5)c4)c4c(c3)-c3cccc5cccc-4c35)cc2)cc1 ; c1ccc(-c2ccc(N(c3ccccc3)c3cc(-c4ccc(-c5nc(-c6ccccc6)nc(-c6ccccc6)n5)cc4)c4c(c3)-c3cccc5cccc-4c35)cc2)cc1 ; c1ccc(-c2ccc(N(c3ccccc3)c3cc(-c4ccc(-c5nc(-c6ccccc6)nc(-c6ccccc6)n5)c5ccccc45)c4c(c3)-c3cccc5cccc-4c35)cc2)cc1]

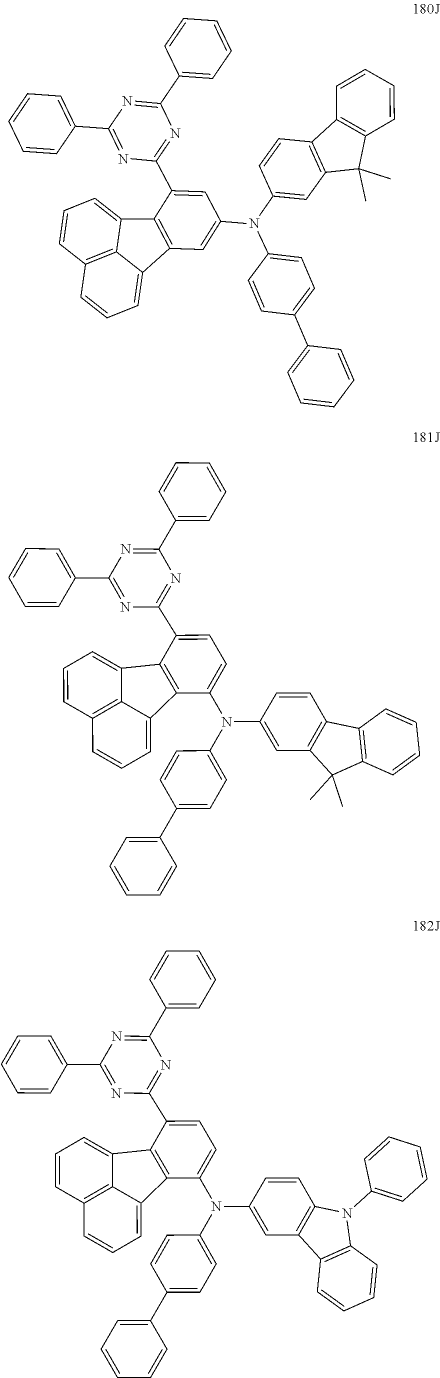 [SMILES: CC1(C)c2ccccc2-c2ccc(N(c3ccc(-c4ccccc4)cc3)c3cc(-c4nc(-c5ccccc5)nc(-c5ccccc5)n4)c4c(c3)-c3cccc5cccc-4c35)cc21 ; CC1(C)c2ccccc2-c2ccc(N(c3ccc(-c4ccccc4)cc3)c3ccc(-c4nc(-c5ccccc5)nc(-c5ccccc5)n4)c4c3-c3cccc5cccc-4c35)cc21 ; c1ccc(-c2ccc(N(c3ccc4c(c3)c3ccccc3n4-c3ccccc3)c3ccc(-c4nc(-c5ccccc5)nc(-c5ccccc5)n4)c4c3-c3cccc5cccc-4c35)cc2)cc1]

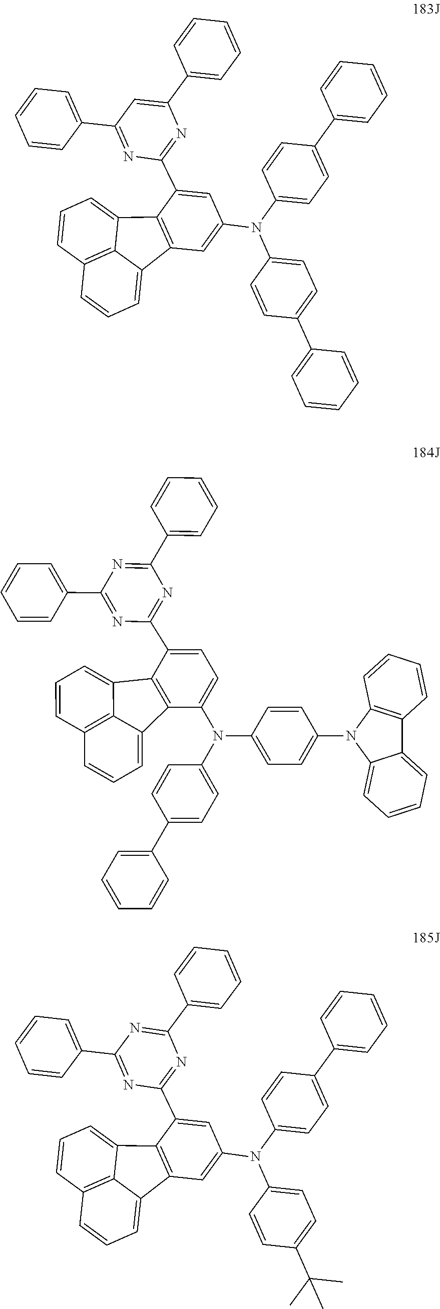 [SMILES: c1ccc(-c2ccc(N(c3ccc(-c4ccccc4)cc3)c3cc(-c4nc(-c5ccccc5)cc(-c5ccccc5)n4)c4c(c3)-c3cccc5cccc-4c35)cc2)cc1 ; c1ccc(-c2ccc(N(c3ccc(-n4c5ccccc5c5ccccc54)cc3)c3ccc(-c4nc(-c5ccccc5)nc(-c5ccccc5)n4)c4c3-c3cccc5cccc-4c35)cc2)cc1 ; CC(C)(C)c1ccc(N(c2ccc(-c3ccccc3)cc2)c2cc(-c3nc(-c4ccccc4)nc(-c4ccccc4)n3)c3c(c2)-c2cccc4cccc-3c24)cc1]

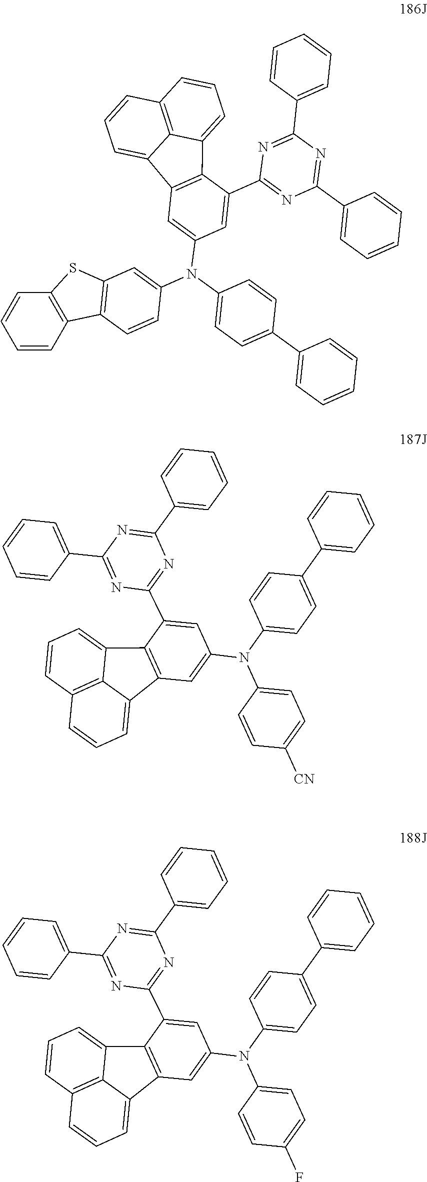 [SMILES: c1ccc(-c2ccc(N(c3cc(-c4nc(-c5ccccc5)nc(-c5ccccc5)n4)c4c(c3)-c3cccc5cccc-4c35)c3ccc4c(c3)sc3ccccc34)cc2)cc1 ; N#Cc1ccc(N(c2ccc(-c3ccccc3)cc2)c2cc(-c3nc(-c4ccccc4)nc(-c4ccccc4)n3)c3c(c2)-c2cccc4cccc-3c24)cc1 ; Fc1ccc(N(c2ccc(-c3ccccc3)cc2)c2cc(-c3nc(-c4ccccc4)nc(-c4ccccc4)n3)c3c(c2)-c2cccc4cccc-3c24)cc1]

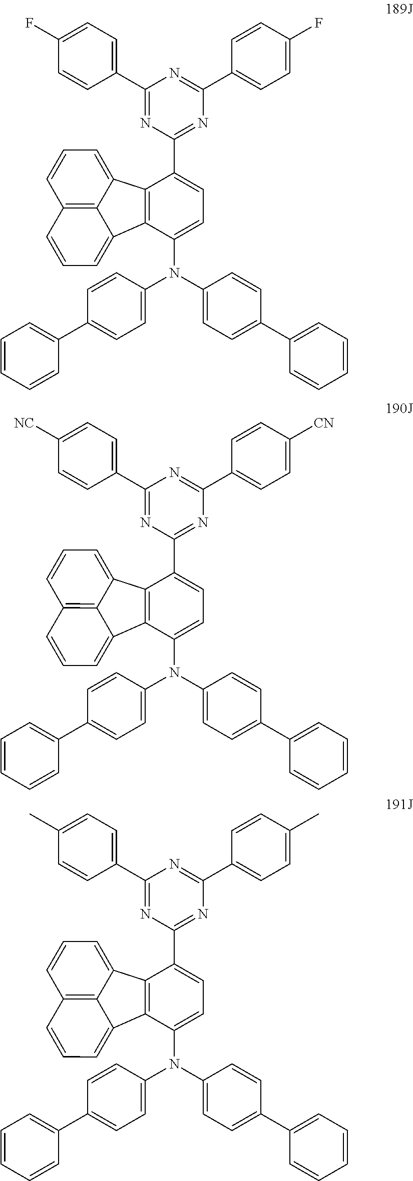

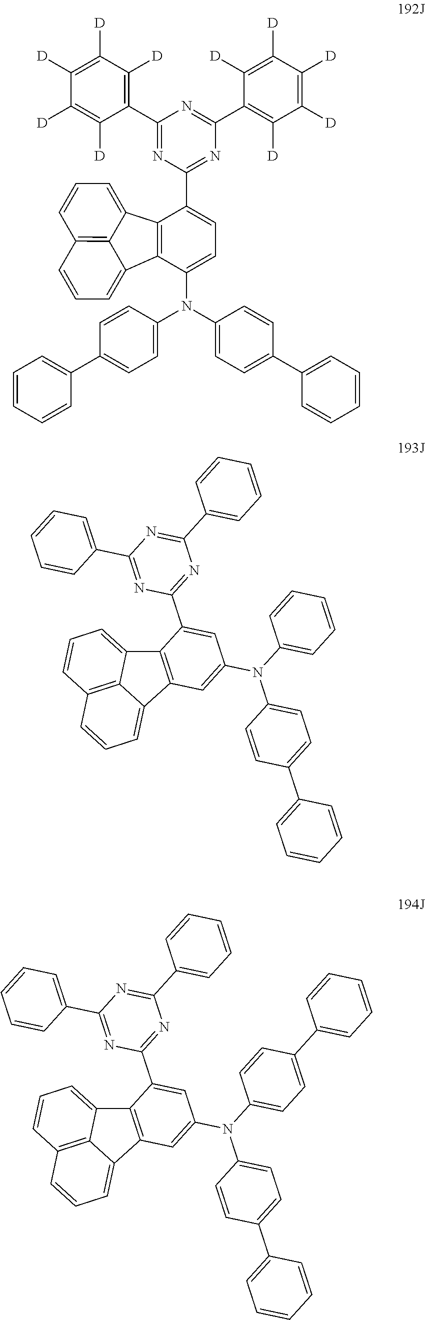 [SMILES: [2H]c1c([2H])c([2H])c(-c2nc(-c3ccc(N(c4ccc(-c5ccccc5)cc4)c4ccc(-c5ccccc5)cc4)c4c3-c3cccc5cccc-4c35)nc(-c3c([2H])c([2H])c([2H])c([2H])c3[2H])n2)c([2H])c1[2H] ; c1ccc(-c2ccc(N(c3ccccc3)c3cc(-c4nc(-c5ccccc5)nc(-c5ccccc5)n4)c4c(c3)-c3cccc5cccc-4c35)cc2)cc1 ; c1ccc(-c2ccc(N(c3ccc(-c4ccccc4)cc3)c3cc(-c4nc(-c5ccccc5)nc(-c5ccccc5)n4)c4c(c3)-c3cccc5cccc-4c35)cc2)cc1]

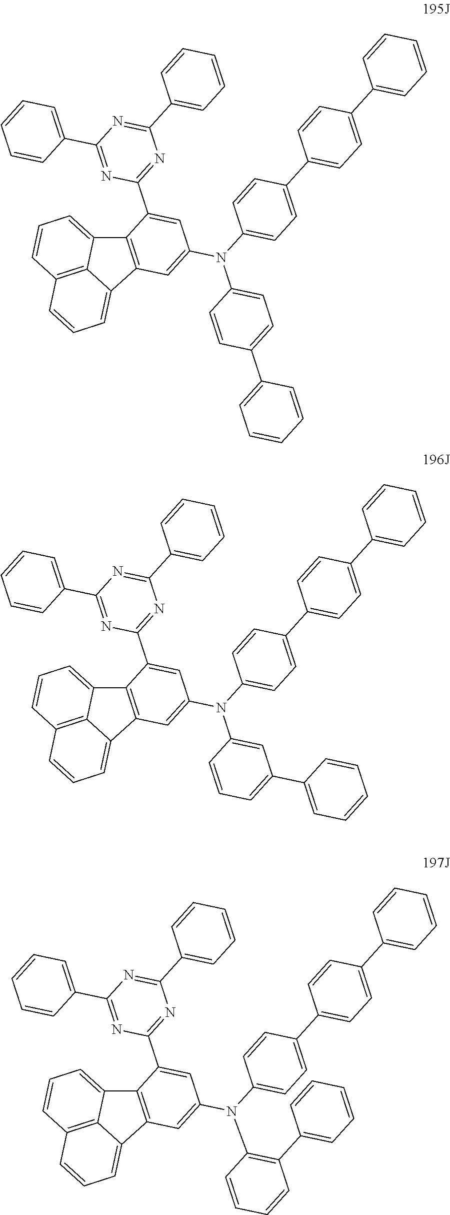 [SMILES: c1ccc(-c2ccc(-c3ccc(N(c4ccc(-c5ccccc5)cc4)c4cc(-c5nc(-c6ccccc6)nc(-c6ccccc6)n5)c5c(c4)-c4cccc6cccc-5c46)cc3)cc2)cc1 ; c1ccc(-c2ccc(-c3ccc(N(c4cccc(-c5ccccc5)c4)c4cc(-c5nc(-c6ccccc6)nc(-c6ccccc6)n5)c5c(c4)-c4cccc6cccc-5c46)cc3)cc2)cc1 ; c1ccc(-c2ccc(-c3ccc(N(c4cc(-c5nc(-c6ccccc6)nc(-c6ccccc6)n5)c5c(c4)-c4cccc6cccc-5c46)c4ccccc4-c4ccccc4)cc3)cc2)cc1]

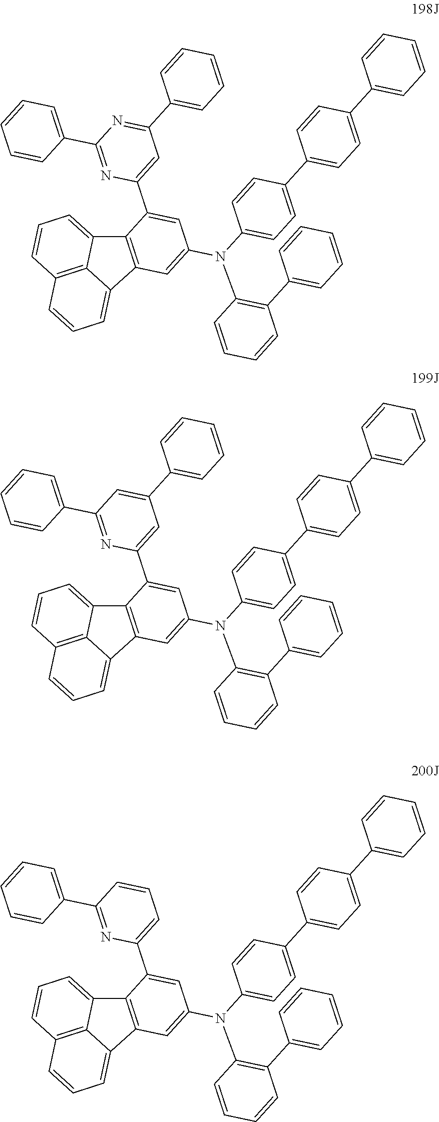 [SMILES: c1ccc(-c2ccc(-c3ccc(N(c4cc(-c5cc(-c6ccccc6)nc(-c6ccccc6)n5)c5c(c4)-c4cccc6cccc-5c46)c4ccccc4-c4ccccc4)cc3)cc2)cc1 ; c1ccc(-c2ccc(-c3ccc(N(c4cc(-c5cc(-c6ccccc6)cc(-c6ccccc6)n5)c5c(c4)-c4cccc6cccc-5c46)c4ccccc4-c4ccccc4)cc3)cc2)cc1 ; c1ccc(-c2ccc(-c3ccc(N(c4cc(-c5cccc(-c6ccccc6)n5)c5c(c4)-c4cccc6cccc-5c46)c4ccccc4-c4ccccc4)cc3)cc2)cc1]

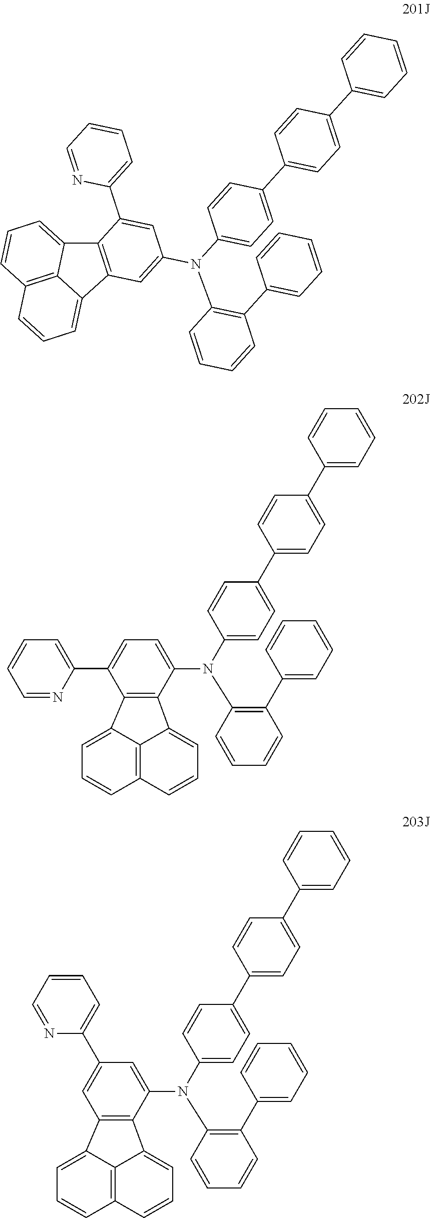 [SMILES: c1ccc(-c2ccc(-c3ccc(N(c4cc(-c5ccccn5)c5c(c4)-c4cccc6cccc-5c46)c4ccccc4-c4ccccc4)cc3)cc2)cc1 ; c1ccc(-c2ccc(-c3ccc(N(c4ccccc4-c4ccccc4)c4ccc(-c5ccccn5)c5c4-c4cccc6cccc-5c46)cc3)cc2)cc1 ; c1ccc(-c2ccc(-c3ccc(N(c4ccccc4-c4ccccc4)c4cc(-c5ccccn5)cc5c4-c4cccc6cccc-5c46)cc3)cc2)cc1]

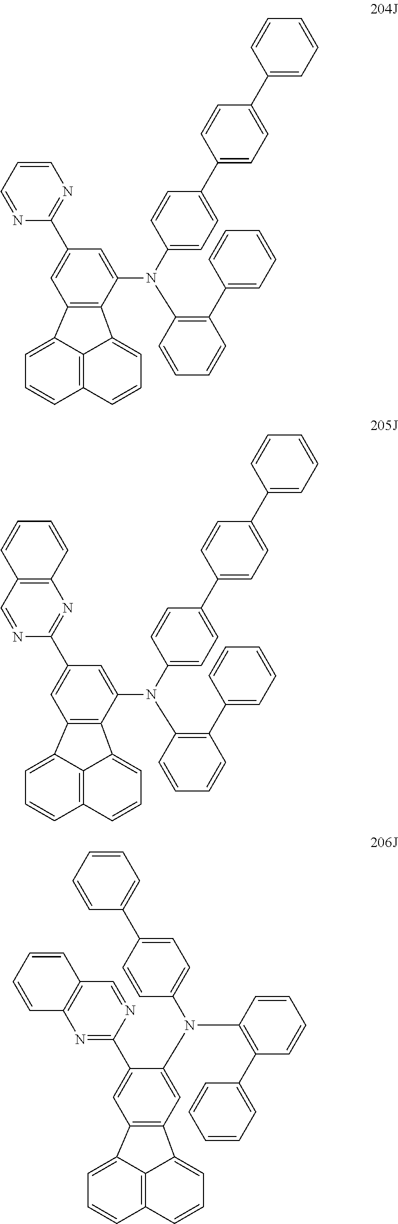

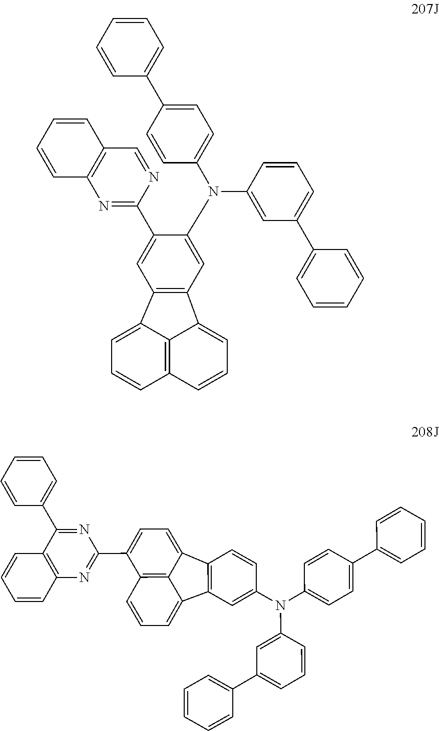 [SMILES: c1ccc(-c2ccc(N(c3cccc(-c4ccccc4)c3)c3cc4c(cc3-c3ncc5ccccc5n3)-c3cccc5cccc-4c35)cc2)cc1 ; c1ccc(-c2ccc(N(c3cccc(-c4ccccc4)c3)c3ccc4c(c3)-c3cccc5c(-c6nc(-c7ccccc7)c7ccccc7n6)ccc-4c35)cc2)cc1]

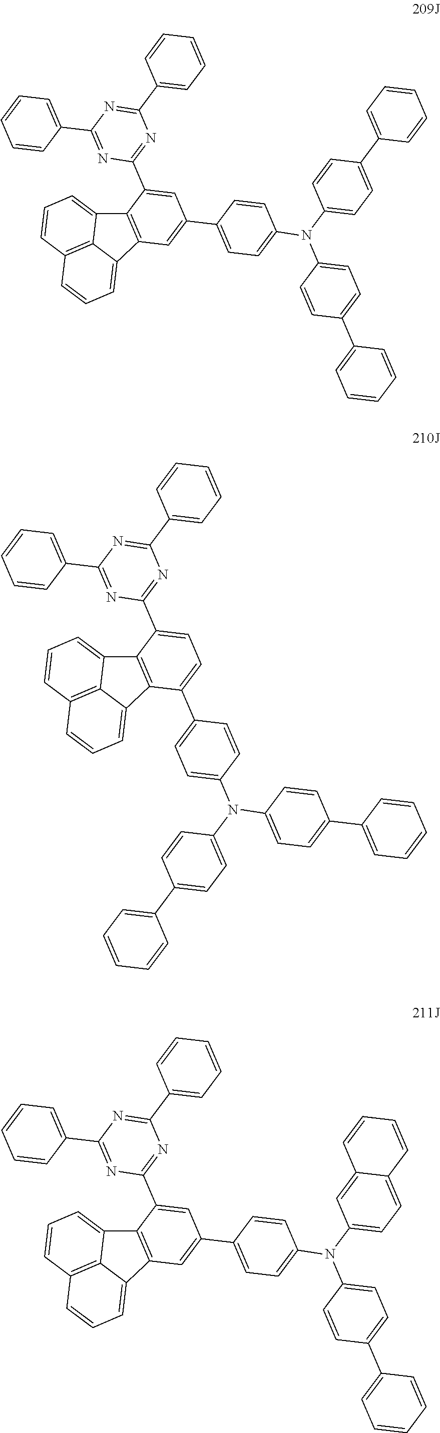 [SMILES: c1ccc(-c2ccc(N(c3ccc(-c4ccccc4)cc3)c3ccc(-c4cc(-c5nc(-c6ccccc6)nc(-c6ccccc6)n5)c5c(c4)-c4cccc6cccc-5c46)cc3)cc2)cc1 ; c1ccc(-c2ccc(N(c3ccc(-c4ccccc4)cc3)c3ccc(-c4ccc(-c5nc(-c6ccccc6)nc(-c6ccccc6)n5)c5c4-c4cccc6cccc-5c46)cc3)cc2)cc1 ; c1ccc(-c2ccc(N(c3ccc(-c4cc(-c5nc(-c6ccccc6)nc(-c6ccccc6)n5)c5c(c4)-c4cccc6cccc-5c46)cc3)c3ccc4ccccc4c3)cc2)cc1]

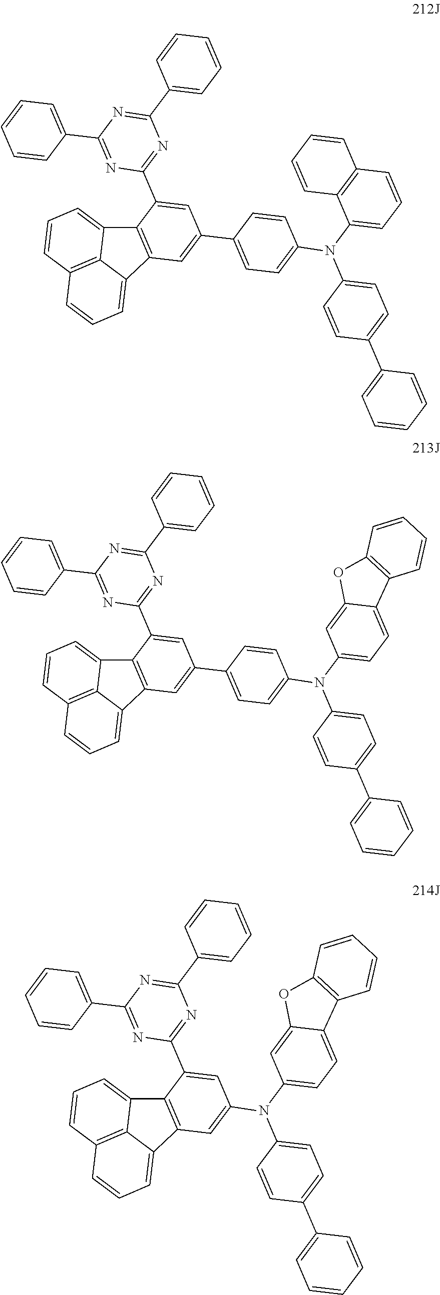 [SMILES: c1ccc(-c2ccc(N(c3ccc(-c4cc(-c5nc(-c6ccccc6)nc(-c6ccccc6)n5)c5c(c4)-c4cccc6cccc-5c46)cc3)c3cccc4ccccc34)cc2)cc1 ; c1ccc(-c2ccc(N(c3ccc(-c4cc(-c5nc(-c6ccccc6)nc(-c6ccccc6)n5)c5c(c4)-c4cccc6cccc-5c46)cc3)c3ccc4c(c3)oc3ccccc34)cc2)cc1 ; c1ccc(-c2ccc(N(c3cc(-c4nc(-c5ccccc5)nc(-c5ccccc5)n4)c4c(c3)-c3cccc5cccc-4c35)c3ccc4c(c3)oc3ccccc34)cc2)cc1]

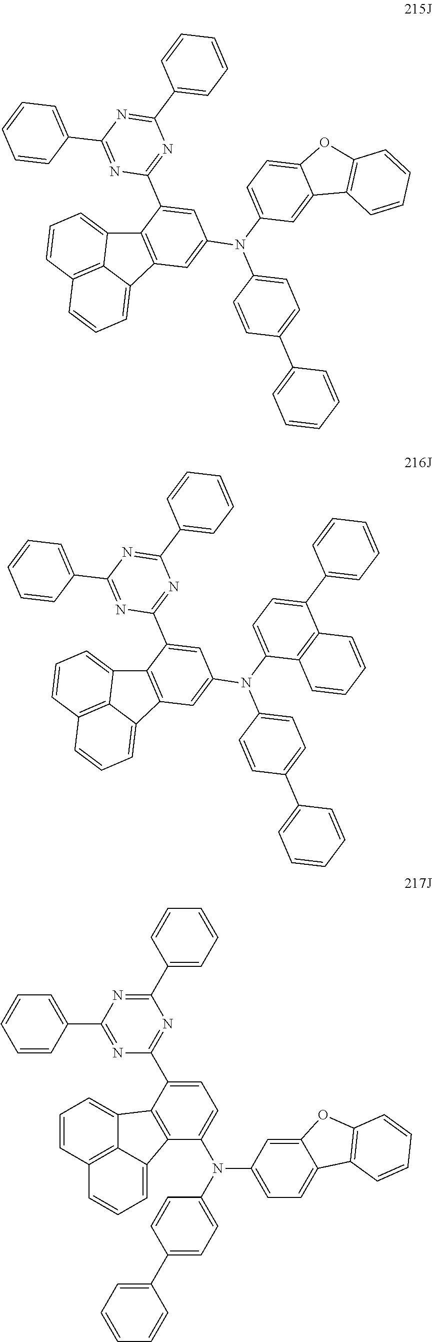 [SMILES: c1ccc(-c2ccc(N(c3cc(-c4nc(-c5ccccc5)nc(-c5ccccc5)n4)c4c(c3)-c3cccc5cccc-4c35)c3ccc4oc5ccccc5c4c3)cc2)cc1 ; c1ccc(-c2ccc(N(c3cc(-c4nc(-c5ccccc5)nc(-c5ccccc5)n4)c4c(c3)-c3cccc5cccc-4c35)c3ccc(-c4ccccc4)c4ccccc34)cc2)cc1 ; c1ccc(-c2ccc(N(c3ccc4c(c3)oc3ccccc34)c3ccc(-c4nc(-c5ccccc5)nc(-c5ccccc5)n4)c4c3-c3cccc5cccc-4c35)cc2)cc1]

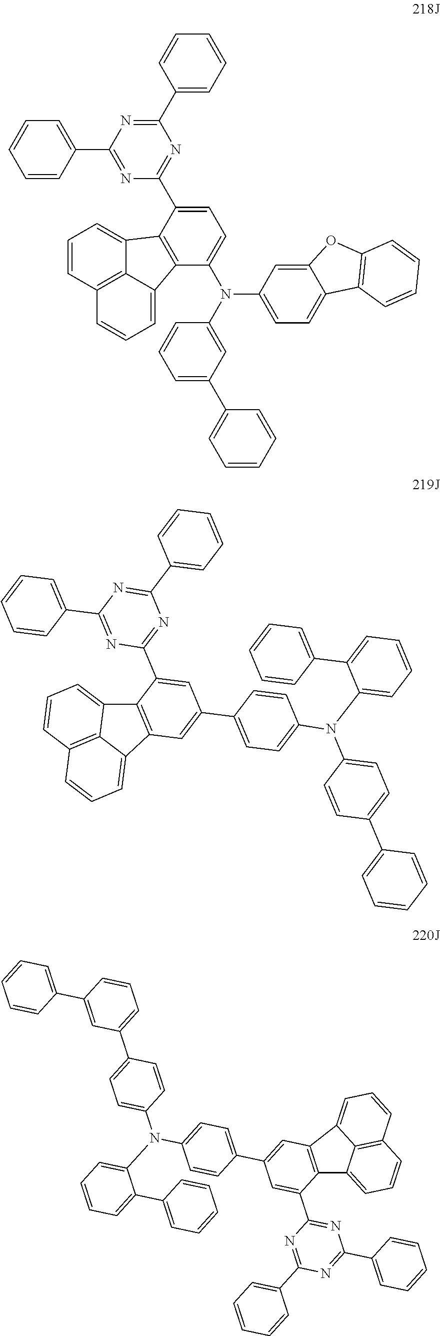 [SMILES: c1ccc(-c2cccc(N(c3ccc4c(c3)oc3ccccc34)c3ccc(-c4nc(-c5ccccc5)nc(-c5ccccc5)n4)c4c3-c3cccc5cccc-4c35)c2)cc1 ; c1ccc(-c2ccc(N(c3ccc(-c4cc(-c5nc(-c6ccccc6)nc(-c6ccccc6)n5)c5c(c4)-c4cccc6cccc-5c46)cc3)c3ccccc3-c3ccccc3)cc2)cc1 ; c1ccc(-c2cccc(-c3ccc(N(c4ccc(-c5cc(-c6nc(-c7ccccc7)nc(-c7ccccc7)n6)c6c(c5)-c5cccc7cccc-6c57)cc4)c4ccccc4-c4ccccc4)cc3)c2)cc1]

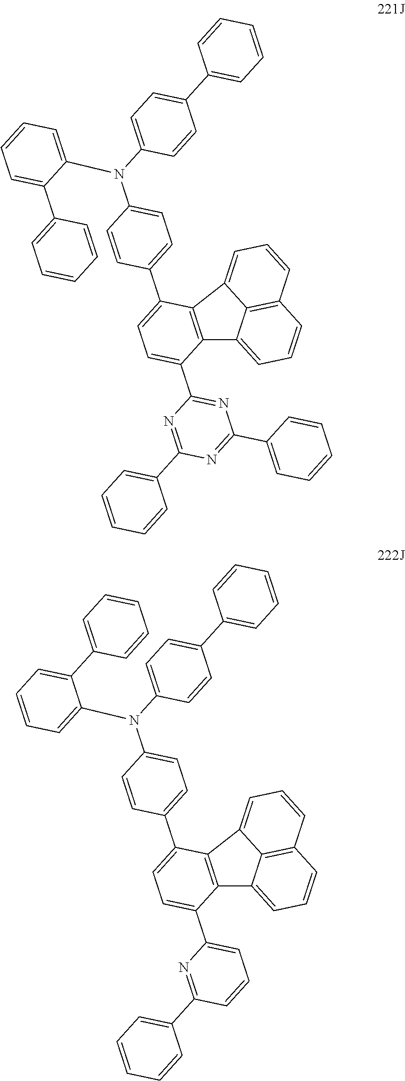 [SMILES: c1ccc(-c2ccc(N(c3ccc(-c4ccc(-c5nc(-c6ccccc6)nc(-c6ccccc6)n5)c5c4-c4cccc6cccc-5c46)cc3)c3ccccc3-c3ccccc3)cc2)cc1 ; c1ccc(-c2ccc(N(c3ccc(-c4ccc(-c5cccc(-c6ccccc6)n5)c5c4-c4cccc6cccc-5c46)cc3)c3ccccc3-c3ccccc3)cc2)cc1]

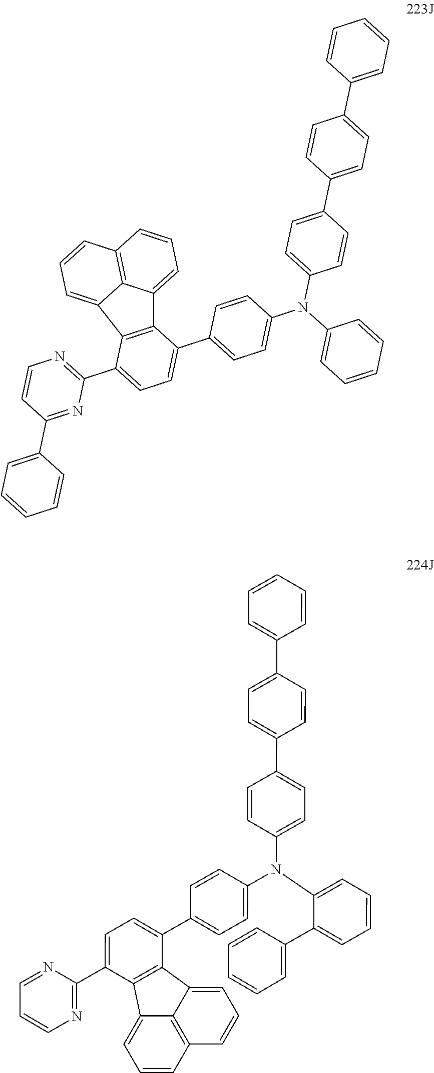 [SMILES: c1ccc(-c2ccc(-c3ccc(N(c4ccccc4)c4ccc(-c5ccc(-c6nccc(-c7ccccc7)n6)c6c5-c5cccc7cccc-6c57)cc4)cc3)cc2)cc1 ; c1ccc(-c2ccc(-c3ccc(N(c4ccc(-c5ccc(-c6ncccn6)c6c5-c5cccc7cccc-6c57)cc4)c4ccccc4-c4ccccc4)cc3)cc2)cc1]

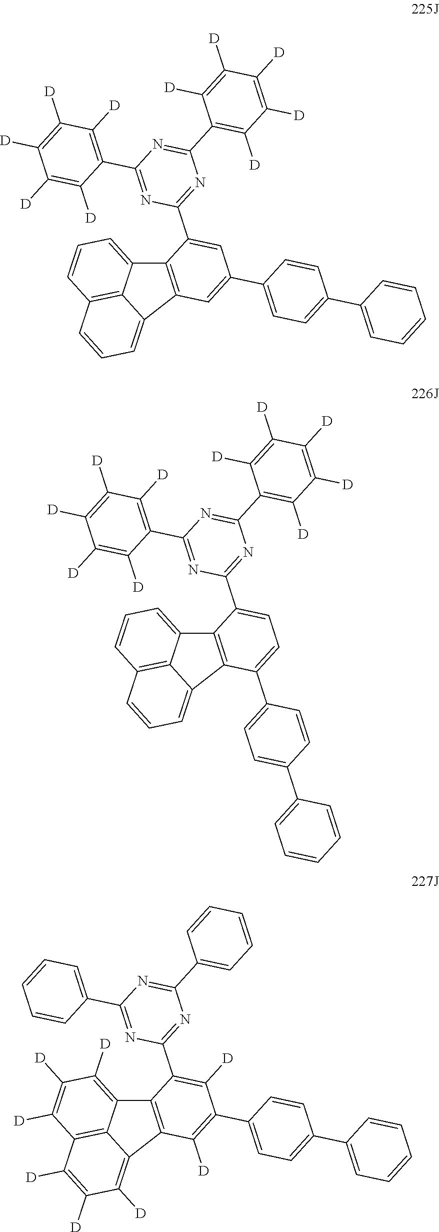 [SMILES: [2H]c1c([2H])c([2H])c(-c2nc(-c3cc(-c4ccc(-c5ccccc5)cc4)cc4c3-c3cccc5cccc-4c35)nc(-c3c([2H])c([2H])c([2H])c([2H])c3[2H])n2)c([2H])c1[2H] ; [2H]c1c([2H])c([2H])c(-c2nc(-c3ccc(-c4ccc(-c5ccccc5)cc4)c4c3-c3cccc5cccc-4c35)nc(-c3c([2H])c([2H])c([2H])c([2H])c3[2H])n2)c([2H])c1[2H] ; [2H]c1c(-c2ccc(-c3ccccc3)cc2)c([2H])c2c(c1-c1nc(-c3ccccc3)nc(-c3ccccc3)n1)-c1c([2H])c([2H])c([2H])c3c([2H])c([2H])c([2H])c-2c13]

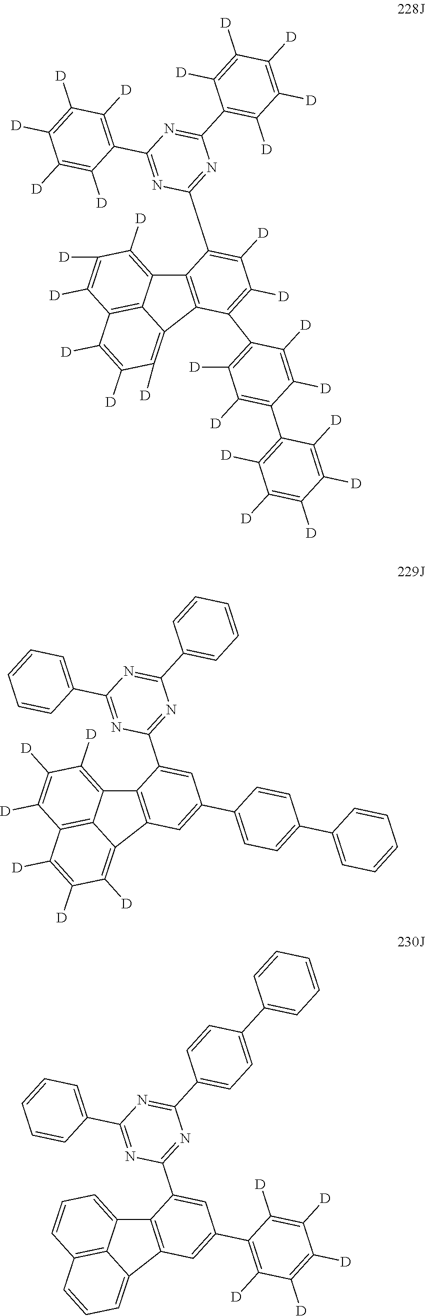 [SMILES: [2H]c1c([2H])c([2H])c(-c2nc(-c3c([2H])c([2H])c([2H])c([2H])c3[2H])nc(-c3c([2H])c([2H])c(-c4c([2H])c([2H])c(-c5c([2H])c([2H])c([2H])c([2H])c5[2H])c([2H])c4[2H])c4c3-c3c([2H])c([2H])c([2H])c5c([2H])c([2H])c([2H])c-4c35)n2)c([2H])c1[2H] ; [2H]c1c([2H])c2c3c(c([2H])c([2H])c([2H])c3c1[2H])-c1c(-c3nc(-c4ccccc4)nc(-c4ccccc4)n3)cc(-c3ccc(-c4ccccc4)cc3)cc1-2 ; [2H]c1c([2H])c([2H])c(-c2cc(-c3nc(-c4ccccc4)nc(-c4ccc(-c5ccccc5)cc4)n3)c3c(c2)-c2cccc4cccc-3c24)c([2H])c1[2H]]

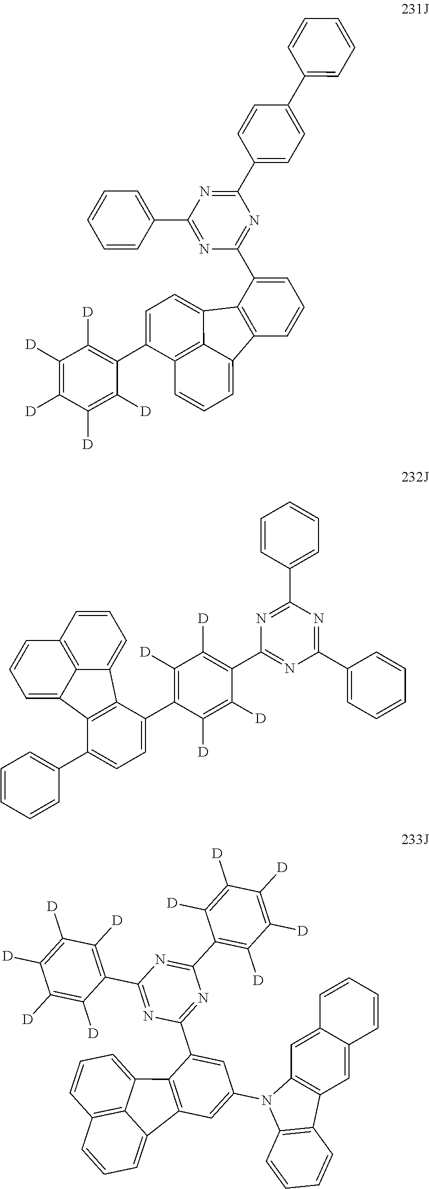 [SMILES: [2H]c1c([2H])c([2H])c(-c2ccc3c4c(cccc24)-c2cccc(-c4nc(-c5ccccc5)nc(-c5ccc(-c6ccccc6)cc5)n4)c2-3)c([2H])c1[2H] ; [2H]c1c([2H])c(-c2ccc(-c3ccccc3)c3c2-c2cccc4cccc-3c24)c([2H])c([2H])c1-c1nc(-c2ccccc2)nc(-c2ccccc2)n1 ; [2H]c1c([2H])c([2H])c(-c2nc(-c3cc(-n4c5ccccc5c5cc6ccccc6cc54)cc4c3-c3cccc5cccc-4c35)nc(-c3c([2H])c([2H])c([2H])c([2H])c3[2H])n2)c([2H])c1[2H]]

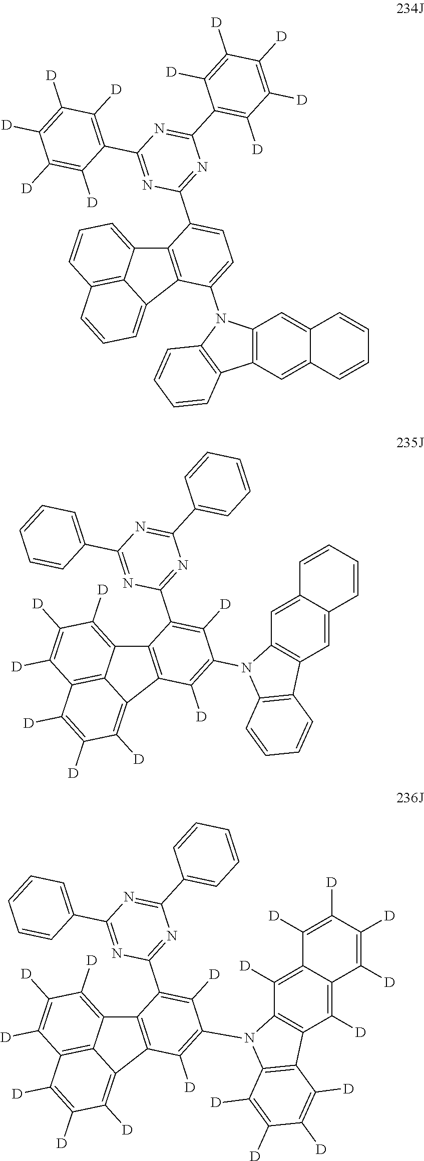 [SMILES: [2H]c1c([2H])c([2H])c(-c2nc(-c3ccc(-n4c5ccccc5c5cc6ccccc6cc54)c4c3-c3cccc5cccc-4c35)nc(-c3c([2H])c([2H])c([2H])c([2H])c3[2H])n2)c([2H])c1[2H] ; [2H]c1c(-c2nc(-c3ccccc3)nc(-c3ccccc3)n2)c2c(c([2H])c1-n1c3ccccc3c3cc4ccccc4cc31)-c1c([2H])c([2H])c([2H])c3c([2H])c([2H])c([2H])c-2c13 ; [2H]c1c(-c2nc(-c3ccccc3)nc(-c3ccccc3)n2)c2c(c([2H])c1-n1c3c([2H])c([2H])c([2H])c([2H])c3c3c([2H])c4c([2H])c([2H])c([2H])c([2H])c4c([2H])c31)-c1c([2H])c([2H])c([2H])c3c([2H])c([2H])c([2H])c-2c13]

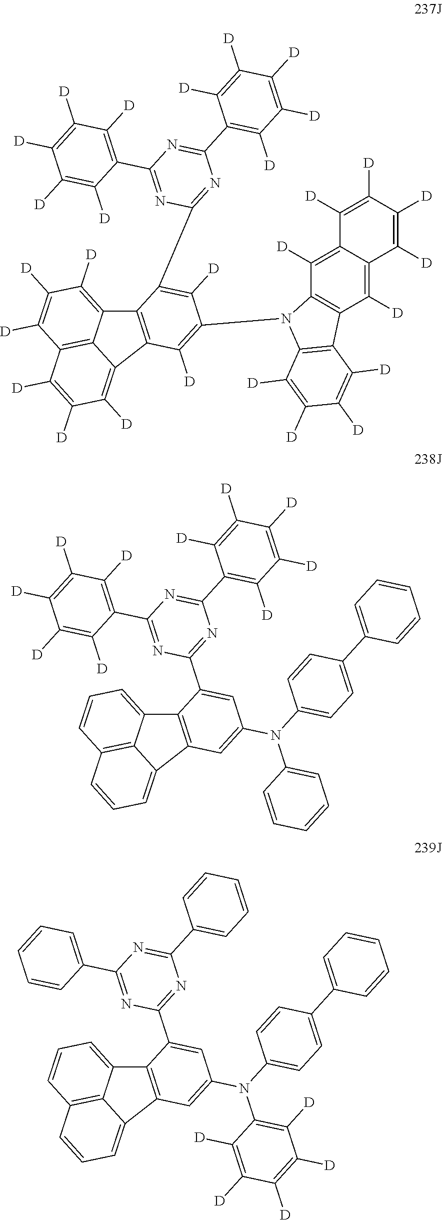

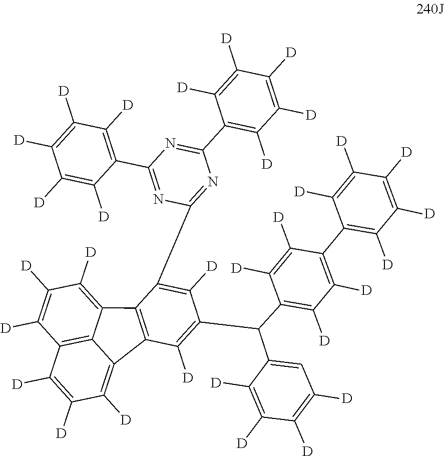 [SMILES: [2H]c1cc(C(c2c([2H])c([2H])c(-c3c([2H])c([2H])c([2H])c([2H])c3[2H])c([2H])c2[2H])c2c([2H])c(-c3nc(-c4c([2H])c([2H])c([2H])c([2H])c4[2H])nc(-c4c([2H])c([2H])c([2H])c([2H])c4[2H])n3)c3c(c2[2H])-c2c([2H])c([2H])c([2H])c4c([2H])c([2H])c([2H])c-3c24)c([2H])c([2H])c1[2H]]

Further, various substituents may be introduced into the structure of Chemical Formula 1 to synthesize a compound having inherent characteristics of a substituent introduced. For example, it is possible to synthesize a material which satisfies conditions required for each organic material layer by introducing a substituent usually used for a hole injection layer material, a material for transporting holes, a light emitting layer material, an electron transport layer material, and a charge generation layer material, which are used for preparing an organic light emitting device, into the core structure.

In addition, it is possible to finely adjust an energy band-gap by introducing various substituents into the structure of Chemical Formula 1, and meanwhile, it is possible to improve characteristics at the interface between organic materials and diversify the use of the material.

Meanwhile, the heterocyclic compound has a high glass transition temperature (Tg) and thus has excellent thermal stability. The increase in thermal stability becomes an important factor for providing a device with driving stability.

The heterocyclic compound according to an exemplary embodiment of the present application may be prepared by a multi-step chemical reaction. Some intermediate compounds are first prepared, and the compound of Chemical Formula 1 may be prepared from the intermediate compounds. More specifically, the heterocyclic compound according to an exemplary embodiment of the present application may be prepared based on the Preparation Examples to be described below.

Another exemplary embodiment of the present application provides an organic light emitting device including the heterocyclic compound represented by Chemical Formula 1. The ‘organic light emitting device” may be expressed by terms such as “organic light emitting diode”, “organic light emitting diodes (OLEDs)”, “OLED device”, and “organic electroluminescence device”.

The heterocyclic compound may be formed as an organic material layer by not only a vacuum deposition method, but also a solution application method when an organic light emitting device is manufactured. Here, the solution application method means spin coating, dip coating, inkjet printing, screen printing, a spray method, roll coating, and the like, but is not limited thereto.

Specifically, the organic light emitting device according to an exemplary embodiment of the present application includes a first electrode, a second electrode and an organic material layer having one or more layers provided between the first electrode and the second electrode, in which one or more layers of the organic material layer include the heterocyclic compound represented by Chemical Formula 1. When the heterocyclic compound represented by Chemical Formula 1 is included in the organic material layer, the light emitting efficiency and service life of the organic light emitting device are excellent.

In an exemplary embodiment of the present application, the first electrode may be a positive electrode, and the second electrode may be a negative electrode.

In another exemplary embodiment, the first electrode may be a negative electrode, and the second electrode may be a positive electrode.

In an exemplary embodiment of the present application, the organic light emitting device may be a green organic light emitting device, and the heterocyclic compound according to Chemical Formula 1 may be used as a material for the green organic light emitting device.

In an exemplary embodiment of the present application, the organic light emitting device may be a red organic light emitting device, and the heterocyclic compound according to Chemical Formula 1 may be used as a material for the red organic light emitting device.

In an exemplary embodiment of the present application, the organic light emitting device may be a blue organic light emitting device, and the heterocyclic compound according to Chemical Formula 1 may be used as a material for the blue organic light emitting device.

Further, the organic material layer includes a hole transport layer, and the hole transport layer includes the heterocyclic compound represented by Chemical Formula 1. When the heterocyclic compound represented by Chemical Formula 1 is included in the hole transport layer among the organic material layers, the light emitting efficiency and service life of the organic light emitting device are better.

Further, the organic material layer includes an electron blocking layer, and the electron blocking layer includes the heterocyclic compound represented by Chemical Formula 1. When the heterocyclic compound represented by Chemical Formula 1 is included in the electron blocking layer among the organic material layers, the light emitting efficiency and service life of the organic light emitting device are better.

In an exemplary embodiment of the present application, provided is an organic light emitting device in which the organic material layer includes a light emitting layer and the light emitting layer includes the heterocyclic compound represented by Chemical Formula 1.

In an exemplary embodiment of the present application, provided is an organic light emitting device in which the organic material layer includes a light emitting layer, the light emitting layer includes a host material, and the host material simultaneously includes the heterocyclic compound represented by Chemical Formula 1.

In an exemplary embodiment of the present application, provided is an organic light emitting device in which the organic material layer includes a light emitting layer, the light emitting layer includes one or more host materials, and at least one of the one or more host materials includes the heterocyclic compound as a host material of a light emitting material.

In an exemplary embodiment of the present application, provided is an organic light emitting device in which the organic material layer includes a light emitting layer, the light emitting layer includes two host materials, and all the two host materials are selected from the heterocyclic compounds.

In the organic light emitting device of the present application, in the light emitting layer, two or more of the heterocyclic compounds represented by Chemical Formula 1 may be used.

In the organic light emitting device of the present application, in the light emitting layer, two or more of the heterocyclic compounds represented by Chemical Formula 1 may be pre-mixed and used.

The pre-mixing means that for the light emitting layer, before two or more host materials are deposited onto an organic material layer, the materials are first mixed and the mixture is contained in one common container and mixed. As described above, since one vapor deposition source is used instead of using two or three types of vapor deposition sources during the pre-mixing, there is an advantage in that the process is more simplified.

The organic light emitting device of the present invention may further include one or two or more layers selected from the group consisting of a light emitting layer, a hole injection layer, a hole transport layer, an electron injection layer, an electron transport layer, a hole auxiliary layer, and a hole blocking layer.

The organic light emitting device according to an exemplary embodiment of the present application may be manufactured by typical manufacturing methods and materials of the organic light emitting device, except that the above-described heterocyclic compound is used to form an organic material layer.

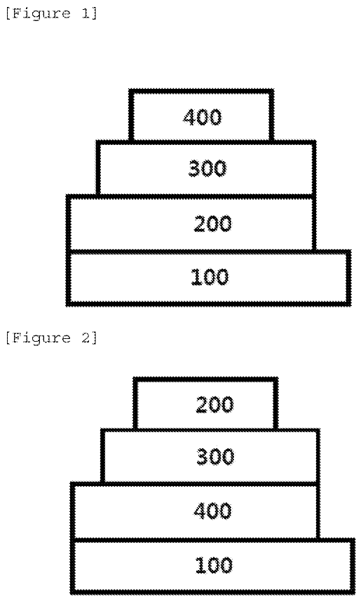

FIGS. 1 to 3 exemplify the stacking sequence of the electrodes and the organic material layer of the organic light emitting device according to an exemplary embodiment of the present application. However, the scope of the present application is not intended to be limited by these drawings, and the structure of the organic light emitting device known in the art may also be applied to the present application.

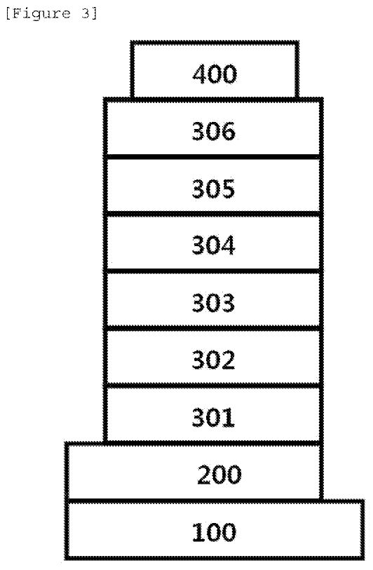

According to FIG. 1, an organic light emitting device in which a positive electrode 200, an organic material layer 300, and a negative electrode 400 are sequentially stacked on a substrate 100 is illustrated. However, the organic light emitting device is not limited only to such a structure, and as in FIG. 2, an organic light emitting device in which a negative electrode, an organic material layer, and a positive electrode are sequentially stacked on a substrate may also be implemented.

FIG. 3 exemplifies a case where an organic material layer is a multilayer. An organic light emitting device according to FIG. 3 includes a hole injection layer 301, a hole transport layer 302, a light emitting layer 303, a hole blocking layer 304, an electron transport layer 305, and an electron injection layer 306. However, the scope of the present application is not limited by the stacking structure as described above, and if necessary, the other layers except for the light emitting layer may be omitted, and another necessary functional layer may be further added.

In the organic light emitting device according to an exemplary embodiment of the present application, materials other than the heterocyclic compound of Chemical Formula 1 will be exemplified below, but these materials are illustrative only and are not for limiting the scope of the present application, and may be replaced with materials publicly known in the art.

As a positive electrode material, materials having a relatively high work function may be used, and a transparent conductive oxide, a metal or a conductive polymer, and the like may be used. Specific examples of the positive electrode material include: a metal such as vanadium, chromium, copper, zinc, and gold, or an alloy thereof; a metal oxide such as zinc oxide, indium oxide, indium tin oxide (ITO), and indium zinc oxide (IZO); a combination of a metal and an oxide, such as ZnO:Al or SnO₂:Sb; a conductive polymer such as poly(3-methylthiophene), poly[3,4-(ethylene-1,2-dioxy)thiophene](PEDOT), polypyrrole, and polyaniline; and the like, but are not limited thereto.

As a negative electrode material, materials having a relatively low work function may be used, and a metal, a metal oxide, or a conductive polymer, and the like may be used. Specific examples of the negative electrode material include: a metal such as magnesium, calcium, sodium, potassium, titanium, indium, yttrium, lithium, gadolinium, aluminum, silver, tin, and lead, or an alloy thereof; a multi-layer structured material, such as LiF/Al or LiO₂/Al; and the like, but are not limited thereto.

As a hole injection material, a publicly-known hole injection material may also be used, and it is possible to use, for example, a phthalocyanine compound such as copper phthalocyanine disclosed in U.S. Pat. No. 4,356,429 or starburst-type amine derivatives described in the document [Advanced Material, 6, p. 677 (1994)], for example, tris(4-carbazoyl-9-ylphenyl)amine (TCTA), 4,4′,4″-tri[phenyl(m-tolyl)amino]triphenylamine (m-MTDATA), 1,3,5-tris[4-(3-methylphenylphenylamino)phenyl]benzene (m-MTDAPB), polyaniline/dodecylbenzenesulfonic acid or poly(3,4-ethylenedioxythiophene)/poly(4-styrenesulfonate), which is a soluble conductive polymer, polyaniline/camphor sulfonic acid or polyaniline/poly(4-styrene-sulfonate), and the like.

As a hole transporting material, a pyrazoline derivative, an arylamine-based derivative, a stilbene derivative, a triphenyldiamine derivative, and the like may be used, and a low-molecular weight or polymer material may also be used.

As an electron transporting material, it is possible to use an oxadiazole derivative, anthraquinodimethane and a derivative thereof, benzoquinone and a derivative thereof, naphthoquinone and a derivative thereof, anthraquinone and a derivative thereof, tetracyanoanthraquinodimethane and a derivative thereof, a fluorenone derivative, diphenyldicyanoethylene and a derivative thereof, a diphenoquinone derivative, a metal complex of 8-hydroxyquinoline and a derivative thereof, and the like, and a low-molecular weight material and a polymer material may also be used.

As an electron injection material, for example, LiF is representatively used in the art, but the present application is not limited thereto.

As a light emitting material, a red, green, or blue light emitting material may be used, and if necessary, two or more light emitting materials may be mixed and used. Further, a fluorescent material may also be used as the light emitting material, but may also be used as a phosphorescent material. As the light emitting material, it is also possible to use alone a material which emits light by combining holes and electrons each injected from a positive electrode and a negative electrode, but materials in which a host material and a dopant material are involved in light emission together may also be used.

The organic light emitting device according to an exemplary embodiment of the present application may be a top emission type, a bottom emission type, or a dual emission type according to the material to be used.

The heterocyclic compound according to an exemplary embodiment of the present application may act even in organic electronic devices including organic solar cells, organic photoconductors, organic transistors, and the like, based on the principle similar to those applied to organic light emitting devices.

Hereinafter, the present specification will be described in more detail through Examples, but these Examples are provided only for exemplifying the present application, and are not intended to limit the scope of the present application.

### <Preparation Example 1> Preparation of Compound 1C

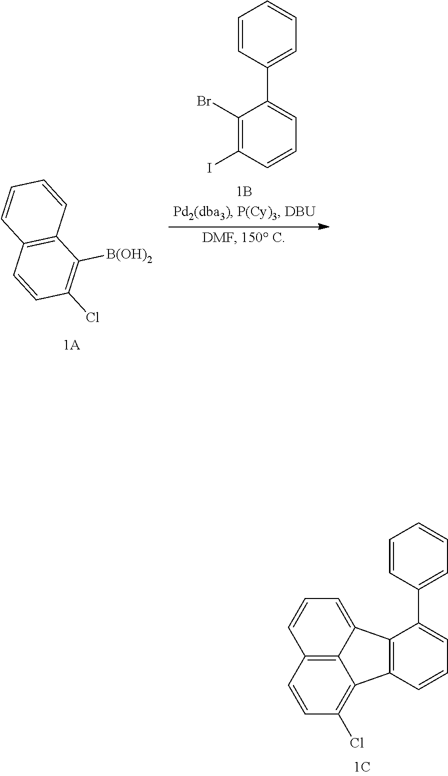 [SMILES: Brc1c(I)cccc1-c1ccccc1 ; OB(O)c1c(Cl)ccc2ccccc12 ; Clc1ccc2cccc3c2c1-c1cccc(-c2ccccc2)c1-3]

### 1) Synthesis of Compound 1C

Compound 1A (4 g, 19.4 mmol), Compound 1B (6.3 g, 17.6 mmol), Pd₂(dba)₃ (1.7 g, 1.9 mmol), P(Cy)₃ (4.2 g, 15 mmol) and 1,8-diazabicyclo[5.4.0]undec-7-ene (hereinafter, referred to as DBU) (2.7 g, 17.6 mmol) were put into a 200-mL Schlenk flask, and the atmosphere was substituted with nitrogen. Thereafter, dimethylformamide (hereinafter, referred to as DMF) (80 mL) was added dropwise thereto. The reaction temperature was increased from room temperature to 155° C. within 10 minutes, and then the resulting mixture was stirred for 24 hours until the reaction was completed. Thereafter, the reaction temperature was lowered to room temperature, the reaction mixture was diluted by adding methylene chloride (hereinafter, referred to as MC) (40 mL) thereto, then the mixture was washed with an aqueous solution of HCl (10%) and an aqueous solution of NaHCO₃, and then the organic layer was extracted with MC.

The extracted organic solvent was dried over Mg₂SO₄, and then concentrated. The concentrated organic solvent was separated by silica-gel column and recrystallized to obtain Compound 1C (2.01 g, 6.68 mmol, 38%) which is a white solid compound.

Compound C in the following Table 1 was synthesized in the same manner as in Preparation Example 1, except that Compound A and Compound B in the following Table 1 were used instead of Compound 1A and Compound 1B, respectively, in the synthesis process of Compound 1C.

TABLE 1

| A | B | C (yield %) |
|---|---|---|
| 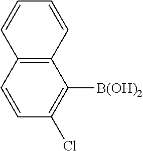 1A | 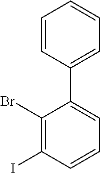 1B | 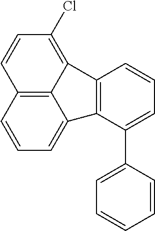 [SMILES: Clc1ccc2cccc3c2c1-c1cccc(-c2ccccc2)c1-3] |
| | | 1C (38%) |
| 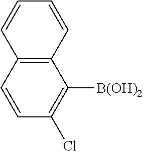 2A | 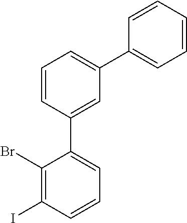 2B | 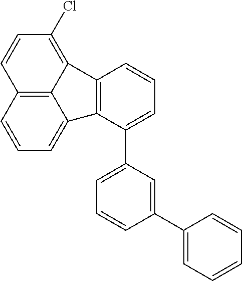 [SMILES: Clc1ccc2cccc3c2c1-c1cccc(-c2cccc(-c4ccccc4)c2)c1-3] |
| | | 2C (23%) |
| 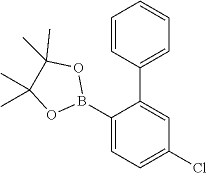 | 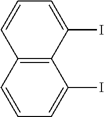 3B | 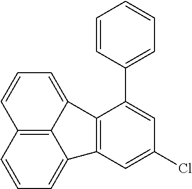 [SMILES: Clc1cc(-c2ccccc2)c2c(c1)-c1cccc3cccc-2c13] |
| 3A | | |
| | | 3C (49%) |
| 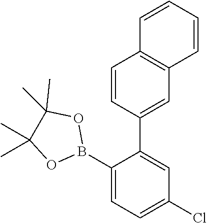 4A | 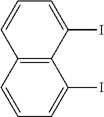 3B | 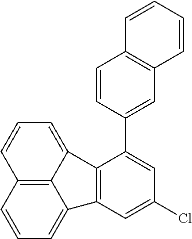 [SMILES: Clc1cc(-c2ccc3ccccc3c2)c2c(c1)-c1cccc3cccc-2c13] |
| | | 4C (44%) |
| 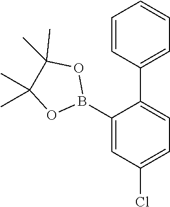 | 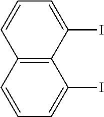 3B | 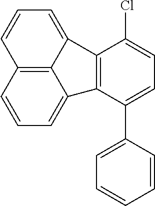 [SMILES: Clc1ccc(-c2ccccc2)c2c1-c1cccc3cccc-2c13] |
| 5A | | 5C (46%) |
| 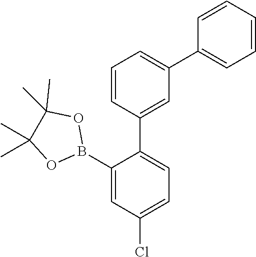 | 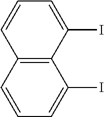 3B | 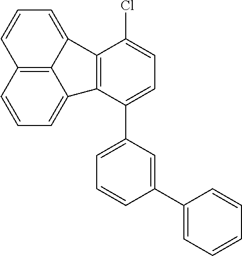 [SMILES: Clc1ccc(-c2cccc(-c3ccccc3)c2)c2c1-c1cccc3cccc-2c13] |
| 6A | | 6C (32%) |
| 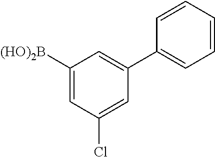 | 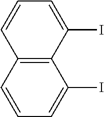 3B | 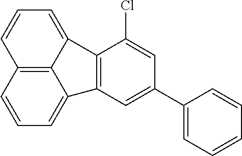 [SMILES: Clc1cc(-c2ccccc2)cc2c1-c1cccc3cccc-2c13] |
| 7A | | 7C (62%) |
| 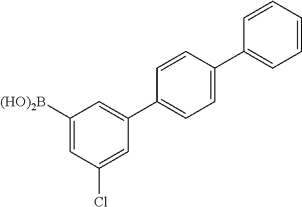 | 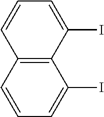 3B | 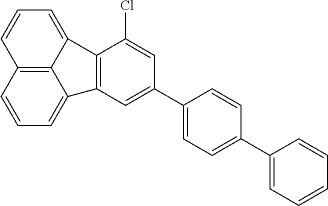 [SMILES: Clc1cc(-c2ccc(-c3ccccc3)cc2)cc2c1-c1cccc3cccc-2c13] |
| 8A | | 8C (56%) |
| 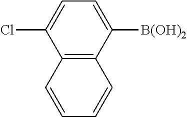 9A | 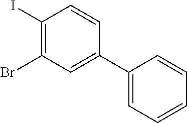 4B | 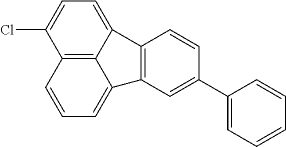 [SMILES: Clc1ccc2c3c(cccc13)-c1cc(-c3ccccc3)ccc1-2] |
| | | 9C (36%) |
| 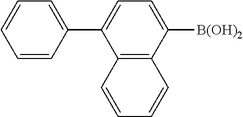 | 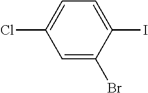 5B | 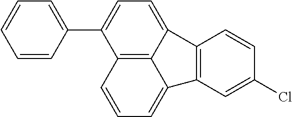 [SMILES: Clc1ccc2c(c1)-c1cccc3c(-c4ccccc4)ccc-2c13] |
| A10 | | 10C (40%) |
| 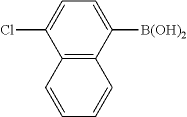 9A | 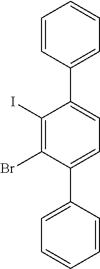 | 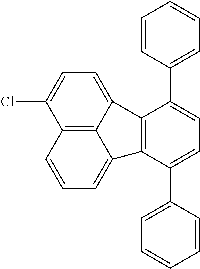 [SMILES: Clc1ccc2c3c(cccc13)-c1c(-c3ccccc3)ccc(-c3ccccc3)c1-2] |
| | 6B | 11C (30%) |
| 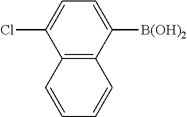 9A | 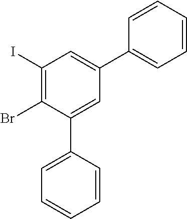 | 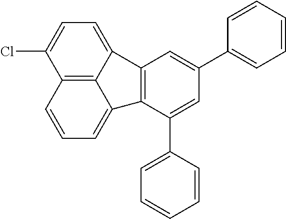 [SMILES: Clc1ccc2c3c(cccc13)-c1c(-c3ccccc3)cc(-c3ccccc3)cc1-2] |
| | 7B | 12C (29%) |
| 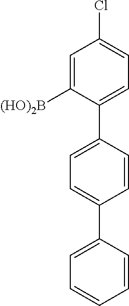 | 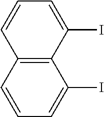 3B | 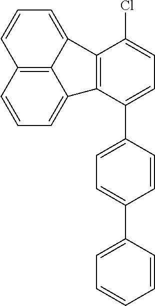 [SMILES: Clc1ccc(-c2ccc(-c3ccccc3)cc2)c2c1-c1cccc3cccc-2c13] |
| 11A | | 13C (36%) |

### <Preparation Example 2> Preparation of Compound 2J

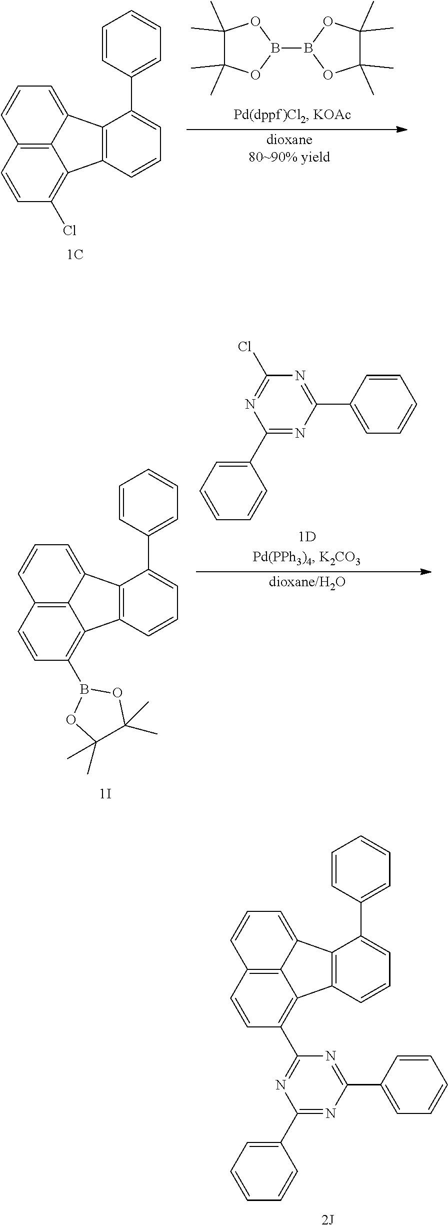

### 1) Synthesis (1) of Compound 1I

Compound 1C (2 g, 6.68 mmol), bis(pinacolato)diboron (2.2 g, 8.7 mmol), Pd(dppf)Cl₂ (0.5 g, 0.7 mmol) and KOAc (1.7 g, 17.4 mmol) were put into a 100-mL round bottom flask, and a nitrogen atmosphere was created. Thereafter, dioxane (60 mL) was added thereto, and the resulting mixture was stirred at 110° C. for 4 hours. After the reaction was completed, the reaction temperature was lowered to room temperature, and the resulting product was washed with water, and then extracted with MC. The extracted organic solvent was dried over Mg₂SO₄, and then concentrated. The concentrated organic solvent was separated by silica-gel column and recrystallized to obtain Compound 1I (2.2 g, 4.45 mmol, 74%) which is a white solid compound.

### 2) Synthesis (1) of Compound 2J

Compound 1I (2 g, 4.95 mmol), Compound 1D (1.6 g, 6 mmol), Pd(PPh₃)₄ (0.3 g, 0.25 mmol) and K₂CO₃ (1.4 g, 10 mmol) were put into a 100-mL round bottom flask, a nitrogen atmosphere was created, and then dioxane (50 mL)/H₂O (10 mL) was added thereto, and the resulting mixture was stirred at 120° C. for 12 hours. After the reaction was completed, the reaction temperature was lowered to room temperature, the resulting product was washed with water, and then extracted with MC. The extracted organic solvent was dried over Mg₂SO₄, and then concentrated. The concentrated organic solvent was separated by silica-gel column and recrystallized to obtain Compound 2J (2.2 g, 4.45 mmol, 90%) which is a yellow solid compound.

Compound J in the following Table 2 was synthesized in the same manner as in Preparation Example 2, except that Compound C in the following Table 2 was used instead of Compound 1C in the synthesis process of Compound 1I and Compound D in the following Table 2 was used instead of Compound 1D in the synthesis process of Compound 2J.

TABLE 2

| C | D | J (yield %) |
|---|---|---|
| 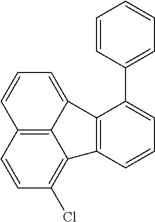 1C | 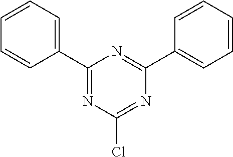 1D | 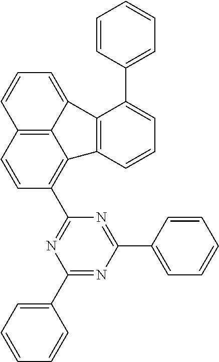 [SMILES: c1ccc(-c2nc(-c3ccccc3)nc(-c3ccc4cccc5c4c3-c3cccc(-c4ccccc4)c3-5)n2)cc1] |
| | | 2J (90%) |
| 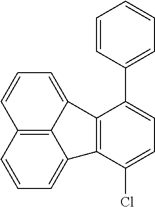 5C | 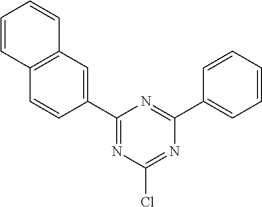 2D | 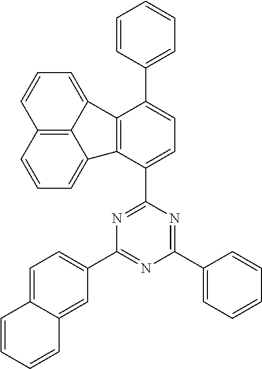 [SMILES: c1ccc(-c2nc(-c3ccc4ccccc4c3)nc(-c3ccc(-c4ccccc4)c4c3-c3cccc5cccc-4c35)n2)cc1] |
| | | 6J (89%) |
| 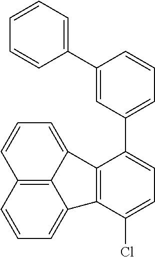 6C | 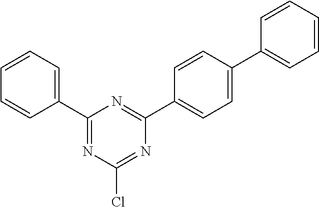 3D | 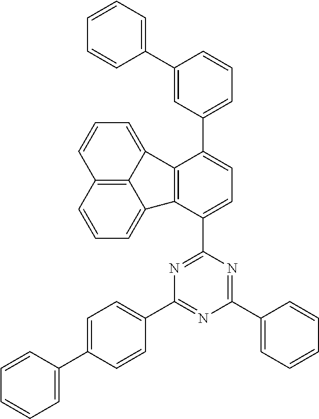 [SMILES: c1ccc(-c2ccc(-c3nc(-c4ccccc4)nc(-c4ccc(-c5cccc(-c6ccccc6)c5)c5c4-c4cccc6cccc-5c46)n3)cc2)cc1] |
| | | 7J (86%) |
| 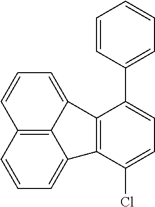 5C | 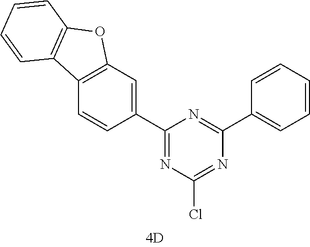 | 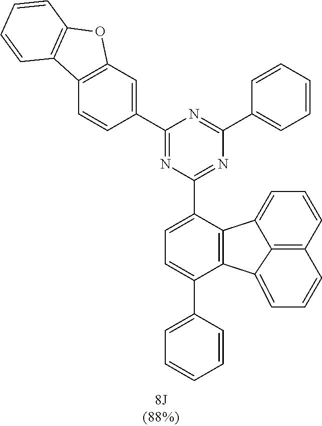 [SMILES: c1ccc(-c2nc(-c3ccc4c(c3)oc3ccccc34)nc(-c3ccc(-c4ccccc4)c4c3-c3cccc5cccc-4c35)n2)cc1] |
| . 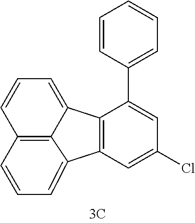 | 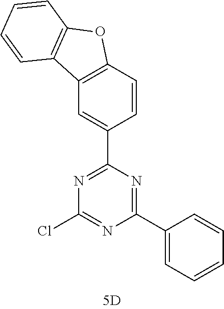 | 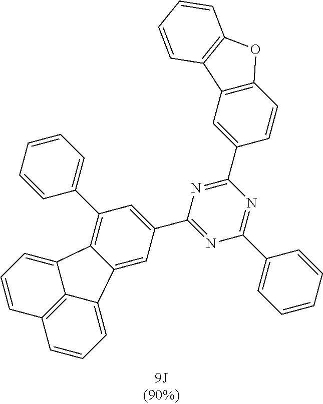 [SMILES: c1ccc(-c2nc(-c3cc(-c4ccccc4)c4c(c3)-c3cccc5cccc-4c35)nc(-c3ccc4oc5ccccc5c4c3)n2)cc1] |
| 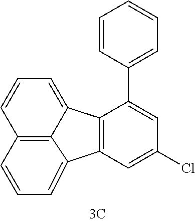 | 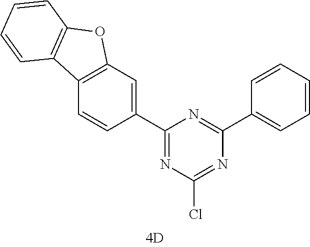 | 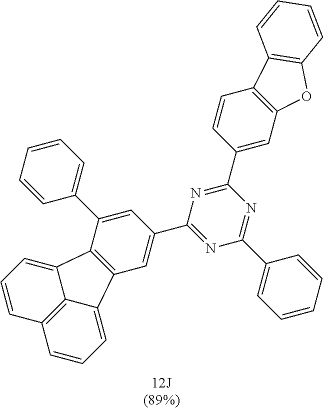 [SMILES: c1ccc(-c2nc(-c3cc(-c4ccccc4)c4c(c3)-c3cccc5cccc-4c35)nc(-c3ccc4c(c3)oc3ccccc34)n2)cc1] |
| 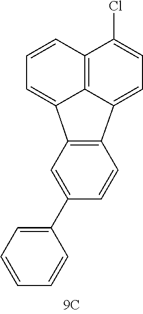 | 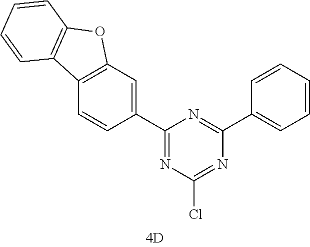 | 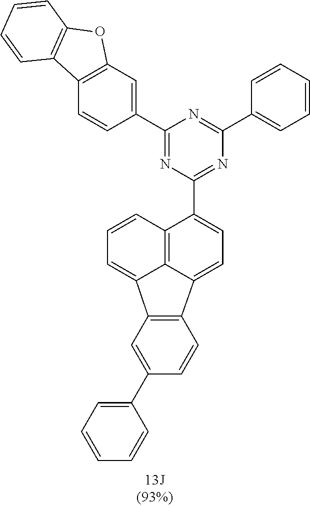 [SMILES: c1ccc(-c2ccc3c(c2)-c2cccc4c(-c5nc(-c6ccccc6)nc(-c6ccc7c(c6)oc6ccccc67)n5)ccc-3c24)cc1] |
| 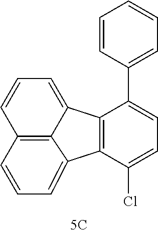 | 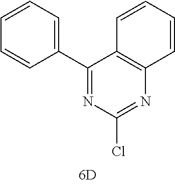 | 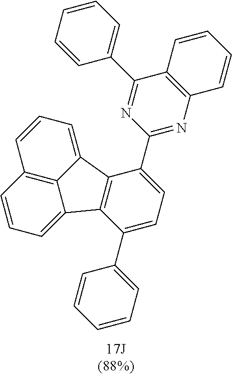 [SMILES: c1ccc(-c2ccc(-c3nc(-c4ccccc4)c4ccccc4n3)c3c2-c2cccc4cccc-3c24)cc1] |
| 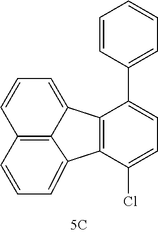 | 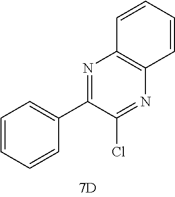 | 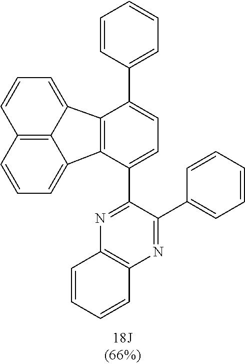 [SMILES: c1ccc(-c2ccc(-c3nc4ccccc4nc3-c3ccccc3)c3c2-c2cccc4cccc-3c24)cc1] |
| 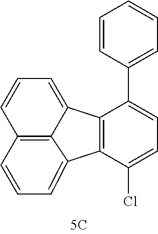 | 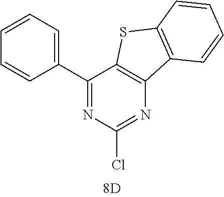 | 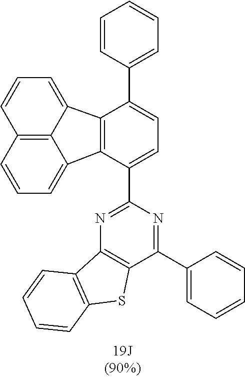 [SMILES: c1ccc(-c2ccc(-c3nc(-c4ccccc4)c4sc5ccccc5c4n3)c3c2-c2cccc4cccc-3c24)cc1] |
| 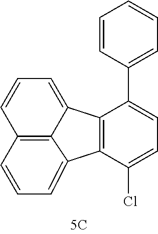 | 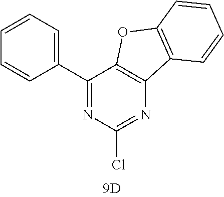 | 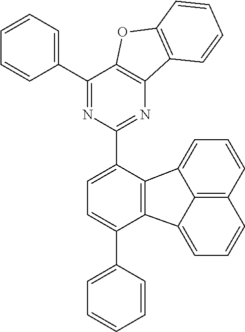 [SMILES: c1ccc(-c2ccc(-c3nc(-c4ccccc4)c4oc5ccccc5c4n3)c3c2-c2cccc4cccc-3c24)cc1] |
| | | 20J (69%) |
| 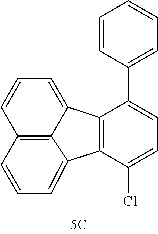 | 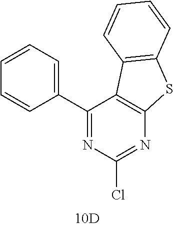 | 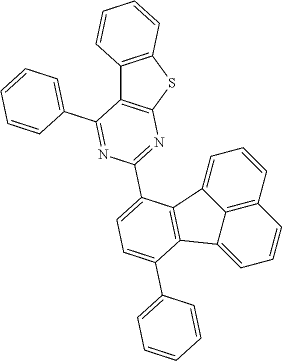 [SMILES: c1ccc(-c2ccc(-c3nc(-c4ccccc4)c4c(n3)sc3ccccc34)c3c2-c2cccc4cccc-3c24)cc1] |
| | | 23J (79%) |
| 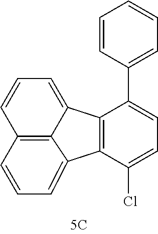 | 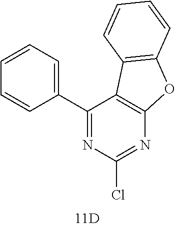 | 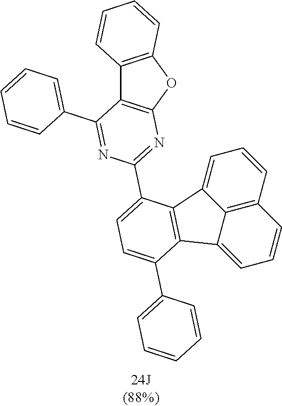 [SMILES: c1ccc(-c2ccc(-c3nc(-c4ccccc4)c4c(n3)oc3ccccc34)c3c2-c2cccc4cccc-3c24)cc1] |
| 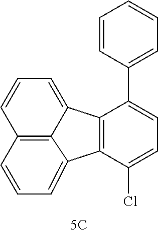 | 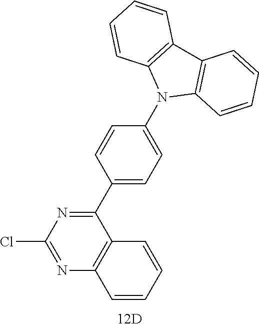 | 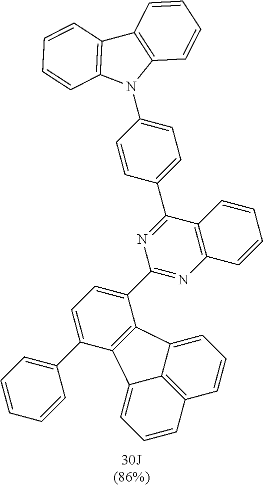 [SMILES: c1ccc(-c2ccc(-c3nc(-c4ccc(-n5c6ccccc6c6ccccc65)cc4)c4ccccc4n3)c3c2-c2cccc4cccc-3c24)cc1] |
| 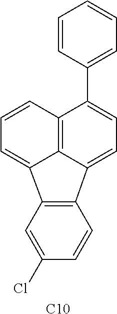 | 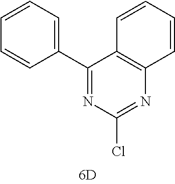 | 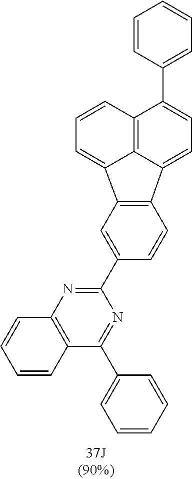 [SMILES: c1ccc(-c2nc(-c3ccc4c(c3)-c3cccc5c(-c6ccccc6)ccc-4c35)nc3ccccc23)cc1] |
| 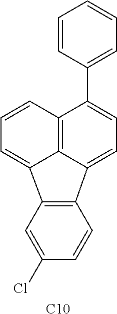 | 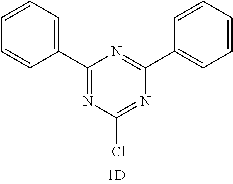 | 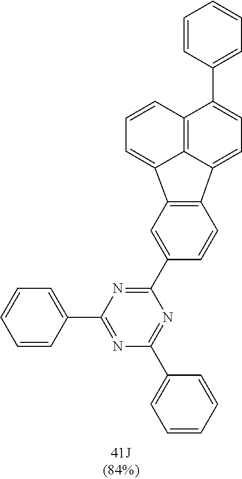 [SMILES: c1ccc(-c2nc(-c3ccccc3)nc(-c3ccc4c(c3)-c3cccc5c(-c6ccccc6)ccc-4c35)n2)cc1] |
| 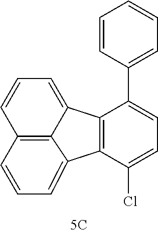 | 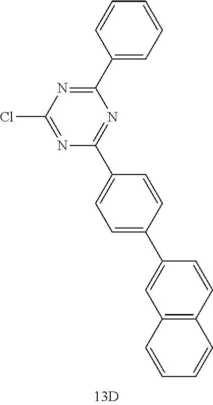 | 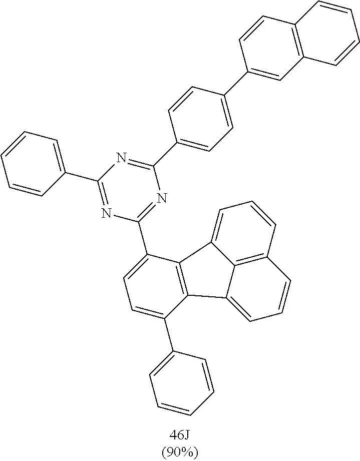 [SMILES: c1ccc(-c2nc(-c3ccc(-c4ccc5ccccc5c4)cc3)nc(-c3ccc(-c4ccccc4)c4c3-c3cccc5cccc-4c35)n2)cc1] |
| 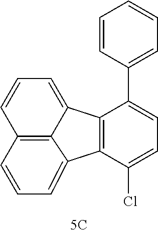 | 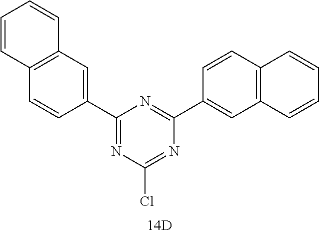 | 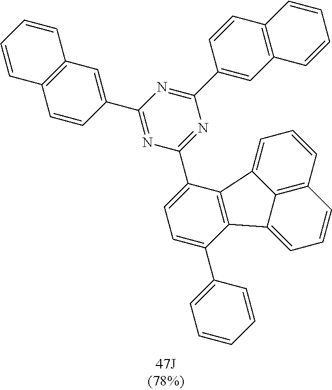 [SMILES: c1ccc(-c2ccc(-c3nc(-c4ccc5ccccc5c4)nc(-c4ccc5ccccc5c4)n3)c3c2-c2cccc4cccc-3c24)cc1] |
| 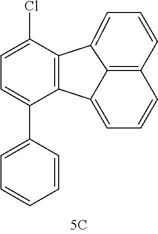 | 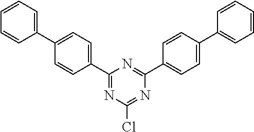 15D | 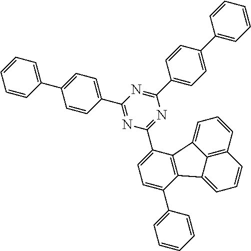 [SMILES: c1ccc(-c2ccc(-c3nc(-c4ccc(-c5ccccc5)cc4)nc(-c4ccc(-c5ccccc5)c5c4-c4cccc6cccc-5c46)n3)cc2)cc1] |
| | | 48J |
| | | (83%) |
| 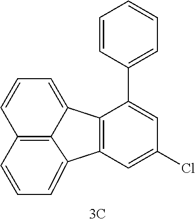 | 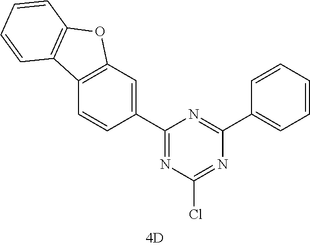 | 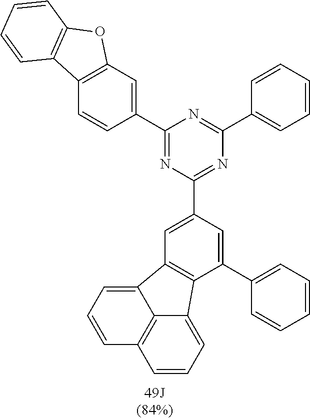 [SMILES: c1ccc(-c2nc(-c3cc(-c4ccccc4)c4c(c3)-c3cccc5cccc-4c35)nc(-c3ccc4c(c3)oc3ccccc34)n2)cc1] |
| 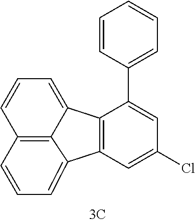 | 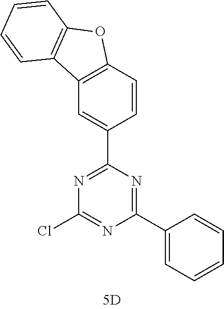 | 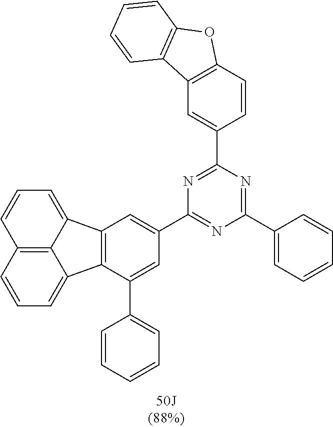 [SMILES: c1ccc(-c2nc(-c3cc(-c4ccccc4)c4c(c3)-c3cccc5cccc-4c35)nc(-c3ccc4oc5ccccc5c4c3)n2)cc1] |
| 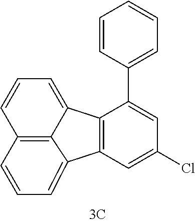 | 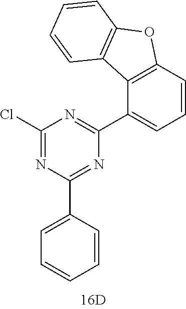 | 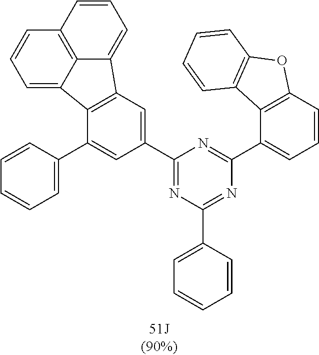 [SMILES: c1ccc(-c2nc(-c3cc(-c4ccccc4)c4c(c3)-c3cccc5cccc-4c35)nc(-c3cccc4oc5ccccc5c34)n2)cc1] |
| 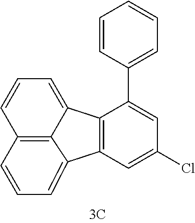 | 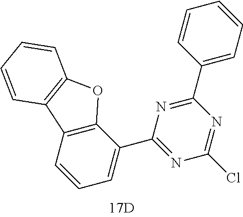 | 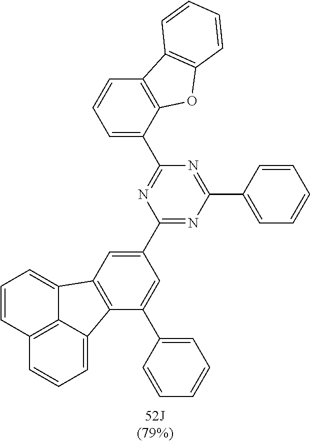 [SMILES: c1ccc(-c2nc(-c3cc(-c4ccccc4)c4c(c3)-c3cccc5cccc-4c35)nc(-c3cccc4c3oc3ccccc34)n2)cc1] |
| 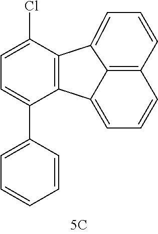 | 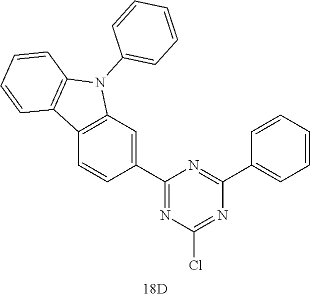 | 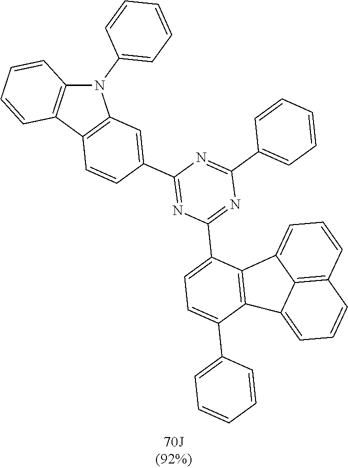 [SMILES: c1ccc(-c2nc(-c3ccc4c5ccccc5n(-c5ccccc5)c4c3)nc(-c3ccc(-c4ccccc4)c4c3-c3cccc5cccc-4c35)n2)cc1] |
| 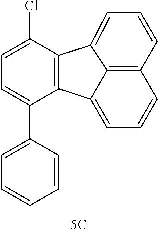 | 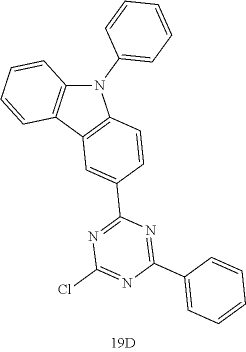 | 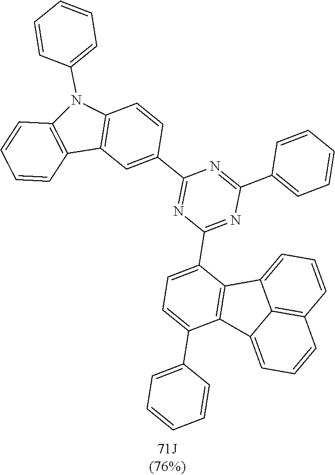 [SMILES: c1ccc(-c2nc(-c3ccc4c(c3)c3ccccc3n4-c3ccccc3)nc(-c3ccc(-c4ccccc4)c4c3-c3cccc5cccc-4c35)n2)cc1] |
| 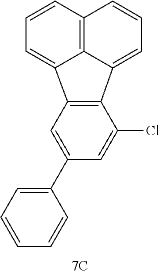 | 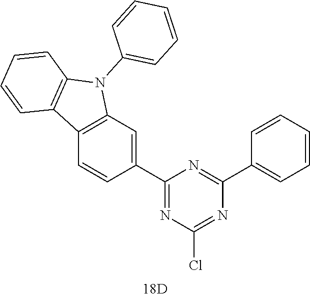 | 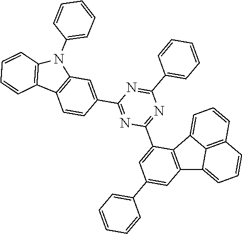 [SMILES: c1ccc(-c2cc(-c3nc(-c4ccccc4)nc(-c4ccc5c6ccccc6n(-c6ccccc6)c5c4)n3)c3c(c2)-c2cccc4cccc-3c24)cc1] 74J (84%) |
| 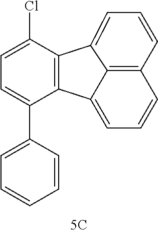 | 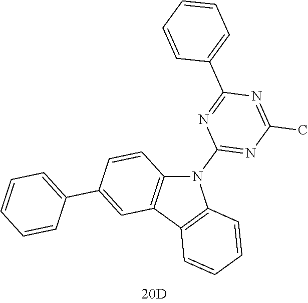 | 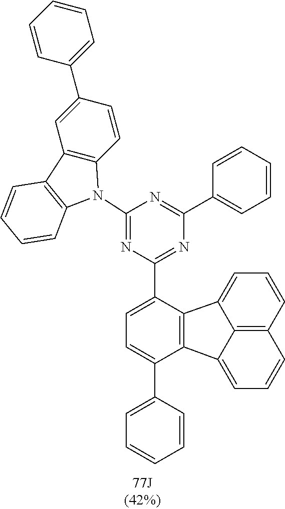 [SMILES: c1ccc(-c2ccc3c(c2)c2ccccc2n3-c2nc(-c3ccccc3)nc(-c3ccc(-c4ccccc4)c4c3-c3cccc5cccc-4c35)n2)cc1] |
| 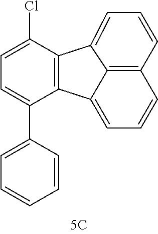 | 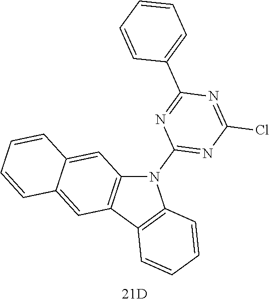 | 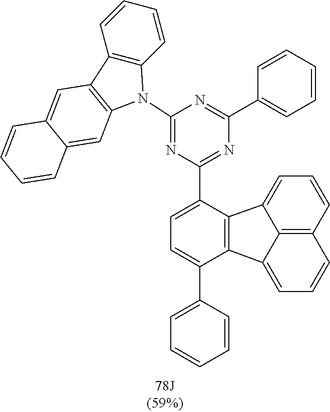 [SMILES: c1ccc(-c2nc(-c3ccc(-c4ccccc4)c4c3-c3cccc5cccc-4c35)nc(-n3c4ccccc4c4cc5ccccc5cc43)n2)cc1] |
| 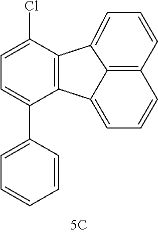 | 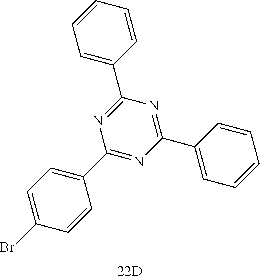 | 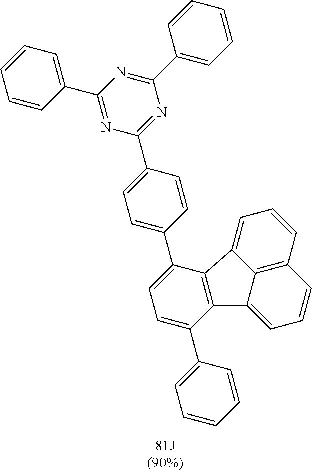 [SMILES: c1ccc(-c2nc(-c3ccccc3)nc(-c3ccc(-c4ccc(-c5ccccc5)c5c4-c4cccc6cccc-5c46)cc3)n2)cc1] |
| 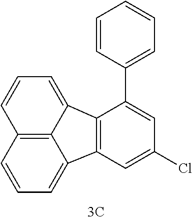 | 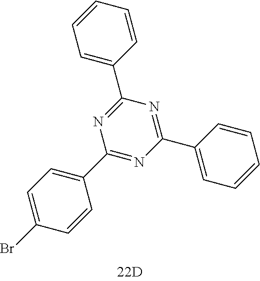 | 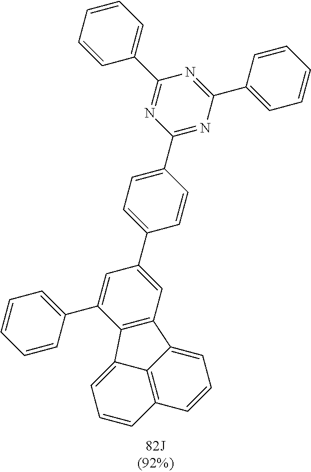 [SMILES: c1ccc(-c2nc(-c3ccccc3)nc(-c3ccc(-c4cc(-c5ccccc5)c5c(c4)-c4cccc6cccc-5c46)cc3)n2)cc1] |
| 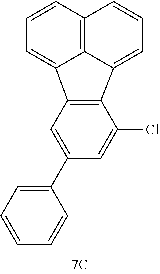 | 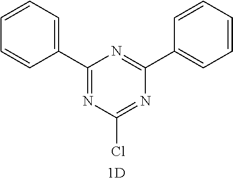 | 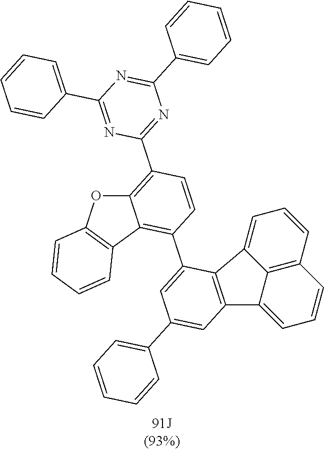 [SMILES: c1ccc(-c2cc3c(c(-c4ccc(-c5nc(-c6ccccc6)nc(-c6ccccc6)n5)c5oc6ccccc6c45)c2)-c2cccc4cccc-3c24)cc1] |
| 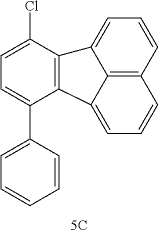 | 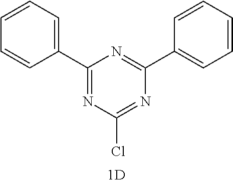 | 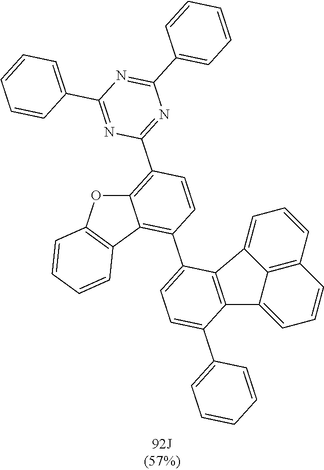 [SMILES: c1ccc(-c2nc(-c3ccccc3)nc(-c3ccc(-c4ccc(-c5ccccc5)c5c4-c4cccc6cccc-5c46)c4c3oc3ccccc34)n2)cc1] |
| 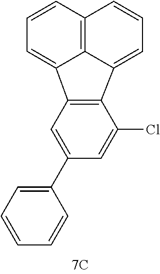 | 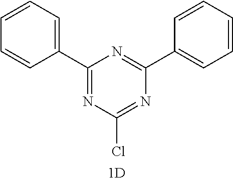 | 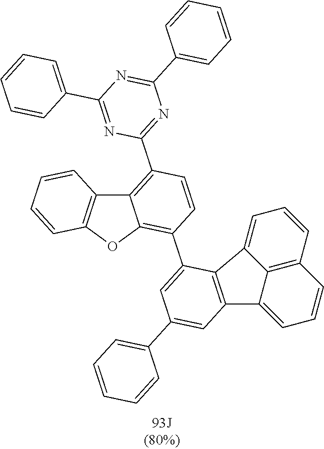 [SMILES: c1ccc(-c2cc(-c3ccc(-c4nc(-c5ccccc5)nc(-c5ccccc5)n4)c4c3oc3ccccc34)c3c(c2)-c2cccc4cccc-3c24)cc1] |
| 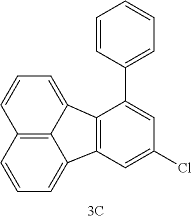 | 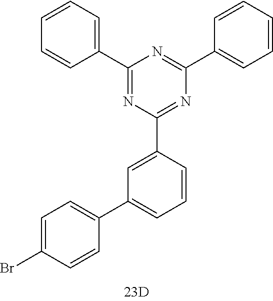 | 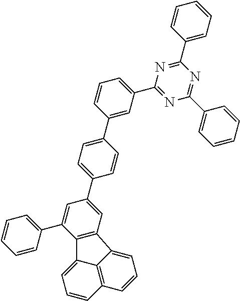 [SMILES: c1ccc(-c2nc(-c3ccccc3)nc(-c3cccc(-c4ccc(-c5cc(-c6ccccc6)c6c(c5)-c5cccc7cccc-6c57)cc4)c3)n2)cc1] |
| | | 98J (94%) |
| 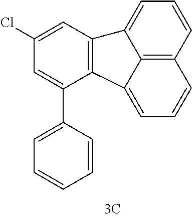 | 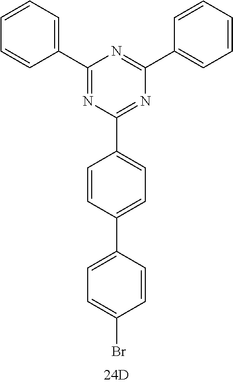 | 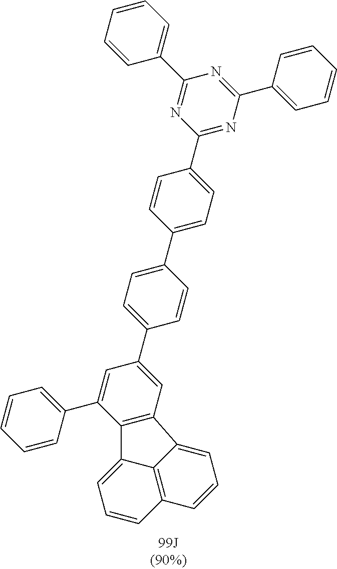 [SMILES: c1ccc(-c2nc(-c3ccccc3)nc(-c3ccc(-c4ccc(-c5cc(-c6ccccc6)c6c(c5)-c5cccc7cccc-6c57)cc4)cc3)n2)cc1] |
| 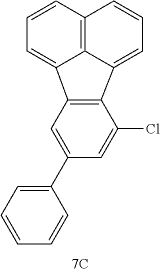 | 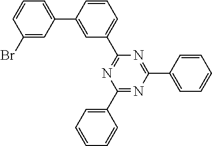 25D | 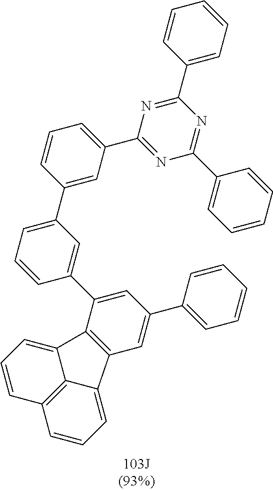 [SMILES: c1ccc(-c2cc(-c3cccc(-c4cccc(-c5nc(-c6ccccc6)nc(-c6ccccc6)n5)c4)c3)c3c(c2)-c2cccc4cccc-3c24)cc1] |
| 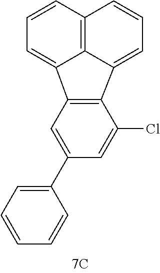 | 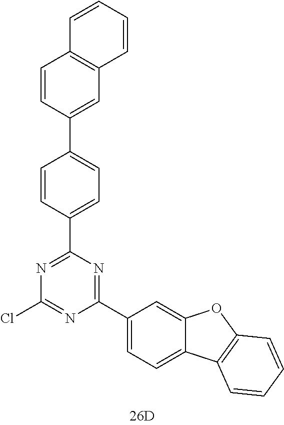 | 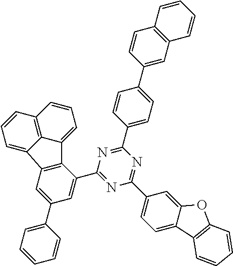 [SMILES: c1ccc(-c2cc(-c3nc(-c4ccc(-c5ccc6ccccc6c5)cc4)nc(-c4ccc5c(c4)oc4ccccc45)n3)c3c(c2)-c2cccc4cccc-3c24)cc1] 110J (88%) |
| 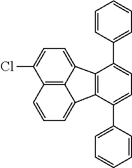 11C | 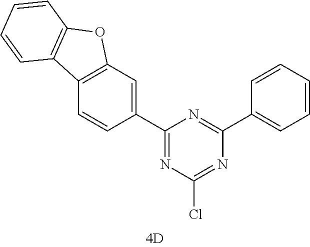 | 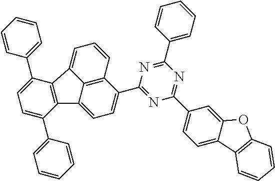 [SMILES: c1ccc(-c2nc(-c3ccc4c(c3)oc3ccccc34)nc(-c3ccc4c5c(cccc35)-c3c(-c5ccccc5)ccc(-c5ccccc5)c3-4)n2)cc1] 117J (69%) |
| 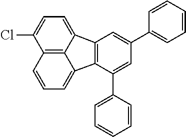 12C | 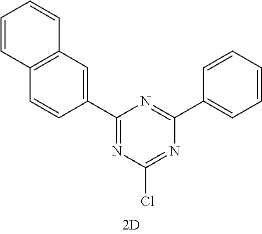 | 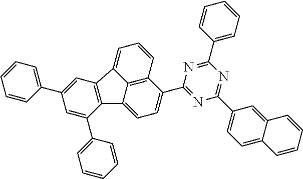 [SMILES: c1ccc(-c2cc(-c3ccccc3)c3c(c2)-c2cccc4c(-c5nc(-c6ccccc6)nc(-c6ccc7ccccc7c6)n5)ccc-3c24)cc1] (90%) |
| 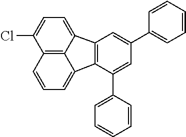 12C | 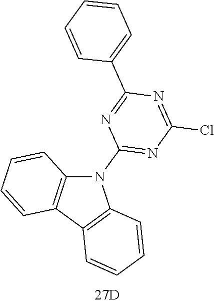 | 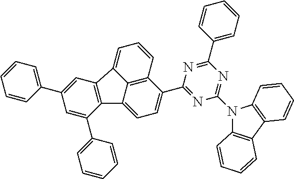 [SMILES: c1ccc(-c2cc(-c3ccccc3)c3c(c2)-c2cccc4c(-c5nc(-c6ccccc6)nc(-n6c7ccccc7c7ccccc76)n5)ccc-3c24)cc1] 126J (89%) |

### <Preparation Example 3> Preparation of Compound 1H

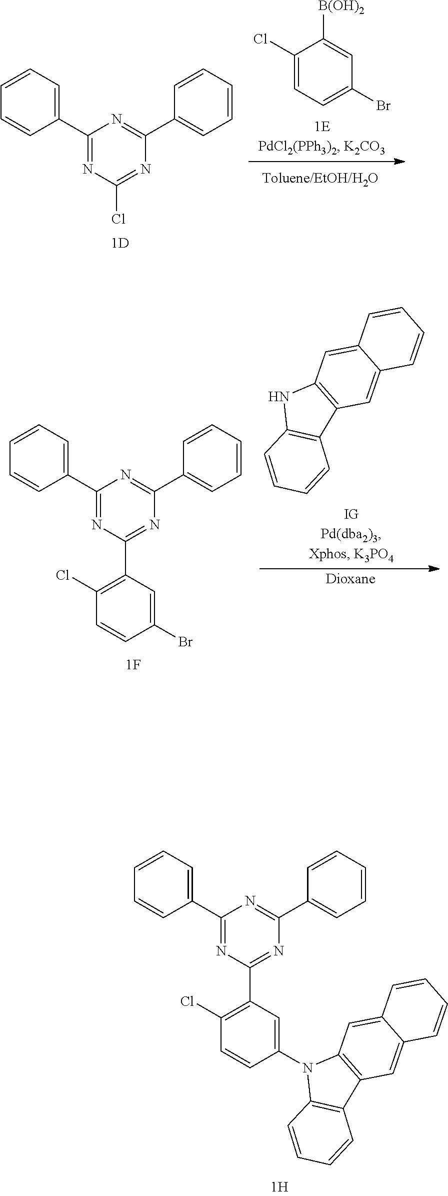

### 1) Synthesis of Compound 1F

Compound 1D (15 g, 56.0 mmol), Compound 1E (15.7 g, 67 mmol), Pd(PPh₃)₂Cl₂ (2.0 g, 2.8 mmol) and K₂CO₃ (15.5 g, 112 mmol) were put into a 500-mL round bottom flask, and a nitrogen atmosphere was created. Thereafter, toluene (200 mL)/EtOH (40 mL)/H₂O (40 mL) was added thereto, and the resulting mixture was stirred at 120° C. for 12 hours. After the reaction was completed, the reaction temperature was lowered to room temperature, and the resulting product was washed with water, and then extracted with Mc. The extracted organic solvent was dried over Mg₂SO₄, and then concentrated. The concentrated organic solvent was separated by silica-gel column and recrystallized to obtain Compound 1F (18.9 g, 44.8 mmol, 80%) which is a white solid compound.

### 2) Synthesis of Compound 1H

Compound 1F (5 g, 12 mmol), Compound 1G (2.8 g, 13.2 mmol), Pd(dba₂)₃ (0.35 g, 0.6 mmol), Xphos (1.1 g, 2.4 mmol) and K₃PO₄ (5 g, 24 mmol) were put into a 250-mL round bottom flask, and a nitrogen atmosphere was created. Thereafter, dioxane (60 mL) was added thereto, and the resulting mixture was stirred at 120° C. for 12 hours. After the reaction was completed, the reaction temperature was lowered to room temperature, and the resulting product was washed with water, and then extracted with MC. The extracted organic solvent was dried over Mg₂SO₄, and then concentrated. The concentrated organic solvent was separated by silica-gel column and recrystallized to obtain Compound 1H (4.7 g, 8.4 mmol, 70%) which is a white solid compound.

Compound H in the following Table 3 was synthesized in the same manner as in Preparation Example 3, except that Compound D and Compound E in the following Table 3 were used instead of Compound 1D and Compound 1E, respectively, in the synthesis process of Compound 1F, and Compounds F and G in the following Table 3 were used instead of Compound 1F and Compound 1G, respectively, in the synthesis process of Compound 1H.

TABLE 3

| D | E | F |
|---|---|---|
| 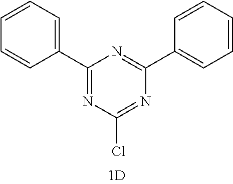 | 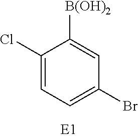 | 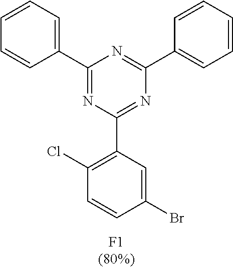 |
| 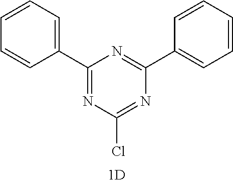 | 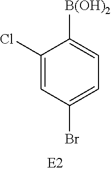 | 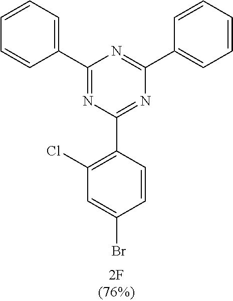 |
| 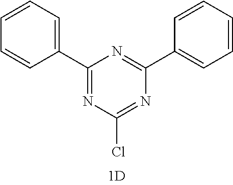 | 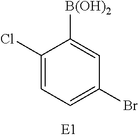 | 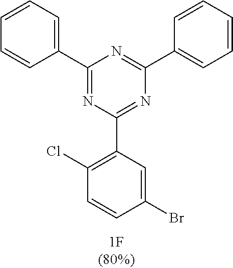 |
| 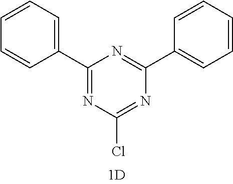 | 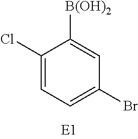 | 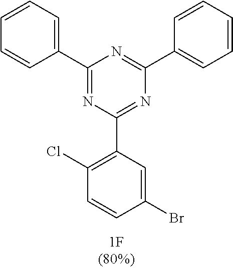 |
| 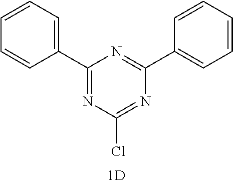 | 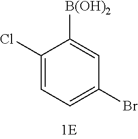 | 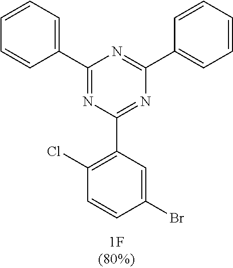 |
| 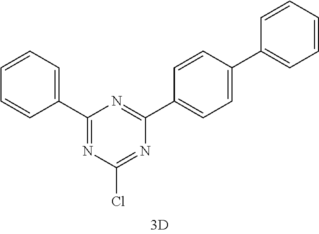 | 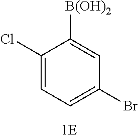 | 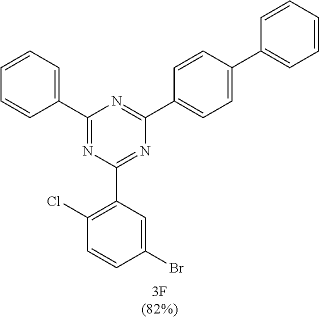 |
| 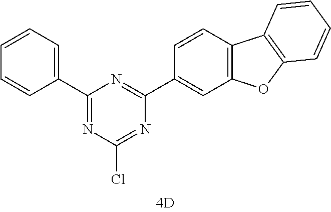 | 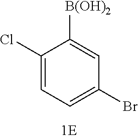 | 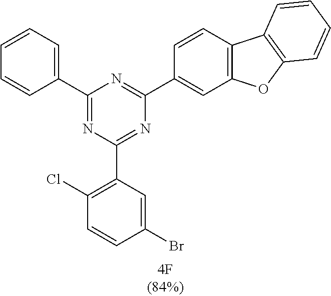 |
| 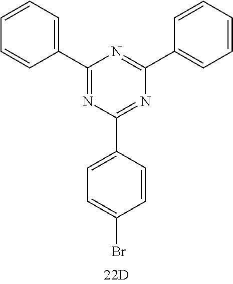 | 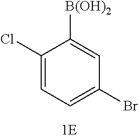 | 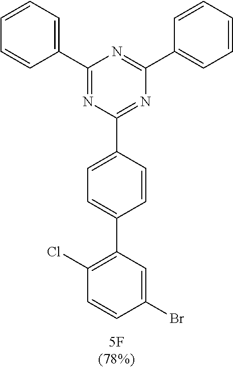 |
| 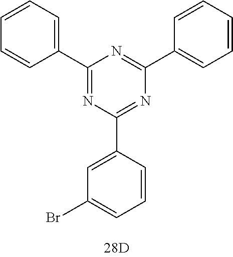 | 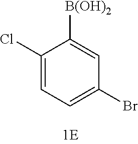 | 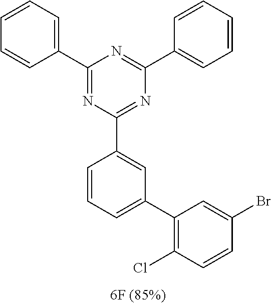 |
| 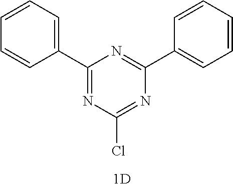 | 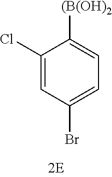 | 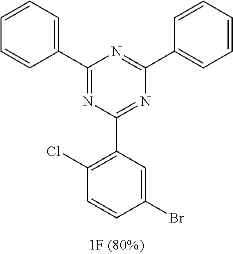 |
| 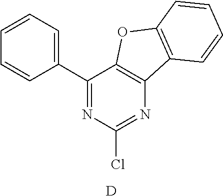 | 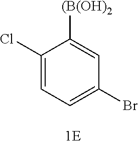 | 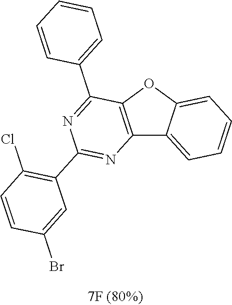 |
| 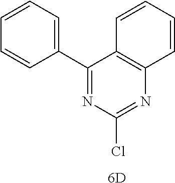 | 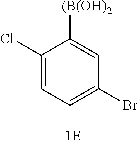 | 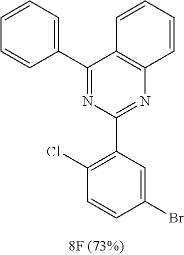 |
| 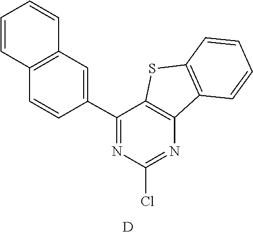 | 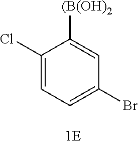 | 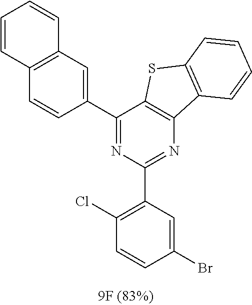 |
| 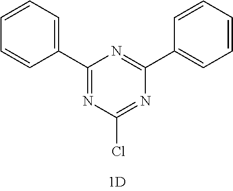 | 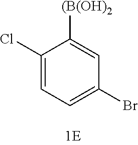 | 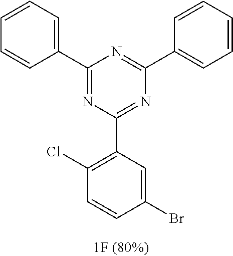 |
| 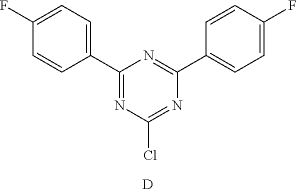 | 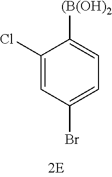 | 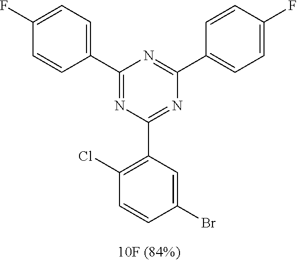 |
| 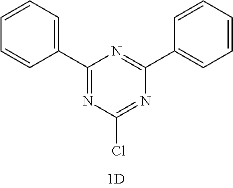 | 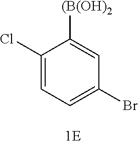 | 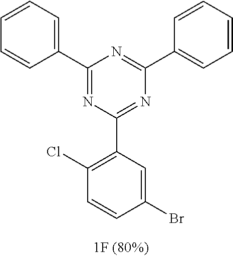 |
| 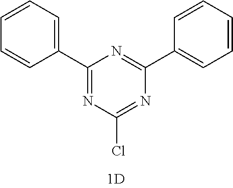 | 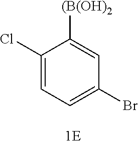 | 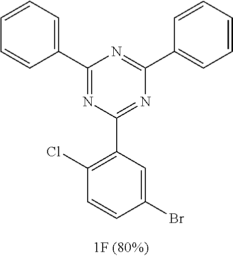 |
| 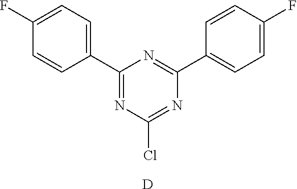 | 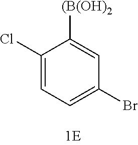 | 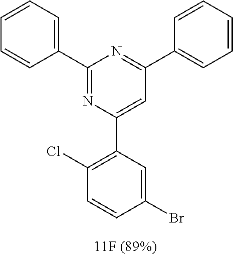 |
| 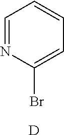 | 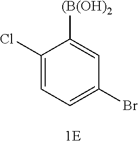 | 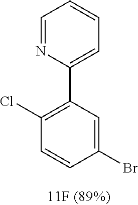 |
| 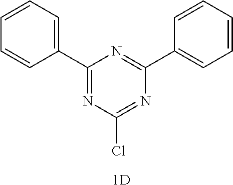 | 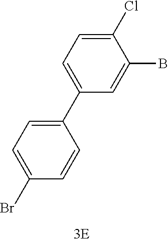 | 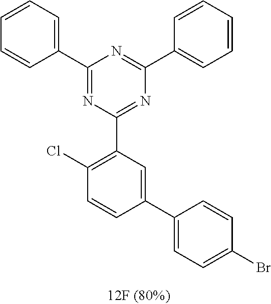 |
| 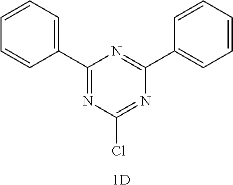 | 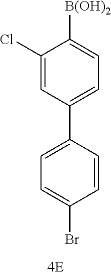 | 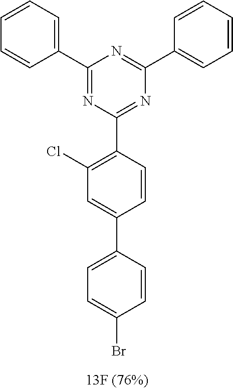 |
| 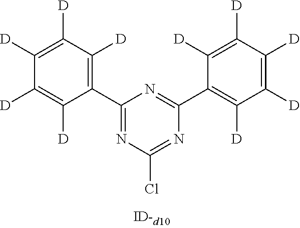 | 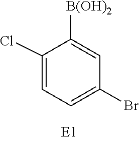 | 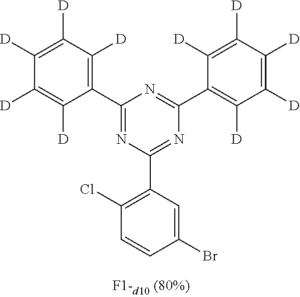 |
| 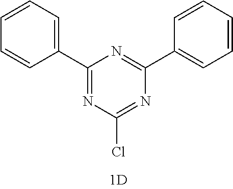 | 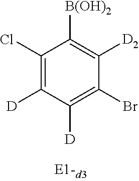 | 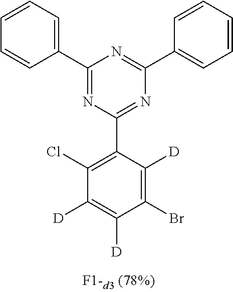 |

| G | H |
|---|---|
| 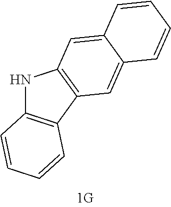 | 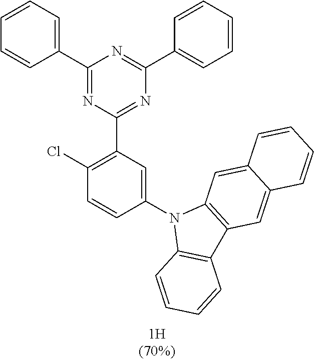 |
| 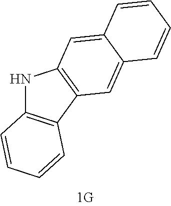 | 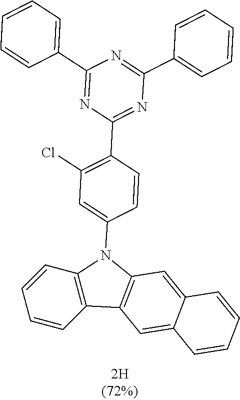 |
| 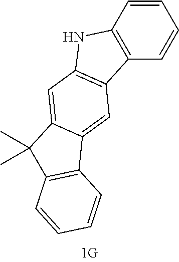 | 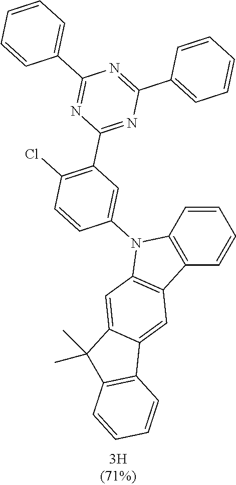 |
| 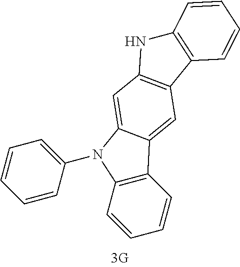 | 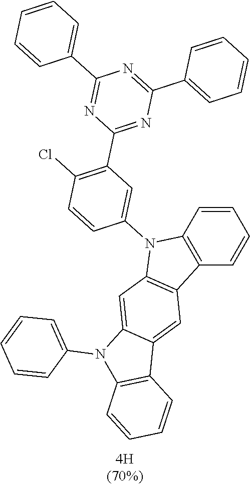 |
| 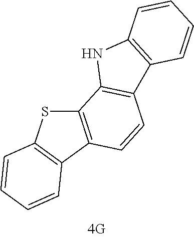 | 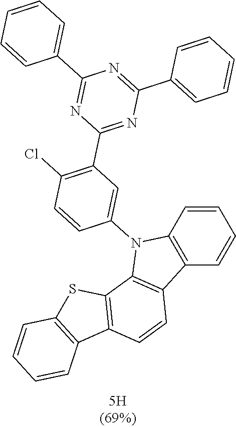 |
| 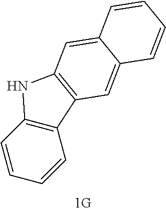 | 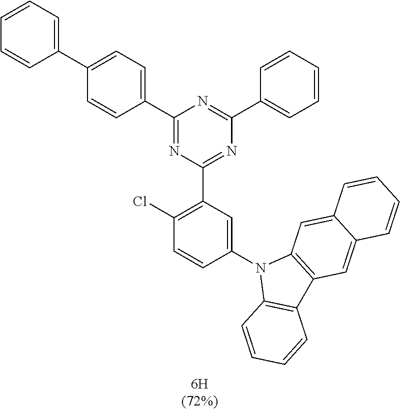 |
| 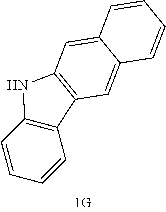 | 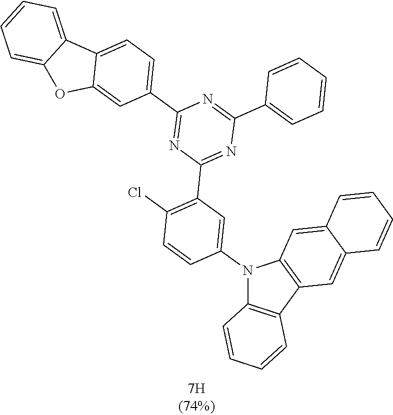 |
| 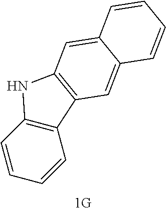 | 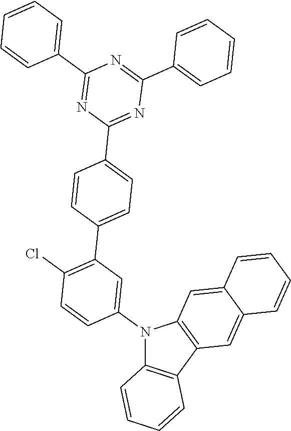 |
| | 8H (80%) |
| 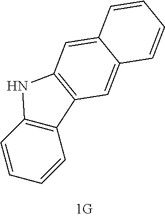 | 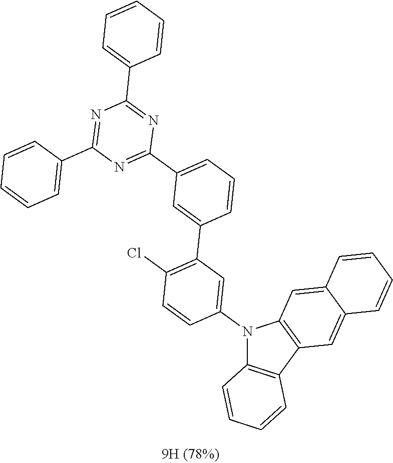 |
| 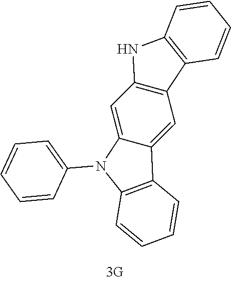 | 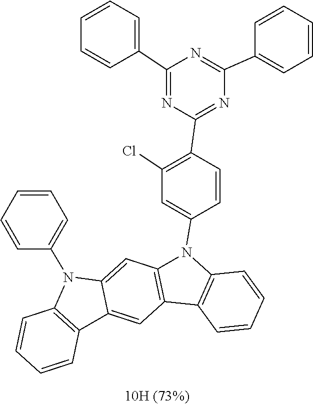 |
| 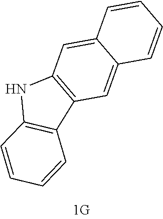 | 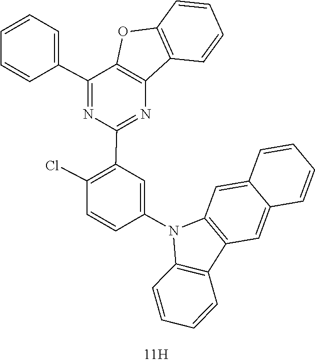 |
| 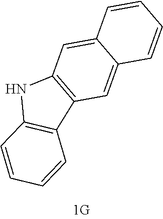 | 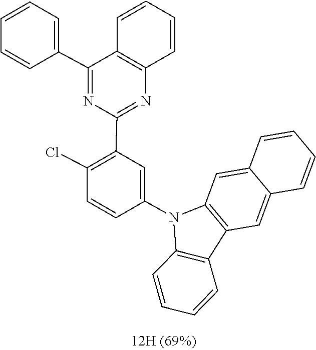 |
| 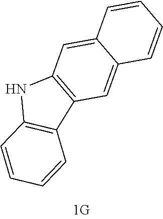 | 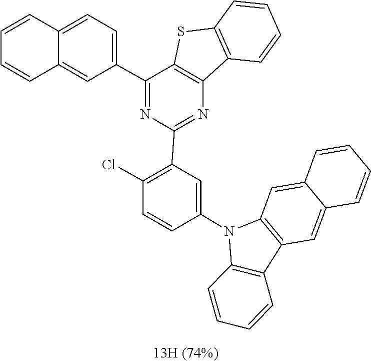 |
| 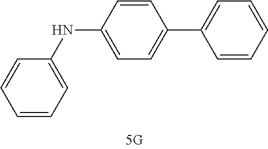 | 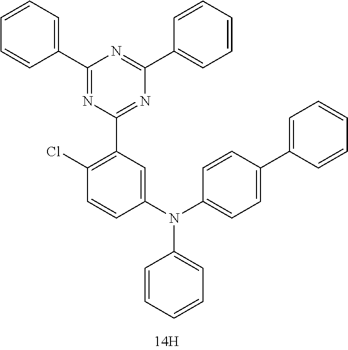 |
| 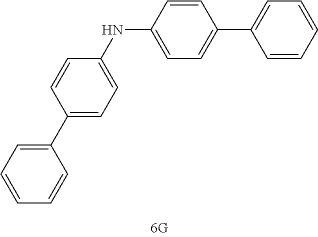 | 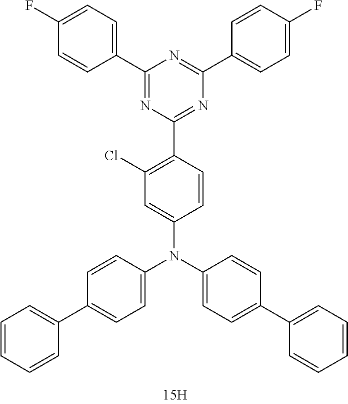 |
| 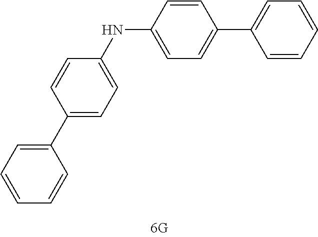 | 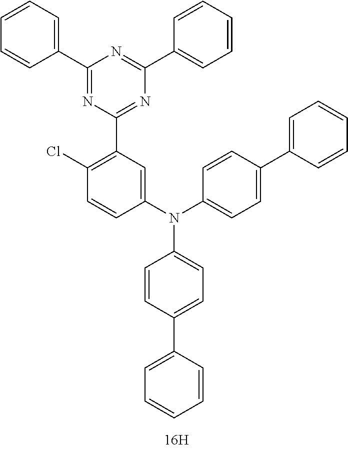 |
| 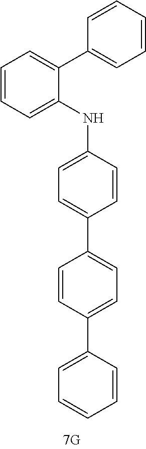 | 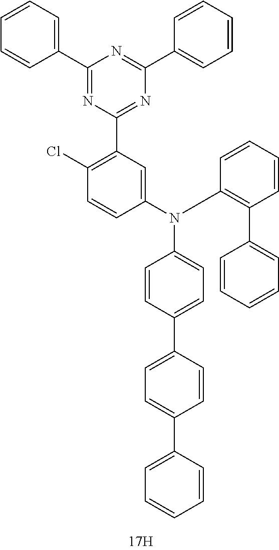 |
| 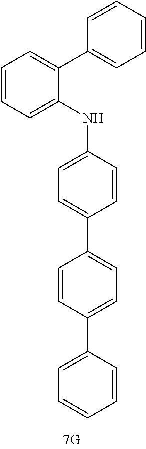 | 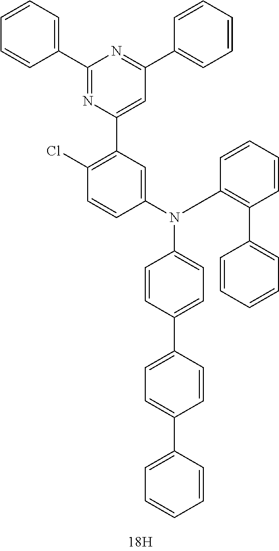 |
| 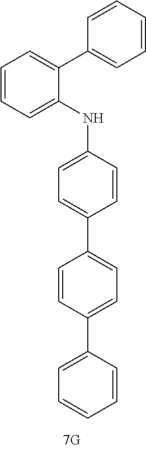 | 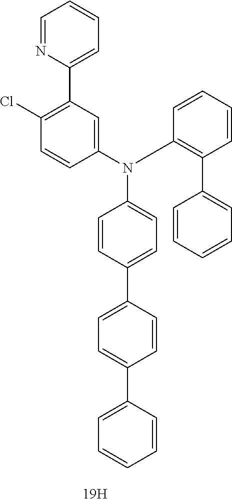 |
| 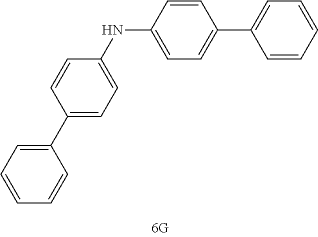 | 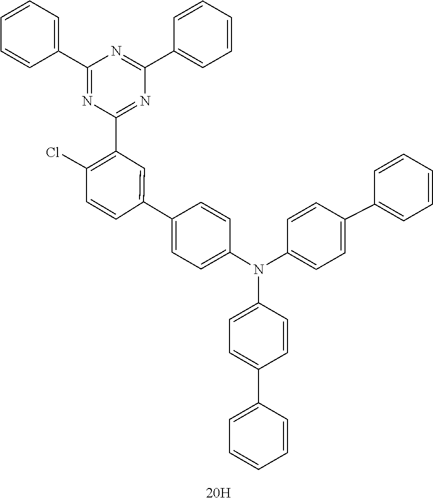 |
| 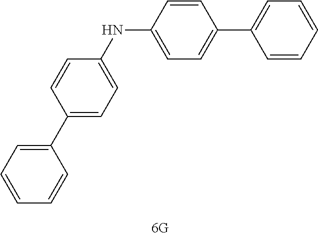 | 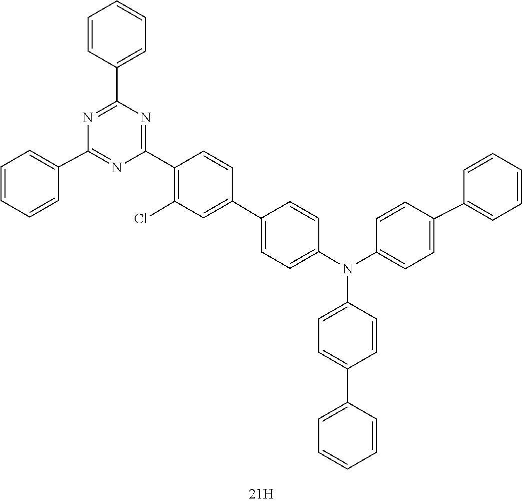 |
| 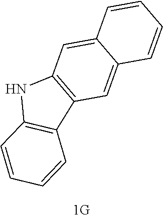 | 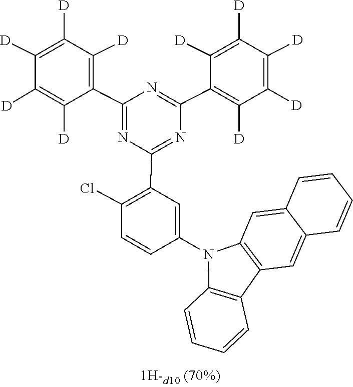 |
| 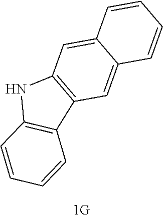 | 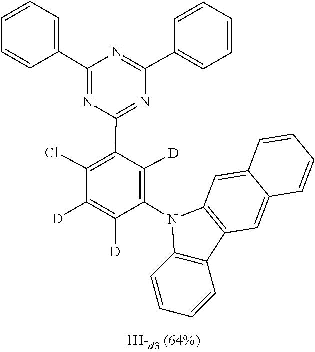 |

### <Preparation Example 4> Preparation of Compound 133J

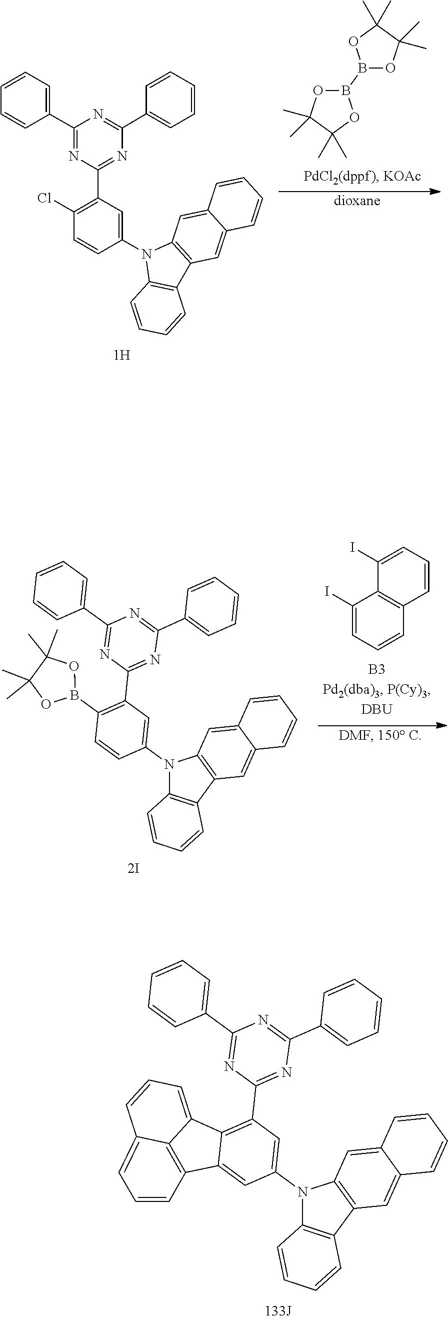

### 1) Synthesis of Compound 2I

Compound 1H (4.7 g, 8.4 mmol), bis(pinacolato)diboron (2.7 g, 10.9 mmol), Pd(dppf)Cl₂ (0.6 g, 0.84 mmol) and KOAc (1.7 g, 16.8 mmol) were put into a 100-mL round bottom flask, and a nitrogen atmosphere was created. Thereafter, dioxane (60 mL) was added thereto, and the resulting mixture was stirred at 110° C. for 4 hours. After the reaction was completed, the reaction temperature was lowered to room temperature, and the resulting product was washed with water, and then extracted with MC. The extracted organic solvent was dried over Mg₂SO₄, and then concentrated. The concentrated organic solvent was separated by silica-gel column and recrystallized to obtain Compound 2I (4.0 g, 6.2 mmol, 74%) which is a white solid compound.

### 2) Synthesis of Compound 133J

Compound 2I (4.0 g, 6.2 mmol), Compound B3 (2.5 g, 6.82 mmol), Pd₂(dba)₃ (0.3 g, 0.31 mmol), PCy3 (0.6 g, 2.48 mmol) and DBU (1 g, 6.2 mmol) were put into a 100-mL round bottom flask, and a nitrogen atmosphere was created. Thereafter, DMF (20 mL) was added thereto, and the resulting mixture was stirred at 150° C. for 24 hours. After the reaction was completed, the reaction temperature was lowered to room temperature, and the resulting product was washed with water, and then extracted with MC. The extracted organic solvent was dried over Mg₂SO₄, and then concentrated. The concentrated organic solvent was separated by silica-gel column and recrystallized to obtain Compound 133J (2.0 g, 3.22 mmol, 52%) which is a white solid compound.

Compound J in the following Table 4 was synthesized in the same manner as in Preparation Example 4, except that Compound H in the following Table 4 was used instead of Compound 1H in the synthesis process of Compound 2I and Compound I in the following Table 4 was used instead of Compound 2I in the synthesis process of Compound 133J.

TABLE 4
H

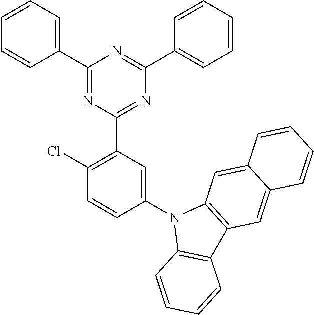 [SMILES: Clc1ccc(-n2c3ccccc3c3cc4ccccc4cc32)cc1-c1nc(-c2ccccc2)nc(-c2ccccc2)n1]

1H

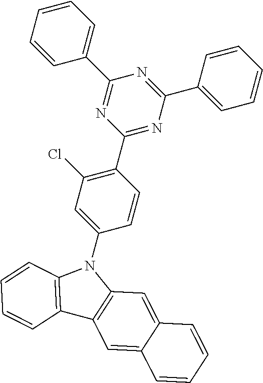 [SMILES: Clc1cc(-n2c3ccccc3c3cc4ccccc4cc32)ccc1-c1nc(-c2ccccc2)nc(-c2ccccc2)n1]

2H

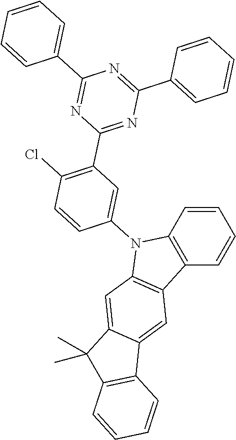 [SMILES: CC1(C)c2ccccc2-c2cc3c4ccccc4n(-c4ccc(Cl)c(-c5nc(-c6ccccc6)nc(-c6ccccc6)n5)c4)c3cc21]

3H

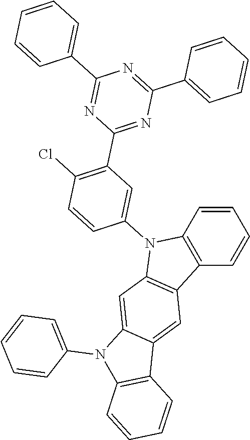 [SMILES: Clc1ccc(-n2c3ccccc3c3cc4c5ccccc5n(-c5ccccc5)c4cc32)cc1-c1nc(-c2ccccc2)nc(-c2ccccc2)n1]

4H

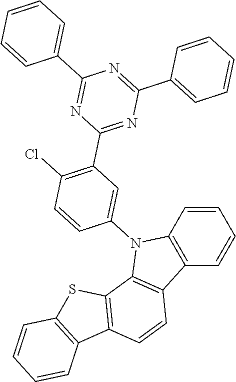 [SMILES: Clc1ccc(-n2c3ccccc3c3ccc4c5ccccc5sc4c32)cc1-c1nc(-c2ccccc2)nc(-c2ccccc2)n1]

5H

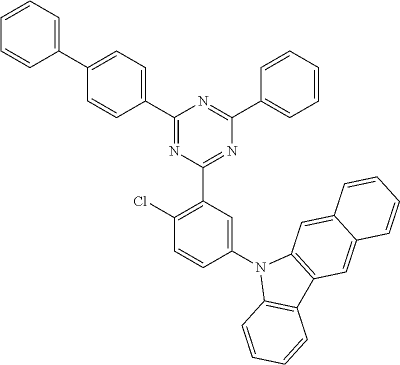 [SMILES: Clc1ccc(-n2c3ccccc3c3cc4ccccc4cc32)cc1-c1nc(-c2ccccc2)nc(-c2ccc(-c3ccccc3)cc2)n1]

6H

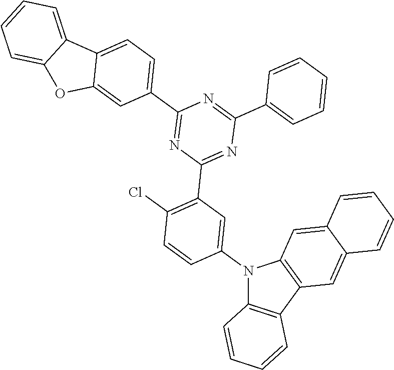 [SMILES: Clc1ccc(-n2c3ccccc3c3cc4ccccc4cc32)cc1-c1nc(-c2ccccc2)nc(-c2ccc3c(c2)oc2ccccc23)n1]

7H

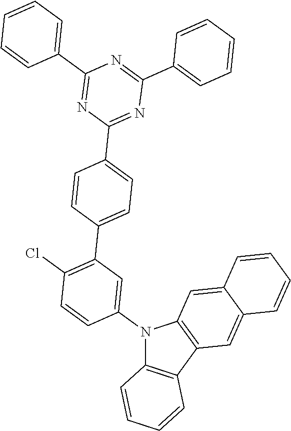 [SMILES: Clc1ccc(-n2c3ccccc3c3cc4ccccc4cc32)cc1-c1ccc(-c2nc(-c3ccccc3)nc(-c3ccccc3)n2)cc1]

8H

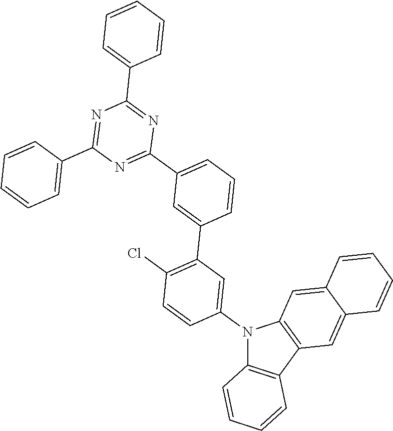 [SMILES: Clc1ccc(-n2c3ccccc3c3cc4ccccc4cc32)cc1-c1cccc(-c2nc(-c3ccccc3)nc(-c3ccccc3)n2)c1]

9H

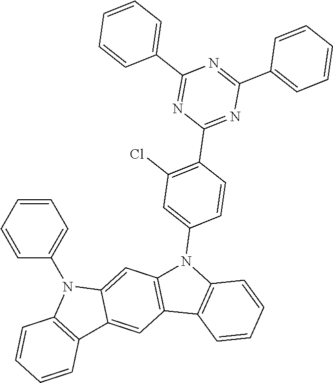 [SMILES: Clc1cc(-n2c3ccccc3c3cc4c5ccccc5n(-c5ccccc5)c4cc32)ccc1-c1nc(-c2ccccc2)nc(-c2ccccc2)n1]

10H

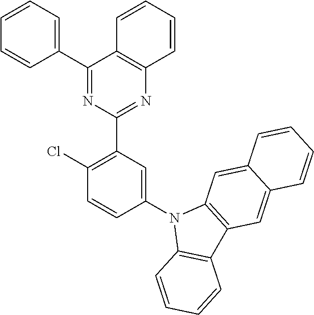 [SMILES: Clc1ccc(-n2c3ccccc3c3cc4ccccc4cc32)cc1-c1nc(-c2ccccc2)c2ccccc2n1]

11H

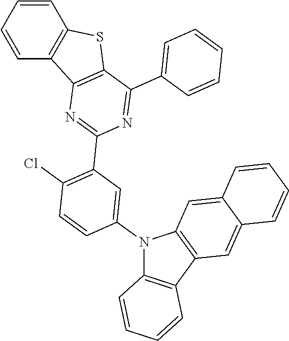 [SMILES: Clc1ccc(-n2c3ccccc3c3cc4ccccc4cc32)cc1-c1nc(-c2ccccc2)c2sc3ccccc3c2n1]

12H

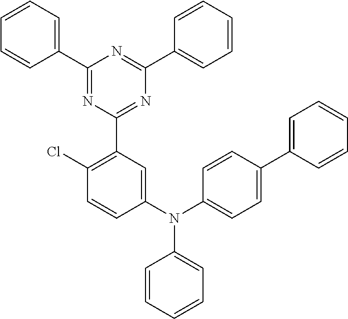 [SMILES: Clc1ccc(N(c2ccccc2)c2ccc(-c3ccccc3)cc2)cc1-c1nc(-c2ccccc2)nc(-c2ccccc2)n1]

13H

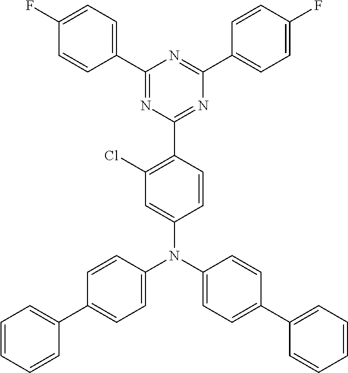 [SMILES: Fc1ccc(-c2nc(-c3ccc(F)cc3)nc(-c3ccc(N(c4ccc(-c5ccccc5)cc4)c4ccc(-c5ccccc5)cc4)cc3Cl)n2)cc1]

14H

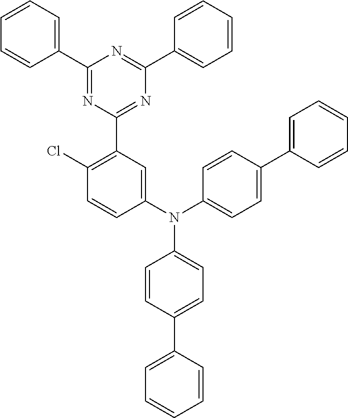 [SMILES: Clc1ccc(N(c2ccc(-c3ccccc3)cc2)c2ccc(-c3ccccc3)cc2)cc1-c1nc(-c2ccccc2)nc(-c2ccccc2)n1]

15H

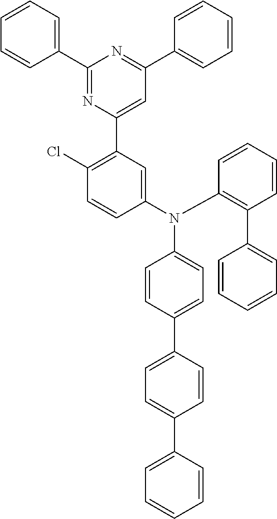 [SMILES: Clc1ccc(N(c2ccc(-c3ccc(-c4ccccc4)cc3)cc2)c2ccccc2-c2ccccc2)cc1-c1cc(-c2ccccc2)nc(-c2ccccc2)n1]

16H

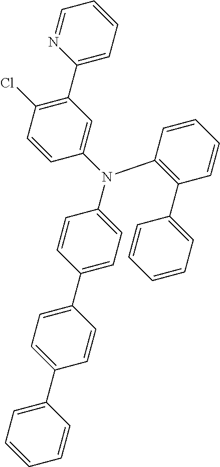 [SMILES: Clc1ccc(N(c2ccc(-c3ccc(-c4ccccc4)cc3)cc2)c2ccccc2-c2ccccc2)cc1-c1ccccn1]

17H

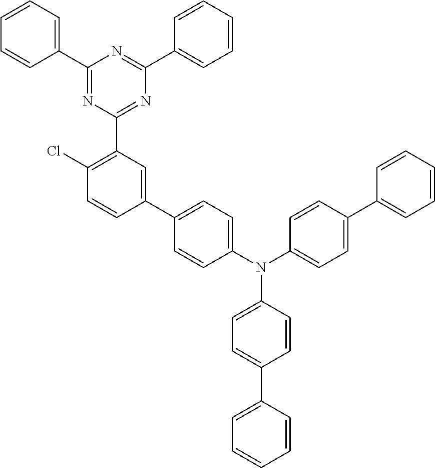 [SMILES: Clc1ccc(-c2ccc(N(c3ccc(-c4ccccc4)cc3)c3ccc(-c4ccccc4)cc3)cc2)cc1-c1nc(-c2ccccc2)nc(-c2ccccc2)n1]

18H

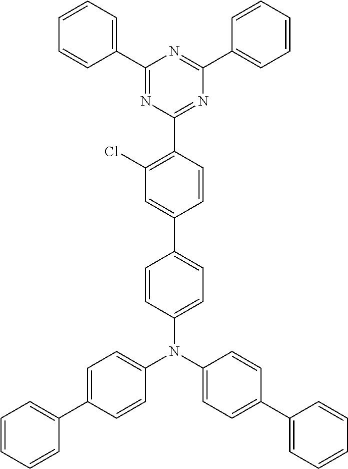 [SMILES: Clc1cc(-c2ccc(N(c3ccc(-c4ccccc4)cc3)c3ccc(-c4ccccc4)cc3)cc2)ccc1-c1nc(-c2ccccc2)nc(-c2ccccc2)n1]

19H

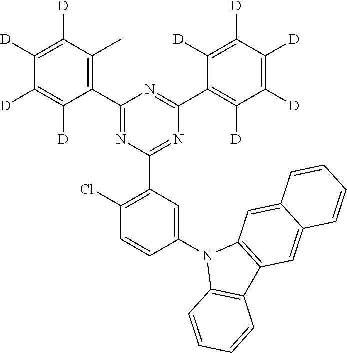 [SMILES: [2H]c1c([2H])c([2H])c(-c2nc(-c3cc(-n4c5ccccc5c5cc6ccccc6cc54)ccc3Cl)nc(-c3c([2H])c([2H])c([2H])c([2H])c3C)n2)c([2H])c1[2H]]

1H-_{d10}

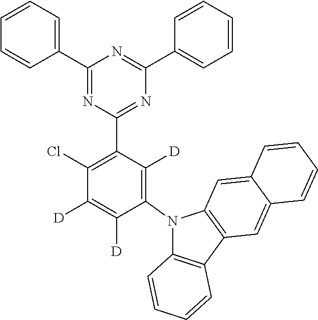 [SMILES: [2H]c1c([2H])c(-n2c3ccccc3c3cc4ccccc4cc32)c([2H])c(-c2nc(-c3ccccc3)nc(-c3ccccc3)n2)c1Cl]

1H-_{d3}
I

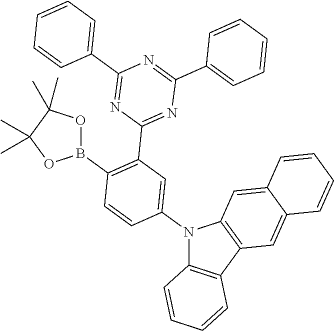 [SMILES: CC1(C)OB(c2ccc(-n3c4ccccc4c4cc5ccccc5cc43)cc2-c2nc(-c3ccccc3)nc(-c3ccccc3)n2)OC1(C)C]

2I (76%)

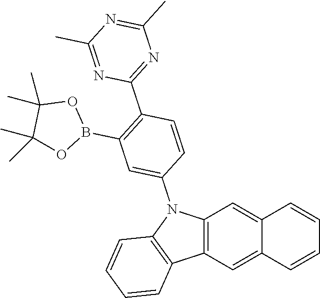 [SMILES: Cc1nc(C)nc(-c2ccc(-n3c4ccccc4c4cc5ccccc5cc43)cc2B2OC(C)(C)C(C)(C)O2)n1]

3I (70%)

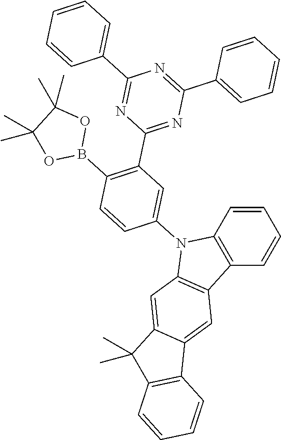 [SMILES: CC1(C)c2ccccc2-c2cc3c4ccccc4n(-c4ccc(B5OC(C)(C)C(C)(C)O5)c(-c5nc(-c6ccccc6)nc(-c6ccccc6)n5)c4)c3cc21]

4I (55%)

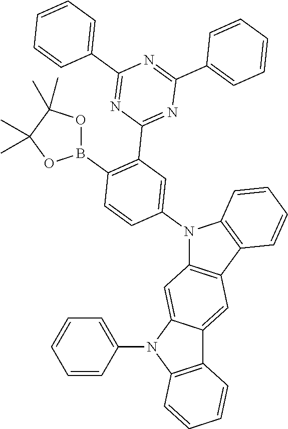 [SMILES: CC1(C)OB(c2ccc(-n3c4ccccc4c4cc5c6ccccc6n(-c6ccccc6)c5cc43)cc2-c2nc(-c3ccccc3)nc(-c3ccccc3)n2)OC1(C)C]

5I (66%)

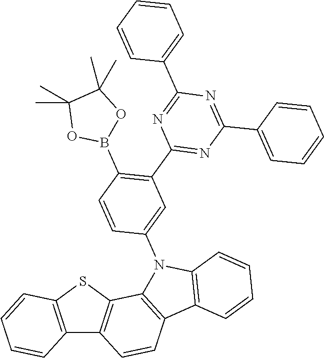 [SMILES: CC1(C)OB(c2ccc(-n3c4ccccc4c4ccc5c6ccccc6sc5c43)cc2-c2nc(-c3ccccc3)nc(-c3ccccc3)n2)OC1(C)C]

6I (62%)

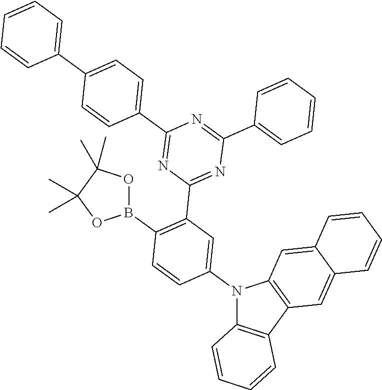 [SMILES: CC1(C)OB(c2ccc(-n3c4ccccc4c4cc5ccccc5cc43)cc2-c2nc(-c3ccccc3)nc(-c3ccc(-c4ccccc4)cc3)n2)OC1(C)C]

7I (59%)

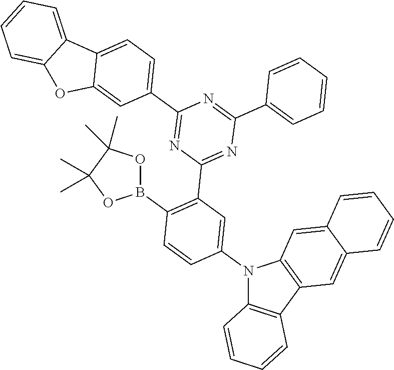 [SMILES: CC1(C)OB(c2ccc(-n3c4ccccc4c4cc5ccccc5cc43)cc2-c2nc(-c3ccccc3)nc(-c3ccc4c(c3)oc3ccccc34)n2)OC1(C)C]

8I (66%)

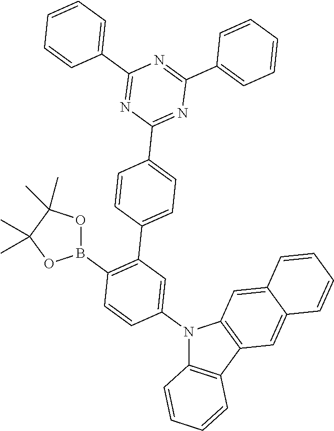 [SMILES: CC1(C)OB(c2ccc(-n3c4ccccc4c4cc5ccccc5cc43)cc2-c2ccc(-c3nc(-c4ccccc4)nc(-c4ccccc4)n3)cc2)OC1(C)C]

9I (59%)

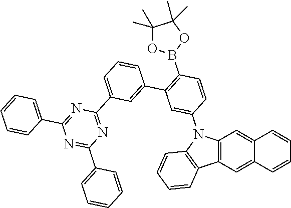 [SMILES: CC1(C)OB(c2ccc(-n3c4ccccc4c4cc5ccccc5cc43)cc2-c2cccc(-c3nc(-c4ccccc4)nc(-c4ccccc4)n3)c2)OC1(C)C]

10I (56%)

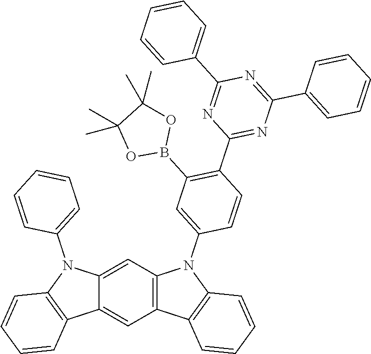 [SMILES: CC1(C)OB(c2cc(-n3c4ccccc4c4cc5c6ccccc6n(-c6ccccc6)c5cc43)ccc2-c2nc(-c3ccccc3)nc(-c3ccccc3)n2)OC1(C)C]

11I (50%)

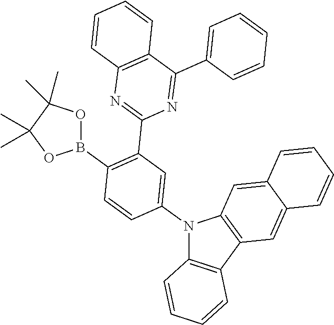 [SMILES: CC1(C)OB(c2ccc(-n3c4ccccc4c4cc5ccccc5cc43)cc2-c2nc(-c3ccccc3)c3ccccc3n2)OC1(C)C]

12I (48%)

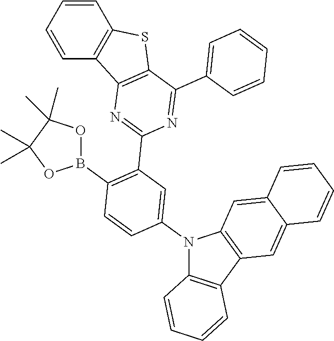 [SMILES: CC1(C)OB(c2ccc(-n3c4ccccc4c4cc5ccccc5cc43)cc2-c2nc(-c3ccccc3)c3sc4ccccc4c3n2)OC1(C)C]

13I (66%)

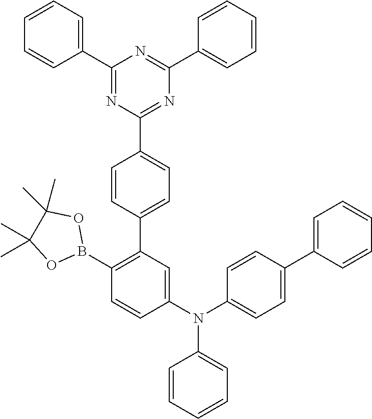 [SMILES: CC1(C)OB(c2ccc(N(c3ccccc3)c3ccc(-c4ccccc4)cc3)cc2-c2ccc(-c3nc(-c4ccccc4)nc(-c4ccccc4)n3)cc2)OC1(C)C]

14I (76%)

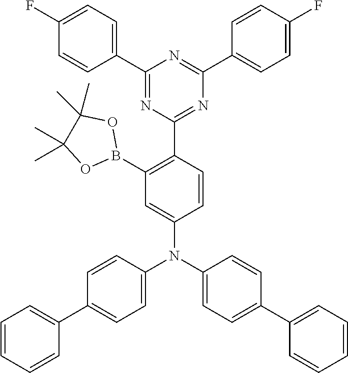 [SMILES: CC1(C)OB(c2cc(N(c3ccc(-c4ccccc4)cc3)c3ccc(-c4ccccc4)cc3)ccc2-c2nc(-c3ccc(F)cc3)nc(-c3ccc(F)cc3)n2)OC1(C)C]

15I (77%)

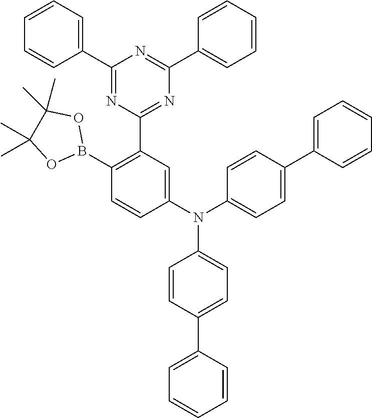 [SMILES: CC1(C)OB(c2ccc(N(c3ccc(-c4ccccc4)cc3)c3ccc(-c4ccccc4)cc3)cc2-c2nc(-c3ccccc3)nc(-c3ccccc3)n2)OC1(C)C]

16I (76%)

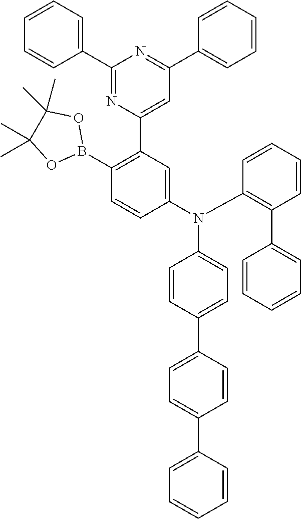 [SMILES: CC1(C)OB(c2ccc(N(c3ccc(-c4ccc(-c5ccccc5)cc4)cc3)c3ccccc3-c3ccccc3)cc2-c2cc(-c3ccccc3)nc(-c3ccccc3)n2)OC1(C)C]

17I (76%)

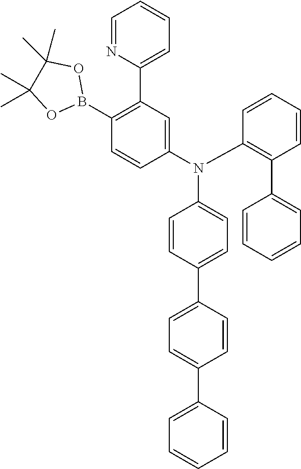 [SMILES: CC1(C)OB(c2ccc(N(c3ccc(-c4ccc(-c5ccccc5)cc4)cc3)c3ccccc3-c3ccccc3)cc2-c2ccccn2)OC1(C)C]

18I (76%)

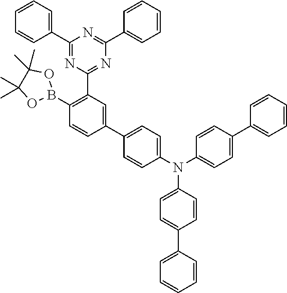 [SMILES: CC1(C)OB(c2ccc(-c3ccc(N(c4ccc(-c5ccccc5)cc4)c4ccc(-c5ccccc5)cc4)cc3)cc2-c2nc(-c3ccccc3)nc(-c3ccccc3)n2)OC1(C)C]

19I (58%)

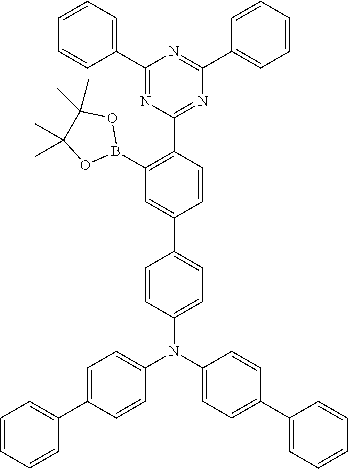 [SMILES: CC1(C)OB(c2cc(-c3ccc(N(c4ccc(-c5ccccc5)cc4)c4ccc(-c5ccccc5)cc4)cc3)ccc2-c2nc(-c3ccccc3)nc(-c3ccccc3)n2)OC1(C)C]

20I (55%)

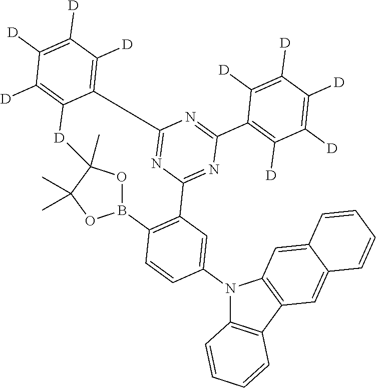 [SMILES: [2H]c1c([2H])c([2H])c(-c2nc(-c3cc(-n4c5ccccc5c5cc6ccccc6cc54)ccc3B3OC(C)(C)C(C)(C)O3)nc(-c3c([2H])c([2H])c([2H])c([2H])c3[2H])n2)c([2H])c1[2H]]

2I-_{d10} (71%)

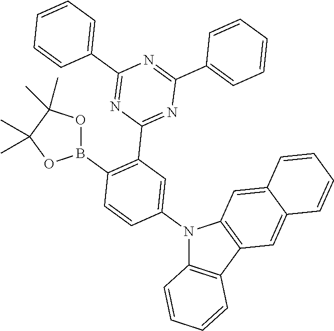 [SMILES: CC1(C)OB(c2ccc(-n3c4ccccc4c4cc5ccccc5cc43)cc2-c2nc(-c3ccccc3)nc(-c3ccccc3)n2)OC1(C)C]

21-_{d3} (73%)
J (yield)

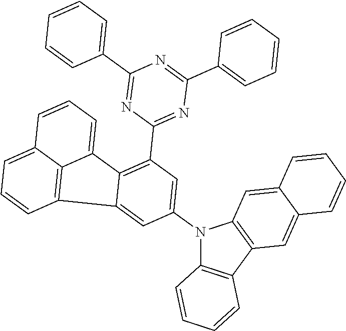 [SMILES: c1ccc(-c2nc(-c3ccccc3)nc(-c3cc(-n4c5ccccc5c5cc6ccccc6cc54)cc4c3-c3cccc5cccc-4c35)n2)cc1]

133J (52%)

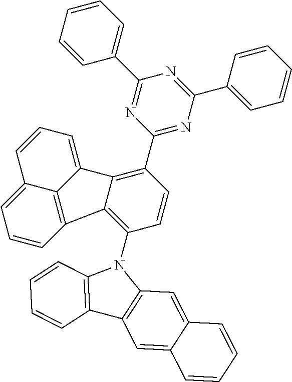 [SMILES: c1ccc(-c2nc(-c3ccccc3)nc(-c3ccc(-n4c5ccccc5c5cc6ccccc6cc54)c4c3-c3cccc5cccc-4c35)n2)cc1]

134J (66%)

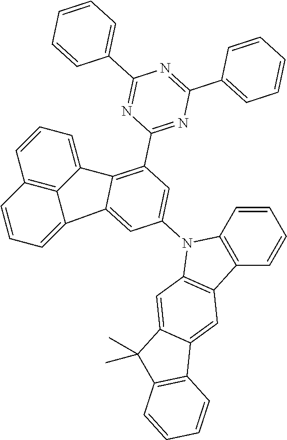 [SMILES: CC1(C)c2ccccc2-c2cc3c4ccccc4n(-c4cc(-c5nc(-c6ccccc6)nc(-c6ccccc6)n5)c5c(c4)-c4cccc6cccc-5c46)c3cc21]

135J (42%)

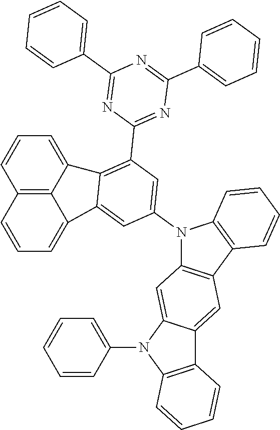 [SMILES: c1ccc(-c2nc(-c3ccccc3)nc(-c3cc(-n4c5ccccc5c5cc6c7ccccc7n(-c7ccccc7)c6cc54)cc4c3-c3cccc5cccc-4c35)n2)cc1]

136J (39%)

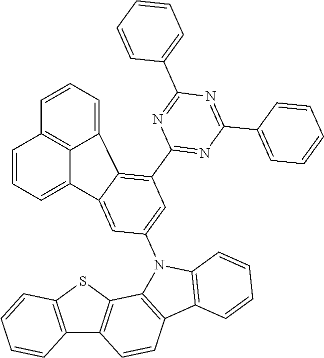 [SMILES: c1ccc(-c2nc(-c3ccccc3)nc(-c3cc(-n4c5ccccc5c5ccc6c7ccccc7sc6c54)cc4c3-c3cccc5cccc-4c35)n2)cc1]

138J (32%)

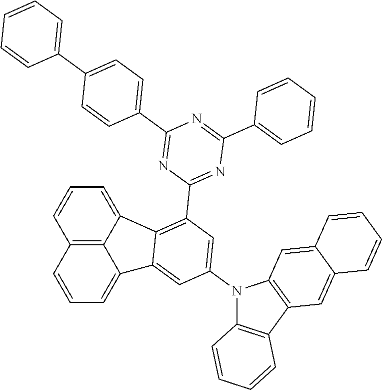 [SMILES: c1ccc(-c2ccc(-c3nc(-c4ccccc4)nc(-c4cc(-n5c6ccccc6c6cc7ccccc7cc65)cc5c4-c4cccc6cccc-5c46)n3)cc2)cc1]

142J (49%)

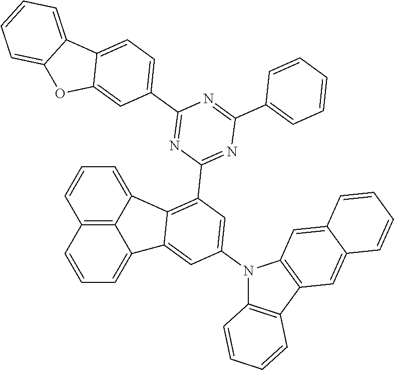 [SMILES: c1ccc(-c2nc(-c3ccc4c(c3)oc3ccccc34)nc(-c3cc(-n4c5ccccc5c5cc6ccccc6cc54)cc4c3-c3cccc5cccc-4c35)n2)cc1]

143J (52%)

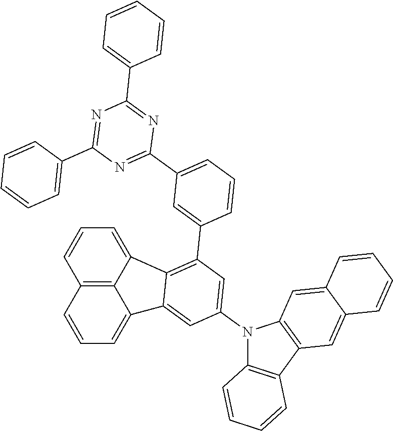 [SMILES: c1ccc(-c2nc(-c3ccccc3)nc(-c3cccc(-c4cc(-n5c6ccccc6c6cc7ccccc7cc65)cc5c4-c4cccc6cccc-5c46)c3)n2)cc1]

145J (39%)

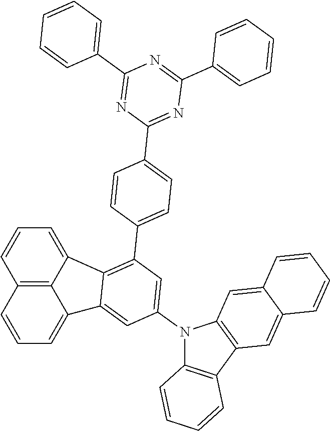 [SMILES: c1ccc(-c2nc(-c3ccccc3)nc(-c3ccc(-c4cc(-n5c6ccccc6c6cc7ccccc7cc65)cc5c4-c4cccc6cccc-5c46)cc3)n2)cc1]

146J (52%)

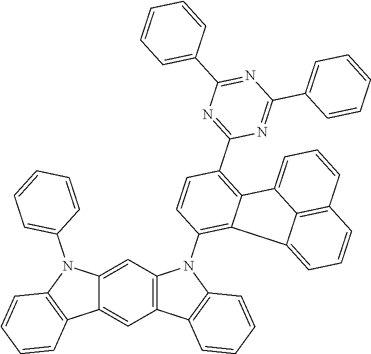 [SMILES: c1ccc(-c2nc(-c3ccccc3)nc(-c3ccc(-n4c5ccccc5c5cc6c7ccccc7n(-c7ccccc7)c6cc54)c4c3-c3cccc5cccc-4c35)n2)cc1]

151J (27%)

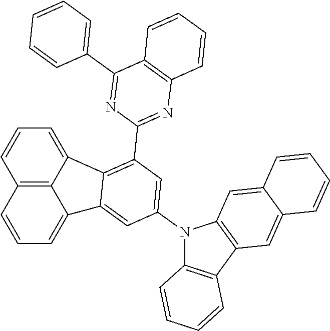 [SMILES: c1ccc(-c2nc(-c3cc(-n4c5ccccc5c5cc6ccccc6cc54)cc4c3-c3cccc5cccc-4c35)nc3ccccc23)cc1]

160J (57%)

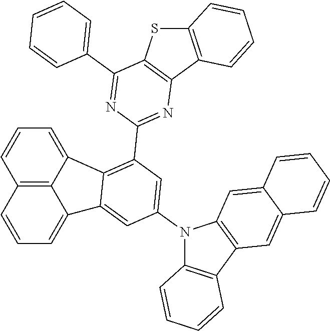 [SMILES: c1ccc(-c2nc(-c3cc(-n4c5ccccc5c5cc6ccccc6cc54)cc4c3-c3cccc5cccc-4c35)nc3c2sc2ccccc23)cc1]

162J (44%)

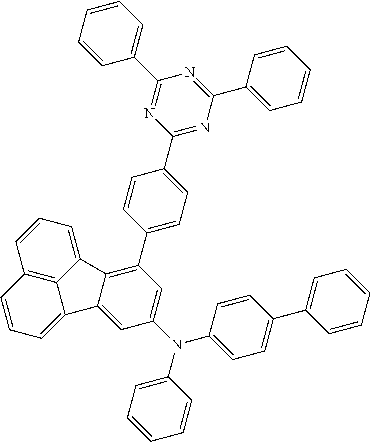 [SMILES: c1ccc(-c2ccc(N(c3ccccc3)c3cc(-c4ccc(-c5nc(-c6ccccc6)nc(-c6ccccc6)n5)cc4)c4c(c3)-c3cccc5cccc-4c35)cc2)cc1]

178J (49%)

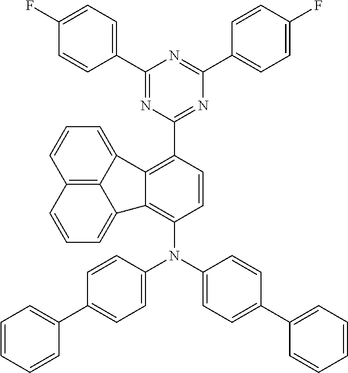 [SMILES: Fc1ccc(-c2nc(-c3ccc(F)cc3)nc(-c3ccc(N(c4ccc(-c5ccccc5)cc4)c4ccc(-c5ccccc5)cc4)c4c3-c3cccc5cccc-4c35)n2)cc1]

189J (37%)

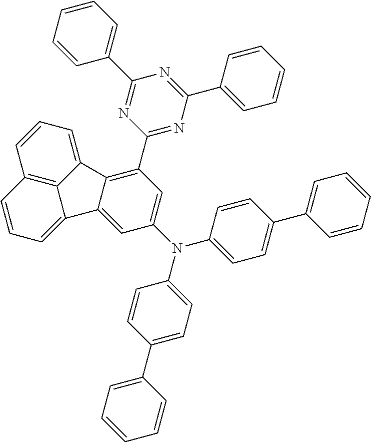 [SMILES: c1ccc(-c2ccc(N(c3ccc(-c4ccccc4)cc3)c3cc(-c4nc(-c5ccccc5)nc(-c5ccccc5)n4)c4c(c3)-c3cccc5cccc-4c35)cc2)cc1]

194J (45%)

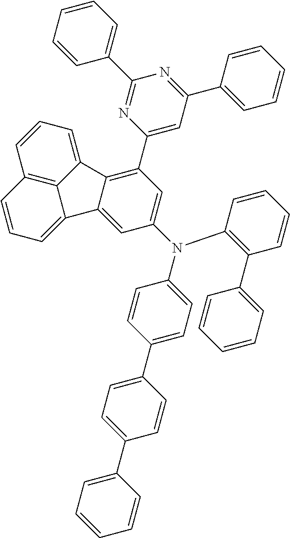 [SMILES: c1ccc(-c2ccc(-c3ccc(N(c4cc(-c5cc(-c6ccccc6)nc(-c6ccccc6)n5)c5c(c4)-c4cccc6cccc-5c46)c4ccccc4-c4ccccc4)cc3)cc2)cc1]

198J (33%)

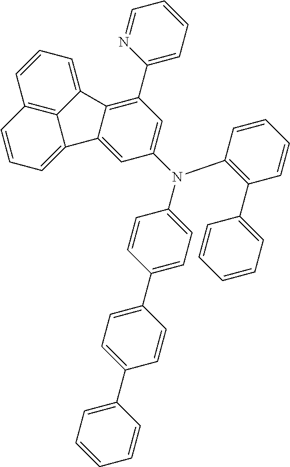 [SMILES: c1ccc(-c2ccc(-c3ccc(N(c4cc(-c5ccccn5)c5c(c4)-c4cccc6cccc-5c46)c4ccccc4-c4ccccc4)cc3)cc2)cc1]

201J (35%)

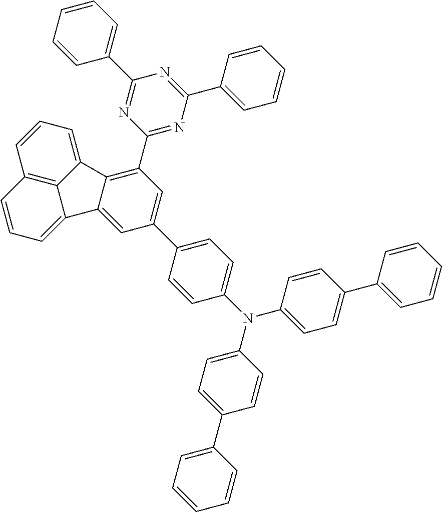 [SMILES: c1ccc(-c2ccc(N(c3ccc(-c4ccccc4)cc3)c3ccc(-c4cc(-c5nc(-c6ccccc6)nc(-c6ccccc6)n5)c5c(c4)-c4cccc6cccc-5c46)cc3)cc2)cc1]

209J (43%)

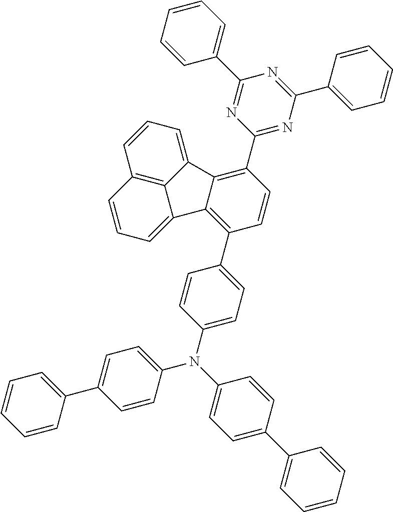 [SMILES: c1ccc(-c2ccc(N(c3ccc(-c4ccccc4)cc3)c3ccc(-c4ccc(-c5nc(-c6ccccc6)nc(-c6ccccc6)n5)c5c4-c4cccc6cccc-5c46)cc3)cc2)cc1]

210J (39%)

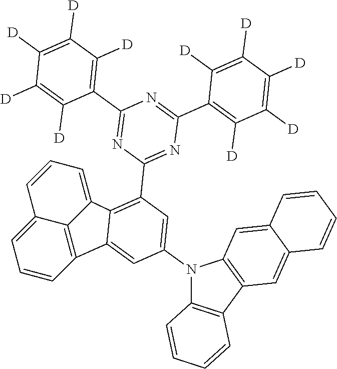 [SMILES: [2H]c1c([2H])c([2H])c(-c2nc(-c3cc(-n4c5ccccc5c5cc6ccccc6cc54)cc4c3-c3cccc5cccc-4c35)nc(-c3c([2H])c([2H])c([2H])c([2H])c3[2H])n2)c([2H])c1[2H]]

233J (52%)

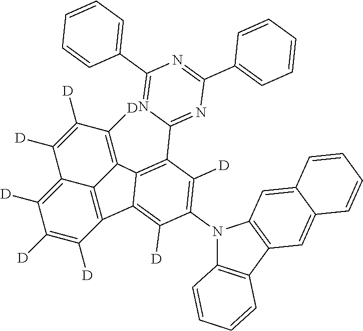 [SMILES: [2H]c1c(-c2nc(-c3ccccc3)nc(-c3ccccc3)n2)c2c(c([2H])c1-n1c3ccccc3c3cc4ccccc4cc31)-c1c([2H])c([2H])c([2H])c3c([2H])c([2H])c([2H])c-2c13]

235J (49%)

### <Preparation Example 5> Preparation of Compound 169J

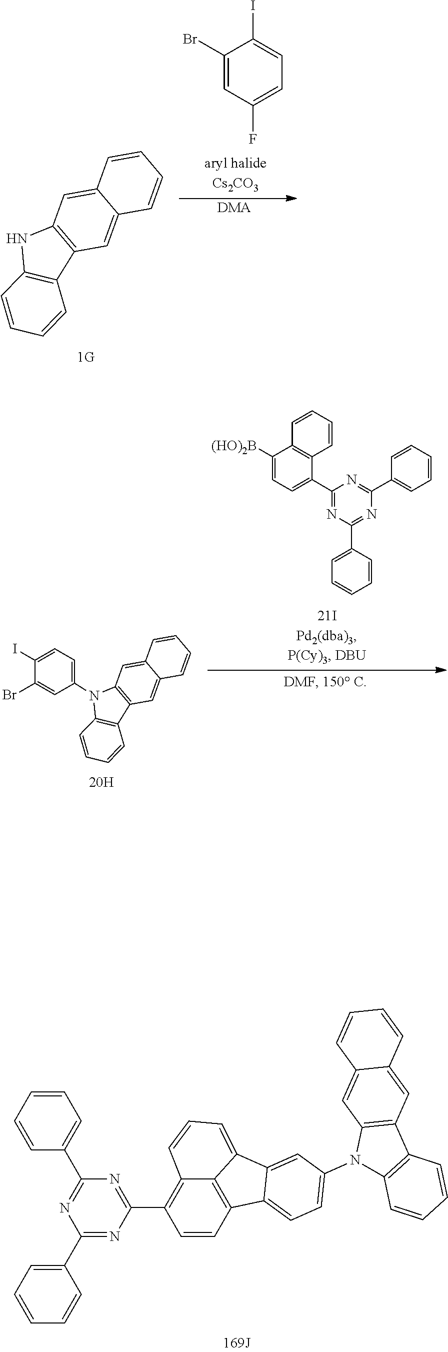

### 1) Synthesis of Compound 20H

Compound 1G (15 g, 69 mmol) and Cs₂CO₃ (48 g, 150 mmol) were put into a 500-mL round bottom flask, and dissolved by adding dimethylacetamide (hereinafter, referred to as DMA) (200 mL) thereto. And then, 2-bromo-4-fluoro-1-iodobenzene (22.8 g, 76 mmol) was diluted in DMA (30 mL) and slowly added dropwise to the reaction mixture. After the resulting mixture was stirred at 140° C. for 3 hours, the reaction temperature was lowered to room temperature, and the resulting product was washed with water, and then extracted with MC. The extracted organic solvent was dried over Mg₂SO₄, and then concentrated. The concentrated organic solvent was separated by silica-gel column and recrystallized to obtain Compound 20H (30.0 g, 60.7 mmol, 89%) which is a white solid compound.

### 2) Synthesis of Compound 169J

Compound 20H (30 g, 60.7 mmol), Compound 21I (27 g, 67 mmol), Pd₂(dba)₃ (3 g, 3.1 mmol), PCy₃ (6 g, 24.8 mmol) and DBU (10 g, 62 mmol) were put into a 1000-mL round bottom flask, and a nitrogen atmosphere was created. Thereafter, DMF (200 mL) was added thereto, and the resulting mixture was refluxed at 150° C. for 36 hours. After the reaction was completed, the reaction temperature was lowered to room temperature, and the resulting product was washed with water, and then extracted with MC. The extracted organic solvent was dried over Mg₂SO₄, and then concentrated. The concentrated organic solvent was separated by silica-gel column and recrystallized to obtain Compound 169J (11.4 g, 17.6 mmol, 29%) which is a yellow solid compound.

Compound J in the following Table 5 was synthesized in the same manner as in Preparation Example 5, except that Compound G in the following Table 5 was used instead of Compound 1G and a compound, which is aryl halide in the following Table 5, was used instead of 2-bromo-4-fluoro-1-iodobenzene in the synthesis process of Compound 20H, and Compound H in the following Table 5 was used instead of Compound 20H in the synthesis process of Compound 169J.

TABLE 5

| | Aryl | H | |
|---|---|---|---|
| G | Halide | (yield) | J (yield) |
| 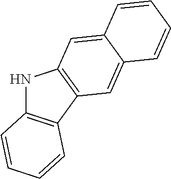 | 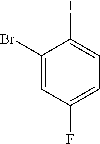 | 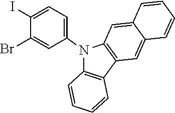 20H (88%) | 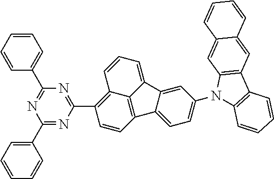 [SMILES: c1ccc(-c2nc(-c3ccccc3)nc(-c3ccc4c5c(cccc35)-c3cc(-n5c6ccccc6c6cc7ccccc7cc65)ccc3-4)n2)cc1] |
| 1G | | | 169J (29%) |
| 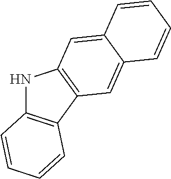 1G | 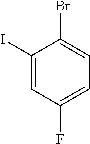 | 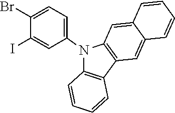 21H (79%) | 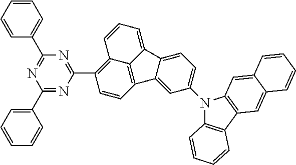 [SMILES: c1ccc(-c2nc(-c3ccccc3)nc(-c3ccc4c5c(cccc35)-c3ccc(-n5c6ccccc6c6cc7ccccc7cc65)cc3-4)n2)cc1] 170J (32%) |

### <Preparation Example 6> Preparation of Compound 228J

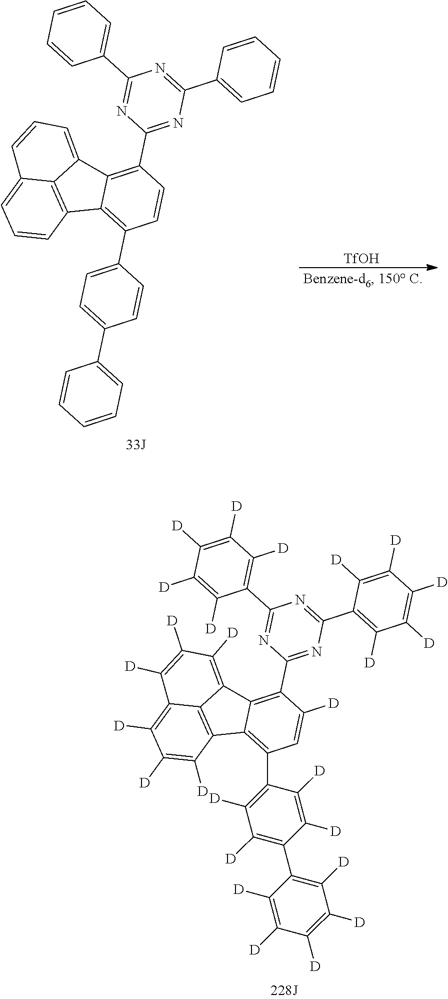 [SMILES: c1ccc(-c2ccc(-c3ccc(-c4nc(-c5ccccc5)nc(-c5ccccc5)n4)c4c3-c3cccc5cccc-4c35)cc2)cc1 ; "TfOH" ; [2H]c1cc(-c2c([2H])c([2H])c(-c3c([2H])c([2H])c([2H])c([2H])c3[2H])c([2H])c2[2H])c2c(c1-c1nc(-c3c([2H])c([2H])c([2H])c([2H])c3[2H])nc(-c3c([2H])c([2H])c([2H])c([2H])c3[2H])n1)-c1c([2H])c([2H])c([2H])c3c([2H])c([2H])c([2H])c-2c13]

### 1) Synthesis of Compound 228J

Compound 33J (4 g, 6.8 mmol) was put into a 100-mL round bottom flask, and the atmosphere in the flask was substituted with a nitrogen atmosphere. Thereafter, after the compound was dissolved in a benzene-d₆ solvent (40 mL), triflic acid (TfOH) (7 mL, 46 mmol) was slowly added dropwise thereto. The temperature was lowered from 60° C. to room temperature, the resulting mixture was stirred for 2 hours, and then the solvent was removed by reducing pressure. The concentrated organic material was washed with water, and extracted with MC. The extracted organic solvent was dried over Mg₂SO₄, and then concentrated. The concentrated organic solvent was separated by silica-gel column and recrystallized to obtain Compound 228J (3.7 g, 6.12 mmol, 90%) which is a yellow solid compound.

Compound J in the following Table 6 was synthesized in the same manner as in Preparation Example 6, except that Compound J in the following Table 6 was used instead of Compound 33J in the synthesis process of Compound 228J.

TABLE 6

| J | J-_{d} (yield) |
|---|---|
| 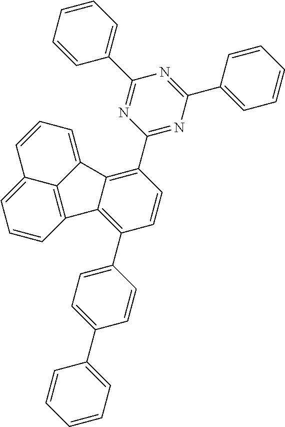 33J | 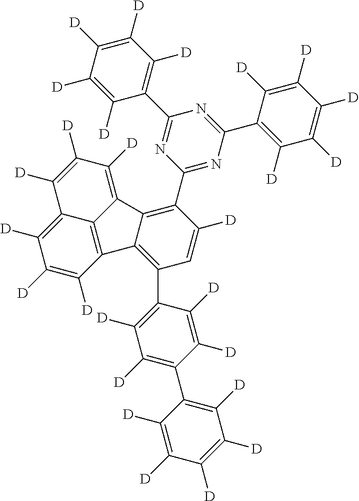 [SMILES: [2H]c1cc(-c2c([2H])c([2H])c(-c3c([2H])c([2H])c([2H])c([2H])c3[2H])c([2H])c2[2H])c2c(c1-c1nc(-c3c([2H])c([2H])c([2H])c([2H])c3[2H])nc(-c3c([2H])c([2H])c([2H])c([2H])c3[2H])n1)-c1c([2H])c([2H])c([2H])c3c([2H])c([2H])c([2H])c-2c13] |
| | 228J (90%) |
| 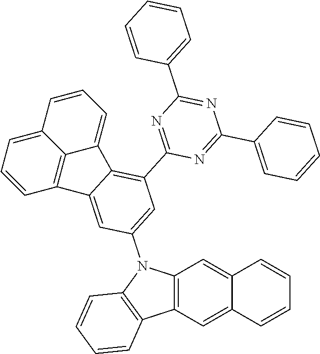 133J | 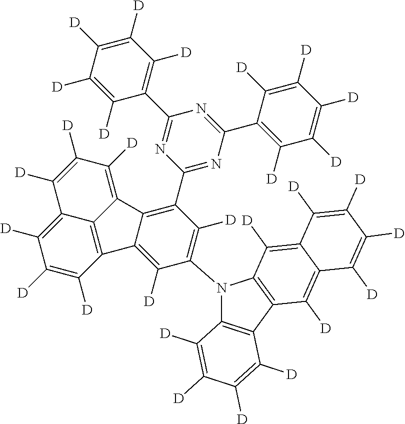 [SMILES: [2H]c1c([2H])c([2H])c(-c2nc(-c3c([2H])c([2H])c([2H])c([2H])c3[2H])nc(-c3c([2H])c(-n4c5c([2H])c([2H])c([2H])c([2H])c5c5c([2H])c6c([2H])c([2H])c([2H])c([2H])c6c([2H])c54)c([2H])c4c3-c3c([2H])c([2H])c([2H])c5c([2H])c([2H])c([2H])c-4c35)n2)c([2H])c1[2H]] |
| | 237J (94%) |

The synthetic confirmation data of the compounds prepared above are as shown in the following Tables 7 and 8.

TABLE 7

| Compound | FD-Mass |
|---|---|
| 1J | m/z = 508.62 |
| | (C38H24N2, 508.19) |
| 2J | m/z = 509.61 |
| | (C37H23N3, 509.19) |
| 3J | m/z = 585.71 |
| | (C43H27N3, 585.22) |
| 4J | m/z = 599.69 |
| | (C43H25N3O, 599.20) |
| 5J | m/z = 508.62 |
| | (C38H24N2, 508.19) |
| 6J | m/z = 559.67 |
| | (C41H25N3, 559.20) |
| 7J | m/z = 661.81 |
| | (C49H31N3, 661.25) |
| 8J | m/z = 599.69 |
| | (C43H25N3O, 599.20) |
| 9J | m/z = 599.69 |
| | (C43H25N3O, 599.20) |
| 10J | m/z = 599.69 |
| | (C43H25N3O, 599.20) |
| 11J | m/z = 599.69 |
| | (C43H25N3O, 599.20) |
| 12J | m/z = 599.69 |
| | (C43H25N3O, 599.20) |
| 13J | m/z = 599.69 |
| | (C43H25N3O, 599.20) |
| 14J | m/z = 599.69 |
| | (C43H25N3O, 599.20) |
| 15J | m/z = 599.69 |
| | (C43H25N3O, 599.20) |
| 16J | m/z = 599.69 |
| | (C43H25N3O, 599.20) |
| 17J | m/z = 482.59 |
| | (C36H22N2, 482.18) |
| 18J | m/z = 482.59 |
| | (C36H22N2, 482.18) |
| 19J | m/z = 538.67 |
| | (C38H22N2S, 538.15) |
| 20J | m/z = 522.61 |
| | (C38H22N2O, 522.17) |
| 21J | m/z = 532.65 |
| | (C40H24N2, 532.19) |
| 22J | m/z = 556.67 |
| | (C42H24N2, 556.19) |
| 23J | m/z = 538.67 |
| | (C38H22N2S, 538.15) |
| 24J | m/z = 522.61 |
| | (C38H22N2O, 522.17) |
| 25J | m/z = 532.65 |
| | (C40H24N2, 532.19) |
| 26J | m/z = 558.68 |
| | (C42H26N2, 558.21) |
| 27J | m/z = 588.73 |
| | (C42H24N2S, 558.17) |
| 28J | m/z = 598.71 |
| | (C44H26N2O, 59820) |
| 29J | m/z = 588.73 |
| | (C42H24N2S, 558.17) |
| 30J | m/z = 647.78 |
| | (C48H29N3, 647.24) |
| 31J | m/z = 572.67 |
| | (C42H24N2O, 572.19) |
| 32J | m/z = 612.69 |
| | (C44H24N2O2, 612.18) |
| 33J | m/z = 585.71 |
| | (C43H27N3, 585.22) |
| 34J | m/z = 558.68 |
| | (C42H26N2, 558.21) |
| 35J | m/z = 614.77 |
| | (C44H26N2S, 614.18) |
| 36J | m/z = 482.59 |
| | (C36H22N2, 482.18) |
| 37J | m/z = 482.59 |
| | (C36H22N2, 482.18) |
| 38J | m/z = 482.59 |
| | (C36H22N2, 482.18) |
| 39J | m/z = 482.59 |
| | (C36H22N2, 482.18) |
| 40J | m/z = 482.59 |
| | (C36H22N2, 482.18) |
| 41J | m/z = 509.61 |
| | (C37H23N3, 509.19) |
| 42J | m/z = 509.61 |
| | (C37H23N3, 509.19) |
| 43J | m/z = 509.61 |
| | (C37H23N3, 509.19) |
| 44J | m/z = 509.61 |
| | (C37H23N3, 509.19) |
| 45J | m/z = 559.67 |
| | (C41H25N3, 559.20) |
| 46J | m/z = 635.77 |
| | (C47H29N3, 635.24) |
| 47J | m/z = 609.73 |
| | (C45H27N3, 609.22) |
| 48J | m/z = 661.81 |
| | (C49H31N3, 661.25) |
| 49J | m/z = 599.69 |
| | (C43H25N3O, 599.20) |
| 50J | m/z = 599.69 |
| | (C43H25N3O, 599.20) |
| 51J | m/z = 599.69 |
| | (C43H25N3O, 599.20) |
| 52J | m/z = 599.69 |
| | (C43H25N3O, 599.20) |
| 53J | m/z = 649.75 |
| | (C47H27N3O, 649.22) |
| 54J | m/z = 615.75 |
| | (C43H25N3S, 615.18) |
| 55J | m/z = 615.75 |
| | (C43H25N3S, 615.18) |
| 56J | m/z = 615.75 |
| | (C43H25N3S, 615.18) |
| 57J | m/z = 649.75 |
| | (C47H27N3O, 649.22) |
| 58J | m/z = 674.81 |
| | (C49H30N4, 674.25) |
| 59J | m/z = 599.69 |
| | (C43H25N3O, 599.20) |
| 60J | m/z = 599.69 |
| | (C43H25N3O, 599.20) |
| 61J | m/z = 675.79 |
| | (C49H29N3O, 675.23) |
| 62J | m/z = 675.79 |
| | (C49H29N3O, 675.23) |
| 63J | m/z = 598.71 |
| | (C43H26N4, 598.22) |
| 64J | m/z = 674.81 |
| | (C49H30N4, 674.25) |
| 65J | m/z = 675.79 |
| | (C49H29N3O, 675.23) |
| 66J | m/z = 675.79 |
| | (C49H29N3O, 675.23) |
| 67J | m/z = 675.79 |
| | (C49H29N3O, 675.23) |
| 68J | m/z = 675.79 |
| | (C49H29N3O, 675.23) |
| 69J | m/z = 625.78 |
| | (C46H31N3, 625.25) |
| 70J | m/z = 674.81 |
| | (C49H30N4, 674.25) |
| 71J | m/z = 674.81 |
| | (C49H30N4, 674.25) |
| 72J | m/z = 674.81 |
| | (C49H30N4, 674.25) |
| 73J | m/z = 625.78 |
| | (C46H31N3, 625.25) |
| 74J | m/z = 674.81 |
| | (C49H30N4, 674.25) |
| 75J | m/z = 689.77 |
| | (C49H27N3O2, 689.21) |
| 76J | m/z = 689.77 |
| | (C49H27N3O2, 689.21) |
| 77J | m/z = 674.81 |
| | (C49H30N4, 674.25) |
| 78J | m/z = 648.77 |
| | (C47H28N4, 648.23) |
| 79J | m/z = 687.81 |
| | (C49H29N5, 687.24) |
| 80J | m/z = 724.87 |
| | (C53H32N4, 724.26) |
| 81J | m/z = 585.71 |
| | (C43H27N3, 585.22) |
| 82J | m/z = 585.71 |
| | (C43H27N3, 585.22) |
| 83J | m/z = 585.71 |
| | (C43H27N3, 585.22) |
| 84J | m/z = 585.71 |
| | (C43H27N3, 585.22) |
| 85J | m/z = 585.71 |
| | (C43H27N3, 585.22) |
| 86J | m/z = 585.71 |
| | (C43H27N3, 585.22) |
| 87J | m/z = 585.71 |
| | (C43H27N3, 585.22) |
| 88J | m/z = 635.77 |
| | (C47H29N3, 635.24) |
| 89J | m/z = 685.83 |
| | (C51H31N3, 685.25) |
| 90J | m/z = 685.83 |
| | (C51H31N3, 685.25) |
| 91J | m/z = 675.79 |
| | (C49H29N3O, 675.23) |
| 92J | m/z = 675.79 |
| | (C49H29N3O, 675.23) |
| 93J | m/z = 675.79 |
| | (C49H29N3O, 675.23) |
| 94J | m/z = 675.79 |
| | (C49H29N3O, 675.23) |
| 95J | m/z = 675.79 |
| | (C49H29N3O, 675.23) |
| 96J | m/z = 675.79 |
| | (C49H29N3O, 675.23) |
| 97J | m/z = 661.81 |
| | (C49H31N3, 661.25) |
| 98J | m/z = 661.81 |
| | (C49H31N3, 661.25) |
| 99J | m/z = 661.81 |
| | (C49H31N3, 661.25) |
| 100J | m/z = 711.87 |
| | (C53H33N3, 711.27) |
| 101J | m/z = 661.81 |
| | (C49H31N3, 661.25) |
| 102J | m/z = 661.81 |
| | (C49H31N3, 661.25) |
| 103J | m/z = 661.81 |
| | (C49H31N3, 661.25) |
| 104J | m/z = 661.81 |
| | (C49H31N3, 661.25) |
| 105J | m/z = 674.81 |
| | (C49H30N4, 674.25) |
| 106J | m/z = 764.89 |
| | (C55H32N4O, 764.26) |
| 107J | m/z = 725.85 |
| | (C53H31N3O, 725.25) |
| 108J | m/z = 725.85 |
| | (C53H31N3O, 725.25) |
| 109J | m/z = 725.85 |
| | (C53H31N3O, 725.25) |
| 110J | m/z = 725.85 |
| | (C53H31N3O, 725.25) |
| 111J | m/z = 649.75 |
| | (C47H27N3O, 649.25) |
| 112J | m/z = 725.85 |
| | (C53H31N3O, 725.25) |
| 113J | m/z = 585.71 |
| | (C43H27N3, 585.22) |
| 114J | m/z = 661.81 |
| | (C49H31N3, 661.25) |
| 115J | m/z = 585.71 |
| | (C43H27N3, 585.22) |
| 116J | m/z = 675.79 |
| | (C49H29N3O, 675.23) |
| 117J | m/z = 675.79 |
| | (C49H29N3O, 675.23) |
| 118J | m/z = 840.99 |
| | (C61H36N4O, 840.29) |
| 119J | m/z = 661.81 |
| | (C49H31N3, 661.25) |
| 120J | m/z = 765.87 |
| | (C55H31N3O2, 765.24) |
| 121J | m/z = 750.91 |
| | (C55H34N4, 750.28) |
| 122J | m/z = 764.89 |
| | (C55H32N4O, 764.26) |
| 123J | m/z = 814.95 |
| | (C59H34N4O, 814.27) |
| 124J | m/z = 840.99 |
| | (C61H36N4O, 840.29) |
| 125J | m/z = 635.77 |
| | (C47H29N3, 635.24) |
| 126J | m/z = 674.81 |
| | (C49H30N4, 674.25) |
| 127J | m/z = 750.91 |
| | (C55H34N4, 750.28) |
| 128J | m/z = 840.99 |
| | (C61H36N4O, 840.29) |
| 129J | m/z = 598.71 |
| | (C44H26N2O, 59820) |
| 130J | m/z = 648.77 |
| | (C47H28N4, 648.23) |
| 131J | m/z = 648.77 |
| | (C47H28N4, 648.23) |
| 132J | m/z = 698.83 |
| | (C51H30N4, 698.25) |
| 133J | m/z = 648.77 |
| | (C47H28N4, 648.23) |
| 134J | m/z = 648.77 |
| | (C47H28N4, 648.23) |
| 135J | m/z = 714.87 |
| | (C52H34N4, 714.87) |
| 136J | m/z = 763.90 |
| | (C55H33N5, 763.27) |
| 137J | m/z = 840.00 |
| | (C61H37N5, 839.30) |
| 138J | m/z = 704.85 |
| | (C49H28N4S, 704.20) |
| 139J | m/z = 674.81 |
| | (C49H30N4, 674.25) |
| 140J | m/z = 763.90 |
| | (C55H33N5, 763.27) |
| 141J | m/z = 754.91 |
| | (C53H30N4S, 754.22) |
| 142J | m/z = 724.87 |
| | (C53H32N4, 724.26) |
| 143J | m/z = 738.85 |
| | (C53H30N4O, 738.24) |
| 144J | m/z = 774.93 |
| | (C57H34N4, 774.28) |
| 145J | m/z = 724.87 |
| | (C53H32N4, 724.26) |
| 146J | m/z = 724.87 |
| | (C53H32N4, 724.26) |
| 147J | m/z = 774.93 |
| | (C57H34N4, 774.28) |
| 148J | m/z = 724.87 |
| | (C53H32N4, 724.26) |
| 149J | m/z = 688.79 |
| | (C49H28N4O, 688.23) |
| 150J | m/z = 714.87 |
| | (C52H34N4, 714.28) |
| 151J | m/z = 763.90 |
| | (C55H33N5, 763.27) |
| 152J | m/z = 840.00 |
| | (C61H37N5, 839.30) |
| 153J | m/z = 648,77 |
| | (C47H28N4, 648.23) |
| 154J | m/z = 800.97 |
| | (C59H36N4, 800.29) |
| 155J | m/z = 748.89 |
| | (C55H32N4, 748.26) |
| 156J | m/z = 698.83 |
| | (C51H30N4, 698.25) |
| 157J | m/z = 661.76 |
| | (C48H27N3O, 661.22) |
| 158J | m/z = 621.7 |
| | (C46H27N3, 621.22) |
| 159J | m/z = 722.85 |
| | (C53H30N4, 722.25) |
| 160J | m/z = 698.83 |
| | (C51H30N4, 698.25) |
| 161J | m/z = 753.92 |
| | (C54H31N3S, 753.22) |
| 162J | m/z = 727.89 |
| | (C52H29N3S, 727.21) |
| 163J | m/z = 698.83 |
| | (C51H30N4, 698.25) |
| 164J | m/z = 724.87 |
| | (C53H32N4, 724.26) |
| 165J | m/z = 698.83 |
| | (C51H30N4, 698.25) |
| 166J | m/z = 724.87 |
| | (C53H32N4, 724.26) |
| 167J | m/z = 724.87 |
| | (C53H32N4, 724.26) |
| 168J | m/z = 754.91 |
| | (C53H30N4S, 754.22) |
| 169J | m/z = 648.77 |
| | (C47H28N4, 648.23) |
| 170J | m/z = 648.77 |
| | (C47H28N4, 648.23) |
| 171J | m/z = 648.77 |
| | (C47H28N4, 648.23) |
| 172J | m/z = 648.77 |
| | (C47H28N4, 648.23) |
| 173J | m/z = 585.71 |
| | (C43H27N3, 585.22) |
| 174J | m/z = 648.77 |
| | (C47H28N4, 648.23) |
| 175J | m/z = 688.79 |
| | (C49H28N4O, 688.23) |
| 176J | m/z = 738.85 |
| | (C53H30N4O, 738.24) |
| 177J | m/z = 752.92 |
| | (C55H36N4, 752.29) |
| 178J | m/z = 752.92 |
| | (C55H36N4, 752.29) |
| 179J | m/z = 802.98 |
| | (C59H38N4, 802.98) |
| 180J | m/z = 792.99 |
| | (C58H40N4, 792.33) |
| 181J | m/z = 792.99 |
| | (C58H40N4, 792.33) |
| 182J | m/z = 842.02 |
| | (C61H39N5, 841.32) |
| 183J | m/z = 751.93 |
| | (C56H37N3, 751.30) |
| 184J | m/z = 842.02 |
| | (C61H39N5, 841.32) |
| 185J | m/z = 732.93 |
| | (C53H40N4, 732.33) |
| 186J | m/z = 782.97 |
| | (C55H34N4S, 782.25) |
| 187J | m/z = 701.83 |
| | (C50H31N5, 701.83) |
| 188J | m/z = 694.81 |
| | (C49H31FN4, 694.25) |
| 189J | m/z = 788.90 |
| | (C55H34F2N4, 788.28) |
| 190J | m/z = 802.94 |
| | (C57H34N6, 802.28) |
| 191JJ | m/z = 780.98 |
| | (C57H40N4, 780.33) |
| 192J | m/z = 762.98 |
| | (C55H26D10N4, 762.36) |
| 193J | m/z = 676.82 |
| | (C49H32N4, 676.26) |
| 194J | m/z = 752.92 |
| | {(C55H36N4, 752.29) |
| 195J | m/z = 829.02 |
| | (C61H40N4, 828.33) |
| 196J | m/z = 829.02 |
| | (C61H40N4, 828.33) |
| 197J | m/z = 829.02 |
| | (C61H40N4, 828.33) |
| 198J | m/z = 828.03 |
| | (C62H41N3, 827.33) |
| 199J | m/z = 827.04 |
| | (C63H42N2, 826.33) |
| 200J | m/z = 750.95 |
| | (C57H38N2, 750.30) |
| 201J | m/z = 674.85 |
| | (C51H34N2, 674.27) |
| 202J | m/z = 674.85 |
| | (C51H34N2, 674.27) |
| 203J | m/z = 674.85 |
| | (C51H34N2, 674.27) |
| 204J | m/z = 675.84 |
| | (C50H33N3, 675.27) |
| 205J | m/z = 725.90 |
| | (C54H35N3, 725.28) |
| 206J | m/z = 649.80 |
| | (C48H31N3, 649.25) |
| 207J | m/z = 649.80 |
| | (C48H31N3, 649.25) |
| 208J | m/z = 725.90 |
| | (C54H35N3, 725.28) |
| 209J | m/z = 829.02 |
| | (C61H40N4, 828.33) |
| 210J | m/z = 829.02 |
| | (C61H40N4, 828.33) |
| 211J | m/z = 802.98 |
| | (C59H38N4, 802.31) |
| 212J | m/z = 802.98 |
| | (C59H38N4, 802.31) |
| 213J | m/z = 843.00 |
| | (C61H38N4O, 842.30) |
| 214J | m/z = 766.90 |
| | (C55H34N4O, 766.27) |
| 215J | m/z = 766.90 |
| | (C55H34N4O, 766.27) |
| 216J | m/z = 802.98 |
| | (C59H38N4, 802.31) |
| 217J | m/z = 766.90 |
| | (C55H34N4O, 766.27) |
| 218J | m/z = 766.90 |
| | (C55H34N4O, 766.27) |
| 219J | m/z = 829.02 |
| | (C61H40N4, 828.33) |
| 220J | m/z = 905.12 |
| | (C67H44N4, 904.36) |
| 221J | m/z = 829.02 |
| | (C61H40N4, 828.33) |
| 222J | m/z = 750.95 |
| | (C57H38N2, 750.30) |
| 223J | m/z = 751.93 |
| | (C56H37N3, 751.30) |
| 224J | m/z = 751.93 |
| | (C56H37N3, 751.30) |
| 225J | m/z = 595.77 |
| | (C43H17D10N3, 595.28) |
| 226J | m/z = 595.77 |
| | (C43H17D10N3, 595.28) |
| 227J | m/z = 593.76 |
| | (C43H19D8N3, 593.27) |
| 228J | m/z = 612.87 |
| | (C43D27N3, 612.39) |
| 229J | m/z = 591.75 |
| | (C43H21D6N3, 591.26) |
| 230J | m/z = 590.74 |
| | (C43H22D5N3, 590.25) |
| 231J | m/z = 590.74 |
| | (C43H22D5N3, 590.25) |
| 232J | m/z = 589.73 |
| | (C43H23D4N3, 589.25) |
| 233J | m/z = 658.83 |
| | (C47H18D10N4, 658.29) |
| 234J | m/z = 658.83 |
| | (C47H18D10N4, 658.29) |
| 235J | m/z = 656.82 |
| | (C47H2OD8N4, 656.28) |
| 236J | m/z = 666.88 |
| | (C47H10D18N4, 666.34) |
| 237J | m/z = 676.94 |
| | (C47D28N4, 676.41) |
| 238J | m/z = 686.88 |
| | (C49H22D10N4, 686.33) |
| 239J | m/z = 681.85 |
| | (C49H27D5N4, 681.29) |
| 240J | m/z = 709.02 |
| | (C49D32N4, 608.46) |

TABLE 8

| Example | ¹H NMR (CDCl₃, 200 Mz) |
|---|---|
| 2J | δ = 9.09 (d, 2H), 8.81 (d, 4H), 8.32 (d, 1H), |
| | 8.29 (d, 1H), 8.23~8.14 (m, 4H), 7.62~7.60 (m, 3H), |
| | 7.59 (d, 1H), 7.58~7.55 (m, 3H), 7.53 (d, 1H), |
| | 7.19 (t, 1H), 7.16 (t, 1H), 6.92 (d, 1H). |
| 6J | δ = 9.06 (d, 2H), 9.02 (d, 1H), 8.87 (s, 1H), 8.78 (d, |
| | 1H), 8.28 (d, 1H), 8.29~8.26 (m, 8H), 7.84 (d, 1H), |
| | 7.68~7.39 (m, 7H), 7.28 (d, 2H), 7.14 (t, 1H). |
| 8J | δ = 9.18 (s, 1H), 9.03 (d, 1H), 8.92~8.86 (m 4H), |
| | 8.60 (d, 1H), 8.16~8.13 (m, 2H), 8.06 (d, 1H), |
| | 8.01~7.98 (m, 2H), 7.74~7.47 (m, 12H), 7.40 (t, 1H). |
| 12J | δ = 9.20 (s, 1H), 9.05 (s, 1H), 8.92~8.86 (m 4H), |
| | 8.60 (d, 1H), 8.16~8.13 (m, 2H), 8.06 (d, 1H), |
| | 8.00 (s, 1H), 7.99 (d, 1H), 7.74~7.49 (m, 12H), |
| | 7.41 (t, 1H). |
| 13J | δ = 9.19 (s, 1H), 8.92~8.85 (m 4H), 8.60 (d, 1H), |
| | 7.58 (s, 1H), 8.16~8.13 (m, 2H), 8.06(d, 1H), |
| | 8.01 (d, 2H), 7.74~7.49 (m, 12H), 7.39 (t, 1H). |
| 17J | δ = 9.32 (d, 1H), 9.09 (d, 1H), 8.47 (d, 1H), 8.31 |
| | (d, 1H), 8.26~8.21 (m, 4H), 8.17 (d, 2H), 8.16 (d, |
| | 1H), 7.69~7.58 (m, 9H), 7.42 (t, 1H), 7.36 (t, 1H). |
| 18J | δ = 8.32 (d, 1H), 8.30 (d, 1H), 8.13~8.09 (d, 4H), |
| | 8.01 (d, 2H), 7.66 (d, 1H), 7.38~7.29 (m, 8H), |
| | 7.18 (t, 1H), 6.89~6.85 (m, 2H). |
| 19J | δ = 9.37 (d, 1H), 8.80 (d, 1H), 8.60~8.57 (m, 3H), |
| | 7.88~7.80 (m, 4H), 7.58(d, 2H), 7.52(d, 1H), |
| | 7.51~7.44 (m, 8H), 7.39 (t, 1H), 7.31 (t, 1H). |
| 20J | δ = 9.29 (d, 1H), 8.76 (d, 1H), 8.58~8.55 (m, 3H), |
| | 7.73~7.62 (m, 4H), 7.57(d, 2H), 7.50 (d, 1H), |
| | 7.49~7.32 (m, 8H), 7.30 (t, 1H), 7.25 (t, 1H). |
| 23J | δ = 8.91 (d, 1H), 8.89 (d, 1H), 8.60~8.58 (m, 2H), |
| | 7.90 (d, 1H), 7.85~7.79 (m, 4H), 7.56 (d, 2H), |
| | 7.52 (d, 1H), 7.50~7.42 (m, 8H), 7.39 (t, 1H), |
| | 7.31 (t, 1H). |
| 24J | δ = 8.84 (d, 1H), 8.74 (d, 1H), 8.57~8.55 (m, 3H), |
| | 7.69~7.65 (m, 4H), 7.56 (d, 2H), 7.49 (d, 1H), |
| | 7.43~7.29 (m, 8H), 7.28 (t, 1H), 7.19 (t, 1H). |
| 30J | δ = 8.86 (d, 2H), 8.26 (d, 2H), 8.17 (t, 1H), |
| | 7.91~7.90 (m, 3H), 7.75(d, 2H), 7.62~7.56 (m, 7H), |
| | 7.46 (d, 1H), 7.42~7.32 (m, 11H) |
| 37J | δ = 8.89 (d, 2H), 8.28 (d, 2H), 8.16 (t, 1H), |
| | 7.92~7.91 (m, 3H), 7.70(d, 2H), 7.56~7.48 (m, 5H), |
| | 7.44 (d, 1H), 7.43~7.37 (m, 4H), 7.29 (t, 1H). |
| 41J | δ = 8.87 (d, 4H), 8.82 (s, 1H), 8.59 (d, 1H), 8.16 |
| | (d, 1H), 7.74~7.71 (m, 5H), 7.51 (d, 2H), |
| | 7.53~7.46 (m, 6H), 7.44 (d, 1H), 7.37 (t, 2H). |
| 45J | δ = 9.40 (d, 1H), 9.32 (s, 1H), 8.87 (s, 1H), 8.86 (d, |
| | 2H), 8.83 (d, 1H), 8.49 (d, 1H), 8.08 (d, 1H), |
| | 7.82~7.79 (m, 5H), 7.62 (d, 1H), 7.54~7.45 (m, 8H), |
| | 7.44 (d, 1H), 7.31 (t, 2H). |
| 46J | δ = 8.93 (s, 1H), 8.90 (d, 2H), 8.84~8.83 (m, 3H), |
| | 8.19 (s, 1H), 8.12 (d, 1H), 8.05 (d, 1H), |
| | 7.99~7.85 (m, 7H), 7.72 (d, 1H), 7.67~7.58 (m, 7H), |
| | 7.54~7.52 (m, 2H), 7.48~7.46 (m, 2H), 7.40 (t, 1H). |
| 47J | δ = 9.46 (s, 2H), 9.31 (s, 1H), 9.27 (s, 1H), 8.94 (d, |
| | 2H), 8.38 (d, 1H), 8.26~8.19 (m, 5H), 7.71~7.67 (m, |
| | 4H), 7.64~7.56 (m, 7H), 7.45~7.25 (m, 3H), 7.11 (t, |
| | 1H). |
| 48J | δ = 9.19 (s, 1H), 9.01 (s, 1H), 8.98 (d, 4H), 8.38 (d, |
| | 1H), 8.26~8.18 (m, 4H), 8.11 (d, 1H), 7.73~7.67 (m, |
| | 6H), 7.64~7.52 (m, 5H), 7.44 (d, 2H), 7.39~7.25 (m, |
| | 5H), 7.09 (t, 1H) |
| 49J | δ = 9.04 (s, 1H), 9.01 (s, 1H), 8.90 (d, 2H), 8.87 (s, |
| | 1H), 8.84~8.83 (m, 3H), 8.28~8.19 (m, 5H), 8.18 (d, |
| | 1H), 7.69~7.66 (m, 3H), 7.62~7.59 (m, 3H), |
| | 7.41~7.35 (m, 4H), 7.12 (t, 1H). |
| 50J | δ = 8.92 (s, 1H), 8.90 (s, 1H), 8.87 (d, 2H), 8.83 (s, |
| | 1H), 8.76~8.70 (m, 3H), 8.29~8.19 (m, 5H), 8.11 (d, |
| | 1H), 7.68~7.61 (m, 3H), 7.60~7.55 (m, 4H), |
| | 7.41~7.33 (m, 3H), 7.21 (t, 1H). |
| 51J | δ = 9.03 (d, 2H), 9.01 (s, 1H), 8.90~8.87 (m, 3H), |
| | 8.84~8.83 (m, 2H), 8.36 (d, 1H), 8.28~8.22 (m, 3H), |
| | 7.69~7.59 (m, 5H), 7.63~7.61 (m, 4H), 7.53~7.51 |
| | (m, 3H), 7.29 (t, 1H). |
| 52J | δ = 9.01 (s, 1H), 8.96 (d, 1H), 8.90~8.88 (d, 2H), |
| | 8.87 (s, 1H), 8.84~8.83 (m, 2H), 8.22 (d, 1H), |
| | 8.20 (d, 1H), 8.06~7.99 (m, 3H), 7.66~7.58 (m, 5H), |
| | 7.63~7.61 (m, 3H), 7.56~7.49 (m, 4H), 7.27 (t, 1H). |
| 70J | δ = 9.22 (s, 1H), 9.18 (d, 1H), 9.01 (s, 1H), 8.88 (d, |
| | 2H), 8.84~8.83 (m, 2H), 8.36 (d, 1H), 8.28~8.22 (m, |
| | 3H), 7.69~7.59 (m, 5H), 7.63~7.61 (m, 4H), |
| | 7.53~7.43 (m, 7H), 7.21 (t, 1H). |
| 71J | δ = 9.36 (s, 1H), 9.17 (d, 1H), 9.02 (s, 1H), 8.92 (d, |
| | 2H), 8.84~8.83 (m, 2H), 8.36 (d, 1H), 8.28~8.22 (m, |
| | 3H), 7.69~7.59 (m, 5H), 7.53~7.47 (m, 4H), |
| | 7.46~7.37 (m, 7H), 7.20 (t, 1H). |
| 74J | δ = 9.27 (s, 1H), 9.16 (d, 1H), 9.03 (s, 1H), 8.88 (d, |
| | 2H), 8.85 (s, 1H), 8.82 (d, 1H), 8.32 (d, 1H), |
| | 8.28~8.24 (m, 3H), 7.69~7.58 (m, 5H), 7.53~7.47 |
| | (m, 4H), 7.46~7.37 (m, 7H), 7.31~7.30 (m, 2H), 7.20 |
| | (t, 1H). |
| 77J | δ = 9.13 (s, 1H), 9.11 (d, 2H), 9.03 (d, 1H), 9.01 (d, |
| | 1H), 8.87 (d, 2H), 8.36 (d, 1H), 8.28~8.24 (m, 5H), |
| | 8.12 (d, 1H), 7.69~7.58 (m, 5H), 7.46~7.37 (m, 7H), |
| | 7.33~7.30 (m, 3H), 7.16 (t, 1H). |
| 78J | δ = 8.92 (s, 1H), 8.84 (d, 2H), 8.68 (d, 1H), 8.66 (s, |
| | 1H), 8.39 (d, 2H), 8.03 (s, 1H), 7.90~7.86 (5H), |
| | 7.78 (t, 1H), 7.69~7.63 (m, 5H), 7.42~7.36 (m, 5H), |
| | 7.33~7.30 (m, 3H), 7.19 (t, 1H). |
| 81J | δ = 9.03 (d, 2H), 8.89 (d, 4H), 8.32 (d, 2H), 8.18 (d, |
| | 1H), 7.90~7.86 (m, 5H), 7.71~7.64 (m, 5H), |
| | 7.43~7.38 (m, 4H), 7.36 (t, 1H), 7.33~7.31 (m, 2H). |
| 82J | δ = 9.05 (d, 2H), 9.01 (s, 1H), 8.87 (d, 4H), 8.89 (s, |
| | 1H), 8.29 (d, 2H), 8.17 (d, 1H), 7.89~7.83 (m, 5H), |
| | 7.71~7.64 (m, 4H), 7.41~7.33 (m, 4H), 7.33~7.32 |
| | (m, 2H), 7.27 (t, 1H). |
| 91J | δ = 8.92 (d, 1H), 8.87 (d, 4H), 8.58 (d, 1H), 8.19 (s, |
| | 1H), 8.17~8.13 (m, 3H), 8.04~7.99 (m, 2H), |
| | 7.84~7.81 (d, 2H), 7.71~7.61 (m, 7H), 7.51 (d, 1H), |
| | 7.44~7.36 (m, 5H), 7.33 (t, 2H), 7.19 (t, 1H). |
| 92J | δ = 8.90 (d, 4H), 8.61 (d, 1H), 8.17~8.11 (m, 4H), |
| | 8.00~7.97 (m, 2H), 7.84~7.80 (d, 2H), 7.70~7.61 (m, |
| | 7H), 7.53 (d, 1H), 7.41~7.38 (m, 5H), 7.33~7.30 (m, |
| | 2H), 7.29 (t, 1H). |
| 93J | δ = 9.18 (d, 1H), 9.03 (d, 1H), 8.89 (d, 4H), 8.66 (d, |
| | 1H), 8.19 (s, 1H), 8.17~8.13 (m, 3H), 8.04~7.99 (m, |
| | 2H), 7.84~7.81 (d, 2H), 7.71~7.62 (m, 6H), |
| | 7.51~7.39 (m, 5H), 7.36~7.34 (m, 2H), 7.19 (t, 1H). |
| 98J | δ = 8.92 (s, 1H), 8.87 (d, 4H), 8.64 (d, 1H), |
| | 7.82~7.79 (m, 3H), 7.64~7.53 (m, 5H), 7.51 (d, 1H), |
| | 7.49~7.44 (m, 4H), 7.36~7.29 (m, 11H), 7.27 (t, 1H). |
| 99J | δ = 9.02 (d, 2H), 8.89 (d, 4H), 7.89 (d, 2H), |
| | 7.67~7.61 (m, 3H), 7.52 (d, 2H), 7.48 (d, 1H), |
| | 7.49~7.43 (m, 4H), 7.37 (d, 1H), 7.34~7.29 (m, 10H), |
| | 7.26 (t, 1H). |
| 103J | δ = 8.90 (s, 1H), 8.87 (d, 4H), 8.64 (d, 1H), |
| | 7.84~7.80 (m, 3H), 7.64~7.51 (m, 5H), 7.49~7.45 |
| | (m, 5H), 7.34~7.29 (m, 11H), 7.27 (t, 1H). |
| 110J | δ = 9.03 (s, 1H), 8.92 (d, 2H), 8.66 (s, 1H), |
| | 8.84~8.83 (m, 3H), 8.18 (s, 1H), 8.12 (d, 1H), |
| | 8.06~8.04 (m, 2H), 7.99~7.85 (m, 8H), 7.70 (d, 2H), |
| | 7.66~7.61 (m, 5H), 7.54~7.51 (m, 4H), 7.36 (t, 1H). |
| 117J | δ = 9.03 (s, 1H), 8.84 (d, 1H), 8.81~8.80 (m, 3H), |
| | 8.13~8.12 (m, 2H), 8.04~8.01 (m, 2H), 7.99~7.84 |
| | (m, 8H), 7.68 (d, 2H), 7.66 (d, 2H), 7.64~7.60 (m, |
| | 3H), 7.54~7.51 (m, 4H), 7.41 (t, 1H). |
| 125J | δ = 9.38 (s, 1H), 8.89~8.65 (m, 3H), 8.77 (d, 1H), |
| | 8.59 (d, 1H), 8.81~8.79 (m, 3H), 8.13~8.12 (m, 2H), |
| | 8.04~7.98 (m, 2H), 7.99~7.87 (m, 6H), 7.68~7.67 |
| | (m, 2H), 7.64~7.60 (m, 3H), 7.54~7.49 (m, 4H), |
| | 7.41~7.39 (m, 2H). |
| 126J | δ = 9.24 (d, 2H), 98.89~8.65 (m, 3H), 8.42 (d, 1H), |
| | 8.26 (d, 2H), 8.13~8.12 (m, 2H), 8.02~7.93 (m, 6H), |
| | 7.68~7.67 (m, 2H), 7.64~7.60 (m, 3H), 7.49~7.46 |
| | (m, 7H), 7.41~7.38 (m, 2H). |
| 133J | δ = 9.05 (s, 1H), 8.82~8.73 (m, 5H), 8.67 (s, 1H), |
| | 8.33 (d, 1H), 8.14 (s, 1H), 8.10 (d, 1H), 7.98 (s, |
| | 1H), 7.91~7.86 (m, 2H), 7.74~7.50 (m, 10H), |
| | 7.48~7.36 (m, 4H), 7.32 (t, 1H). |
| 134J | δ = 9.41 (s, 1H), 8.92 (d, 4H), 8.73 (d, 1H), 8.70 (s, |
| | 1H), 8.36 (d, 1H), 8.13~8.11 (m, 1H), 8.02 (d, 1H), |
| | 7.89 (d, 1H), 7.84 (s, 1H), 7.72~7.62 (m, 8H), |
| | 7.53~7.49 (m, 2H), 7.46~7.36 (m, 6H). |
| 135J | δ = 9.37 (s, 1H), 8.86 (d, 4H), 8.63 (s, 1H), 8.52 (s, |
| | 1H), 8.26 (d, 1H), 8.24 (d, 1H), 8.18 (d, 1H), |
| | 7.99 (d, 2H), 7.78~7.58 (m, 11H), 7.48~7.39 (m, 4H), |
| | 7.31 (t, 1H), 1.59 (s, 6H). |
| 136J | δ = 9.21 (s, 1H), 8.92 (s, 1H), 8.83 (d, 4H), 8.66 (d, |
| | 1H), 8.36 (d, 1H), 8.34 (d, 1H), 7.88 (s, 1H), |
| | 7.79~7.60 (m, 8H), 7.54~7.48 (m, 4H), 7.41~7.31 |
| | (m, 9H), 7.01 (d, 1H), 6.98 (s, 1H). |
| 138J | δ = 8.81 (d, 1H), 8.80 (d, 4H), 8.69 (d, 1H), |
| | 8.32~8.25 (m, 3H), 8.17 (d, 1H), 8.07 (s, 1H), |
| | 7.76 (d, 1H), 7.67~7.58 (m, 5H), 7.54~7.46 (m, 8H), |
| | 7.45~7.33 (m, 3H). |
| 142J | δ = 9.04 (s, 1H), 8.81 (d, 2H), 8.76 (d, 2H), 8.54 (s, |
| | 1H), 8.45 (d, 1H), 8.19 (d, 1H), 7.88 (s, 1H), |
| | 7.81~7.72 (m, 6H), 7.66~7.59 (m, 7H), 7.55~7.28 |
| | (m, 9H), 7.22 (t, 1H). |
| 143J | δ = 9.27 (s, 1H), 9.09 (s, 1H), 8.79~8.78 (m, 3H), |
| | 8.51 (s, 1H), 8.47 (d, 1H), 8.16 (d, 1H), 7.88 (s, |
| | 1H), 7.82~7.74 (m, 6H), 7.66~7.60(m, 6H), |
| | 7.53~7.28 (m, 8H), 7.21 (t, 1H). |
| 145J | δ = 9.18 (s, 1H), 8.96 (d, 1H), 8.74 (d, 4H), 8.66 (s, |
| | 1H), 8.16 (s, 1H), 7.88 (d, 1H), 7.72 (s, 1H), |
| | 7.71~7.66 (m, 4H), 7.66~7.57 (m, 6H), 7.53~7.38 |
| | (m, 7H), 7.36~7.34 (m, 3H), 7.28~7.24 (m, 2H). |
| 146J | δ = 9.12 (d, 2H), 8.92 (d, 4H), 8.21 (d, 2H), 8.19 (s, |
| | 1H), 7.84~7.79 (m, 3H), 7.72 (s, 1H), 7.71~7.66 (m, |
| | 3H), 7.66~7.57 (m, 6H), 7.53~7.42 (m, 5H), |
| | 7.36~7.33 (m, 3H), 7.28~7.25 (m, 2H). |
| 151J | δ = 9.22 (d, 1H), 8.91 (s, 1H), 8.83 (d, 4H), 8.64 (d, |
| | 1H), 8.37 (d, 1H), 8.36 (d, 1H), 7.90 (d, 1H), |
| | 7.88 (d, 1H), 7.86 (d, 1H), 7.72~7.68 (m, 6H), |
| | 7.54~7.48 (m, 5H), 7.43~7.30 (m, 8H), 7.12 (d, 1H), |
| | 7.01 (s, 1H). |
| 160J | δ = 9.48 (s, 1H), 9.04~9.01 (m, 1H), 8.89 (d, 4H), |
| | 8.79 (d, 1H), 7.76~7.60 (m, 8H), 7.58 (d, 1H), |
| | 7.54~7.49 (m, 4H), 7.48 (t, 1H), 7.44~7.32 (m, 8H), |
| | 7.28 (t, 1H). |
| 162J | δ = 9.37 (s, 1H), 8.96 (s, 1H), 8.42 (d, 1H), 8.31 (d, |
| | 1H), 8.28~8.24 (m, 1H), 8.15~8.09 (m, 4H), 7.79 (s, |
| | 1H), 7.78~7.66 (m, 6H), 7.62~7.54 (m, 6H), |
| | 7.52~7.31 (m, 6H). |
| 169J | δ = 9.02 (d, 1H), 8.83 (d, 4H), 8.81 (d, 1H), 8.27 (s, |
| | 1H), 8.18 (d, 1H), 8.16 (d, 1H), 7.78~7.69 (m, 5H), |
| | 7.61 (s, 1H), 7.59~7.52 (m, 6H), 7.50~7.31 (m, 6H), |
| | 7.29 (s, 1H). |
| 170J | δ = 9.08 (d, 1H), 8.86 (d, 4H), 8.82 (d, 1H), 8.29 (s, |
| | 1H), 8.18~8.16 (m, 2H), 7.78~7.66 (m, 5H), |
| | 7.60~7.52 (m, 7H), 7.50~7.32 (m, 6H), 7.29(s, 1H). |
| 178J | δ = 8.87 (d, 4H), 8.72 (d, 2H), 7.88 (d, 1H), |
| | 7.70~7.53 (m, 12H), 7.46 (d, 1H), 7.41~7.36 (m, 6H), |
| | 7.31~7.24 (m, 8H), 7.13 (t, 1H). |
| 189J | δ = 8.84~8.82 (m, 4H), 8.54 (d, 1H), 8.40 (d, 1H), |
| | 7.83~8.82 (m, 2H), 7.65(t, 1H), 7.6 (d, 2H), 7.74 (m, |
| | 1H), 7.30~7.27 (m, 3H), 7.17~7,15 (m, 6H). |
| 194J | δ = 8.87 (s, 1H), 8.70 (d, 4H), 8.53 (s, 1H), 8.12 (d, |
| | 1H), 8.10 (d, 1H), 7.68~7.48 (m, 14H), 7.46~7.40 (m, |
| | 7H), 7.33~7.22 (m, 7H). |
| 198J | δ = 8.92~8.91 (m, 2H), 8.76~8.75 (m, 2H), 8.67 (s, |
| | 1H), 8.12 (d, 1H), 8.11 (d, 1H), 7.89 (d, 1H), |
| | 7.74~7.52 (m, 12H), 7.45~7.34 (m, 11H), 7.29 (s, |
| | 1H), 7.22~7.10 (m, 7H), 6.97~6.91 (m 2H). |
| 201J | δ = 8.72 (d, 1H), 8.68 (d, 1H), 8.37 (s, 1H), |
| | 8.12 (d, 1H), 8.11 (d, 1H), 7.89 (d, 1H), |
| | 7.74~7.52 (m, 12H), 7.45~7.34 (m, 11H), 7.29 (s, |
| | 1H), 7.22~7.10 (m, 7H), 6.95~6.89 (m 2H). |
| 209J | δ = 8.86 (s, 1H), 8.83 (d, 4H), 8.12 (d, 2H), 7.89 (s, |
| | 1H), 7.74~7.52 (m, 12H), 7.45~7.34 (m, 11H), 7.29 |
| | (s, 1H), 7.22~7.10 (m, 6H), 6.97~6.91 (m 2H). |
| 210J | δ = 8.88 (d, 2H), 8.84 (d, 4H), 8.10 (d, 2H), 7.86 (d, |
| | 1H), 7.71~7.57 (m, 12H), 7.42~7.33 (m, 11H), |
| | 7.24~7.21 (m, 2H), 7.19~7.12 (m, 4H), 6.92~6.86 |
| | (m 2H). |
| 233J | δ = 9.05 (s, 1H), 8.75 (d, 1H), 8.67 (s, 1H), 8.33 (d, |
| | 1H), 8.14 (s, 1H), 8.10 (d, 1H), 7.98 (s, 1H), |
| | 7.91~7.86 (m, 2H), 7.69~7.52 (m, 6H), 7.41~7.36 |
| | (m, 2H), 7.32 (t, 1H). |
| 235J | δ = 9.05 (s, 1H), 8.82~8.73 (m, 4H), 8.33 (d, 1H), |
| | 8.10 (d, 1H), 7.98 (s, 1H), 7.91~7.88 (m, 1H), |
| | 7.74~7.53 (m, 6H), 7.48~7.39 (m, 2H), 7.32(t, 1H). |

### 1) Manufacture of Organic Light Emitting Device (Red Host)

A glass substrate thinly coated with ITO to have a thickness of 1,500 Å was ultrasonically washed with distilled water. When the washing with distilled water was finished, the glass substrate was ultrasonically washed with a solvent such as acetone, methanol, and isopropyl alcohol, dried and then was subjected to UVO treatment for 5 minutes using UV in a UV cleaning machine. Thereafter, the substrate was transferred to a plasma washing machine (PT), and then was subjected to plasma treatment in a vacuum state for an ITO work function and in order to remove a residual film, and was transferred to a thermal deposition apparatus for organic deposition.

A hole injection layer 4,4′,4″-tris[2-naphthyl(phenyl)amino]triphenylamine (2-TNATA), a hole transport layer N,N′-Di(1-naphthyl)-N,N′-diphenyl-(1,1′-biphenyl)-4,4′-diamine (NPB) and an electron blocking layer cyclohexylidenebis[N,N-bis(4-methylphenyl)benzenamine](TAPC) or an exciton blocking layer tris(4-carbazoyl-9-ylphenyl)amine (TCTA), which are common layers, were formed on the ITO transparent electrode (positive electrode).

A light emitting layer was thermally vacuum deposited thereon as follows. The light emitting layer was deposited to have a thickness of 400 Å by depositing one or two types of the compounds described in the following Table 9 as a red host from a single supply source or two supply sources and doping the host with an Ir compound at 3 wt % using (piq)₂(Ir)_{(acac)} as a red phosphorescent dopant. Thereafter, Bphen as a hole blocking layer was deposited to have a thickness of 30 Å, and TPBI as an electron transport layer was deposited to have a thickness of 250 Å thereon. Finally, lithium fluoride (LiF) was deposited to have a thickness of 10 Å on the electron transport layer to form an electron injection layer, and then aluminum (Al) negative electrode was deposited to have a thickness of 1,200 Å on the electron injection layer to form a negative electrode, thereby manufacturing an organic electroluminescence device.

### 2) Driving Voltage and Light Emitting Efficiency of Organic Electroluminescence Device

For the organic electroluminescence device manufactured as described above, electroluminescence (EL) characteristics were measured by M7000 manufactured by McScience Inc., and based on the measurement result thereof, T₉₀ was measured by a service life measurement equipment (M6000) manufactured by McScience Inc., when the reference luminance was 6,000 cd/m². Characteristics of the organic electroluminescence device of the present invention as shown in the following Table 9.

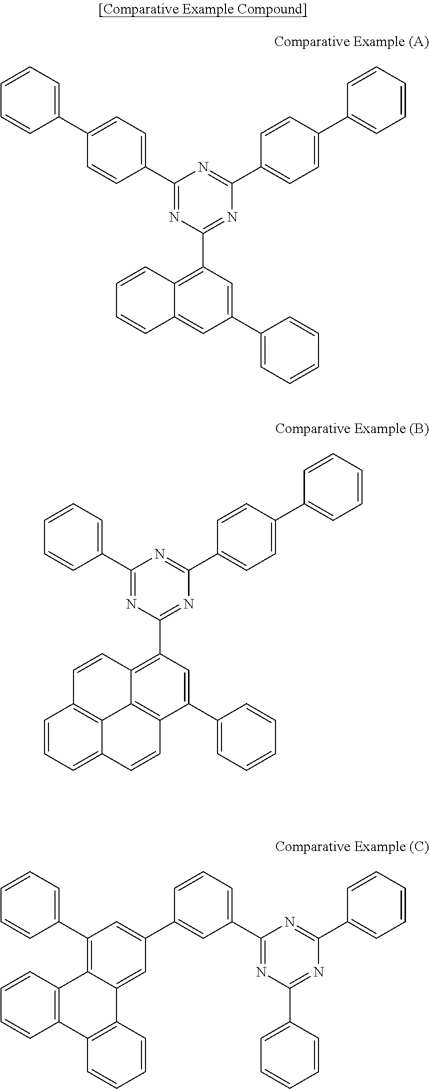 [SMILES: c1ccc(-c2ccc(-c3nc(-c4ccc(-c5ccccc5)cc4)nc(-c4cc(-c5ccccc5)cc5ccccc45)n3)cc2)cc1 ; c1ccc(-c2ccc(-c3nc(-c4ccccc4)nc(-c4cc(-c5ccccc5)c5ccc6cccc7ccc4c5c67)n3)cc2)cc1 ; c1ccc(-c2nc(-c3ccccc3)nc(-c3cccc(-c4cc(-c5ccccc5)c5c6ccccc6c6ccccc6c5c4)c3)n2)cc1]

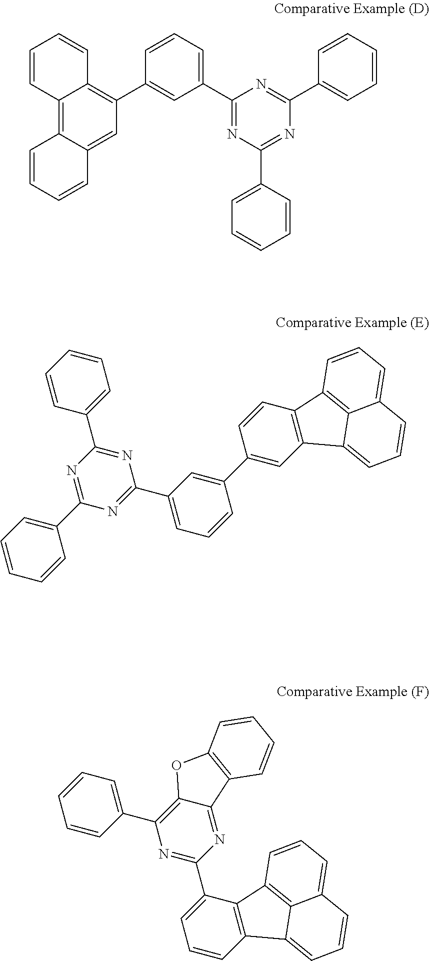 [SMILES: c1ccc(-c2nc(-c3ccccc3)nc(-c3cccc(-c4cc5ccccc5c5ccccc45)c3)n2)cc1 ; c1ccc(-c2nc(-c3ccccc3)nc(-c3cccc(-c4ccc5c(c4)-c4cccc6cccc-5c46)c3)n2)cc1 ; c1ccc(-c2nc(-c3cccc4c3-c3cccc5cccc-4c35)nc3c2oc2ccccc23)cc1]

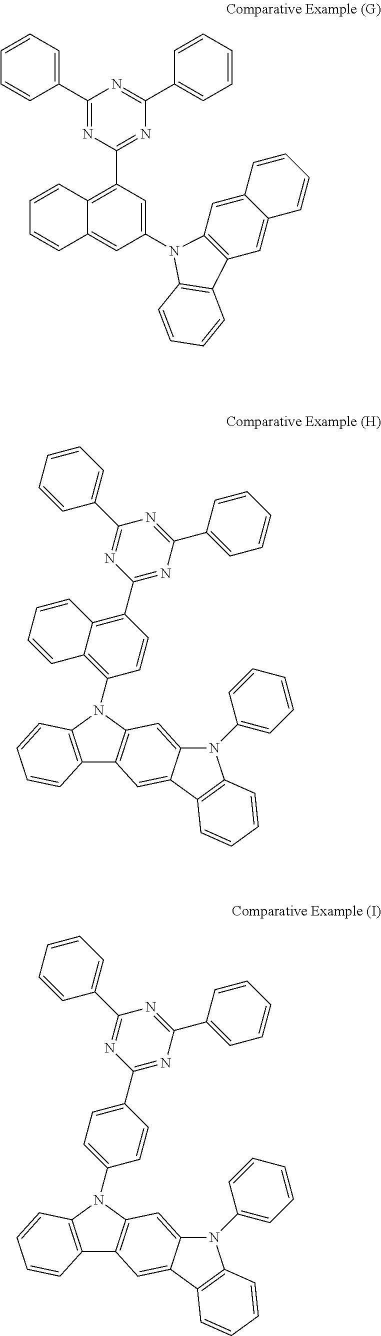 [SMILES: c1ccc(-c2nc(-c3ccccc3)nc(-c3cc(-n4c5ccccc5c5cc6ccccc6cc54)cc4ccccc34)n2)cc1 ; c1ccc(-c2nc(-c3ccccc3)nc(-c3ccc(-n4c5ccccc5c5cc6c7ccccc7n(-c7ccccc7)c6cc54)c4ccccc34)n2)cc1 ; c1ccc(-c2nc(-c3ccccc3)nc(-c3ccc(-n4c5ccccc5c5cc6c7ccccc7n(-c7ccccc7)c6cc54)cc3)n2)cc1]

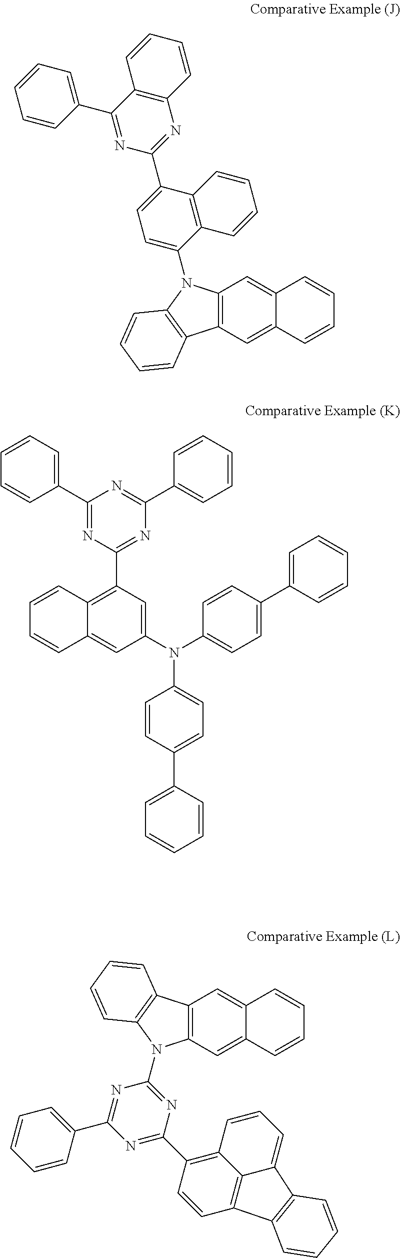 [SMILES: c1ccc(-c2nc(-c3ccc(-n4c5ccccc5c5cc6ccccc6cc54)c4ccccc34)nc3ccccc23)cc1 ; c1ccc(-c2ccc(N(c3ccc(-c4ccccc4)cc3)c3cc(-c4nc(-c5ccccc5)nc(-c5ccccc5)n4)c4ccccc4c3)cc2)cc1 ; c1ccc(-c2nc(-c3ccc4c5c(cccc35)-c3ccccc3-4)nc(-n3c4ccccc4c4cc5ccccc5cc43)n2)cc1]

TABLE 9

| | | | Driving | Effi- | Color | Service |
|---|---|---|---|---|---|---|
| | Com- | | voltage | ciency | coordinate | life |
| | pound | | (V) | (cd/A) | (x, y) | (T₉₀) |
| Comparative | (A) | | 4.13 | 14.1 | (0.682, | 52 |
| Example 1 | | | | | 0.318) | |
| Comparative | (B) | | 5.09 | 11.0 | (0.687, | 19 |
| Example 2 | | | | | 0.313) | |
| Comparative | (C) | | 6.23 | 28.2 | (0.687, | N/D |
| Example 3 | | | | | 0.313) | |
| Comparative | (D) | | 4.01 | 20.6 | (0.682, | 55 |
| Example 4 | | | | | 0.318) | |
| Comparative | (E) | | 3.71 | 23.1 | (0.682, | 49 |
| Example 5 | | | | | 0.318) | |
| Comparative | (F) | | 4.32 | 21.9 | (0.682, | 11 |
| Example 6 | | | | | 0.318) | |
| Comparative | (G) | | 4.22 | 23.4 | (0.682, | 77 |
| Example 7 | | | | | 0.318) | |
| Comparative | (H) | | 4.48 | 21.1 | (0.682, | 102 |
| Example 8 | | | | | 0.318) | |
| Comparative | (I) | | 4.61 | 21.2 | (0.682, | 75 |
| Example 9 | | | | | 0.318) | |
| Comparative | (J) | | 4.36 | 16.6 | (0.684, | 82 |
| Example 10 | | | | | 0.318) | |
| Comparative | (K) | | 3.61 | 26.1 | (0.687, | 89 |
| Example 11 | | | | | 0.313) | |
| Comparative | (L) | | 4.24 | 22.4 | (0.685, | 68 |
| Example 12 | | | | | 0.315) | |
| Comparative | (A):(K) | 2:1 | 4.13 | 16.1 | (0.682, | 71 |
| Example 13 | | | | | 0.318) | |
| | | 1:1 | 4.00 | 25.1 | (0.682, | 112 |
| | | | | | 0.318) | |
| | | 1:2 | 3.82 | 25.8 | (0.682, | 63 |
| | | | | | 0.318) | |
| | | 1:3 | 3.62 | 26.1 | (0.687, | 39 |

| | | | | | |
|---|---|---|---|---|---|
| | | | | 0.313) | |
| Comparative | 210J:(A) = | 3.89 | 33.8 | (0.682, | 158 |
| Example 14 | 1:1 | | | 0.318) | |
| Comparative | (D):(K) = | 3.92 | 25.1 | (0.682, | 68 |
| Example 15 | 1:1 | | | 0.318) | |
| Comparative | (G):(K) = | 4.11 | 25.6 | (0.682, | 51 |
| Example 16 | 1:1 | | | 0.318) | |
| Comparative | (K):45J = | 3.98 | 27.1 | (0.682, | 202 |
| Example 17 | 1:1 | | | 0.318) | |
| Comparative | (K):(F) = | 3.66 | 26.0 | (0.687, | 36 |
| Example 18 | 1:1 | | | 0.313) | |
| Example 1 | 2J | 3.82 eV | 26.3 | (0.685, | 112 |
| | | | | 0.315) | |
| Example 2 | 6J | 3.76 eV | 30.1 | (0.685, | 143 |
| | | | | 0.315) | |
| Example 3 | 8J | 3.68 eV | 33.2 | (0.685, | 180 |
| | | | | 0.315) | |
| Example 4 | 12J | 3.68 eV | 36.2 | (0.685, | 220 |
| | | | | 0.315) | |
| Example 5 | 13J | 3.68 ev | 33.2 | (0.685, | 160 |
| | | | | 0.315) | |
| Example 6 | 17J | 3.91 eV | 28.7 | (0.686, | 171 |
| | | | | 0.314) | |
| Example 7 | 18J | 4.01 eV | 26.9 | (0.686, | 179 |
| | | | | 0.314) | |
| Example 8 | 19J | 4.11 eV | 25.9 | (0.686, | 168 |
| | | | | 0.314) | |
| Example 9 | 20J | 3.98 eV | 27.3 | (0.686, | 159 |
| | | | | 0.314) | |
| Example 10 | 23J | 4.02 eV | 25.3 | (0.686, | 149 |
| | | | | 0.314) | |
| Example 11 | 24J | 3.92 eV | 26.3 | (0.686, | 166 |
| | | | | 0.314) | |
| Example 12 | 30J | 3.98 eV | 27.3 | (0.686, | 180 |
| | | | | 0.314) | |
| Example 13 | 37J | 3.89 eV | 26.9 | (0.686, | 178 |
| | | | | 0.314) | |
| Example 14 | 41J | 3.75 eV | 27.4 | (0.682, | 202 |
| | | | | 0.318) | |
| Example 15 | 45J | 3.75 eV | 27.4 | (0.682, | 222 |
| | | | | 0.318) | |
| Example 16 | 46J | 3.69 eV | 28.9 | (0.681, | 232 |
| | | | | 0.319) | |
| Example 17 | 47J | 3.69 eV | 29.2 | (0.681, | 229 |
| | | | | 0.319) | |
| Example 18 | 48J | 3.70 eV | 30.2 | (0.681, | 233 |
| | | | | 0.319) | |
| Example 19 | 49J | 3.72 eV | 31.2 | (0.682, | 223 |
| | | | | 0.318) | |
| Example 20 | 50J | 3.80 eV | 30.8 | (0.682, | 189 |
| | | | | 0.318) | |
| Example 21 | 51J | 3.99 eV | 29.9 | (0.682, | 188 |
| | | | | 0.318) | |
| Example 22 | 52J | 4.04 eV | 30.9 | (0.682, | 138 |
| | | | | 0.318) | |
| Example 23 | 70J | 4.01 eV | 25.9 | (0.682, | 178 |
| | | | | 0.318) | |
| Example 24 | 71J | 4.02 eV | 25.8 | (0.682, | 138 |
| | | | | 0.318) | |
| Example 25 | 74J | 3.97 eV | 28.6 | (0.682, | 148 |
| | | | | 0.318) | |
| Example 26 | 77J | 3.89 eV | 29.1 | (0.682, | 212 |
| | | | | 0.318) | |
| Example 27 | 78J | 3.89 eV | 29.6 | (0.682, | 231 |
| | | | | 0.318) | |
| Example 28 | 81J | 3.97 eV | 30.1 | (0.682, | 256 |
| | | | | 0.318) | |
| Example 29 | 82J | 4.01 eV | 31.9 | (0.682, | 276 |
| | | | | 0.318) | |
| Example 30 | 91J | 4.24 eV | 33.6 | (0.682, | 76 |
| | | | | 0.318) | |
| Example 31 | 92J | 4.28 eV | 33.8 | (0.682, | 68 |
| | | | | 0.318) | |
| Example 32 | 93J | 4.08 eV | 34.2 | (0.682, | 182 |
| | | | | 0.318) | |
| Example 33 | 98J | 4.12 eV | 32.8 | (0.682, | 112 |
| | | | | 0.318) | |
| Example 34 | 99J | 4.16 eV | 31.9 | (0.682, | 118 |
| | | | | 0.318) | |
| Example 35 | 103J | 4.09 eV | 33.1 | (0.682, | 162 |
| | | | | 0.318) | |
| Example 36 | 110J | 3.96 eV | 34.2 | (0.682, | 229 |
| | | | | 0.318) | |
| Example 37 | 117J | 3.89 eV | 19.8 | (0.682, | 112 |
| | | | | 0.318) | |
| Example 38 | 125J | 3.78 eV | 27.9 | (0.682, | 183 |
| | | | | 0.318) | |
| Example 39 | 126J | 3.98 eV | 30.9 | (0.684, | 232 |
| | | | | 0.316) | |
| Example 40 | 133J | 3.95 eV | 36.8 | (0.685, | 295 |
| | | | | 0.315) | |
| Example 41 | 134J | 4.02 eV | 32.8 | (0.685, | 212 |
| | | | | 0.315) | |
| Example 42 | 135J | 4.09 eV | 33.8 | (0.685, | 198 |
| | | | | 0.315) | |
| Example 43 | 136J | 4.12 eV | 33.8 | (0.685, | 221 |
| | | | | 0.315) | |
| Example 44 | 138J | 4.18 eV | 33.5 | (0.684, | 241 |
| | | | | 0.316) | |
| Example 45 | 143J | 3.92 eV | 36.1 | (0.685, | 269 |
| | | | | 0.315) | |
| Example 46 | 143J | 4.06 eV | 37.0 | (0.685, | 278 |
| | | | | 0.315) | |
| Example 47 | 145J | 4.14 eV | 30.5 | (0.685, | 178 |
| | | | | 0.315) | |
| Example 48 | 146J | 4.18 eV | 29.9 | (0.685, | 188 |
| | | | | 0.315) | |
| Example 49 | 151J | 4.12 eV | 33.5 | (0.684, | 275 |
| | | | | 0.316) | |
| Example 50 | 160J | 4.27 eV | 28.6 | (0.684, | 178 |
| | | | | 0.316) | |
| Example 51 | 162J | 4.19 eV | 23.8 | (0.685, | 139 |
| | | | | 0.315) | |
| Example 52 | 169J | 3.69 eV | 30.8 | (0.685, | 99 |
| | | | | 0.315) | |
| Example 53 | 170J | 3.71 eV | 28.9 | (0.685, | 108 |
| | | | | 0.315) | |
| Example 54 | 178J | 3.42 eV | 33.8 | (0.685, | 111 |
| | | | | 0.315) | |
| Example 55 | 189J | 4.58 eV | 31.9 | (0.689, | 78 |
| | | | | 0.311) | |
| Example 56 | 190J | 3.44 eV | 37.2 | (0.685, | 88 |
| | | | | 0.315) | |
| Example 57 | 198J | 3.98 eV | 33.8 | (0.685, | 78 |
| | | | | 0.315) | |
| Example 58 | 198J:45J = | 4.12 eV | 35.5 | (0.685, | 242 |
| | 1:1 | | | 0.315) | |
| Example 59 | 201J | 4.02 eV | 36.8 | (0.685, | 75 |
| | | | | 0.315) | |
| Example 60 | 201J:45J = | 4.16 eV | 37.6 | (0.685, | 238 |
| | 1:1 | | | 0.315) | |
| Example 61 | 209J | 3.78 eV | 36.8 | (0.685, | 202 |
| | | | | 0.315) | |
| Example 62 | 210J | 3.82 eV | 35.9 | (0.685, | 190 |
| | | | | 0.315) | |
| Example 63 | 33J | 3.87 eV | 28.3 | (0.686, | 172 |
| | | | | 0.314) | |
| Example 64 | 228J | 3.87 eV | 27.6 | (0.686, | 180 |
| | | | | 0.314) | |
| Example 65 | 233J | 3.94 eV | 35.9 | (0.685, | 223 |
| | | | | 0.315) | |
| Example 66 | 235J | 3.94 eV | 36.1 | (0.686, | 278 |
| | | | | 0.314) | |
| Example 67 | 237J | 3.93 eV | 36.6 | (0.687, | 293 |
| | | | | 0.313) | |

As can be seen from Table 9, it could be confirmed from the comparison of Comparative Examples 1 to 18 and Examples 1 to 67 that the efficiency and service life of the device were excellent when a fluoranthene compound simultaneously having hole/electron characteristics in one molecule was used as a material for an organic material layer of an organic light emitting devices as in the compound represented by Chemical Formula 1 of the present application. Specifically, it could be confirmed that when the fluoranthene compound was used as a host material, the effect was excellent, meaning that the compound represented by Chemical Formula 1 of the present application is a material which easily adjusts the balance between holes and electrons, which is possessed by a single host.

Further, it was confirmed that when a p-type organic material was commonly used (co-dep) to adjust the balance of the single host from Examples 58 and 60, the service life of the device could be further improved.

Specifically, Comparative Examples Compounds A to F have a structure with an extended n-n conjugation, which is similar to that of the present invention, but correspond to fluoranthene compounds in which substituents such as naphthalene, phenanthrene, pyrene and triphenylene are not substituted, unlike the present invention. As can be seen from Table 9, it could be confirmed that the performance of the device, in which the compound represented by Chemical Formula 1 of the present application was used, was better than those of the devices in which these Comparative Example Compounds A to F were used.

Specifically, it could be confirmed that Comparative Example Compound A, which is a compound having a naphthalene skeleton used in Comparative Examples 1 and 2, and Comparative Example Compound B, which is a compound having a pyrene skeleton, did not show efficiency as an n-type host. The same was applied to the case where Comparative Example Compound D, which is a compound having a phenanthrene skeleton, was used.

In addition, although Comparative Example Compound C used in Comparative Example 3 had a molecular structure having hole characteristics as a triphenylene skeleton, the drive was increased due to the extended band-gap, and the service life was not measured.

In particular, although Comparative Example Compounds E and F used in Comparative Examples 5 and 6 were fluoranthene compounds, it could be confirmed from comparison with the compound represented by Chemical Formula 1 of the present application that Compounds E and F are compounds in which a specific substituent is not substituted, and the service life of the device was not good because the balance between holes and electrons was not made, unlike the compound represented by Chemical Formula 1 of the present application.

Comparative Example Compounds G to L are materials corresponding to a host having bipolarity, into which an electron donor substituent and an electron acceptor substituent are introduced, and it could be confirmed that the organic light emitting devices, in which Comparative Example Compounds G to L were used, had a service life which was slightly increased, compared to the organic light emitting devices in which Comparative Example Compounds A to F were used. However, the degree was not large, and it could be confirmed from comparison with the case where the compound represented by Chemical Formula 1 of the present application was used for the device that the organic light emitting devices, in which Comparative Example Compounds G to L were used, had an efficiency of 16 cd/A to 26 cd/A and a service life of 75 to 102 hours, but the organic light emitting device, in which the compound represented by Chemical Formula 1 of the present application was used, had even better efficiency and service life.

Comparative Example Compounds G to L have phenyl and naphthalene linkers or have a fluoranthene skeleton, and thus have a part which is structurally similar to the compound represented by Chemical Formula 1 of the present application, but it could be confirmed that when the compound represented by Chemical Formula 1 of the present application was used, the organic light emitting device had better efficiency and service life.

Additionally, in Comparative Example 13, Comparative Example Compound A was used as an n-type host and Comparative Example Compound K was used as a p-type host, and as a result of evaluating the performance of the device while adjusting each weight ratio, it could be confirmed that when the weight ratio was 1:1, the service life was improved compared to the case where Comparative Example Compound K was used alone, but the efficiency was reduced.

In addition, in the case of Comparative Example 14, as a result of using Compound 210J as an n-type host and Comparative Example A as a p-type host, it could be confirmed that compared to the case where Compound 210J was used as a single host, the efficiency or service life of the device was not improved. Likewise, it could be confirmed that even in the case of Comparative Examples 15 to 18, the efficiency or service life of the device was not significantly improved.

In contrast, as a result of evaluating the performance of Examples 1 to 39 and 63 in which the compound represented by Chemical Formula 1 of the present application was used as a single host of an n-type molecular structure of an organic light emitting device, it could be confirmed that even though an electron donor functional group such as a carbazole group or an amine group was not introduced, an excellent performance was exhibited by showing an efficiency more than 20 cd/A and a service life of 100 hours or more on average.

Furthermore, it could be confirmed that in Examples 40 to 62 in which a material corresponding to a structure into which electron donor and electron acceptor substituents were introduced was used as a host of the organic light emitting device by using a fluoranthene compound among the compounds represented by Chemical Formula 1 of the present application as a linker, the service life was typically improved compared to Examples 1 to 39.

Exceptionally, in the case of Examples 55 to 57 and 58 in which a functional group that attracts electrons is introduced into an electron accepting functional group as in Compounds 189J, 190J, 198J and 201J, or a compound having a small number of N (element) was used, a phenomenon, in which the injection of intramolecular electrons and electrons meet with holes to prevent the recombination, occurs, so that a problem in that the efficiency of the device is good but the service life of the device is relatively poor may occur.

However, it was confirmed that such a problem could be solved by co-dep with the n-type molecular structure as in Examples 57 and 60.

In Examples 64 to 67, the compound substituted with deuterium was used as a host of the organic light emitting device, and it could be confirmed that the efficiency and service life of the device were improved when the compound was substituted with deuterium. Since the dissociation energy of C-D bonds is generally 5 KJ/mol higher than the dissociation energy of C—H bonds of 410 KJ/mol, this is determined to be a phenomenon that occurs because the molecular stability is high when receiving electrical or thermal energy. This point could be confirmed by comparing the results of Examples 63 and 64 in which Compound 33J and Compound 228J having the same structure except for being substituted with deuterium were used, respectively.

Furthermore, as a result of comparing Compounds 233J, 235J and 237J having the same structure except that deuterium at another position was substituted in order to confirm the effect of the molecular stability on device results, it could be confirmed that when a fluoranthene group and a carbazole group used as a linker were substituted with deuterium, the service life of the device was further improved, compared to the case where triazine was substituted with deuterium.

### EXPLANATION OF REFERENCE NUMERALS AND SYMBOLS

100: Substrate
200: Positive electrode
300: Organic material layer
301: Hole injection layer
302: Hole transport layer
303: Light emitting layer
304: Hole blocking layer
305: Electron transport layer
306: Electron injection layer
400: Negative electrode

1. A heterocyclic compound represented by the following Chemical Formula 1: 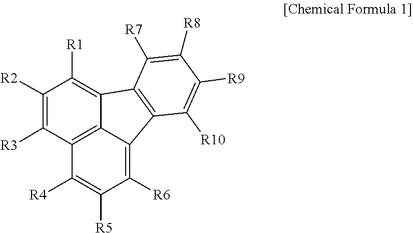
in Chemical Formula 1,
R1 to R10 are the same as or different from each other, and are each independently hydrogen; deuterium; -(L1)a-Ar1; or -(L2)b-Ar2, at least one of R1 to R10 is -(L1)a-Ar1, and at least one of the others is -(L2)b-Ar2,
L1 and L2 are the same as or different from each other, and are each independently a direct bond; a substituted or unsubstituted arylene group having 6 to 60 carbon atoms; or a substituted or unsubstituted heteroarylene group having 2 to 60 carbon atoms, a and b are an integer from 0 to 3, and when a and b are each 2 or higher, substituents in the parenthesis are each independent,
Ar1 is a monocyclic or polycyclic heterocyclic group which is substituted or unsubstituted, and comprises one or more N's, and
Ar2 is —NAr3Ar4; a substituted or unsubstituted aryl group having 6 to 60 carbon atoms; or a substituted or unsubstituted heteroaryl group having 2 to 60 carbon atoms, and Ar3 and Ar4 are the same as or different from each other, and are each independently a substituted or unsubstituted aryl group having 6 to 60 carbon atoms; or a substituted or unsubstituted heteroaryl group having 2 to 60 carbon atoms.

2. The heterocyclic compound of claim 1, wherein Ar1 is represented by the following Chemical Formula 2: 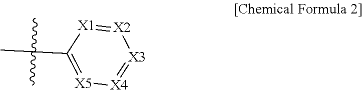
in Chemical Formula 2,
X1 is CR21 or N, X2 is CR22 or N, X3 is CR23 or N, X4 is CR24 or N, and X5 is CR25 or N, and
R21 to R25 are the same as or different from each other, and are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or hetero ring, and here, [symbol] is a site which is linked to L1.

3. The heterocyclic compound of claim 2, wherein Chemical Formula 2 is represented by any one of the following Chemical Formulae 3 to 6: 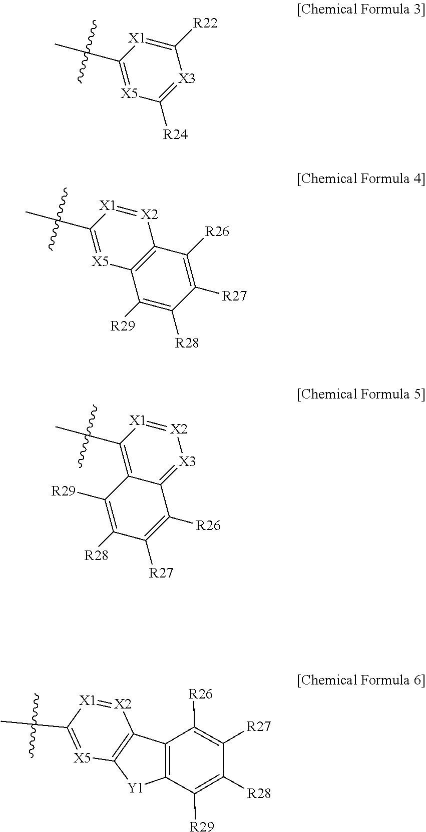
in Chemical Formula 3, one or more of X1, X3 and X5 are N, and the others are the same as those defined in Chemical Formula 2,
in Chemical Formula 4, one or more of X1, X2 and X5 are N, and the others are the same as those defined in Chemical Formula 2,
in Chemical Formula 5, one or more of X1 to X3 are N, and the others are the same as those defined in Chemical Formula 2,
in Chemical Formula 6, one or more of X1, X2 and X5 are N, the others are the same as those defined in Chemical Formula 2, and Y1 is O; or S, and
R22, R24 and R26 to R29 are the same as or different from each other, and are each independently selected from the group consisting of hydrogen; deuterium; a halogen; a cyano group; a substituted or unsubstituted alkyl group having 1 to 60 carbon atoms; a substituted or unsubstituted alkenyl group having 2 to 60 carbon atoms; a substituted or unsubstituted alkynyl group having 2 to 60 carbon atoms; a substituted or unsubstituted alkoxy group having 1 to 20 carbon atoms; a substituted or unsubstituted cycloalkyl group having 3 to 60 carbon atoms; a substituted or unsubstituted heterocycloalkyl group having 2 to 60 carbon atoms; a substituted or unsubstituted aryl group having 6 to 60 carbon atoms; a substituted or unsubstituted heteroaryl group having 2 to 60 carbon atoms; a substituted or unsubstituted phosphine oxide group; and a substituted or unsubstituted amine group, or two or more adjacent groups are bonded to each other to form a substituted or unsubstituted aliphatic or aromatic hydrocarbon ring or hetero ring, and here, [symbol] is a site which is linked to L1.

4. The heterocyclic compound of claim 1, wherein Chemical Formula 1 is represented by any one of the following Chemical Formulae 1-1 to 1-4: 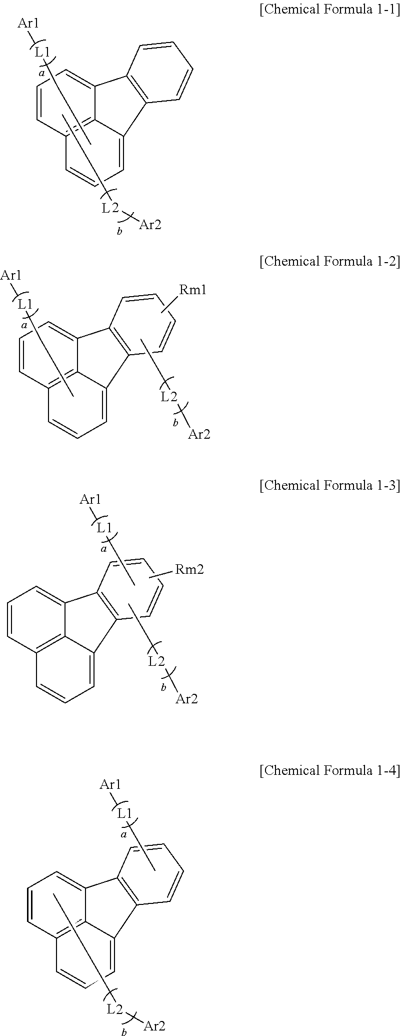
in Chemical Formulae 1-1 to 1-4,
hydrogen is unsubstituted or substituted with deuterium, and
Rm1 and Rm2 are the same as or different from each other, and are each independently hydrogen; deuterium; an aryl group having 6 to 60 carbon atoms; or a substituted or unsubstituted heteroaryl group having 2 to 60 carbon atoms, and the definitions of L1, L2, Ar1 Ar2, a and b are the same as those in Chemical Formula 1.

5. The heterocyclic compound of claim 1, wherein a deuterium content of the heterocyclic compound represented by Chemical Formula 1 is 0% to 100%.

6. The heterocyclic compound of claim 1, wherein Chemical Formula 1 is represented by any one of the following compounds:

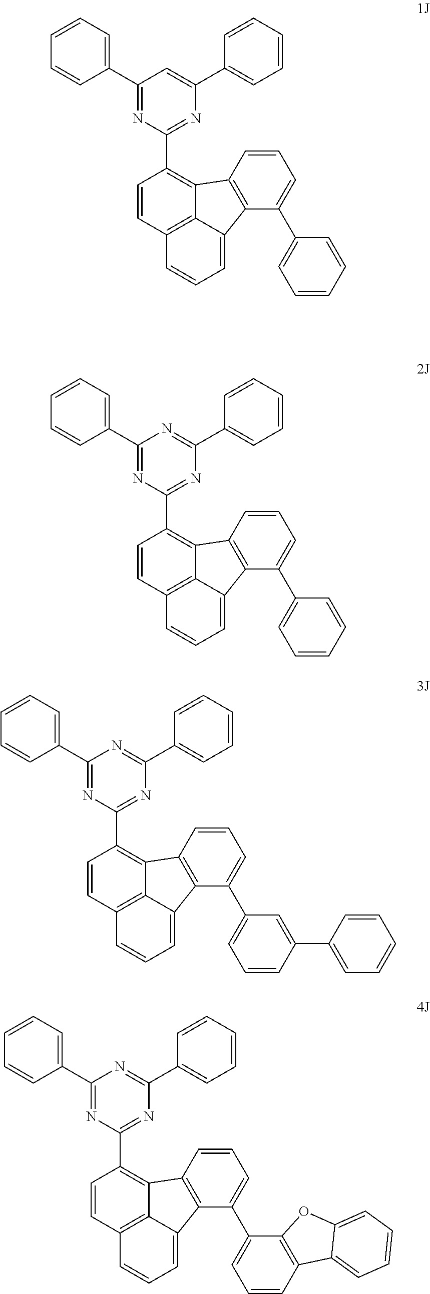 [SMILES: c1ccc(-c2cc(-c3ccccc3)nc(-c3ccc4cccc5c4c3-c3cccc(-c4ccccc4)c3-5)n2)cc1 ; c1ccc(-c2nc(-c3ccccc3)nc(-c3ccc4cccc5c4c3-c3cccc(-c4ccccc4)c3-5)n2)cc1 ; c1ccc(-c2cccc(-c3cccc4c3-c3cccc5ccc(-c6nc(-c7ccccc7)nc(-c7ccccc7)n6)c-4c35)c2)cc1 ; c1ccc(-c2nc(-c3ccccc3)nc(-c3ccc4cccc5c4c3-c3cccc(-c4cccc6c4oc4ccccc46)c3-5)n2)cc1]

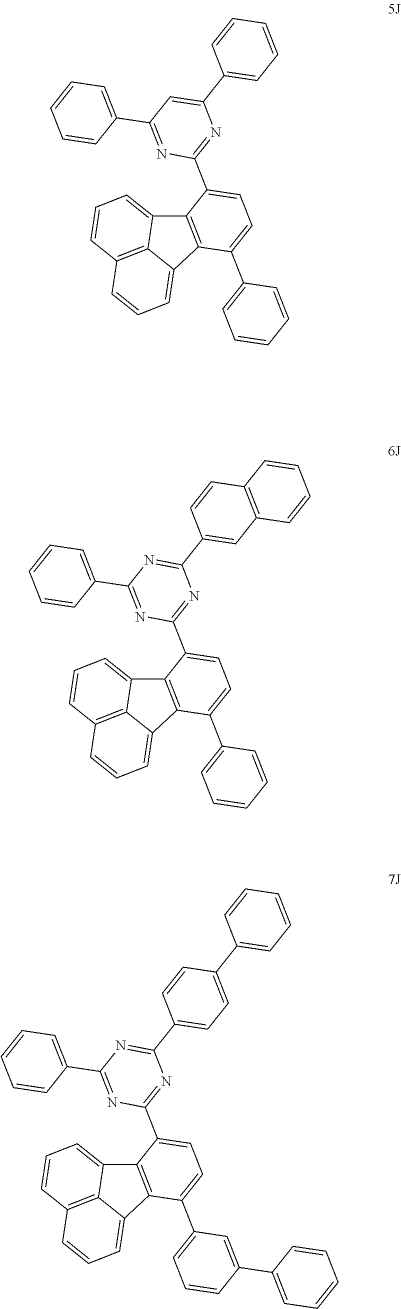 [SMILES: c1ccc(-c2cc(-c3ccccc3)nc(-c3ccc(-c4ccccc4)c4c3-c3cccc5cccc-4c35)n2)cc1 ; c1ccc(-c2nc(-c3ccc4ccccc4c3)nc(-c3ccc(-c4ccccc4)c4c3-c3cccc5cccc-4c35)n2)cc1 ; c1ccc(-c2ccc(-c3nc(-c4ccccc4)nc(-c4ccc(-c5cccc(-c6ccccc6)c5)c5c4-c4cccc6cccc-5c46)n3)cc2)cc1]

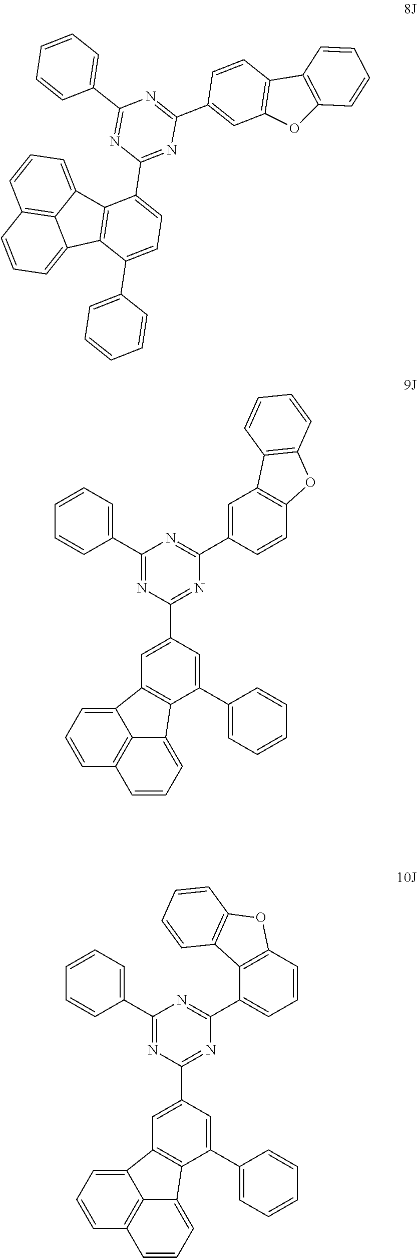 [SMILES: c1ccc(-c2nc(-c3ccc4c(c3)oc3ccccc34)nc(-c3ccc(-c4ccccc4)c4c3-c3cccc5cccc-4c35)n2)cc1 ; c1ccc(-c2nc(-c3cc(-c4ccccc4)c4c(c3)-c3cccc5cccc-4c35)nc(-c3ccc4oc5ccccc5c4c3)n2)cc1 ; c1ccc(-c2nc(-c3cc(-c4ccccc4)c4c(c3)-c3cccc5cccc-4c35)nc(-c3cccc4oc5ccccc5c34)n2)cc1]

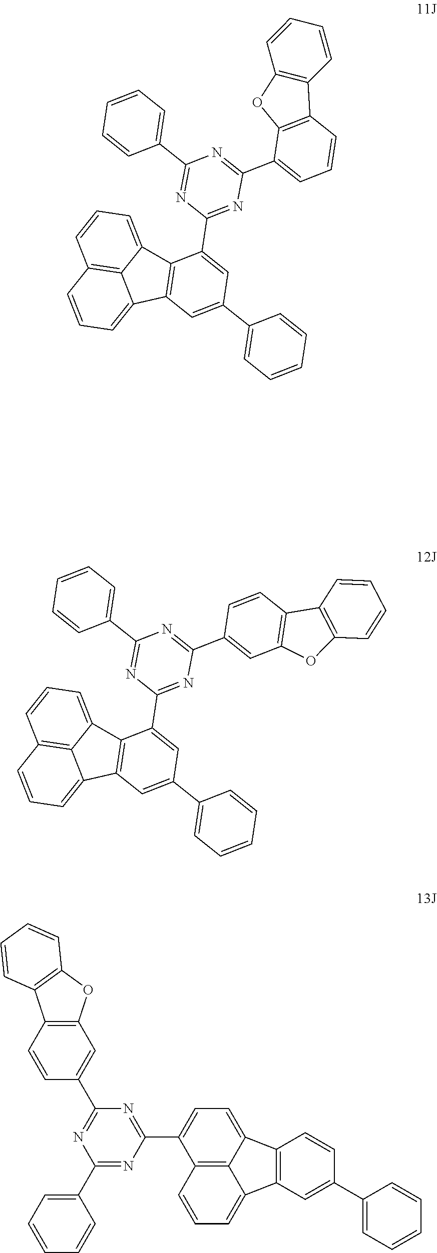 [SMILES: c1ccc(-c2cc(-c3nc(-c4ccccc4)nc(-c4cccc5c4oc4ccccc45)n3)c3c(c2)-c2cccc4cccc-3c24)cc1 ; c1ccc(-c2cc(-c3nc(-c4ccccc4)nc(-c4ccc5c(c4)oc4ccccc45)n3)c3c(c2)-c2cccc4cccc-3c24)cc1 ; c1ccc(-c2ccc3c(c2)-c2cccc4c(-c5nc(-c6ccccc6)nc(-c6ccc7c(c6)oc6ccccc67)n5)ccc-3c24)cc1]

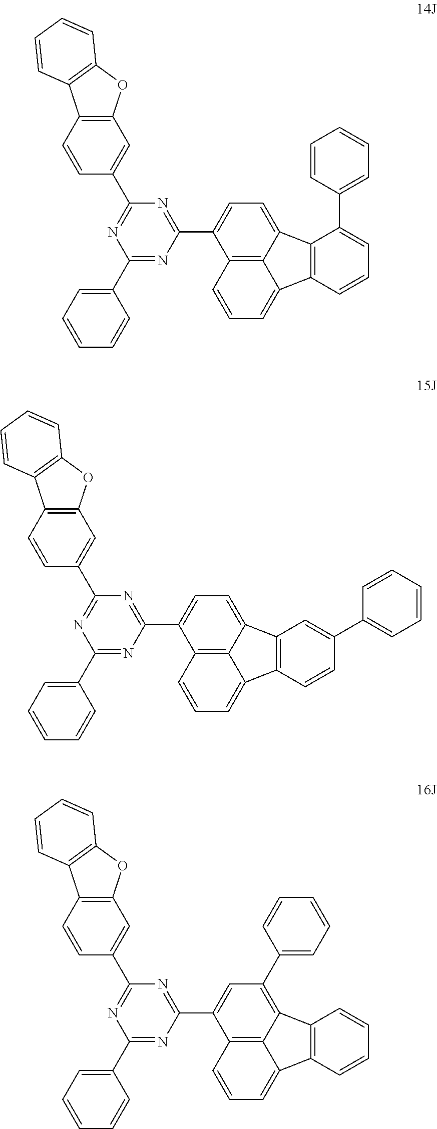 [SMILES: c1ccc(-c2nc(-c3ccc4c(c3)oc3ccccc34)nc(-c3ccc4c5c(cccc35)-c3cccc(-c5ccccc5)c3-4)n2)cc1 ; c1ccc(-c2ccc3c(c2)-c2ccc(-c4nc(-c5ccccc5)nc(-c5ccc6c(c5)oc5ccccc56)n4)c4cccc-3c24)cc1 ; c1ccc(-c2nc(-c3ccc4c(c3)oc3ccccc34)nc(-c3cc(-c4ccccc4)c4c5c(cccc35)-c3ccccc3-4)n2)cc1]

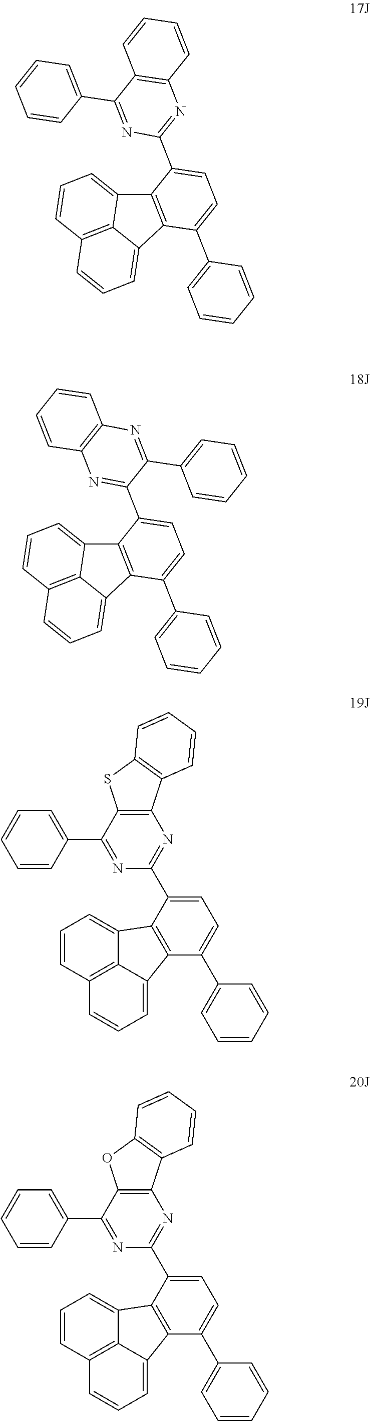

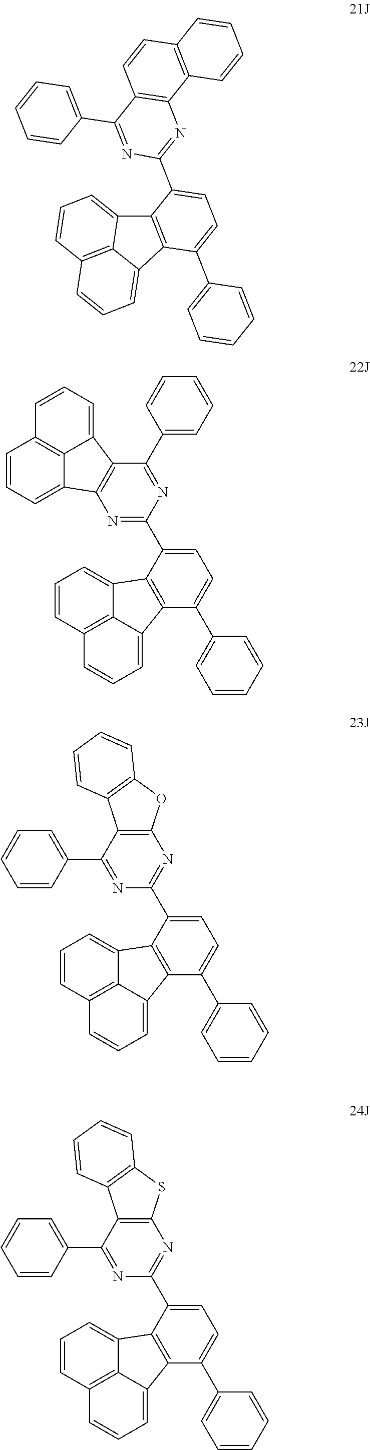

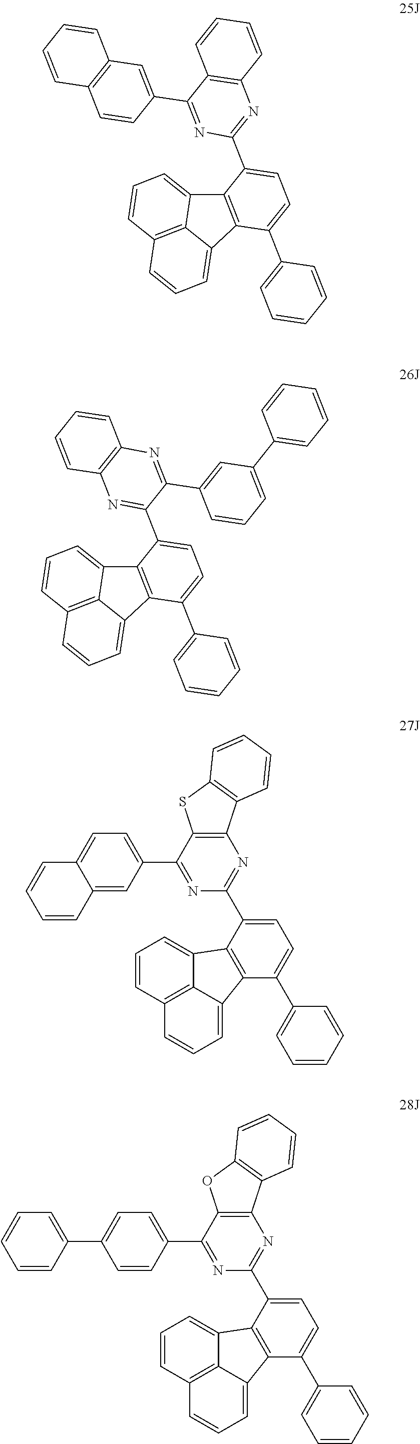

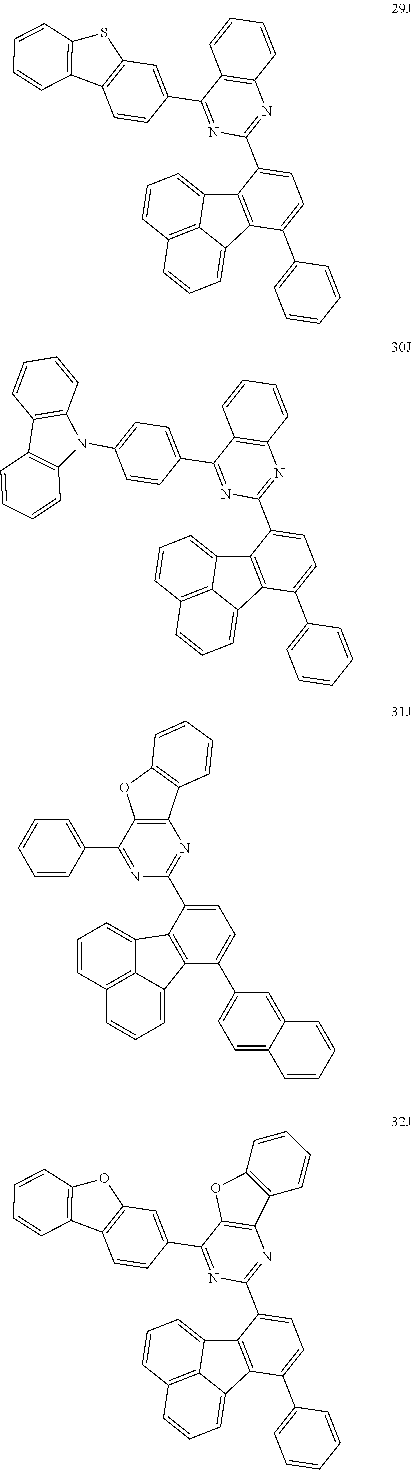 [SMILES: c1ccc(-c2ccc(-c3nc(-c4ccc5c(c4)sc4ccccc45)c4ccccc4n3)c3c2-c2cccc4cccc-3c24)cc1 ; c1ccc(-c2ccc(-c3nc(-c4ccc(-n5c6ccccc6c6ccccc65)cc4)c4ccccc4n3)c3c2-c2cccc4cccc-3c24)cc1 ; c1ccc(-c2nc(-c3ccc(-c4ccc5ccccc5c4)c4c3-c3cccc5cccc-4c35)nc3c2oc2ccccc23)cc1 ; c1ccc(-c2ccc(-c3nc(-c4ccc5c(c4)oc4ccccc45)c4oc5ccccc5c4n3)c3c2-c2cccc4cccc-3c24)cc1]

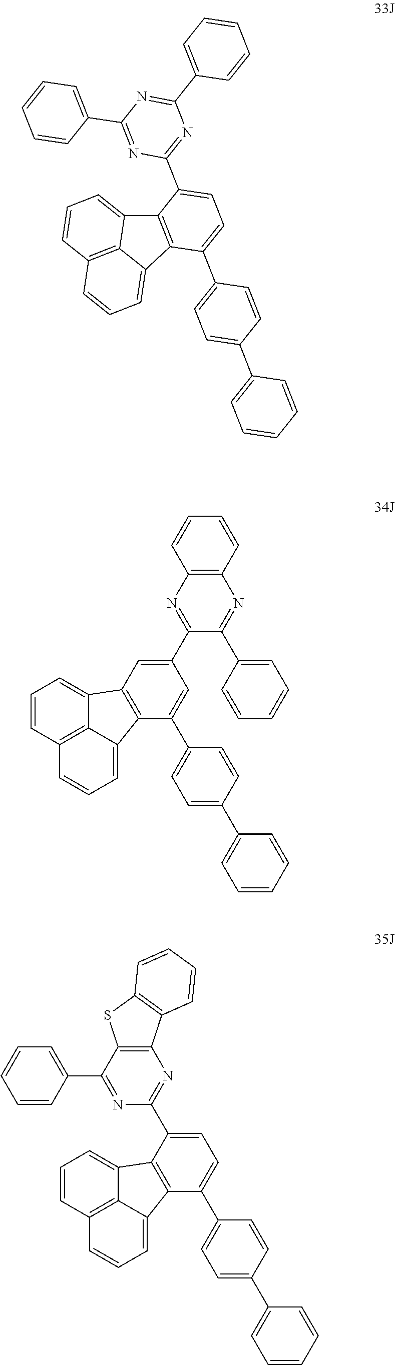 [SMILES: c1ccc(-c2ccc(-c3ccc(-c4nc(-c5ccccc5)nc(-c5ccccc5)n4)c4c3-c3cccc5cccc-4c35)cc2)cc1 ; c1ccc(-c2ccc(-c3cc(-c4nc5ccccc5nc4-c4ccccc4)cc4c3-c3cccc5cccc-4c35)cc2)cc1 ; c1ccc(-c2ccc(-c3ccc(-c4nc(-c5ccccc5)c5sc6ccccc6c5n4)c4c3-c3cccc5cccc-4c35)cc2)cc1]

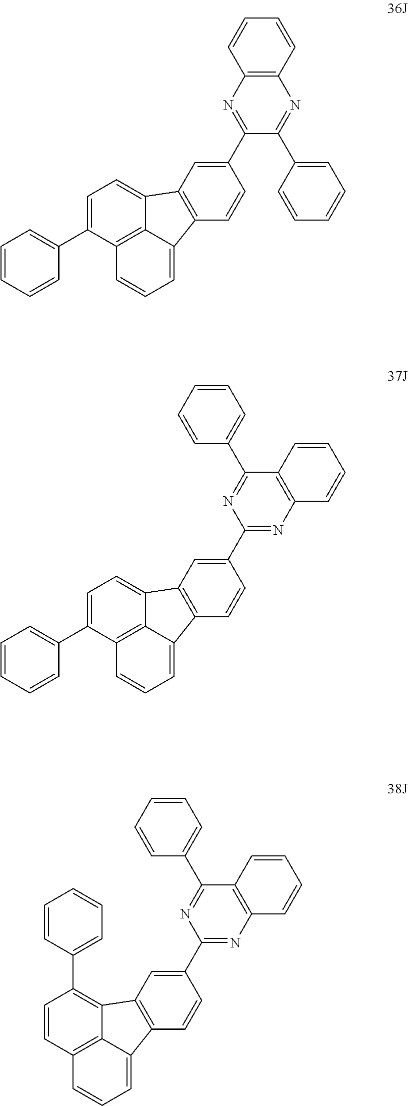 [SMILES: c1ccc(-c2nc3ccccc3nc2-c2ccc3c(c2)-c2ccc(-c4ccccc4)c4cccc-3c24)cc1 ; c1ccc(-c2nc(-c3ccc4c(c3)-c3ccc(-c5ccccc5)c5cccc-4c35)nc3ccccc23)cc1 ; c1ccc(-c2ccc3cccc4c3c2-c2cc(-c3nc(-c5ccccc5)c5ccccc5n3)ccc2-4)cc1]

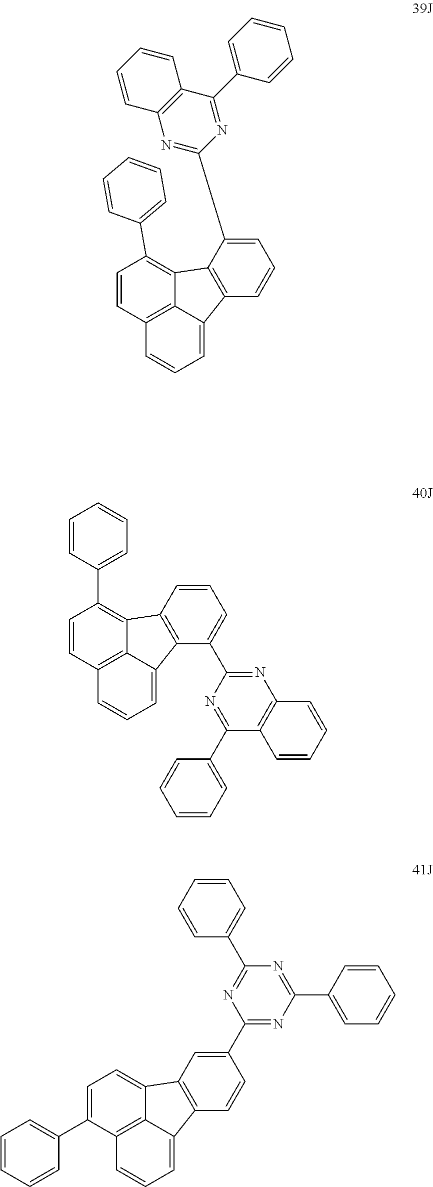 [SMILES: c1ccc(-c2ccc3cccc4c3c2-c2c(-c3nc(-c5ccccc5)c5ccccc5n3)cccc2-4)cc1 ; c1ccc(-c2ccc3cccc4c3c2-c2cccc(-c3nc(-c5ccccc5)c5ccccc5n3)c2-4)cc1 ; c1ccc(-c2nc(-c3ccccc3)nc(-c3ccc4c(c3)-c3ccc(-c5ccccc5)c5cccc-4c35)n2)cc1]

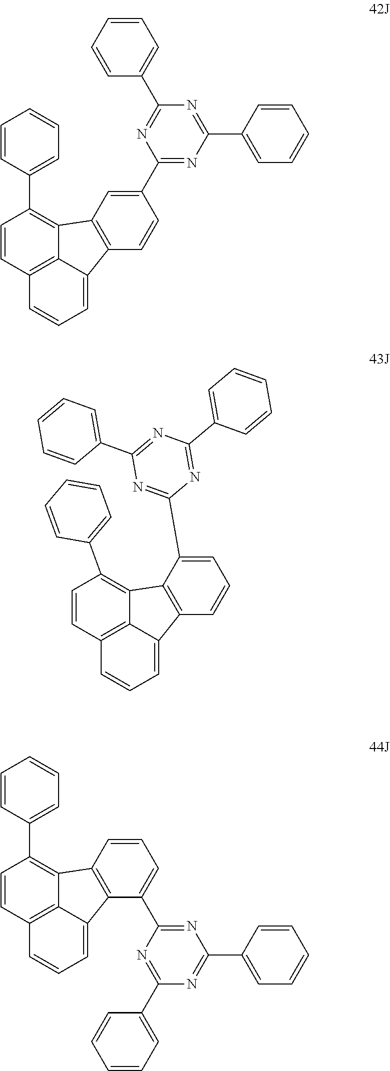 [SMILES: c1ccc(-c2nc(-c3ccccc3)nc(-c3ccc4c(c3)-c3c(-c5ccccc5)ccc5cccc-4c35)n2)cc1 ; c1ccc(-c2nc(-c3ccccc3)nc(-c3cccc4c3-c3c(-c5ccccc5)ccc5cccc-4c35)n2)cc1 ; c1ccc(-c2nc(-c3ccccc3)nc(-c3cccc4c3-c3cccc5ccc(-c6ccccc6)c-4c35)n2)cc1]

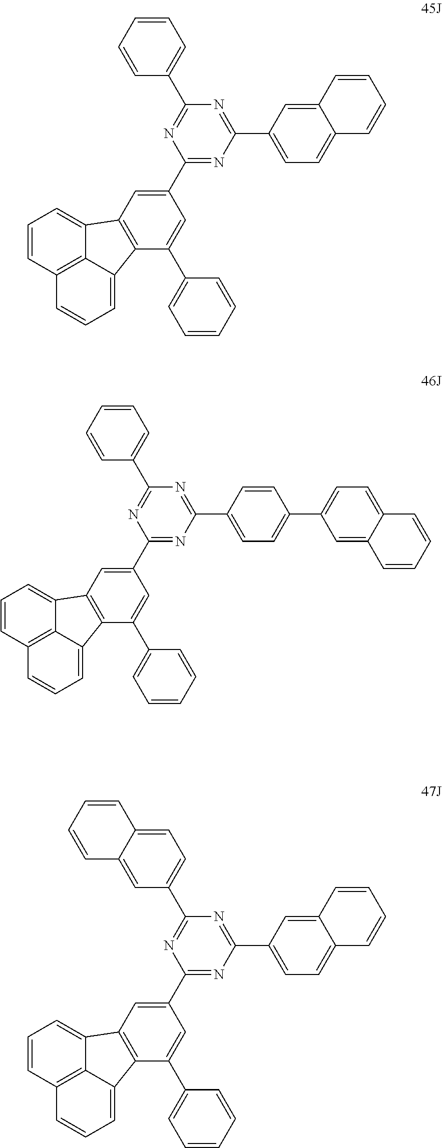 [SMILES: c1ccc(-c2nc(-c3cc(-c4ccccc4)c4c(c3)-c3cccc5cccc-4c35)nc(-c3ccc4ccccc4c3)n2)cc1 ; c1ccc(-c2nc(-c3ccc(-c4ccc5ccccc5c4)cc3)nc(-c3cc(-c4ccccc4)c4c(c3)-c3cccc5cccc-4c35)n2)cc1 ; c1ccc(-c2cc(-c3nc(-c4ccc5ccccc5c4)nc(-c4ccc5ccccc5c4)n3)cc3c2-c2cccc4cccc-3c24)cc1]

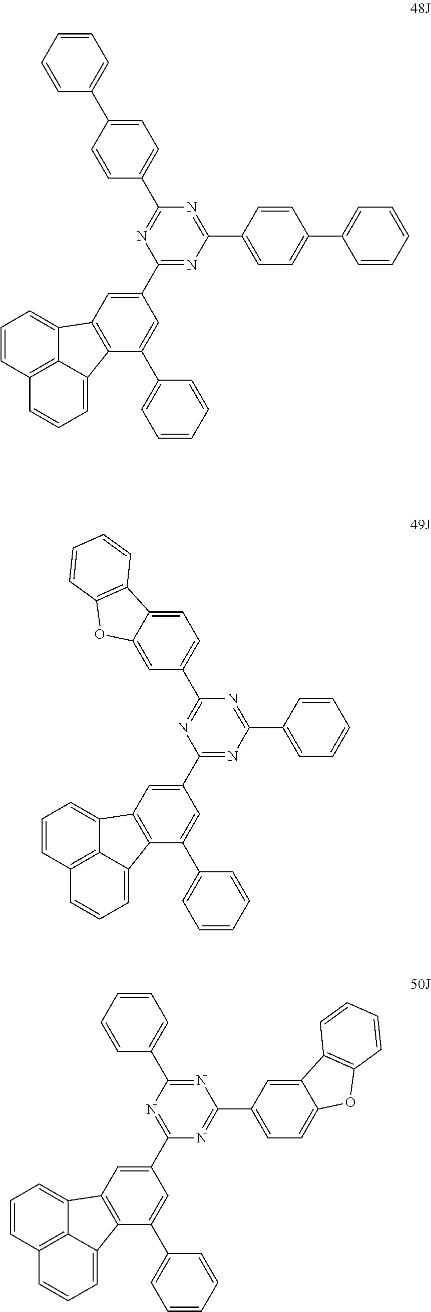 [SMILES: c1ccc(-c2ccc(-c3nc(-c4ccc(-c5ccccc5)cc4)nc(-c4cc(-c5ccccc5)c5c(c4)-c4cccc6cccc-5c46)n3)cc2)cc1 ; c1ccc(-c2nc(-c3cc(-c4ccccc4)c4c(c3)-c3cccc5cccc-4c35)nc(-c3ccc4c(c3)oc3ccccc34)n2)cc1 ; c1ccc(-c2nc(-c3cc(-c4ccccc4)c4c(c3)-c3cccc5cccc-4c35)nc(-c3ccc4oc5ccccc5c4c3)n2)cc1]

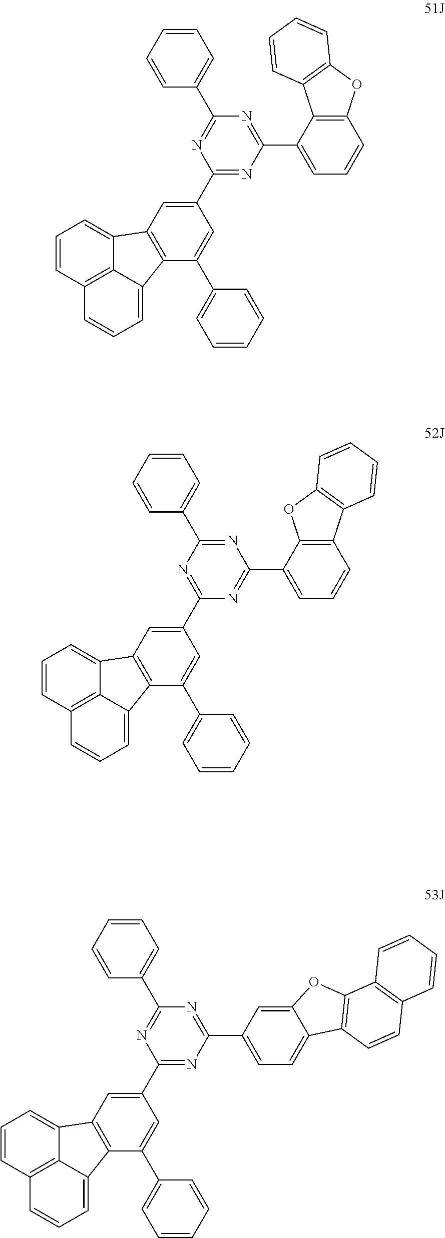 [SMILES: c1ccc(-c2nc(-c3cc(-c4ccccc4)c4c(c3)-c3cccc5cccc-4c35)nc(-c3cccc4oc5ccccc5c34)n2)cc1 ; c1ccc(-c2nc(-c3cc(-c4ccccc4)c4c(c3)-c3cccc5cccc-4c35)nc(-c3cccc4c3oc3ccccc34)n2)cc1 ; c1ccc(-c2nc(-c3cc(-c4ccccc4)c4c(c3)-c3cccc5cccc-4c35)nc(-c3ccc4c(c3)oc3c5ccccc5ccc43)n2)cc1]

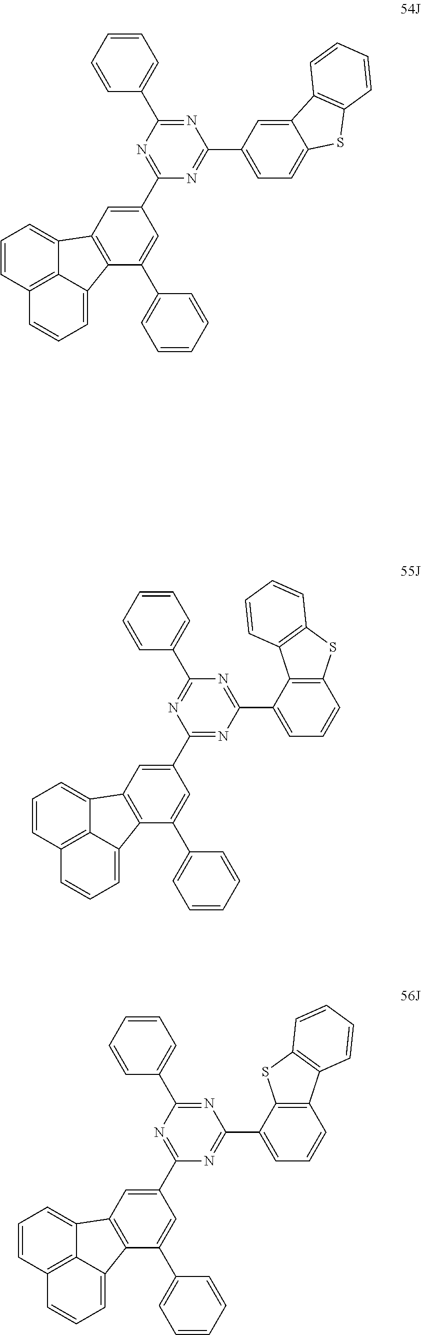 [SMILES: c1ccc(-c2nc(-c3cc(-c4ccccc4)c4c(c3)-c3cccc5cccc-4c35)nc(-c3ccc4sc5ccccc5c4c3)n2)cc1 ; c1ccc(-c2nc(-c3cc(-c4ccccc4)c4c(c3)-c3cccc5cccc-4c35)nc(-c3cccc4sc5ccccc5c34)n2)cc1 ; c1ccc(-c2nc(-c3cc(-c4ccccc4)c4c(c3)-c3cccc5cccc-4c35)nc(-c3cccc4c3sc3ccccc34)n2)cc1]

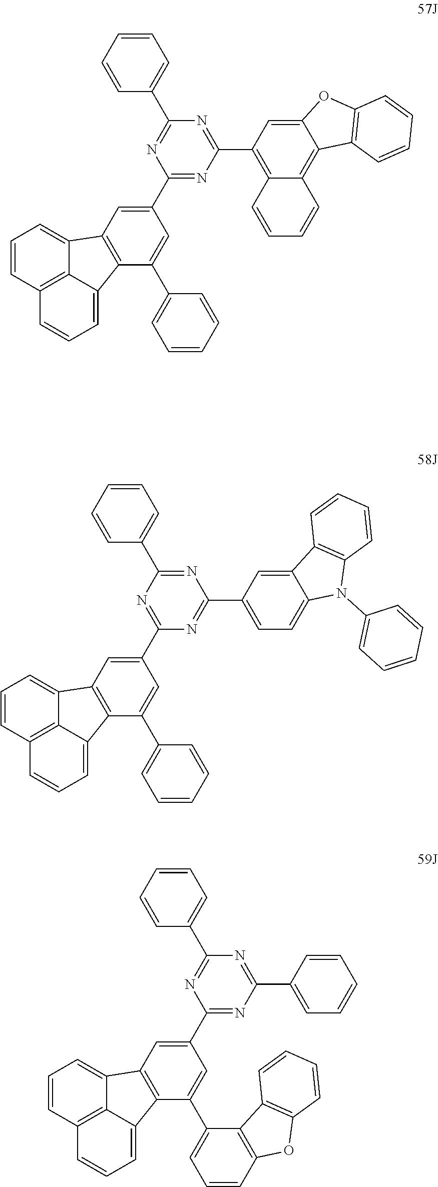 [SMILES: c1ccc(-c2nc(-c3cc(-c4ccccc4)c4c(c3)-c3cccc5cccc-4c35)nc(-c3cc4oc5ccccc5c4c4ccccc34)n2)cc1 ; c1ccc(-c2nc(-c3cc(-c4ccccc4)c4c(c3)-c3cccc5cccc-4c35)nc(-c3ccc4c(c3)c3ccccc3n4-c3ccccc3)n2)cc1 ; c1ccc(-c2nc(-c3ccccc3)nc(-c3cc4c(c(-c5cccc6oc7ccccc7c56)c3)-c3cccc5cccc-4c35)n2)cc1]

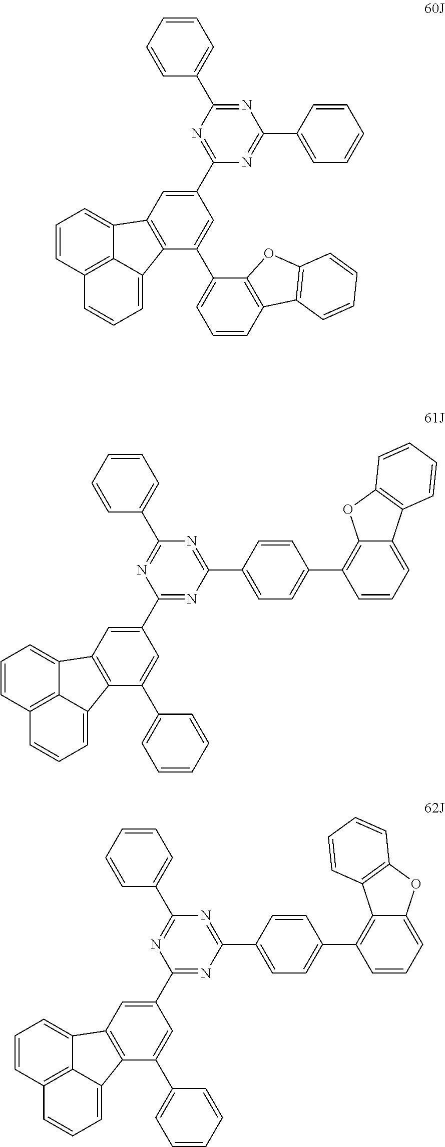 [SMILES: c1ccc(-c2nc(-c3ccccc3)nc(-c3cc(-c4cccc5c4oc4ccccc45)c4c(c3)-c3cccc5cccc-4c35)n2)cc1 ; c1ccc(-c2nc(-c3ccc(-c4cccc5c4oc4ccccc45)cc3)nc(-c3cc(-c4ccccc4)c4c(c3)-c3cccc5cccc-4c35)n2)cc1 ; c1ccc(-c2nc(-c3ccc(-c4cccc5oc6ccccc6c45)cc3)nc(-c3cc(-c4ccccc4)c4c(c3)-c3cccc5cccc-4c35)n2)cc1]

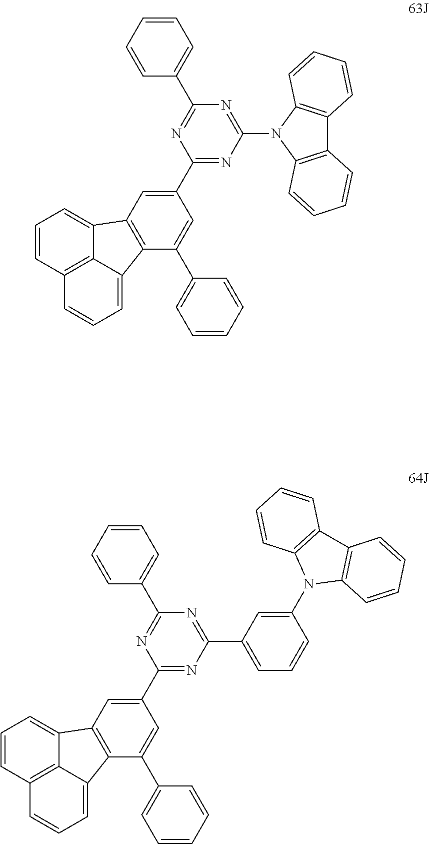 [SMILES: c1ccc(-c2nc(-c3cc(-c4ccccc4)c4c(c3)-c3cccc5cccc-4c35)nc(-n3c4ccccc4c4ccccc43)n2)cc1 ; c1ccc(-c2nc(-c3cccc(-n4c5ccccc5c5ccccc54)c3)nc(-c3cc(-c4ccccc4)c4c(c3)-c3cccc5cccc-4c35)n2)cc1]

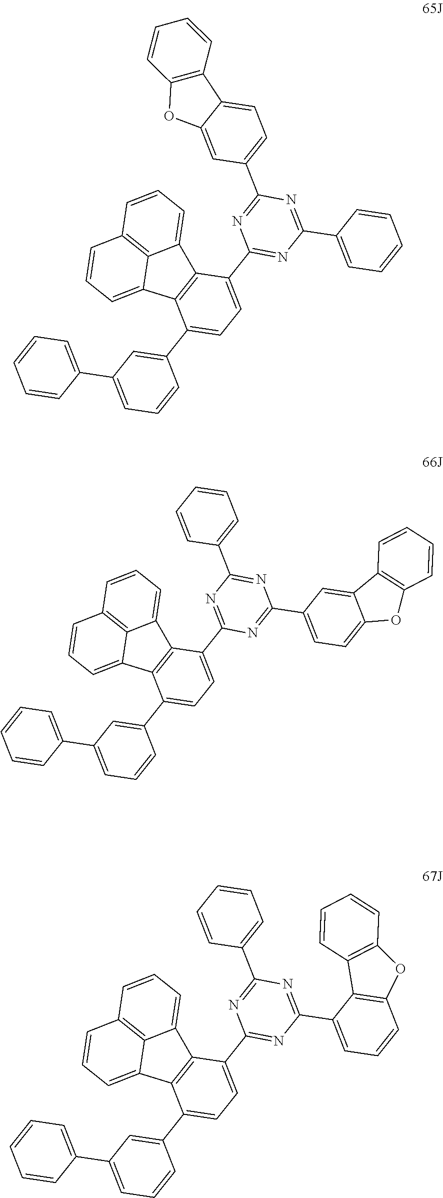 [SMILES: c1ccc(-c2cccc(-c3ccc(-c4nc(-c5ccccc5)nc(-c5ccc6c(c5)oc5ccccc56)n4)c4c3-c3cccc5cccc-4c35)c2)cc1 ; c1ccc(-c2cccc(-c3ccc(-c4nc(-c5ccccc5)nc(-c5ccc6oc7ccccc7c6c5)n4)c4c3-c3cccc5cccc-4c35)c2)cc1 ; c1ccc(-c2cccc(-c3ccc(-c4nc(-c5ccccc5)nc(-c5cccc6oc7ccccc7c56)n4)c4c3-c3cccc5cccc-4c35)c2)cc1]

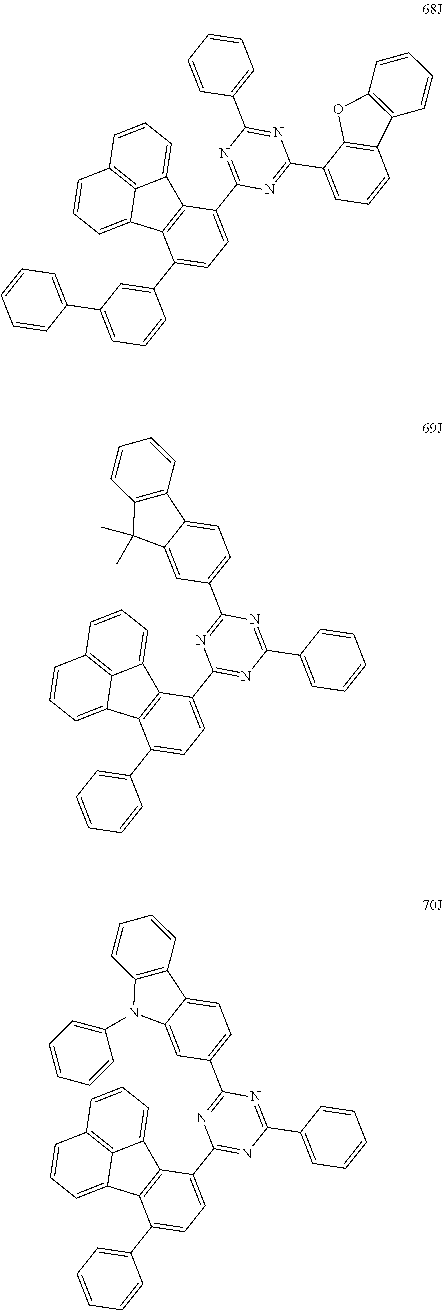 [SMILES: c1ccc(-c2cccc(-c3ccc(-c4nc(-c5ccccc5)nc(-c5cccc6c5oc5ccccc56)n4)c4c3-c3cccc5cccc-4c35)c2)cc1 ; CC1(C)c2ccccc2-c2ccc(-c3nc(-c4ccccc4)nc(-c4ccc(-c5ccccc5)c5c4-c4cccc6cccc-5c46)n3)cc21 ; c1ccc(-c2nc(-c3ccc4c5ccccc5n(-c5ccccc5)c4c3)nc(-c3ccc(-c4ccccc4)c4c3-c3cccc5cccc-4c35)n2)cc1]

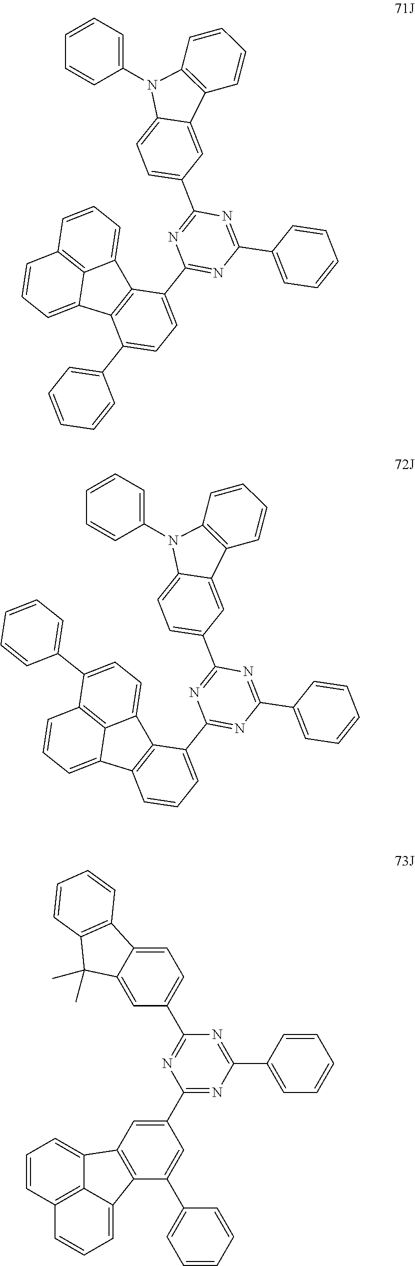 [SMILES: c1ccc(-c2nc(-c3ccc4c(c3)c3ccccc3n4-c3ccccc3)nc(-c3ccc(-c4ccccc4)c4c3-c3cccc5cccc-4c35)n2)cc1 ; c1ccc(-c2nc(-c3ccc4c(c3)c3ccccc3n4-c3ccccc3)nc(-c3cccc4c3-c3ccc(-c5ccccc5)c5cccc-4c35)n2)cc1 ; CC1(C)c2ccccc2-c2ccc(-c3nc(-c4ccccc4)nc(-c4cc(-c5ccccc5)c5c(c4)-c4cccc6cccc-5c46)n3)cc21]

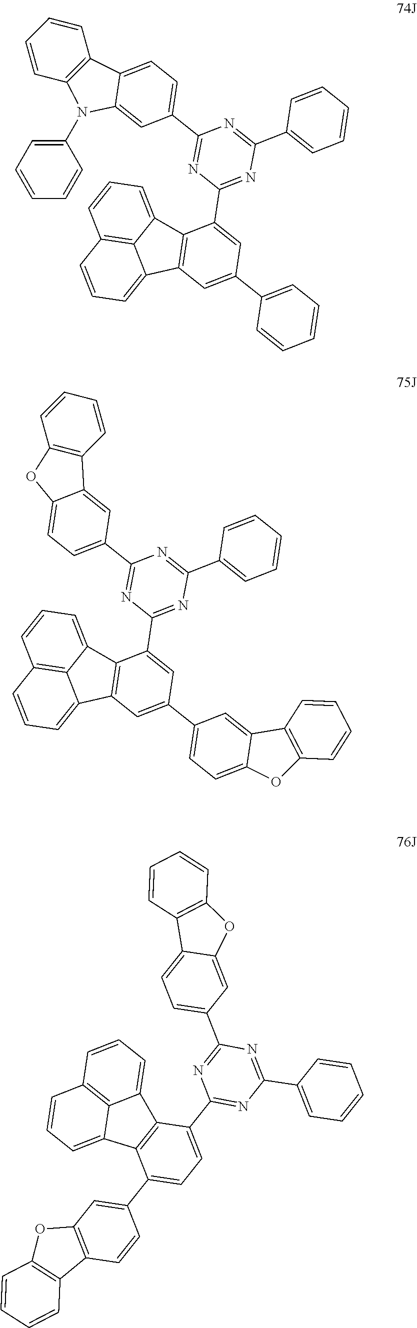 [SMILES: c1ccc(-c2cc(-c3nc(-c4ccccc4)nc(-c4ccc5c6ccccc6n(-c6ccccc6)c5c4)n3)c3c(c2)-c2cccc4cccc-3c24)cc1 ; c1ccc(-c2nc(-c3ccc4oc5ccccc5c4c3)nc(-c3cc(-c4ccc5oc6ccccc6c5c4)cc4c3-c3cccc5cccc-4c35)n2)cc1 ; c1ccc(-c2nc(-c3ccc4c(c3)oc3ccccc34)nc(-c3ccc(-c4ccc5c(c4)oc4ccccc45)c4c3-c3cccc5cccc-4c35)n2)cc1]

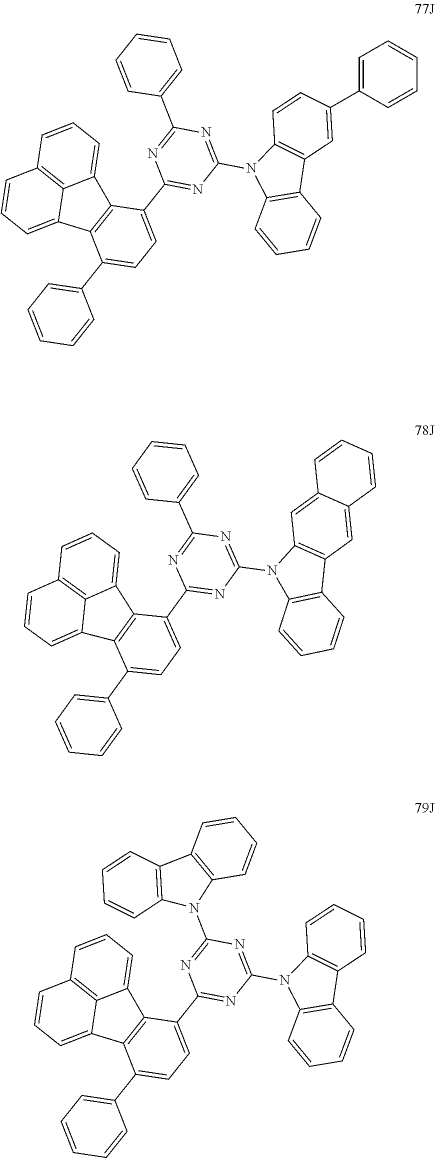 [SMILES: c1ccc(-c2ccc3c(c2)c2ccccc2n3-c2nc(-c3ccccc3)nc(-c3ccc(-c4ccccc4)c4c3-c3cccc5cccc-4c35)n2)cc1 ; c1ccc(-c2nc(-c3ccc(-c4ccccc4)c4c3-c3cccc5cccc-4c35)nc(-n3c4ccccc4c4cc5ccccc5cc43)n2)cc1 ; c1ccc(-c2ccc(-c3nc(-n4c5ccccc5c5ccccc54)nc(-n4c5ccccc5c5ccccc54)n3)c3c2-c2cccc4cccc-3c24)cc1]

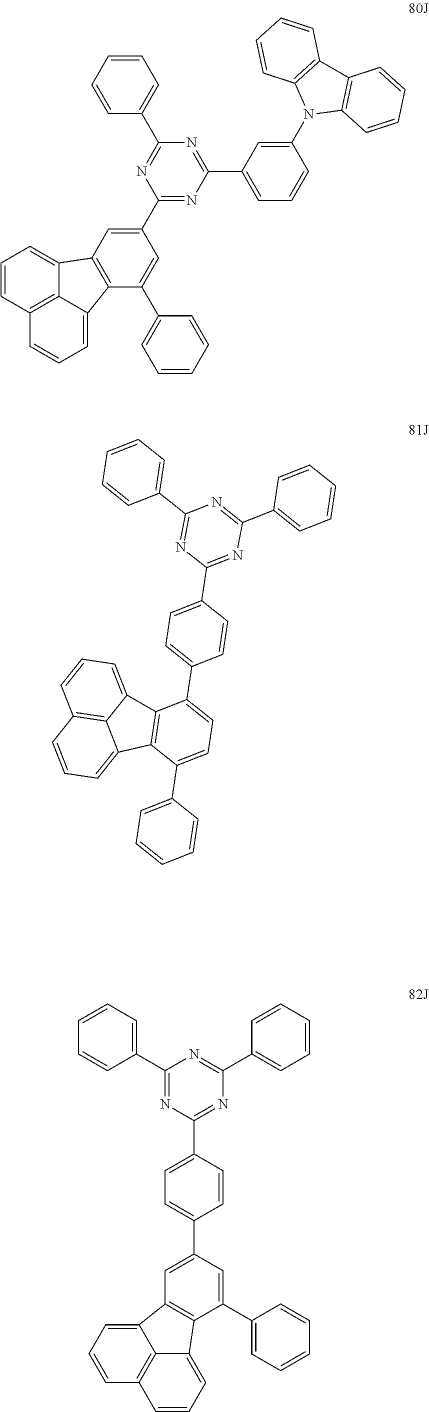 [SMILES: c1ccc(-c2nc(-c3cccc(-n4c5ccccc5c5ccccc54)c3)nc(-c3cc(-c4ccccc4)c4c(c3)-c3cccc5cccc-4c35)n2)cc1 ; c1ccc(-c2nc(-c3ccccc3)nc(-c3ccc(-c4ccc(-c5ccccc5)c5c4-c4cccc6cccc-5c46)cc3)n2)cc1 ; c1ccc(-c2nc(-c3ccccc3)nc(-c3ccc(-c4cc(-c5ccccc5)c5c(c4)-c4cccc6cccc-5c46)cc3)n2)cc1]

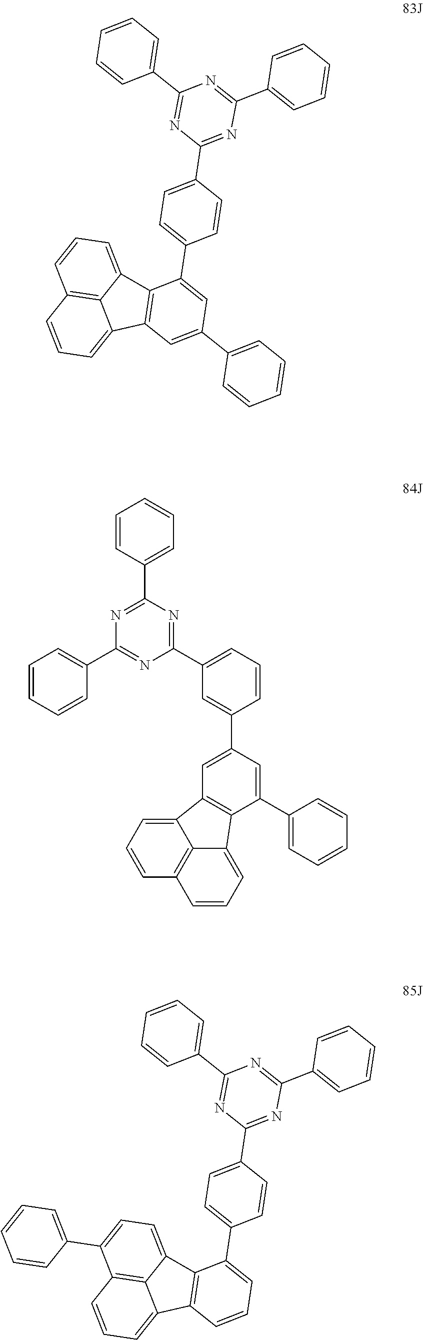 [SMILES: c1ccc(-c2cc(-c3ccc(-c4nc(-c5ccccc5)nc(-c5ccccc5)n4)cc3)c3c(c2)-c2cccc4cccc-3c24)cc1 ; c1ccc(-c2nc(-c3ccccc3)nc(-c3cccc(-c4cc(-c5ccccc5)c5c(c4)-c4cccc6cccc-5c46)c3)n2)cc1 ; c1ccc(-c2nc(-c3ccccc3)nc(-c3ccc(-c4cccc5c4-c4ccc(-c6ccccc6)c6cccc-5c46)cc3)n2)cc1]

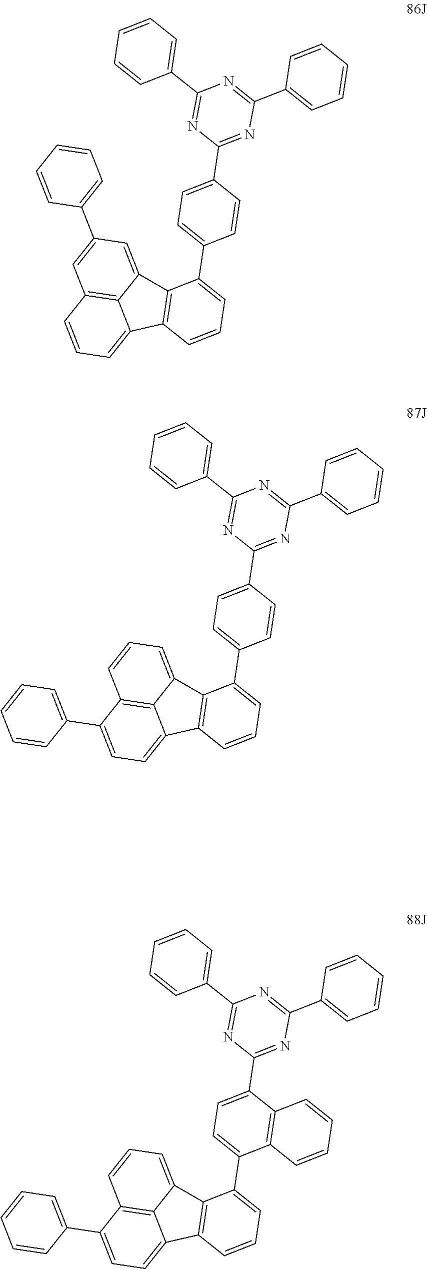 [SMILES: c1ccc(-c2cc3c4c(cccc4c2)-c2cccc(-c4ccc(-c5nc(-c6ccccc6)nc(-c6ccccc6)n5)cc4)c2-3)cc1 ; c1ccc(-c2nc(-c3ccccc3)nc(-c3ccc(-c4cccc5c4-c4cccc6c(-c7ccccc7)ccc-5c46)cc3)n2)cc1 ; c1ccc(-c2nc(-c3ccccc3)nc(-c3ccc(-c4cccc5c4-c4cccc6c(-c7ccccc7)ccc-5c46)c4ccccc34)n2)cc1]

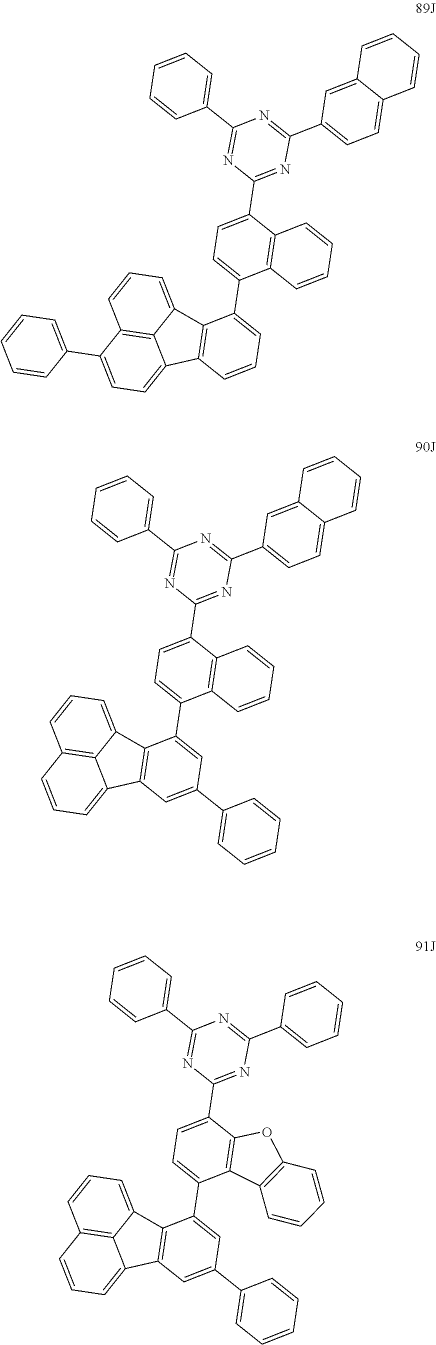 [SMILES: c1ccc(-c2nc(-c3ccc4ccccc4c3)nc(-c3ccc(-c4cccc5c4-c4cccc6c(-c7ccccc7)ccc-5c46)c4ccccc34)n2)cc1 ; c1ccc(-c2cc(-c3ccc(-c4nc(-c5ccccc5)nc(-c5ccc6ccccc6c5)n4)c4ccccc34)c3c(c2)-c2cccc4cccc-3c24)cc1 ; c1ccc(-c2cc3c(c(-c4ccc(-c5nc(-c6ccccc6)nc(-c6ccccc6)n5)c5oc6ccccc6c45)c2)-c2cccc4cccc-3c24)cc1]

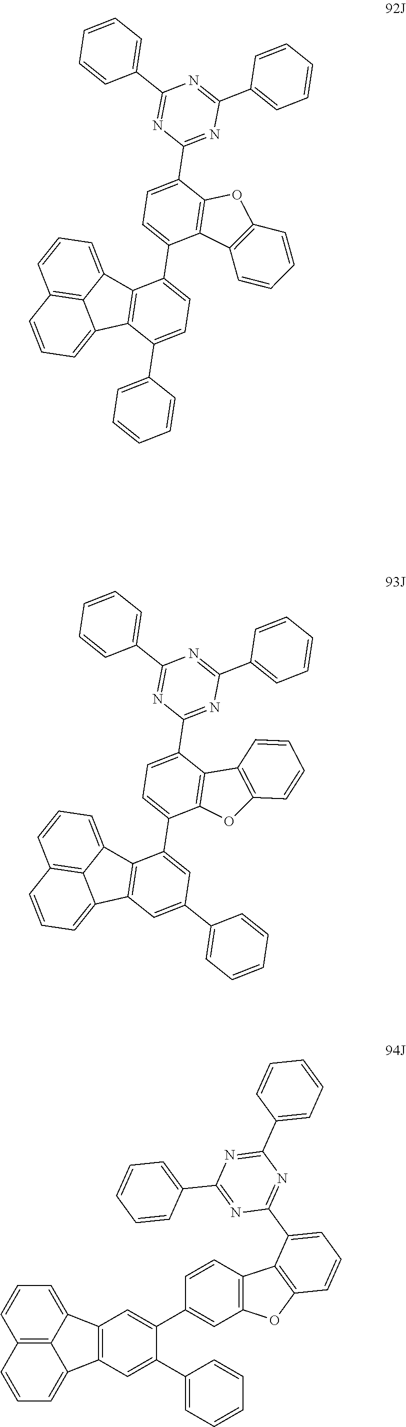 [SMILES: c1ccc(-c2nc(-c3ccccc3)nc(-c3ccc(-c4ccc(-c5ccccc5)c5c4-c4cccc6cccc-5c46)c4c3oc3ccccc34)n2)cc1 ; c1ccc(-c2cc(-c3ccc(-c4nc(-c5ccccc5)nc(-c5ccccc5)n4)c4c3oc3ccccc34)c3c(c2)-c2cccc4cccc-3c24)cc1 ; c1ccc(-c2nc(-c3ccccc3)nc(-c3cccc4oc5cc(-c6cc7c(cc6-c6ccccc6)-c6cccc8cccc-7c68)ccc5c34)n2)cc1]

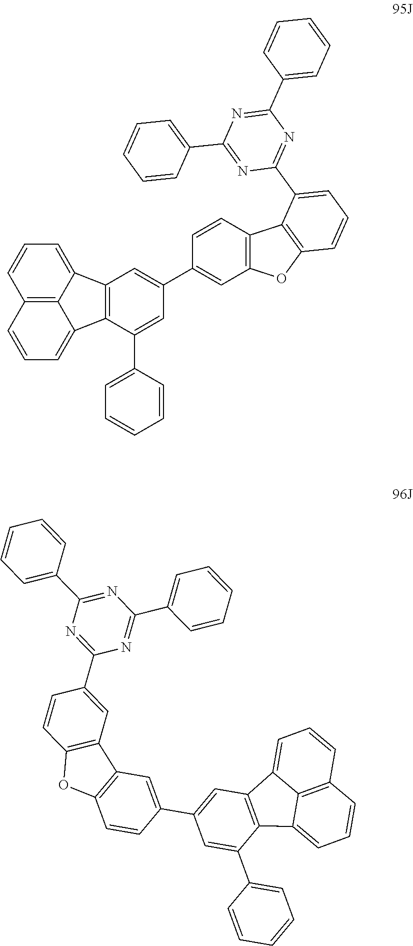 [SMILES: c1ccc(-c2nc(-c3ccccc3)nc(-c3cccc4oc5cc(-c6cc(-c7ccccc7)c7c(c6)-c6cccc8cccc-7c68)ccc5c34)n2)cc1 ; c1ccc(-c2nc(-c3ccccc3)nc(-c3ccc4oc5ccc(-c6cc(-c7ccccc7)c7c(c6)-c6cccc8cccc-7c68)cc5c4c3)n2)cc1]

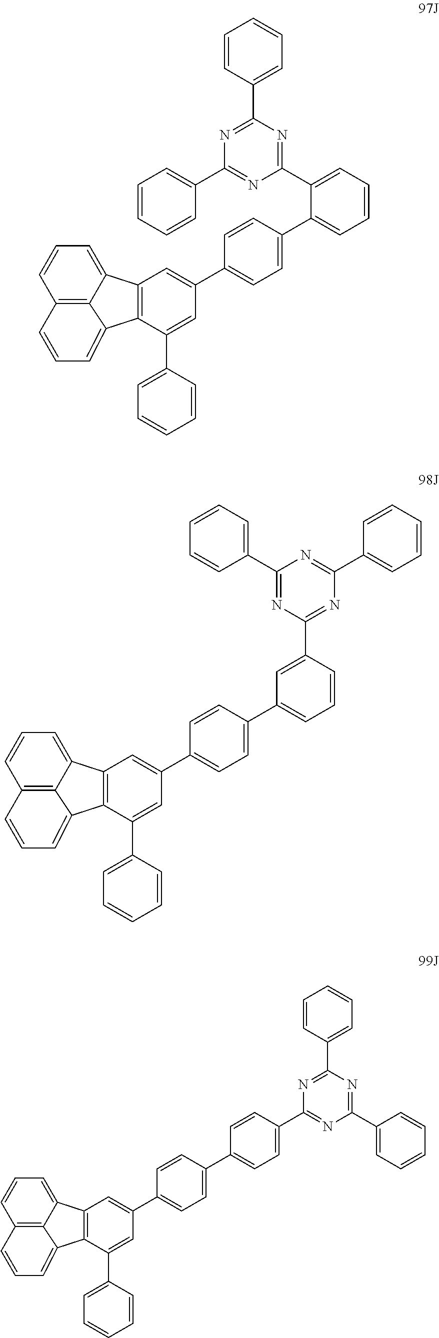 [SMILES: c1ccc(-c2nc(-c3ccccc3)nc(-c3ccccc3-c3ccc(-c4cc(-c5ccccc5)c5c(c4)-c4cccc6cccc-5c46)cc3)n2)cc1 ; c1ccc(-c2nc(-c3ccccc3)nc(-c3cccc(-c4ccc(-c5cc(-c6ccccc6)c6c(c5)-c5cccc7cccc-6c57)cc4)c3)n2)cc1 ; c1ccc(-c2nc(-c3ccccc3)nc(-c3ccc(-c4ccc(-c5cc(-c6ccccc6)c6c(c5)-c5cccc7cccc-6c57)cc4)cc3)n2)cc1]

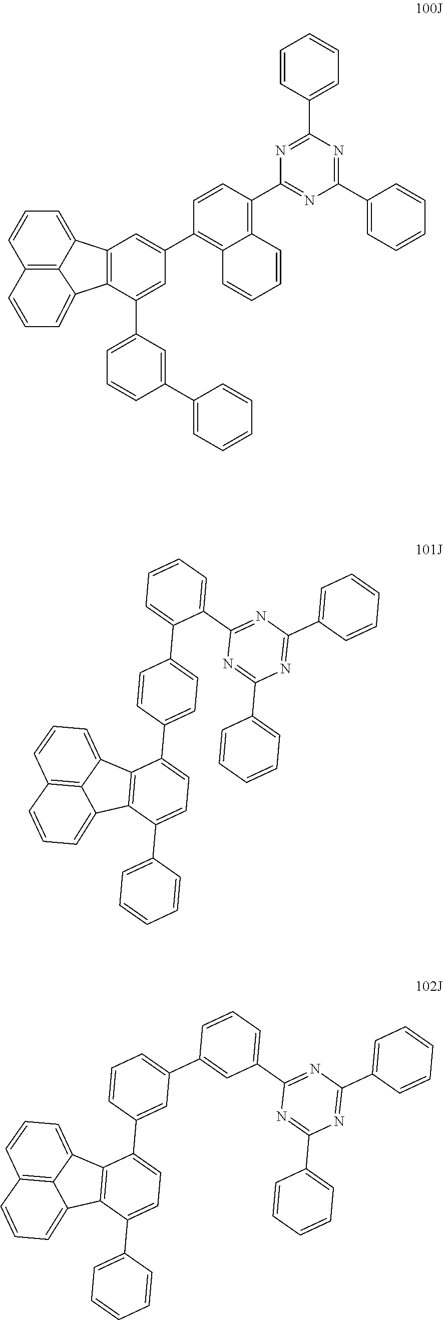 [SMILES: c1ccc(-c2cccc(-c3cc(-c4ccc(-c5nc(-c6ccccc6)nc(-c6ccccc6)n5)c5ccccc45)cc4c3-c3cccc5cccc-4c35)c2)cc1 ; c1ccc(-c2nc(-c3ccccc3)nc(-c3ccccc3-c3ccc(-c4ccc(-c5ccccc5)c5c4-c4cccc6cccc-5c46)cc3)n2)cc1 ; c1ccc(-c2nc(-c3ccccc3)nc(-c3cccc(-c4cccc(-c5ccc(-c6ccccc6)c6c5-c5cccc7cccc-6c57)c4)c3)n2)cc1]

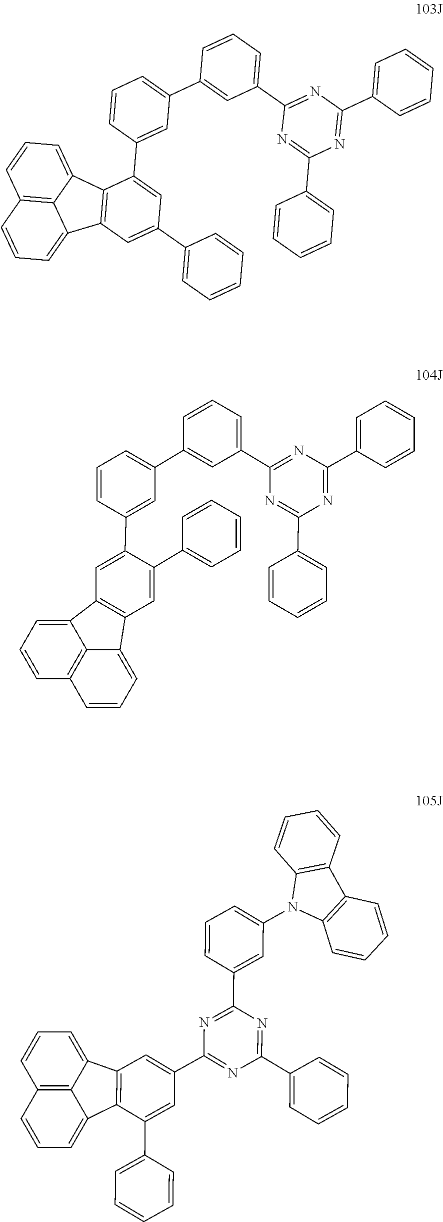 [SMILES: c1ccc(-c2cc(-c3cccc(-c4cccc(-c5nc(-c6ccccc6)nc(-c6ccccc6)n5)c4)c3)c3c(c2)-c2cccc4cccc-3c24)cc1 ; c1ccc(-c2nc(-c3ccccc3)nc(-c3cccc(-c4cccc(-c5cc6c(cc5-c5ccccc5)-c5cccc7cccc-6c57)c4)c3)n2)cc1 ; c1ccc(-c2nc(-c3cccc(-n4c5ccccc5c5ccccc54)c3)nc(-c3cc(-c4ccccc4)c4c(c3)-c3cccc5cccc-4c35)n2)cc1]

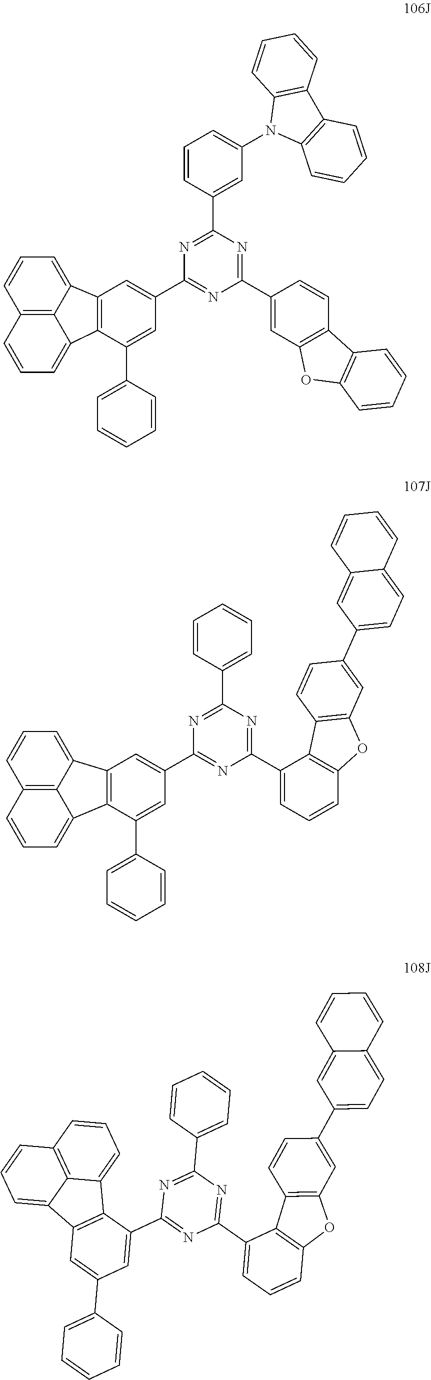 [SMILES: c1ccc(-c2cc(-c3nc(-c4cccc(-n5c6ccccc6c6ccccc65)c4)nc(-c4ccc5c(c4)oc4ccccc45)n3)cc3c2-c2cccc4cccc-3c24)cc1 ; c1ccc(-c2nc(-c3cc(-c4ccccc4)c4c(c3)-c3cccc5cccc-4c35)nc(-c3cccc4oc5cc(-c6ccc7ccccc7c6)ccc5c34)n2)cc1 ; c1ccc(-c2cc(-c3nc(-c4ccccc4)nc(-c4cccc5oc6cc(-c7ccc8ccccc8c7)ccc6c45)n3)c3c(c2)-c2cccc4cccc-3c24)cc1]

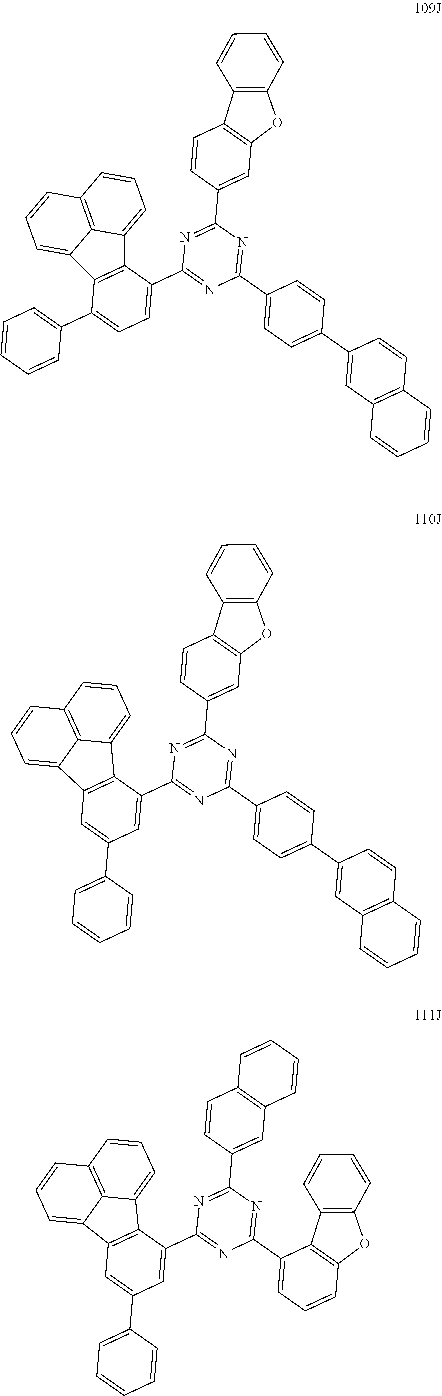 [SMILES: c1ccc(-c2ccc(-c3nc(-c4ccc(-c5ccc6ccccc6c5)cc4)nc(-c4ccc5c(c4)oc4ccccc45)n3)c3c2-c2cccc4cccc-3c24)cc1 ; c1ccc(-c2cc(-c3nc(-c4ccc(-c5ccc6ccccc6c5)cc4)nc(-c4ccc5c(c4)oc4ccccc45)n3)c3c(c2)-c2cccc4cccc-3c24)cc1 ; c1ccc(-c2cc(-c3nc(-c4ccc5ccccc5c4)nc(-c4cccc5oc6ccccc6c45)n3)c3c(c2)-c2cccc4cccc-3c24)cc1]

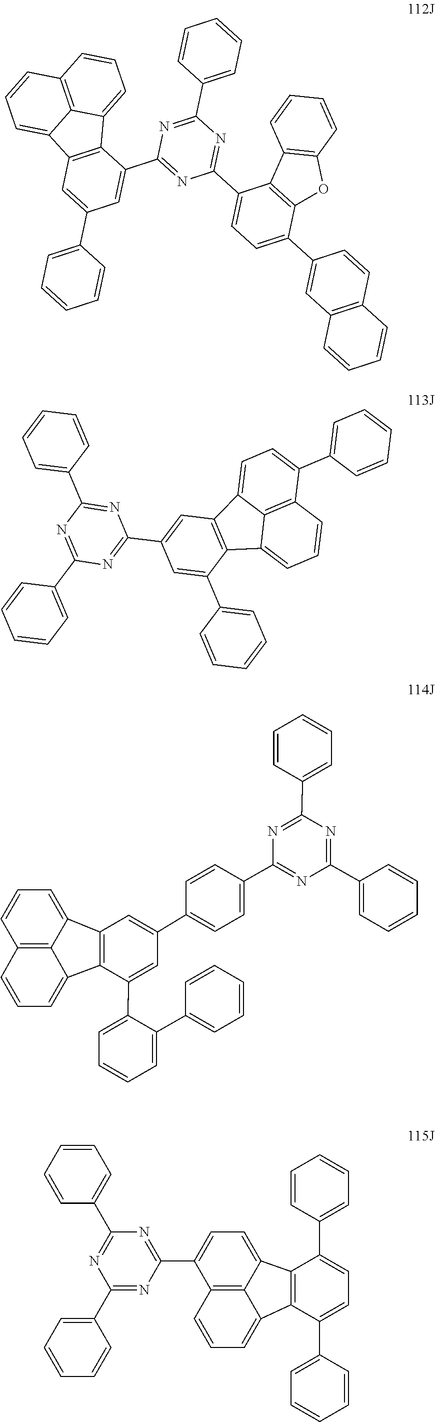

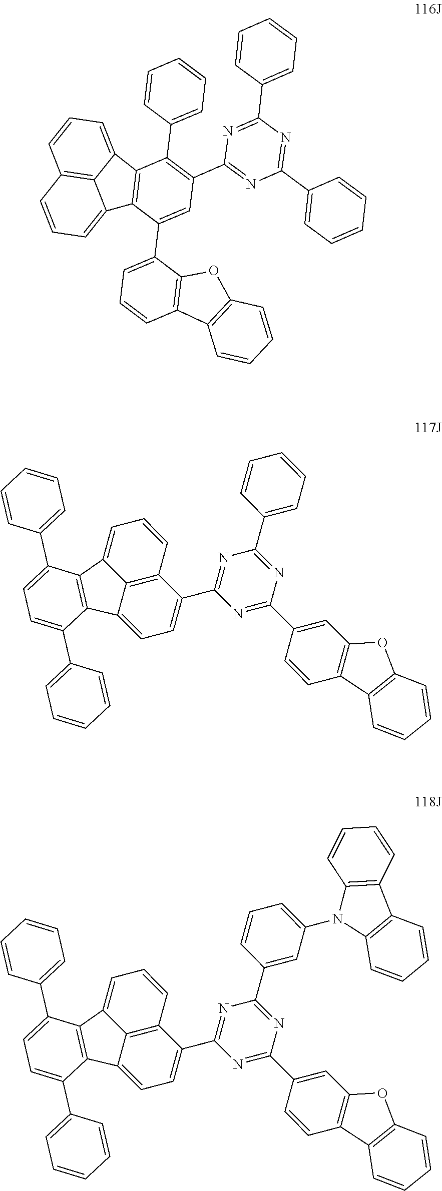 [SMILES: c1ccc(-c2nc(-c3ccccc3)nc(-c3cc(-c4cccc5c4oc4ccccc45)c4c(c3-c3ccccc3)-c3cccc5cccc-4c35)n2)cc1 ; c1ccc(-c2nc(-c3ccc4c(c3)oc3ccccc34)nc(-c3ccc4c5c(cccc35)-c3c(-c5ccccc5)ccc(-c5ccccc5)c3-4)n2)cc1 ; c1ccc(-c2ccc(-c3ccccc3)c3c2-c2cccc4c(-c5nc(-c6cccc(-n7c8ccccc8c8ccccc87)c6)nc(-c6ccc7c(c6)oc6ccccc67)n5)ccc-3c24)cc1]

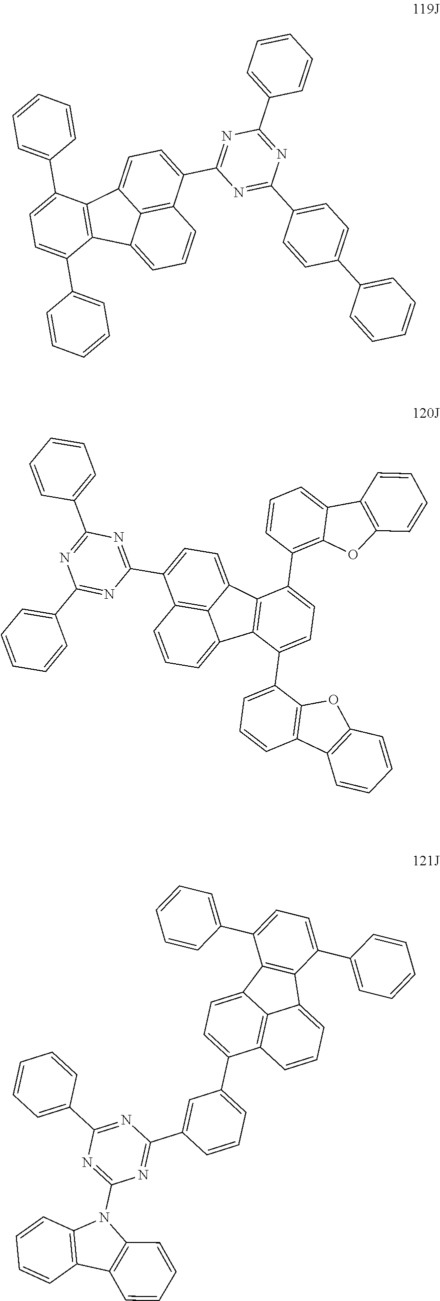 [SMILES: c1ccc(-c2ccc(-c3nc(-c4ccccc4)nc(-c4ccc5c6c(cccc46)-c4c(-c6ccccc6)ccc(-c6ccccc6)c4-5)n3)cc2)cc1 ; c1ccc(-c2nc(-c3ccccc3)nc(-c3ccc4c5c(cccc35)-c3c(-c5cccc6c5oc5ccccc56)ccc(-c5cccc6c5oc5ccccc56)c3-4)n2)cc1 ; c1ccc(-c2nc(-c3cccc(-c4ccc5c6c(cccc46)-c4c(-c6ccccc6)ccc(-c6ccccc6)c4-5)c3)nc(-n3c4ccccc4c4ccccc43)n2)cc1]

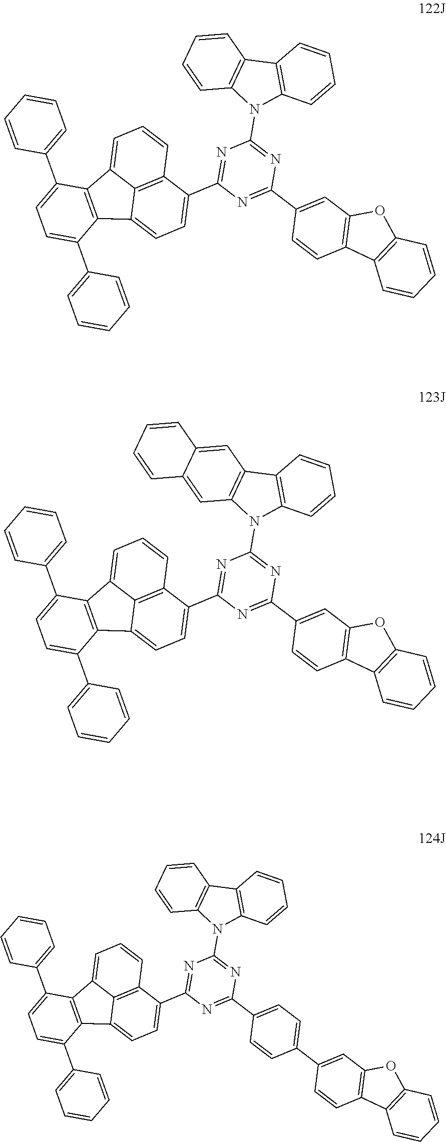 [SMILES: c1ccc(-c2ccc(-c3ccccc3)c3c2-c2cccc4c(-c5nc(-c6ccc7c(c6)oc6ccccc67)nc(-n6c7ccccc7c7ccccc76)n5)ccc-3c24)cc1 ; c1ccc(-c2ccc(-c3ccccc3)c3c2-c2cccc4c(-c5nc(-c6ccc7c(c6)oc6ccccc67)nc(-n6c7ccccc7c7cc8ccccc8cc76)n5)ccc-3c24)cc1 ; c1ccc(-c2ccc(-c3ccccc3)c3c2-c2cccc4c(-c5nc(-c6ccc(-c7ccc8c(c7)oc7ccccc78)cc6)nc(-n6c7ccccc7c7ccccc76)n5)ccc-3c24)cc1]

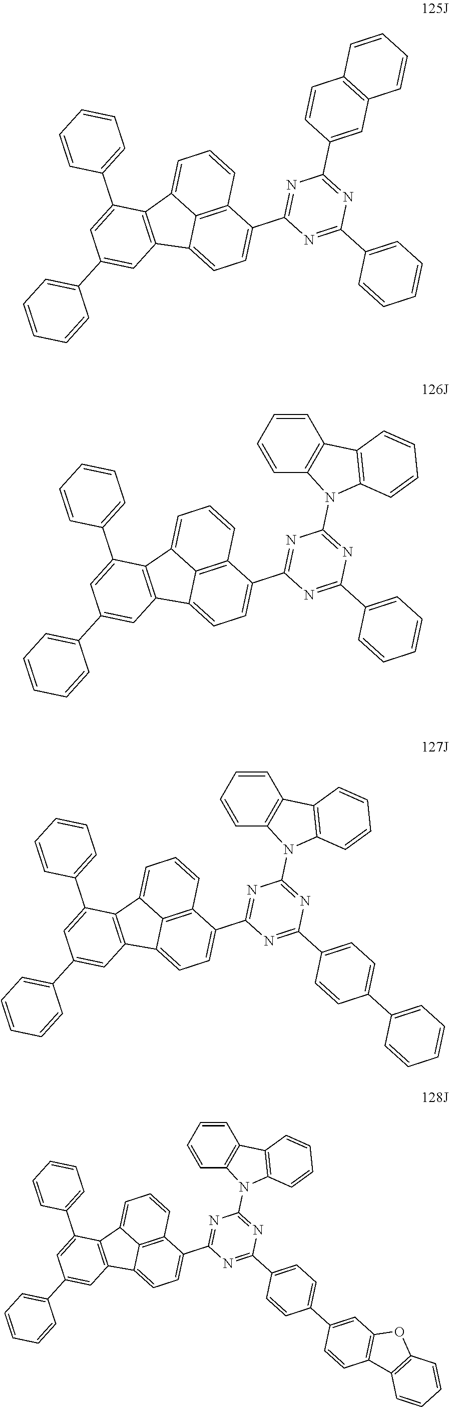 [SMILES: c1ccc(-c2cc(-c3ccccc3)c3c(c2)-c2ccc(-c4nc(-c5ccccc5)nc(-c5ccc6ccccc6c5)n4)c4cccc-3c24)cc1 ; c1ccc(-c2cc(-c3ccccc3)c3c(c2)-c2ccc(-c4nc(-c5ccccc5)nc(-n5c6ccccc6c6ccccc65)n4)c4cccc-3c24)cc1 ; c1ccc(-c2ccc(-c3nc(-c4ccc5c6c(cccc46)-c4c(-c6ccccc6)cc(-c6ccccc6)cc4-5)nc(-n4c5ccccc5c5ccccc54)n3)cc2)cc1 ; c1ccc(-c2cc(-c3ccccc3)c3c(c2)-c2ccc(-c4nc(-c5ccc(-c6ccc7c(c6)oc6ccccc67)cc5)nc(-n5c6ccccc6c6ccccc65)n4)c4cccc-3c24)cc1]

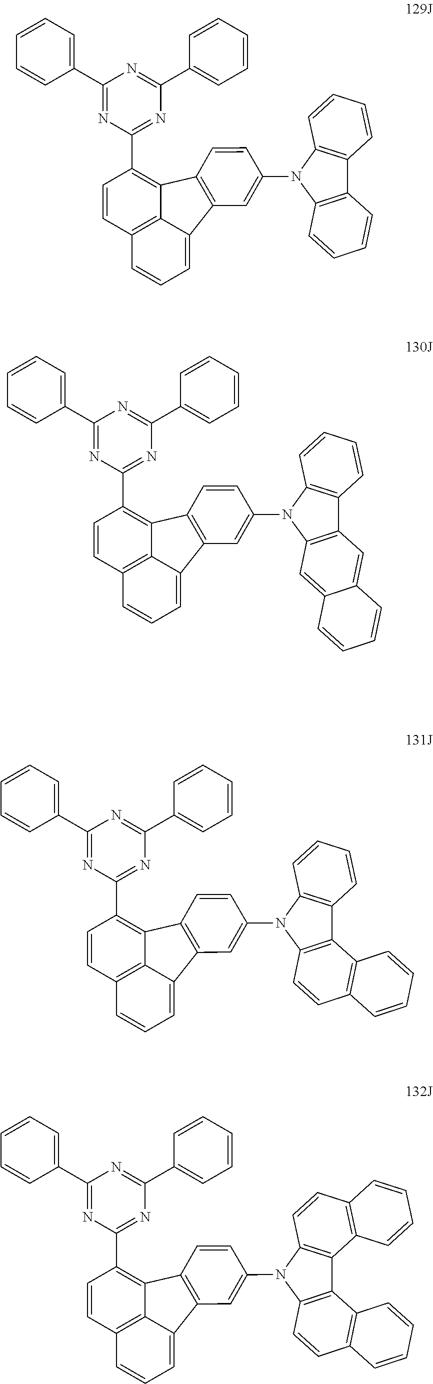 [SMILES: c1ccc(-c2nc(-c3ccccc3)nc(-c3ccc4cccc5c4c3-c3ccc(-n4c6ccccc6c6ccccc64)cc3-5)n2)cc1 ; c1ccc(-c2nc(-c3ccccc3)nc(-c3ccc4cccc5c4c3-c3ccc(-n4c6ccccc6c6cc7ccccc7cc64)cc3-5)n2)cc1 ; c1ccc(-c2nc(-c3ccccc3)nc(-c3ccc4cccc5c4c3-c3ccc(-n4c6ccccc6c6c7ccccc7ccc64)cc3-5)n2)cc1 ; c1ccc(-c2nc(-c3ccccc3)nc(-c3ccc4cccc5c4c3-c3ccc(-n4c6ccc7ccccc7c6c6c7ccccc7ccc64)cc3-5)n2)cc1]

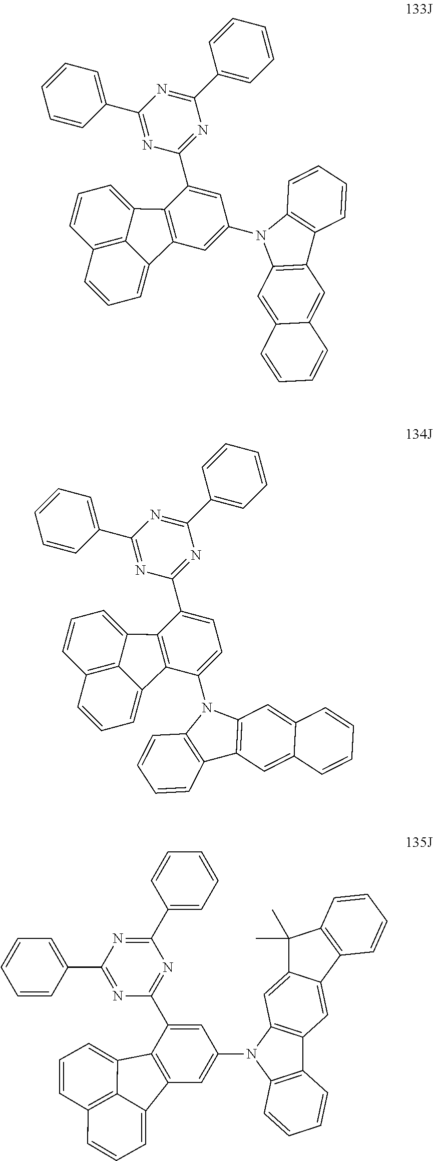 [SMILES: c1ccc(-c2nc(-c3ccccc3)nc(-c3cc(-n4c5ccccc5c5cc6ccccc6cc54)cc4c3-c3cccc5cccc-4c35)n2)cc1 ; c1ccc(-c2nc(-c3ccccc3)nc(-c3ccc(-n4c5ccccc5c5cc6ccccc6cc54)c4c3-c3cccc5cccc-4c35)n2)cc1 ; CC1(C)c2ccccc2-c2cc3c4ccccc4n(-c4cc(-c5nc(-c6ccccc6)nc(-c6ccccc6)n5)c5c(c4)-c4cccc6cccc-5c46)c3cc21]

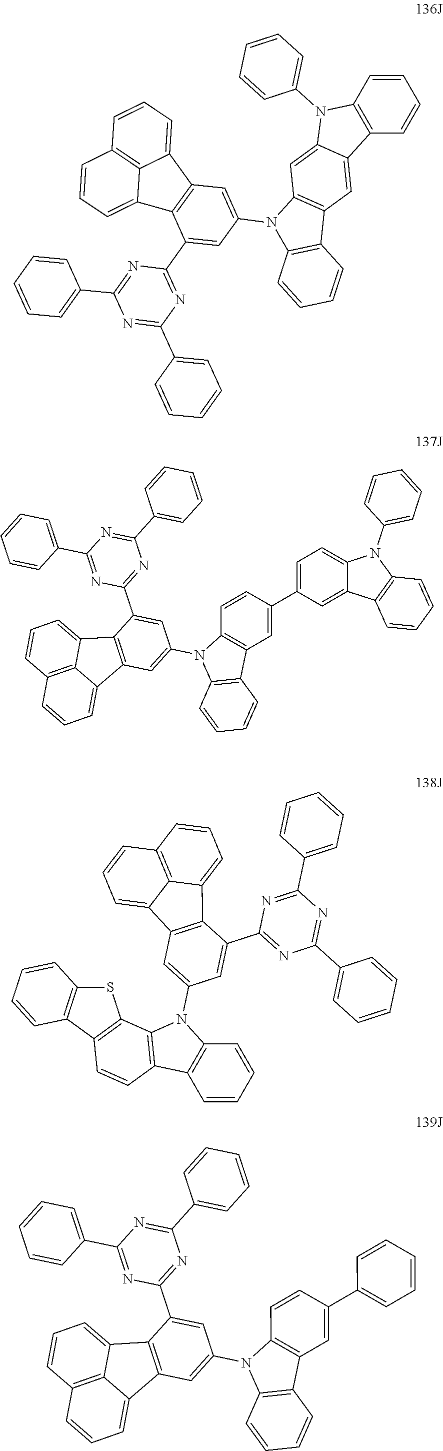

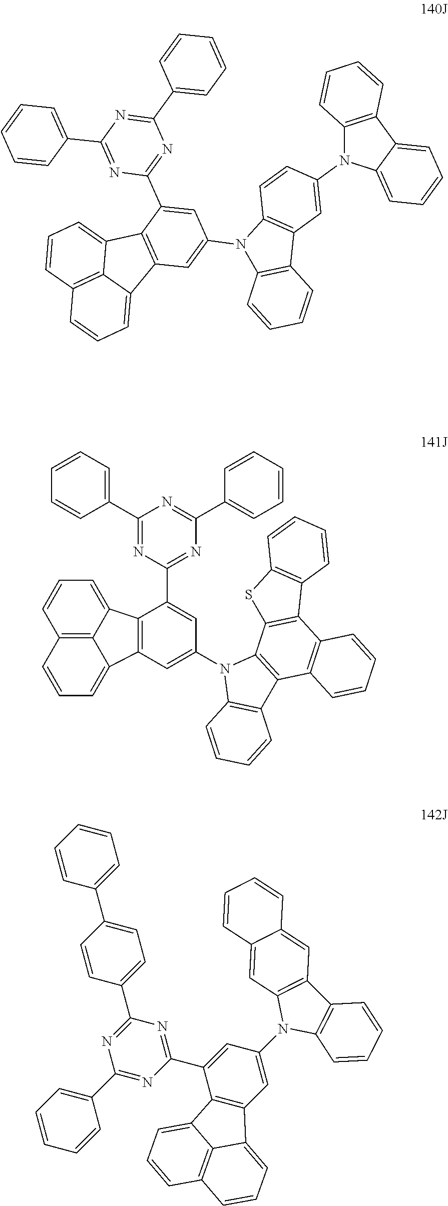 [SMILES: c1ccc(-c2nc(-c3ccccc3)nc(-c3cc(-n4c5ccccc5c5cc(-n6c7ccccc7c7ccccc76)ccc54)cc4c3-c3cccc5cccc-4c35)n2)cc1 ; c1ccc(-c2nc(-c3ccccc3)nc(-c3cc(-n4c5ccccc5c5c6ccccc6c6c7ccccc7sc6c54)cc4c3-c3cccc5cccc-4c35)n2)cc1 ; c1ccc(-c2ccc(-c3nc(-c4ccccc4)nc(-c4cc(-n5c6ccccc6c6cc7ccccc7cc65)cc5c4-c4cccc6cccc-5c46)n3)cc2)cc1]

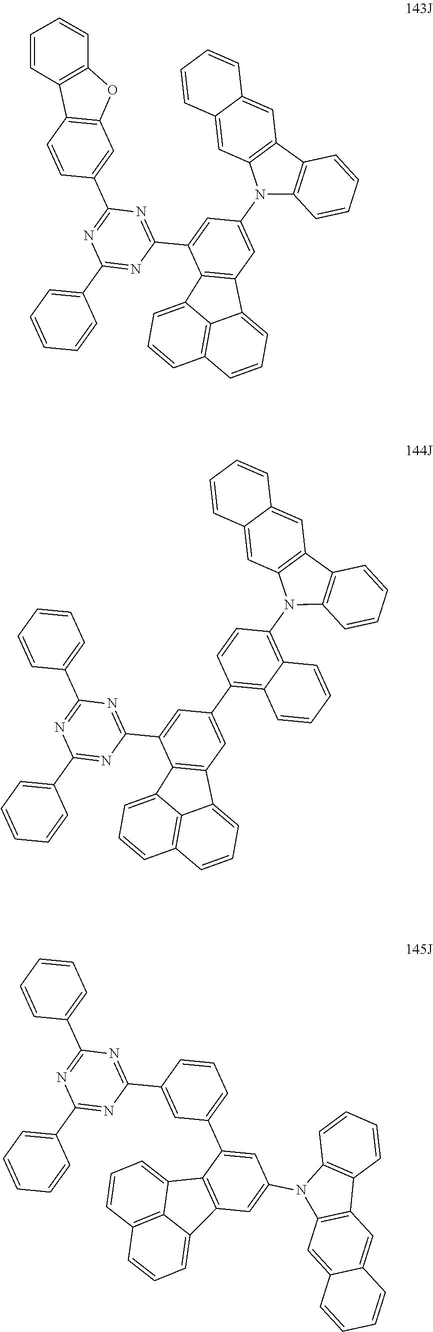 [SMILES: c1ccc(-c2nc(-c3ccc4c(c3)oc3ccccc34)nc(-c3cc(-n4c5ccccc5c5cc6ccccc6cc54)cc4c3-c3cccc5cccc-4c35)n2)cc1 ; c1ccc(-c2nc(-c3ccccc3)nc(-c3cc(-c4ccc(-n5c6ccccc6c6cc7ccccc7cc65)c5ccccc45)cc4c3-c3cccc5cccc-4c35)n2)cc1 ; c1ccc(-c2nc(-c3ccccc3)nc(-c3cccc(-c4cc(-n5c6ccccc6c6cc7ccccc7cc65)cc5c4-c4cccc6cccc-5c46)c3)n2)cc1]

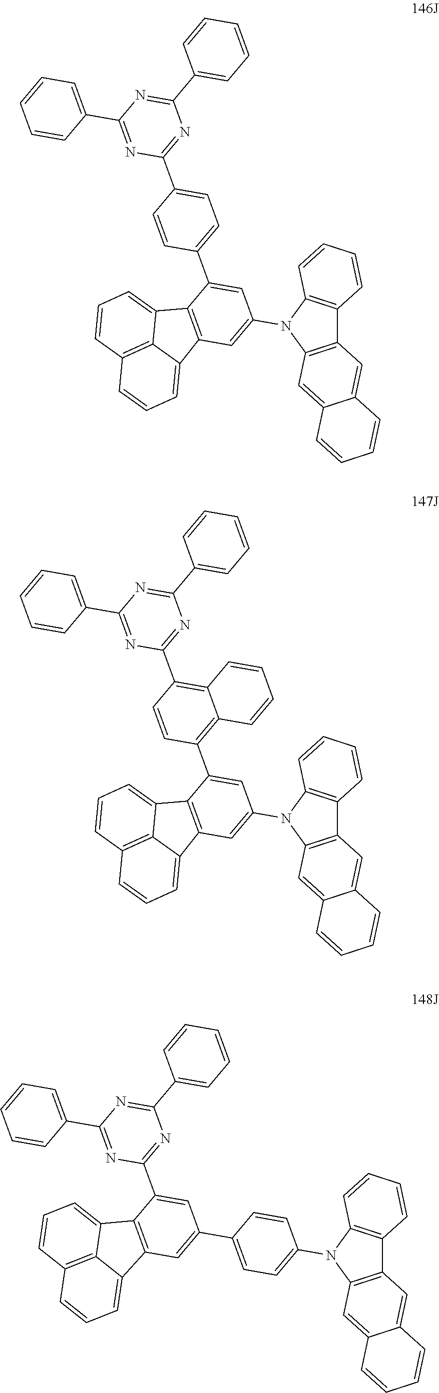 [SMILES: c1ccc(-c2nc(-c3ccccc3)nc(-c3ccc(-c4cc(-n5c6ccccc6c6cc7ccccc7cc65)cc5c4-c4cccc6cccc-5c46)cc3)n2)cc1 ; c1ccc(-c2nc(-c3ccccc3)nc(-c3ccc(-c4cc(-n5c6ccccc6c6cc7ccccc7cc65)cc5c4-c4cccc6cccc-5c46)c4ccccc34)n2)cc1 ; c1ccc(-c2nc(-c3ccccc3)nc(-c3cc(-c4ccc(-n5c6ccccc6c6cc7ccccc7cc65)cc4)cc4c3-c3cccc5cccc-4c35)n2)cc1]

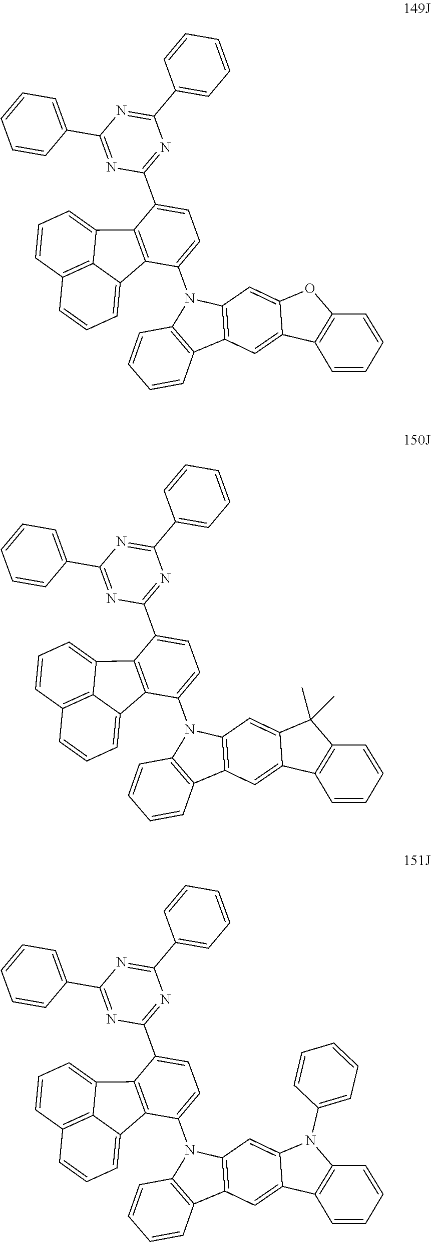 [SMILES: c1ccc(-c2nc(-c3ccccc3)nc(-c3ccc(-n4c5ccccc5c5cc6c(cc54)oc4ccccc46)c4c3-c3cccc5cccc-4c35)n2)cc1 ; CC1(C)c2ccccc2-c2cc3c4ccccc4n(-c4ccc(-c5nc(-c6ccccc6)nc(-c6ccccc6)n5)c5c4-c4cccc6cccc-5c46)c3cc21 ; c1ccc(-c2nc(-c3ccccc3)nc(-c3ccc(-n4c5ccccc5c5cc6c7ccccc7n(-c7ccccc7)c6cc54)c4c3-c3cccc5cccc-4c35)n2)cc1]

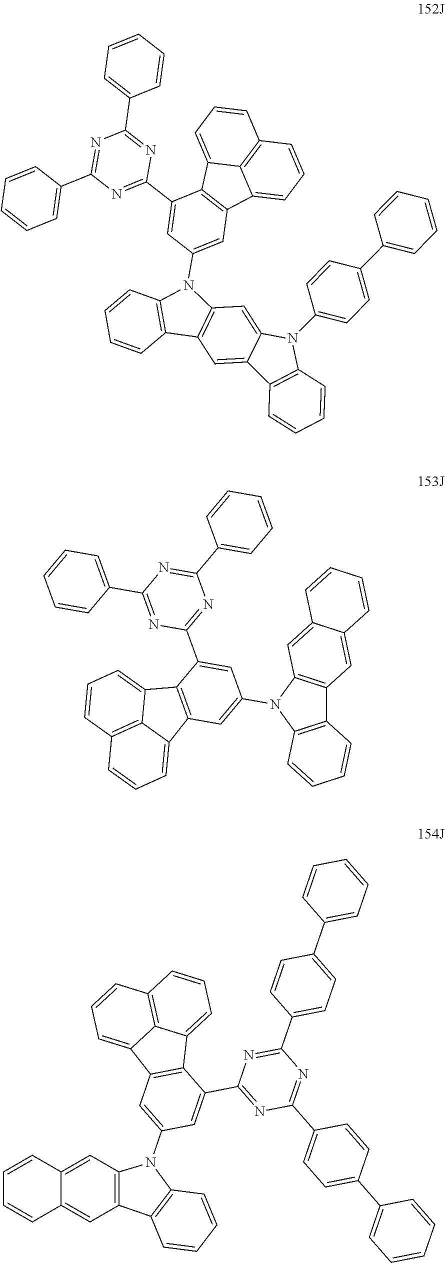 [SMILES: c1ccc(-c2ccc(-n3c4ccccc4c4cc5c6ccccc6n(-c6cc(-c7nc(-c8ccccc8)nc(-c8ccccc8)n7)c7c(c6)-c6cccc8cccc-7c68)c5cc43)cc2)cc1 ; c1ccc(-c2nc(-c3ccccc3)nc(-c3cc(-n4c5ccccc5c5cc6ccccc6cc54)cc4c3-c3cccc5cccc-4c35)n2)cc1 ; c1ccc(-c2ccc(-c3nc(-c4ccc(-c5ccccc5)cc4)nc(-c4cc(-n5c6ccccc6c6cc7ccccc7cc65)cc5c4-c4cccc6cccc-5c46)n3)cc2)cc1]

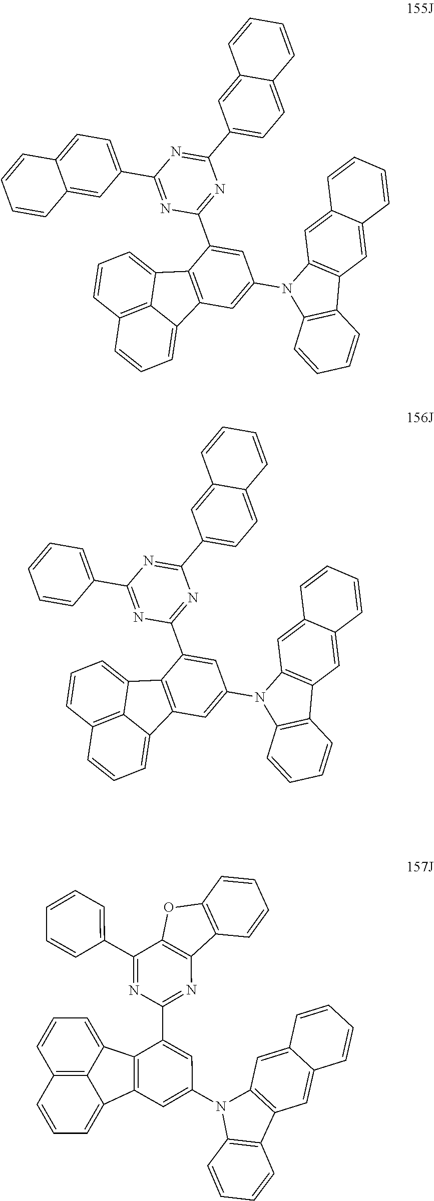 [SMILES: c1ccc2cc(-c3nc(-c4ccc5ccccc5c4)nc(-c4cc(-n5c6ccccc6c6cc7ccccc7cc65)cc5c4-c4cccc6cccc-5c46)n3)ccc2c1 ; c1ccc(-c2nc(-c3ccc4ccccc4c3)nc(-c3cc(-n4c5ccccc5c5cc6ccccc6cc54)cc4c3-c3cccc5cccc-4c35)n2)cc1 ; c1ccc(-c2nc(-c3cc(-n4c5ccccc5c5cc6ccccc6cc54)cc4c3-c3cccc5cccc-4c35)nc3c2oc2ccccc23)cc1]

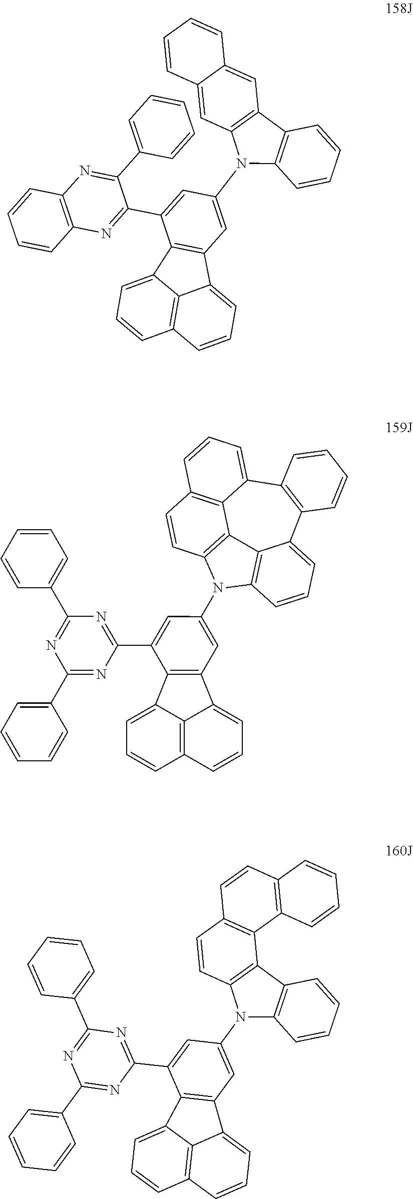 [SMILES: c1ccc(-c2nc3ccccc3nc2-c2cc(-n3c4ccccc4c4cc5ccccc5cc43)cc3c2-c2cccc4cccc-3c24)cc1 ; c1ccc(-c2nc(-c3ccccc3)nc(-c3cc(-n4c5cccc6c5c5c7c(cccc7ccc54)-c4ccccc4-6)cc4c3-c3cccc5cccc-4c35)n2)cc1 ; c1ccc(-c2nc(-c3ccccc3)nc(-c3cc(-n4c5ccccc5c5c6c(ccc7ccccc76)ccc54)cc4c3-c3cccc5cccc-4c35)n2)cc1]

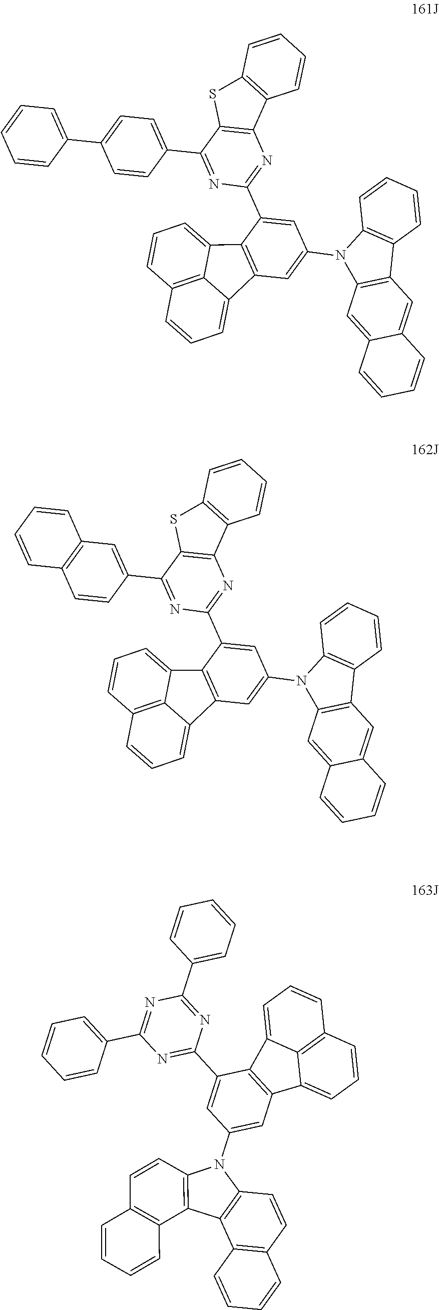 [SMILES: c1ccc(-c2ccc(-c3nc(-c4cc(-n5c6ccccc6c6cc7ccccc7cc65)cc5c4-c4cccc6cccc-5c46)nc4c3sc3ccccc34)cc2)cc1 ; c1ccc2cc(-c3nc(-c4cc(-n5c6ccccc6c6cc7ccccc7cc65)cc5c4-c4cccc6cccc-5c46)nc4c3sc3ccccc34)ccc2c1 ; c1ccc(-c2nc(-c3ccccc3)nc(-c3cc(-n4c5ccc6ccccc6c5c5c6ccccc6ccc54)cc4c3-c3cccc5cccc-4c35)n2)cc1]

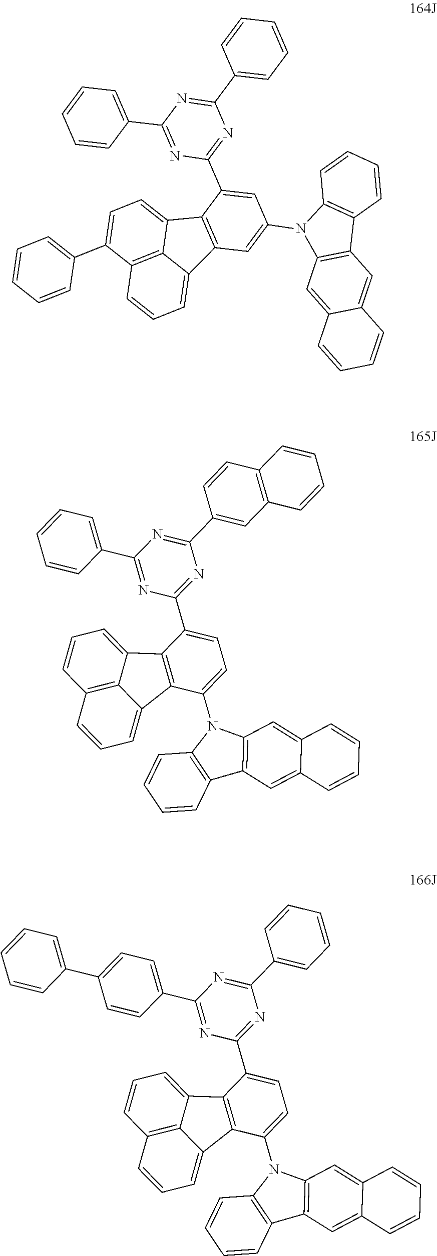 [SMILES: c1ccc(-c2nc(-c3ccccc3)nc(-c3cc(-n4c5ccccc5c5cc6ccccc6cc54)cc4c3-c3ccc(-c5ccccc5)c5cccc-4c35)n2)cc1 ; c1ccc(-c2nc(-c3ccc4ccccc4c3)nc(-c3ccc(-n4c5ccccc5c5cc6ccccc6cc54)c4c3-c3cccc5cccc-4c35)n2)cc1 ; c1ccc(-c2ccc(-c3nc(-c4ccccc4)nc(-c4ccc(-n5c6ccccc6c6cc7ccccc7cc65)c5c4-c4cccc6cccc-5c46)n3)cc2)cc1]

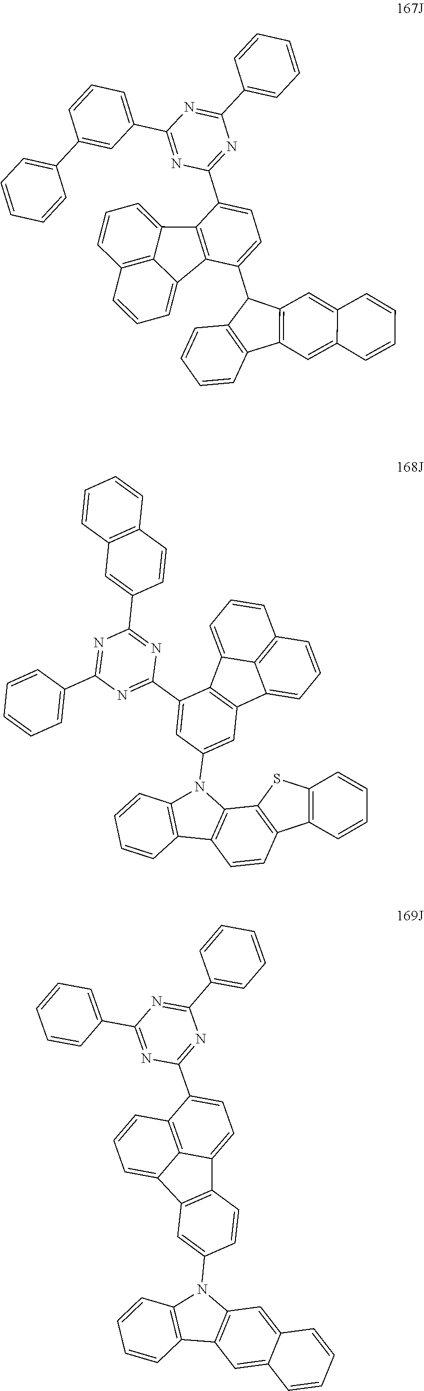 [SMILES: c1ccc(-c2cccc(-c3nc(-c4ccccc4)nc(-c4ccc(C5c6ccccc6-c6cc7ccccc7cc65)c5c4-c4cccc6cccc-5c46)n3)c2)cc1 ; c1ccc(-c2nc(-c3ccc4ccccc4c3)nc(-c3cc(-n4c5ccccc5c5ccc6c7ccccc7sc6c54)cc4c3-c3cccc5cccc-4c35)n2)cc1 ; c1ccc(-c2nc(-c3ccccc3)nc(-c3ccc4c5c(cccc35)-c3cc(-n5c6ccccc6c6cc7ccccc7cc65)ccc3-4)n2)cc1]

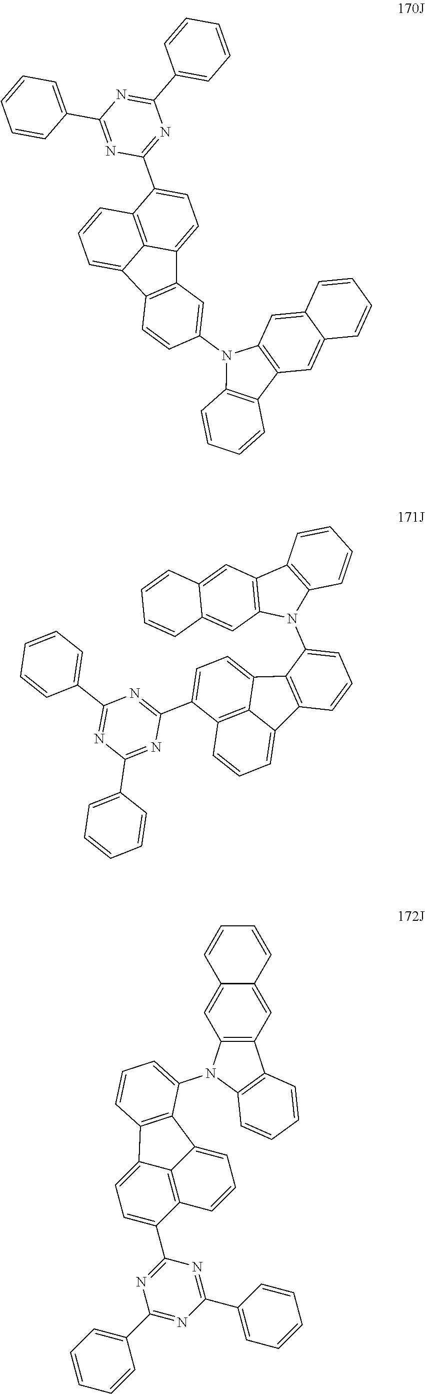 [SMILES: c1ccc(-c2nc(-c3ccccc3)nc(-c3ccc4c5c(cccc35)-c3ccc(-n5c6ccccc6c6cc7ccccc7cc65)cc3-4)n2)cc1 ; c1ccc(-c2nc(-c3ccccc3)nc(-c3ccc4c5c(cccc35)-c3cccc(-n5c6ccccc6c6cc7ccccc7cc65)c3-4)n2)cc1 ; c1ccc(-c2nc(-c3ccccc3)nc(-c3ccc4c5c(cccc35)-c3c-4cccc3-n3c4ccccc4c4cc5ccccc5cc43)n2)cc1]

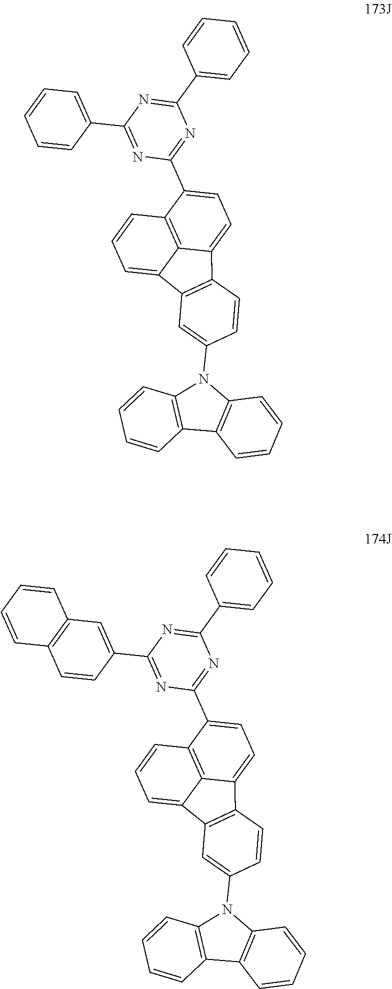 [SMILES: c1ccc(-c2nc(-c3ccccc3)nc(-c3ccc4c5c(cccc35)-c3cc(-n5c6ccccc6c6ccccc65)ccc3-4)n2)cc1 ; c1ccc(-c2nc(-c3ccc4ccccc4c3)nc(-c3ccc4c5c(cccc35)-c3cc(-n5c6ccccc6c6ccccc65)ccc3-4)n2)cc1]

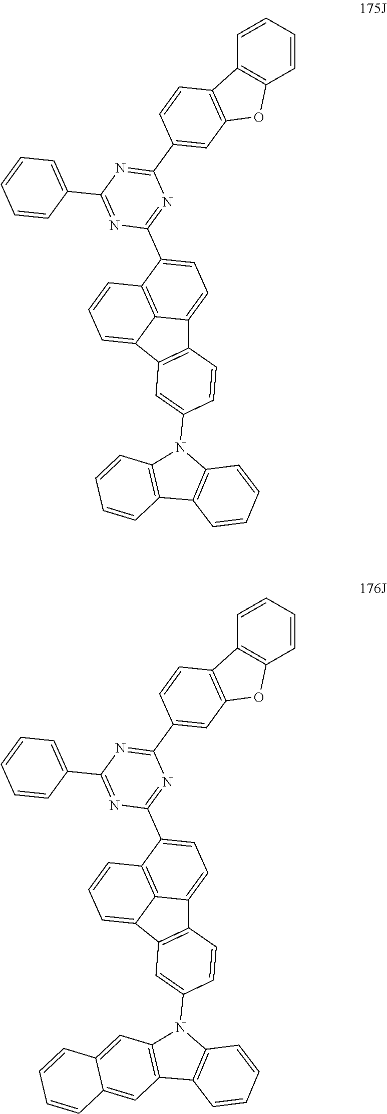 [SMILES: c1ccc(-c2nc(-c3ccc4c(c3)oc3ccccc34)nc(-c3ccc4c5c(cccc35)-c3cc(-n5c6ccccc6c6ccccc65)ccc3-4)n2)cc1 ; c1ccc(-c2nc(-c3ccc4c(c3)oc3ccccc34)nc(-c3ccc4c5c(cccc35)-c3cc(-n5c6ccccc6c6cc7ccccc7cc65)ccc3-4)n2)cc1]

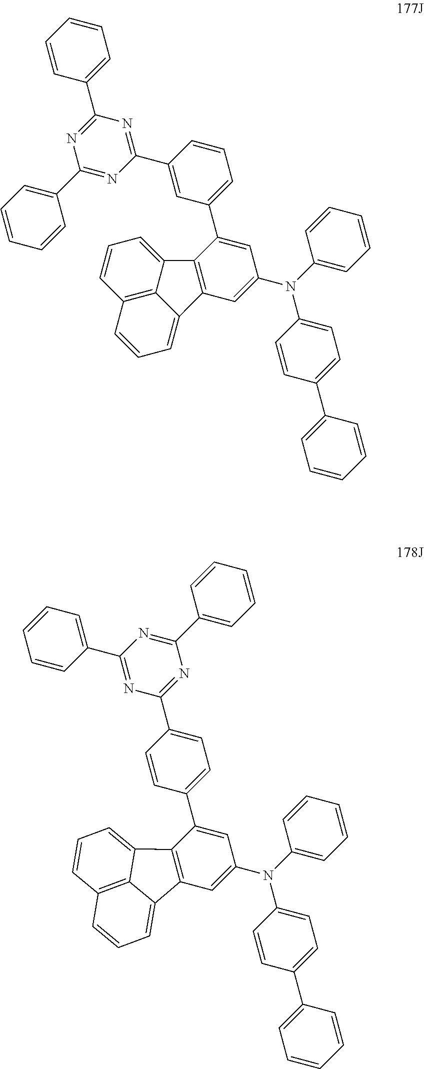 [SMILES: c1ccc(-c2ccc(N(c3ccccc3)c3cc(-c4cccc(-c5nc(-c6ccccc6)nc(-c6ccccc6)n5)c4)c4c(c3)-c3cccc5cccc-4c35)cc2)cc1 ; c1ccc(-c2ccc(N(c3ccccc3)c3cc(-c4ccc(-c5nc(-c6ccccc6)nc(-c6ccccc6)n5)cc4)c4c(c3)-c3cccc5cccc-4c35)cc2)cc1]

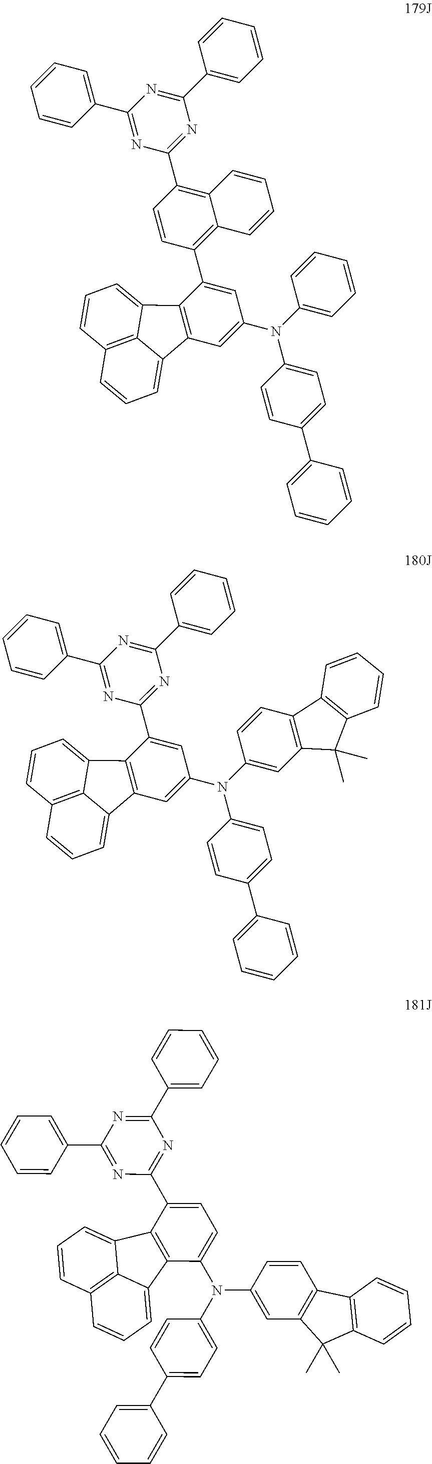 [SMILES: c1ccc(-c2ccc(N(c3ccccc3)c3cc(-c4ccc(-c5nc(-c6ccccc6)nc(-c6ccccc6)n5)c5ccccc45)c4c(c3)-c3cccc5cccc-4c35)cc2)cc1 ; CC1(C)c2ccccc2-c2ccc(N(c3ccc(-c4ccccc4)cc3)c3cc(-c4nc(-c5ccccc5)nc(-c5ccccc5)n4)c4c(c3)-c3cccc5cccc-4c35)cc21 ; CC1(C)c2ccccc2-c2ccc(N(c3ccc(-c4ccccc4)cc3)c3ccc(-c4nc(-c5ccccc5)nc(-c5ccccc5)n4)c4c3-c3cccc5cccc-4c35)cc21]

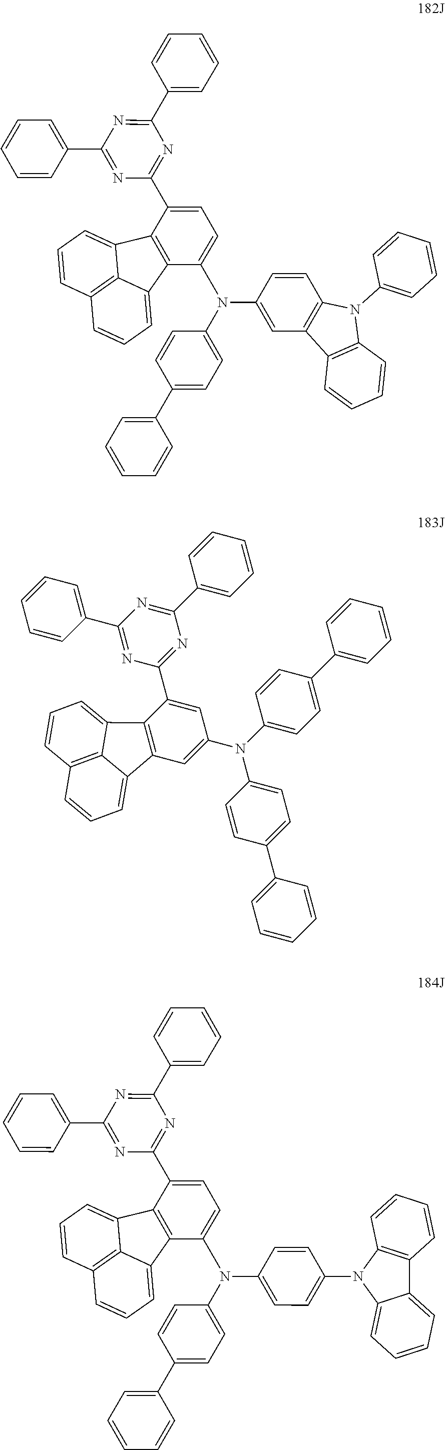 [SMILES: c1ccc(-c2ccc(N(c3ccc4c(c3)c3ccccc3n4-c3ccccc3)c3ccc(-c4nc(-c5ccccc5)nc(-c5ccccc5)n4)c4c3-c3cccc5cccc-4c35)cc2)cc1 ; c1ccc(-c2ccc(N(c3ccc(-c4ccccc4)cc3)c3cc(-c4nc(-c5ccccc5)nc(-c5ccccc5)n4)c4c(c3)-c3cccc5cccc-4c35)cc2)cc1 ; c1ccc(-c2ccc(N(c3ccc(-n4c5ccccc5c5ccccc54)cc3)c3ccc(-c4nc(-c5ccccc5)nc(-c5ccccc5)n4)c4c3-c3cccc5cccc-4c35)cc2)cc1]

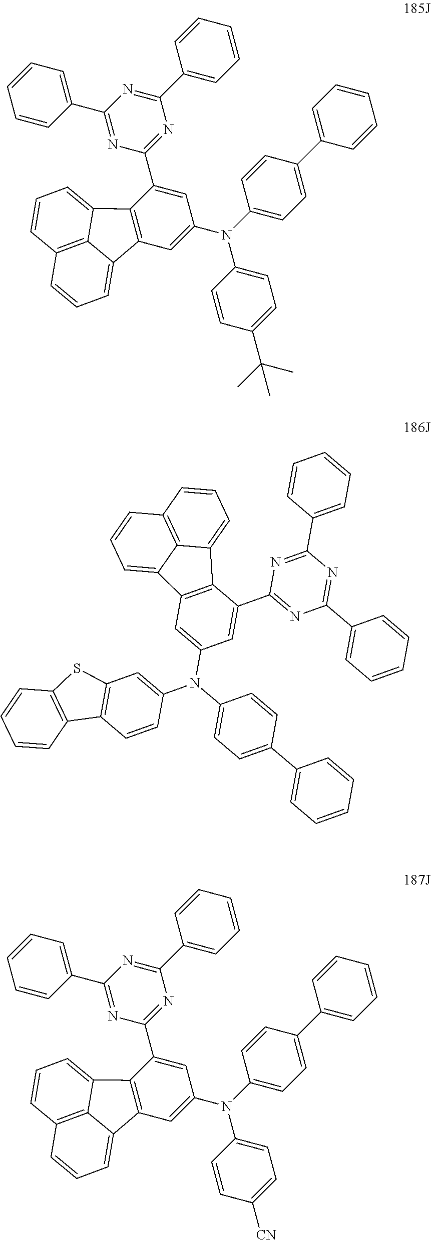 [SMILES: CC(C)(C)c1ccc(N(c2ccc(-c3ccccc3)cc2)c2cc(-c3nc(-c4ccccc4)nc(-c4ccccc4)n3)c3c(c2)-c2cccc4cccc-3c24)cc1 ; c1ccc(-c2ccc(N(c3cc(-c4nc(-c5ccccc5)nc(-c5ccccc5)n4)c4c(c3)-c3cccc5cccc-4c35)c3ccc4c(c3)sc3ccccc34)cc2)cc1 ; N#Cc1ccc(N(c2ccc(-c3ccccc3)cc2)c2cc(-c3nc(-c4ccccc4)nc(-c4ccccc4)n3)c3c(c2)-c2cccc4cccc-3c24)cc1]

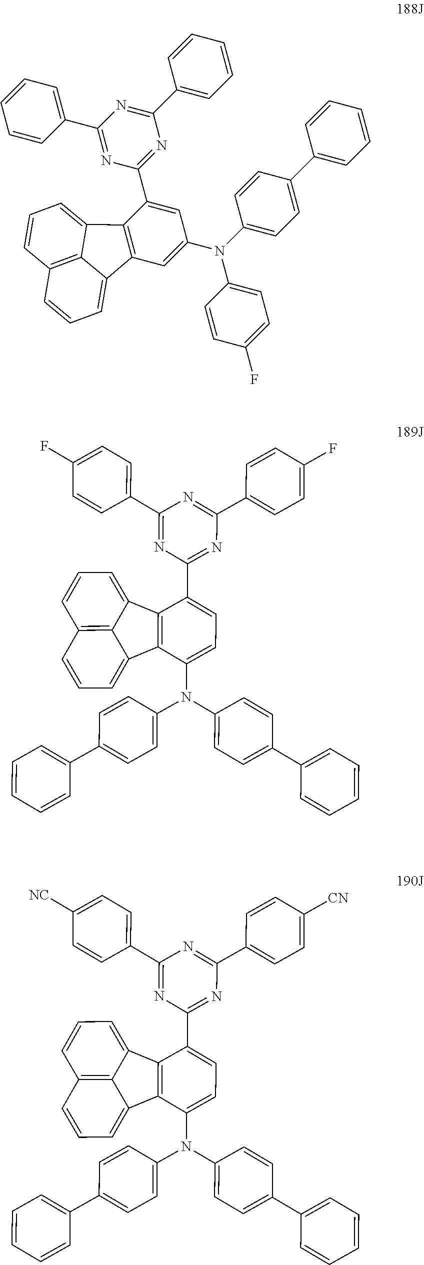 [SMILES: Fc1ccc(N(c2ccc(-c3ccccc3)cc2)c2cc(-c3nc(-c4ccccc4)nc(-c4ccccc4)n3)c3c(c2)-c2cccc4cccc-3c24)cc1 ; Fc1ccc(-c2nc(-c3ccc(F)cc3)nc(-c3ccc(N(c4ccc(-c5ccccc5)cc4)c4ccc(-c5ccccc5)cc4)c4c3-c3cccc5cccc-4c35)n2)cc1 ; N#Cc1ccc(-c2nc(-c3ccc(C#N)cc3)nc(-c3ccc(N(c4ccc(-c5ccccc5)cc4)c4ccc(-c5ccccc5)cc4)c4c3-c3cccc5cccc-4c35)n2)cc1]

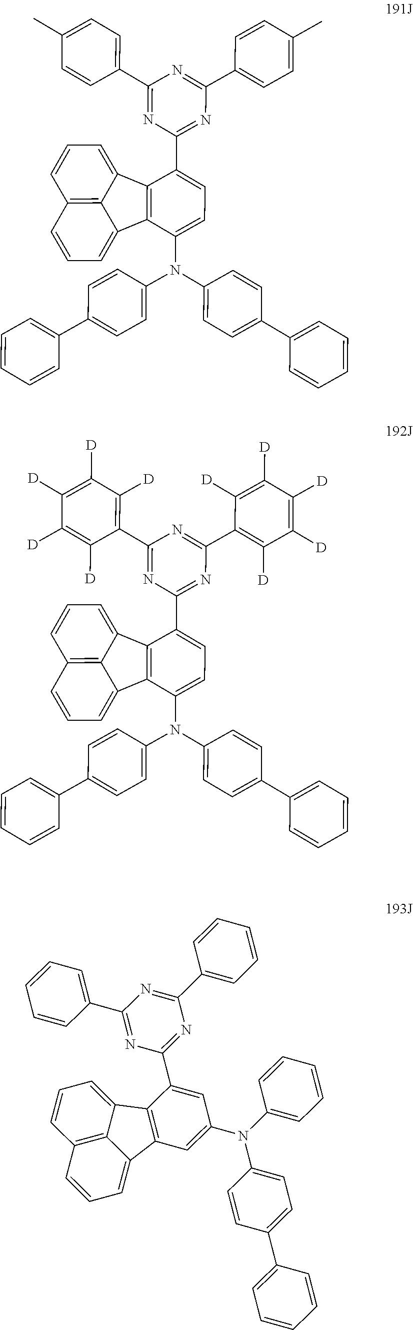 [SMILES: Cc1ccc(-c2nc(-c3ccc(C)cc3)nc(-c3ccc(N(c4ccc(-c5ccccc5)cc4)c4ccc(-c5ccccc5)cc4)c4c3-c3cccc5cccc-4c35)n2)cc1 ; [2H]c1c([2H])c([2H])c(-c2nc(-c3ccc(N(c4ccc(-c5ccccc5)cc4)c4ccc(-c5ccccc5)cc4)c4c3-c3cccc5cccc-4c35)nc(-c3c([2H])c([2H])c([2H])c([2H])c3[2H])n2)c([2H])c1[2H] ; c1ccc(-c2ccc(N(c3ccccc3)c3cc(-c4nc(-c5ccccc5)nc(-c5ccccc5)n4)c4c(c3)-c3cccc5cccc-4c35)cc2)cc1]

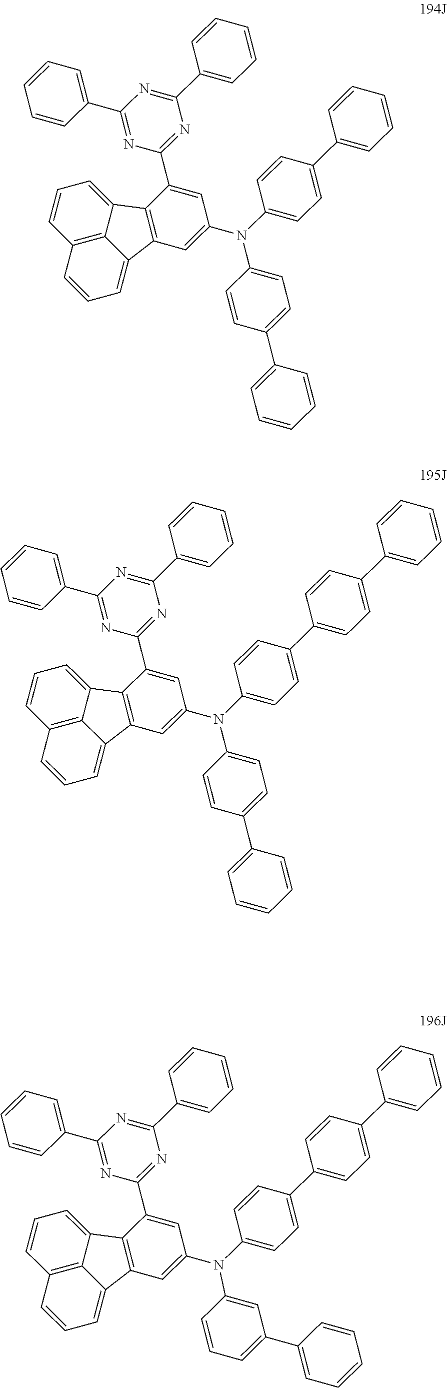 [SMILES: c1ccc(-c2ccc(N(c3ccc(-c4ccccc4)cc3)c3cc(-c4nc(-c5ccccc5)nc(-c5ccccc5)n4)c4c(c3)-c3cccc5cccc-4c35)cc2)cc1 ; c1ccc(-c2ccc(-c3ccc(N(c4ccc(-c5ccccc5)cc4)c4cc(-c5nc(-c6ccccc6)nc(-c6ccccc6)n5)c5c(c4)-c4cccc6cccc-5c46)cc3)cc2)cc1 ; c1ccc(-c2ccc(-c3ccc(N(c4cccc(-c5ccccc5)c4)c4cc(-c5nc(-c6ccccc6)nc(-c6ccccc6)n5)c5c(c4)-c4cccc6cccc-5c46)cc3)cc2)cc1]

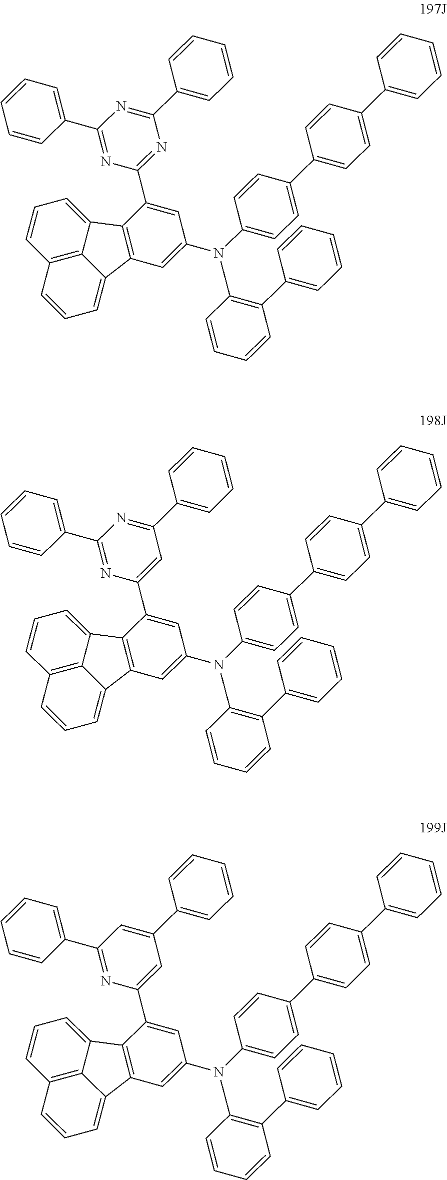 [SMILES: c1ccc(-c2ccc(-c3ccc(N(c4cc(-c5nc(-c6ccccc6)nc(-c6ccccc6)n5)c5c(c4)-c4cccc6cccc-5c46)c4ccccc4-c4ccccc4)cc3)cc2)cc1 ; c1ccc(-c2ccc(-c3ccc(N(c4cc(-c5cc(-c6ccccc6)nc(-c6ccccc6)n5)c5c(c4)-c4cccc6cccc-5c46)c4ccccc4-c4ccccc4)cc3)cc2)cc1 ; c1ccc(-c2ccc(-c3ccc(N(c4cc(-c5cc(-c6ccccc6)cc(-c6ccccc6)n5)c5c(c4)-c4cccc6cccc-5c46)c4ccccc4-c4ccccc4)cc3)cc2)cc1]

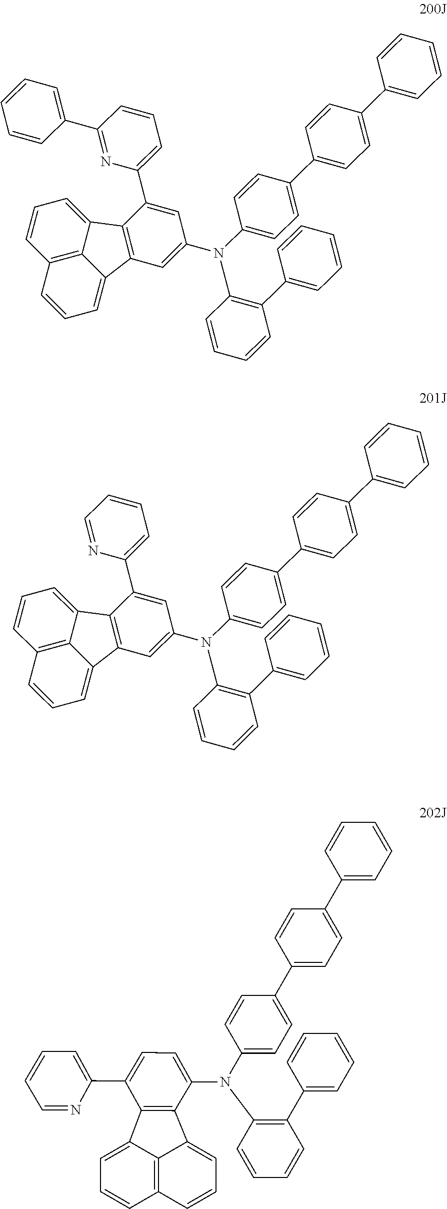 [SMILES: c1ccc(-c2ccc(-c3ccc(N(c4cc(-c5cccc(-c6ccccc6)n5)c5c(c4)-c4cccc6cccc-5c46)c4ccccc4-c4ccccc4)cc3)cc2)cc1 ; c1ccc(-c2ccc(-c3ccc(N(c4cc(-c5ccccn5)c5c(c4)-c4cccc6cccc-5c46)c4ccccc4-c4ccccc4)cc3)cc2)cc1 ; c1ccc(-c2ccc(-c3ccc(N(c4ccccc4-c4ccccc4)c4ccc(-c5ccccn5)c5c4-c4cccc6cccc-5c46)cc3)cc2)cc1]

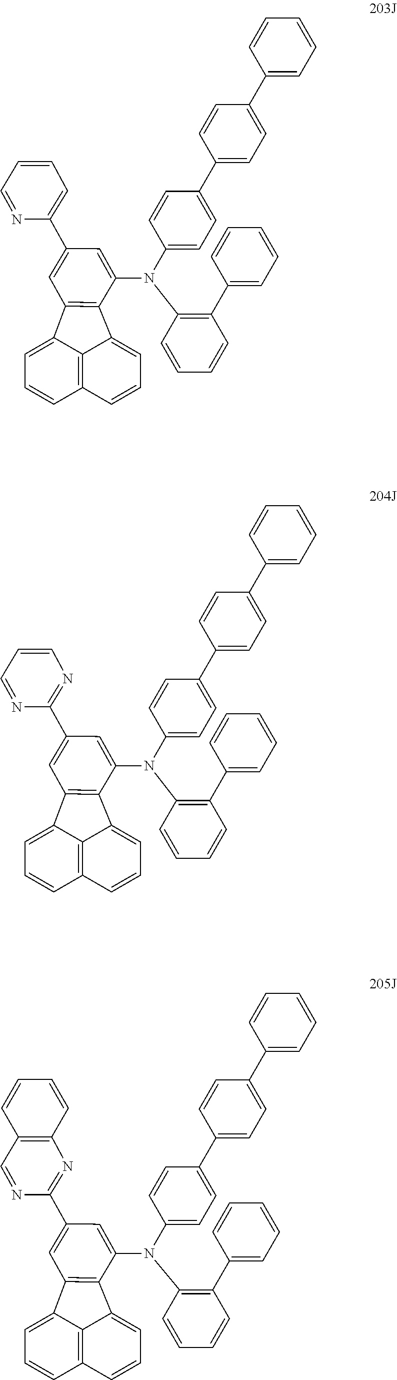 [SMILES: c1ccc(-c2ccc(-c3ccc(N(c4ccccc4-c4ccccc4)c4cc(-c5ccccn5)cc5c4-c4cccc6cccc-5c46)cc3)cc2)cc1 ; c1ccc(-c2ccc(-c3ccc(N(c4ccccc4-c4ccccc4)c4cc(-c5ncccn5)cc5c4-c4cccc6cccc-5c46)cc3)cc2)cc1 ; c1ccc(-c2ccc(-c3ccc(N(c4ccccc4-c4ccccc4)c4cc(-c5ncc6ccccc6n5)cc5c4-c4cccc6cccc-5c46)cc3)cc2)cc1]

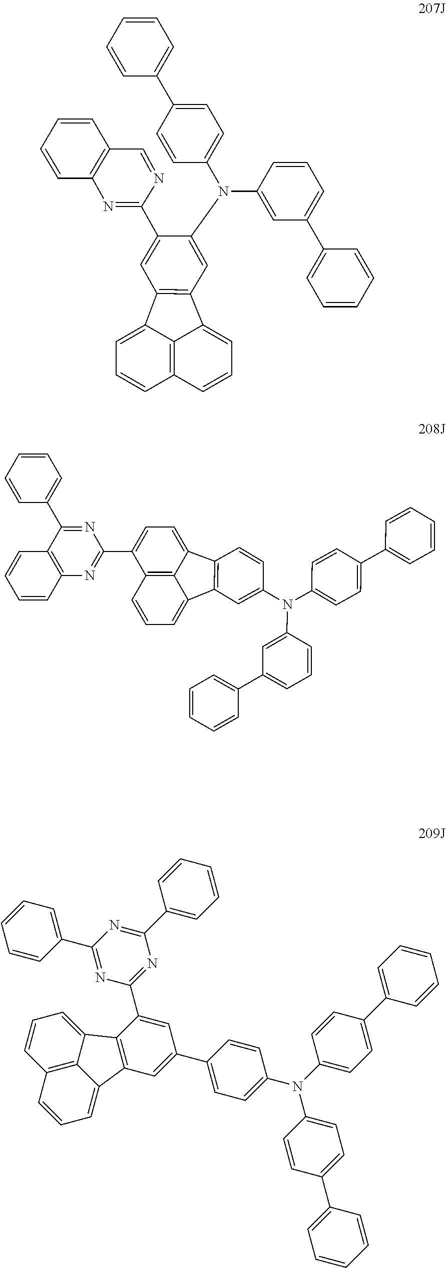 [SMILES: c1ccc(-c2ccc(N(c3cccc(-c4ccccc4)c3)c3cc4c(cc3-c3ncc5ccccc5n3)-c3cccc5cccc-4c35)cc2)cc1 ; c1ccc(-c2ccc(N(c3cccc(-c4ccccc4)c3)c3ccc4c(c3)-c3cccc5c(-c6nc(-c7ccccc7)c7ccccc7n6)ccc-4c35)cc2)cc1 ; c1ccc(-c2ccc(N(c3ccc(-c4ccccc4)cc3)c3ccc(-c4cc(-c5nc(-c6ccccc6)nc(-c6ccccc6)n5)c5c(c4)-c4cccc6cccc-5c46)cc3)cc2)cc1]

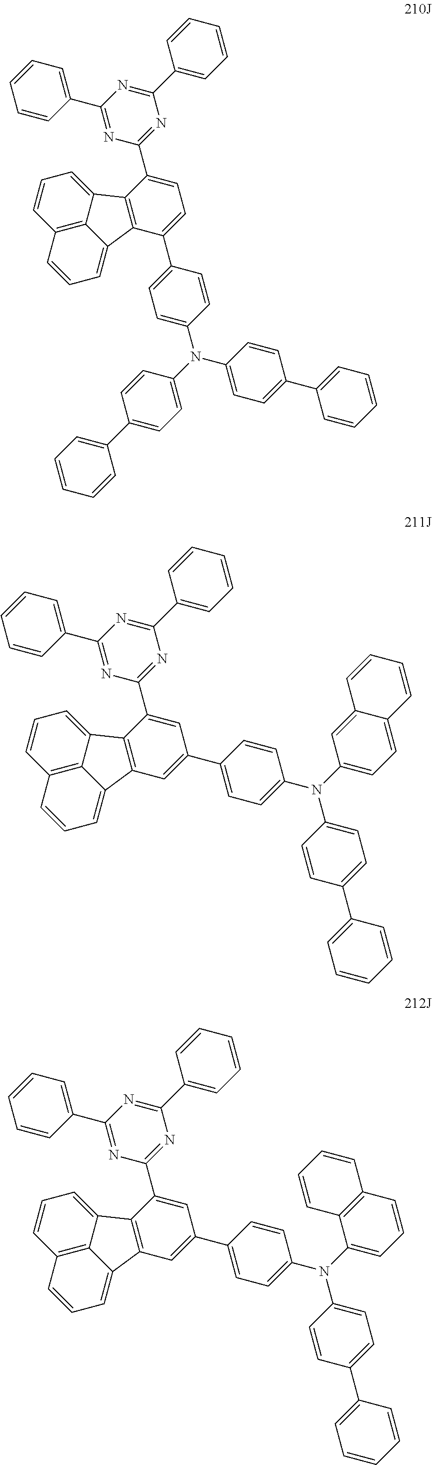 [SMILES: c1ccc(-c2ccc(N(c3ccc(-c4ccccc4)cc3)c3ccc(-c4ccc(-c5nc(-c6ccccc6)nc(-c6ccccc6)n5)c5c4-c4cccc6cccc-5c46)cc3)cc2)cc1 ; c1ccc(-c2ccc(N(c3ccc(-c4cc(-c5nc(-c6ccccc6)nc(-c6ccccc6)n5)c5c(c4)-c4cccc6cccc-5c46)cc3)c3ccc4ccccc4c3)cc2)cc1 ; c1ccc(-c2ccc(N(c3ccc(-c4cc(-c5nc(-c6ccccc6)nc(-c6ccccc6)n5)c5c(c4)-c4cccc6cccc-5c46)cc3)c3cccc4ccccc34)cc2)cc1]

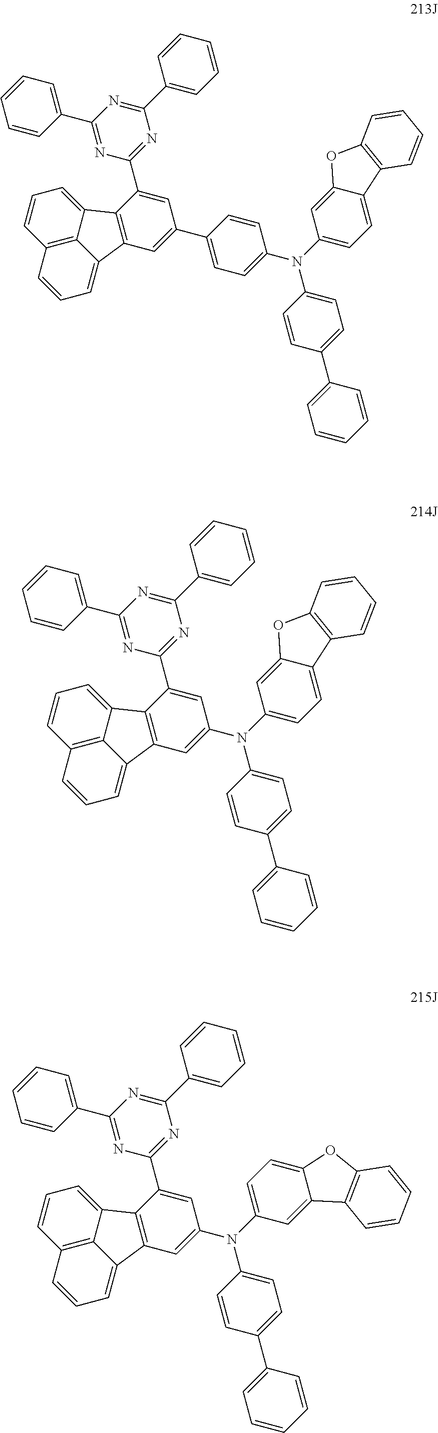 [SMILES: c1ccc(-c2ccc(N(c3ccc(-c4cc(-c5nc(-c6ccccc6)nc(-c6ccccc6)n5)c5c(c4)-c4cccc6cccc-5c46)cc3)c3ccc4c(c3)oc3ccccc34)cc2)cc1 ; c1ccc(-c2ccc(N(c3cc(-c4nc(-c5ccccc5)nc(-c5ccccc5)n4)c4c(c3)-c3cccc5cccc-4c35)c3ccc4c(c3)oc3ccccc34)cc2)cc1 ; c1ccc(-c2ccc(N(c3cc(-c4nc(-c5ccccc5)nc(-c5ccccc5)n4)c4c(c3)-c3cccc5cccc-4c35)c3ccc4oc5ccccc5c4c3)cc2)cc1]

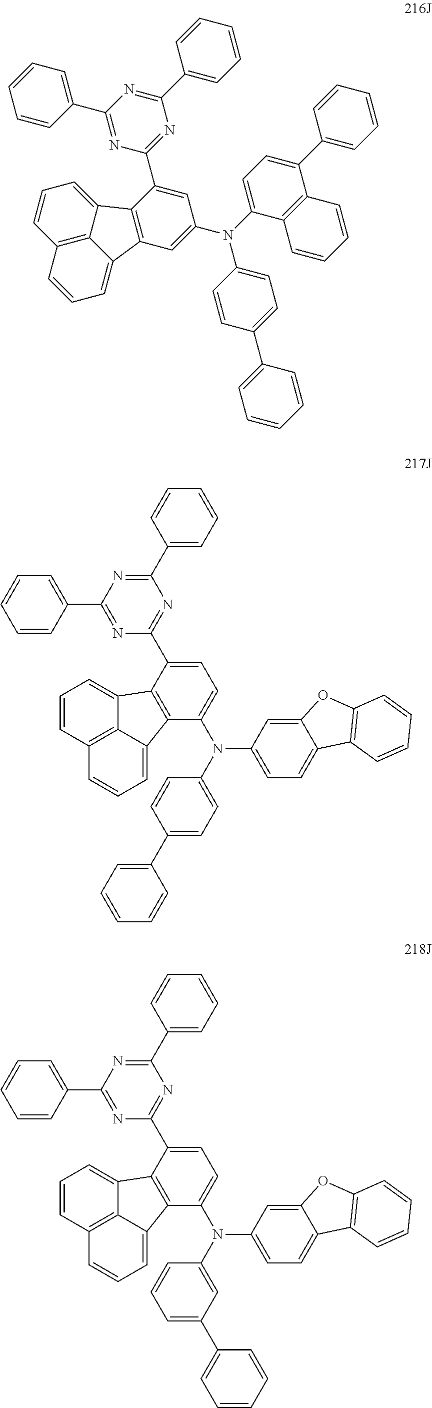 [SMILES: c1ccc(-c2ccc(N(c3cc(-c4nc(-c5ccccc5)nc(-c5ccccc5)n4)c4c(c3)-c3cccc5cccc-4c35)c3ccc(-c4ccccc4)c4ccccc34)cc2)cc1 ; c1ccc(-c2ccc(N(c3ccc4c(c3)oc3ccccc34)c3ccc(-c4nc(-c5ccccc5)nc(-c5ccccc5)n4)c4c3-c3cccc5cccc-4c35)cc2)cc1 ; c1ccc(-c2cccc(N(c3ccc4c(c3)oc3ccccc34)c3ccc(-c4nc(-c5ccccc5)nc(-c5ccccc5)n4)c4c3-c3cccc5cccc-4c35)c2)cc1]

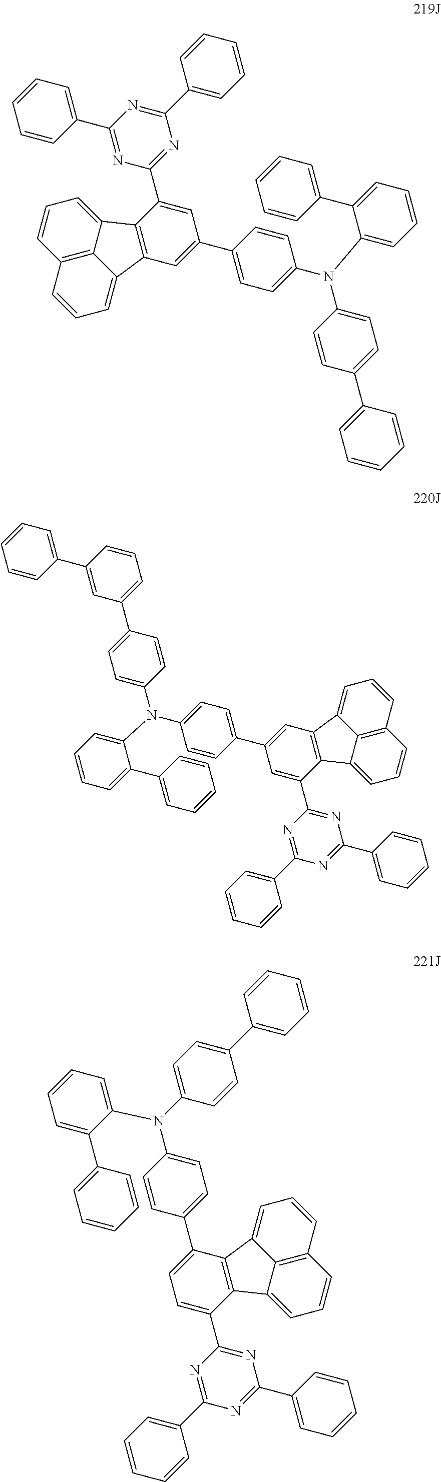

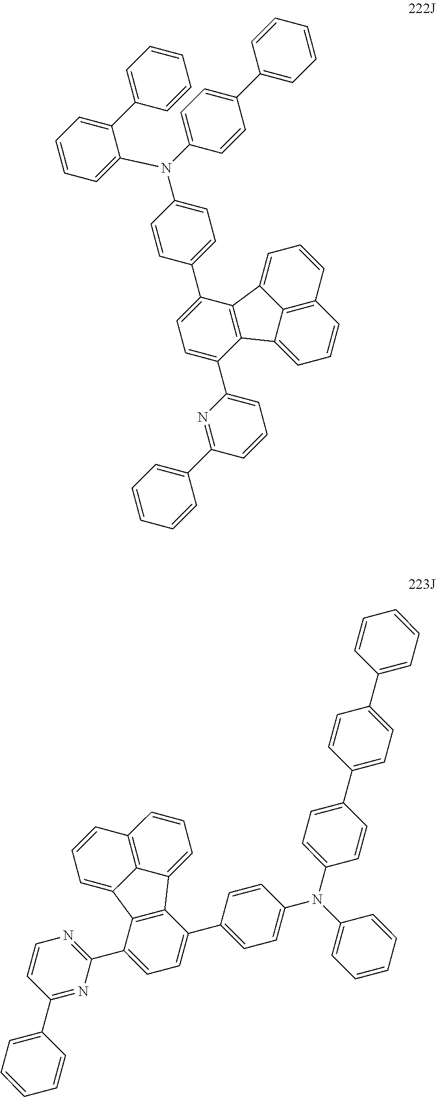 [SMILES: c1ccc(-c2ccc(N(c3ccc(-c4ccc(-c5cccc(-c6ccccc6)n5)c5c4-c4cccc6cccc-5c46)cc3)c3ccccc3-c3ccccc3)cc2)cc1 ; c1ccc(-c2ccc(-c3ccc(N(c4ccccc4)c4ccc(-c5ccc(-c6nccc(-c7ccccc7)n6)c6c5-c5cccc7cccc-6c57)cc4)cc3)cc2)cc1]

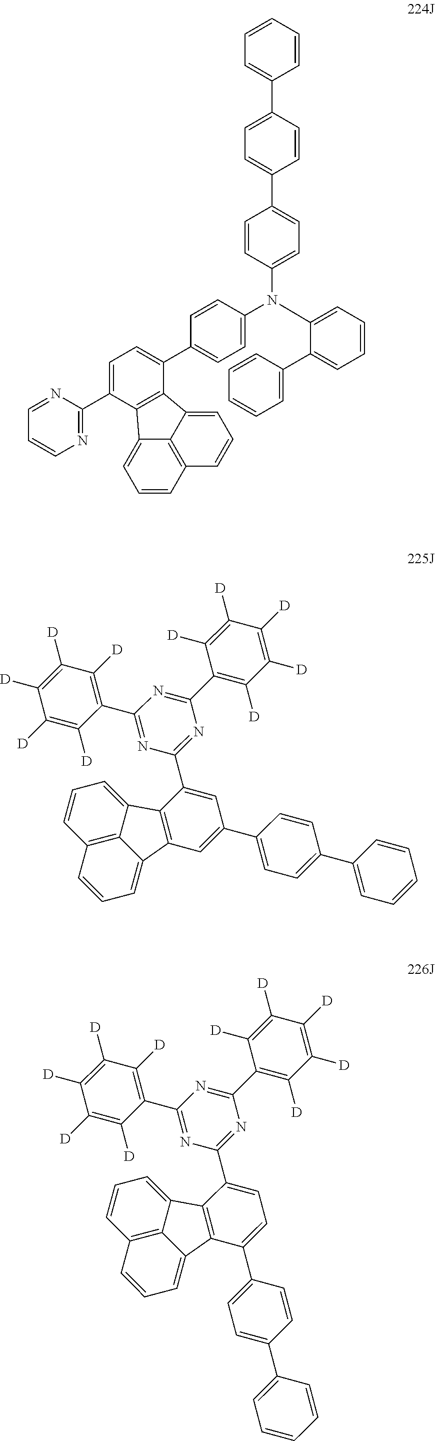 [SMILES: c1ccc(-c2ccc(-c3ccc(N(c4ccc(-c5ccc(-c6ncccn6)c6c5-c5cccc7cccc-6c57)cc4)c4ccccc4-c4ccccc4)cc3)cc2)cc1 ; [2H]c1c([2H])c([2H])c(-c2nc(-c3cc(-c4ccc(-c5ccccc5)cc4)cc4c3-c3cccc5cccc-4c35)nc(-c3c([2H])c([2H])c([2H])c([2H])c3[2H])n2)c([2H])c1[2H] ; [2H]c1c([2H])c([2H])c(-c2nc(-c3ccc(-c4ccc(-c5ccccc5)cc4)c4c3-c3cccc5cccc-4c35)nc(-c3c([2H])c([2H])c([2H])c([2H])c3[2H])n2)c([2H])c1[2H]]

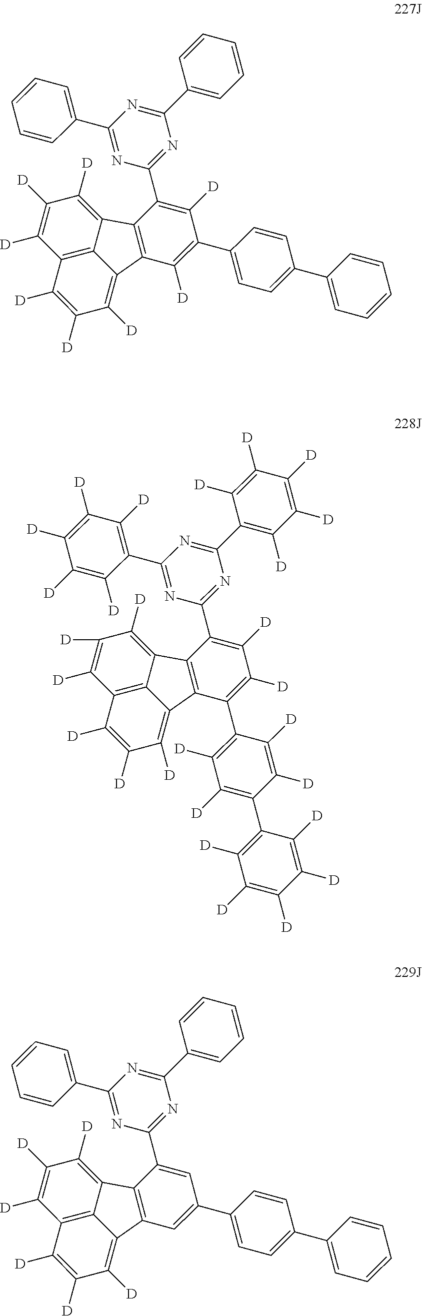 [SMILES: [2H]c1c(-c2ccc(-c3ccccc3)cc2)c([2H])c2c(c1-c1nc(-c3ccccc3)nc(-c3ccccc3)n1)-c1c([2H])c([2H])c([2H])c3c([2H])c([2H])c([2H])c-2c13 ; [2H]c1c([2H])c([2H])c(-c2nc(-c3c([2H])c([2H])c([2H])c([2H])c3[2H])nc(-c3c([2H])c([2H])c(-c4c([2H])c([2H])c(-c5c([2H])c([2H])c([2H])c([2H])c5[2H])c([2H])c4[2H])c4c3-c3c([2H])c([2H])c([2H])c5c([2H])c([2H])c([2H])c-4c35)n2)c([2H])c1[2H] ; [2H]c1c([2H])c2c3c(c([2H])c([2H])c([2H])c3c1[2H])-c1c(-c3nc(-c4ccccc4)nc(-c4ccccc4)n3)cc(-c3ccc(-c4ccccc4)cc3)cc1-2]

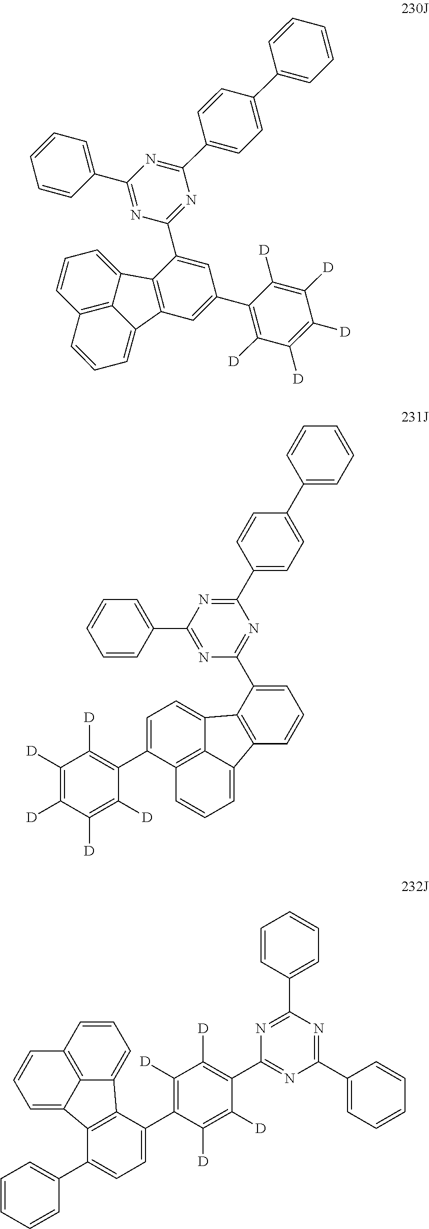 [SMILES: [2H]c1c([2H])c([2H])c(-c2cc(-c3nc(-c4ccccc4)nc(-c4ccc(-c5ccccc5)cc4)n3)c3c(c2)-c2cccc4cccc-3c24)c([2H])c1[2H] ; [2H]c1c([2H])c([2H])c(-c2ccc3c4c(cccc24)-c2cccc(-c4nc(-c5ccccc5)nc(-c5ccc(-c6ccccc6)cc5)n4)c2-3)c([2H])c1[2H] ; [2H]c1c([2H])c(-c2ccc(-c3ccccc3)c3c2-c2cccc4cccc-3c24)c([2H])c([2H])c1-c1nc(-c2ccccc2)nc(-c2ccccc2)n1]

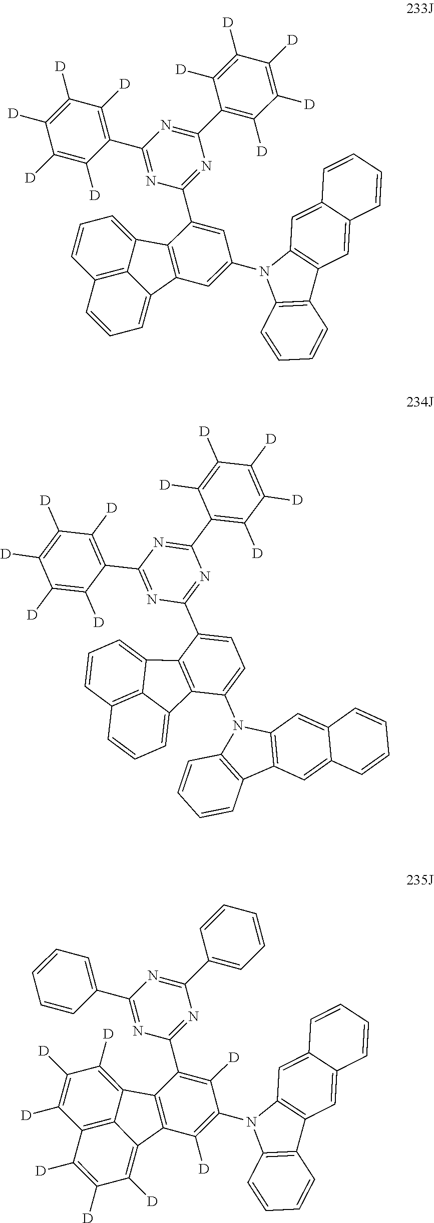 [SMILES: [2H]c1c([2H])c([2H])c(-c2nc(-c3cc(-n4c5ccccc5c5cc6ccccc6cc54)cc4c3-c3cccc5cccc-4c35)nc(-c3c([2H])c([2H])c([2H])c([2H])c3[2H])n2)c([2H])c1[2H] ; [2H]c1c([2H])c([2H])c(-c2nc(-c3ccc(-n4c5ccccc5c5cc6ccccc6cc54)c4c3-c3cccc5cccc-4c35)nc(-c3c([2H])c([2H])c([2H])c([2H])c3[2H])n2)c([2H])c1[2H] ; [2H]c1c(-c2nc(-c3ccccc3)nc(-c3ccccc3)n2)c2c(c([2H])c1-n1c3ccccc3c3cc4ccccc4cc31)-c1c([2H])c([2H])c([2H])c3c([2H])c([2H])c([2H])c-2c13]

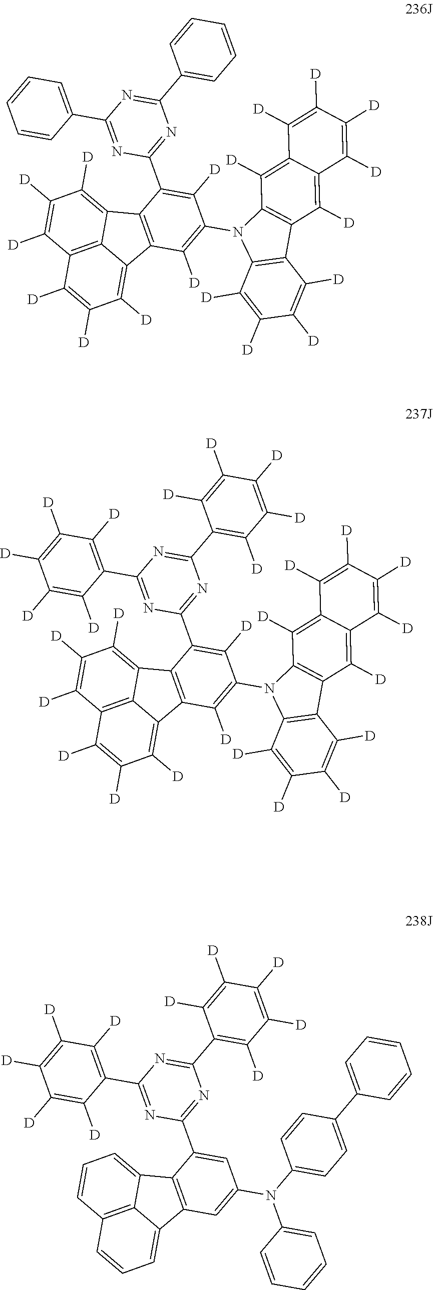

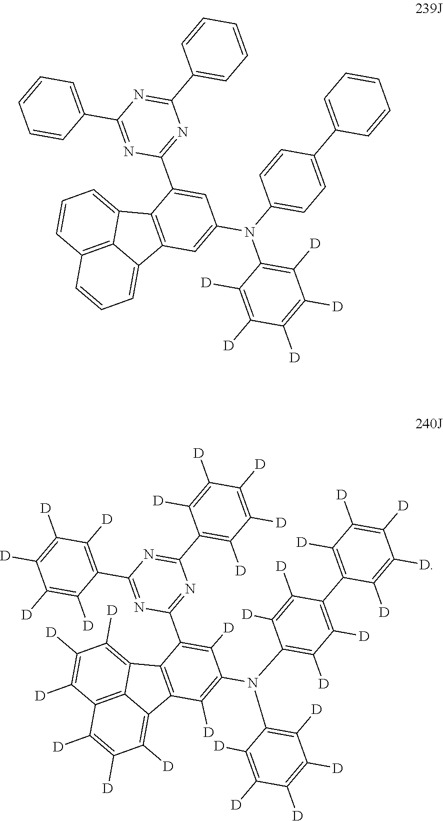

7. An organic light emitting device comprising:
a first electrode;
a second electrode; and
an organic material layer having one or more layers provided between the first electrode and the second electrode,
wherein one or more layers of the organic material layer comprise the heterocyclic compound according to claim 1.

8. The organic light emitting device of claim 7, wherein the organic material layer comprises a light emitting layer, and the light emitting layer comprises the heterocyclic compound.

9. The organic light emitting device of claim 8, wherein the light emitting layer comprises a host material, and the host material comprises the heterocyclic compound.

10. The organic light emitting device of claim 8, wherein in the light emitting layer, two or more of the heterocyclic compounds represented by Chemical Formula 1 are used.

11. The organic light emitting device of claim 7, further comprising one or two or more layers selected from the group consisting of a light emitting layer, a hole injection layer, a hole transport layer, an electron injection layer, an electron transport layer, a hole auxiliary layer, and a hole blocking layer.
